SEQUENCE LISTING (1083 sequences)

SEQ ID NO: 1 - RNA, 19 nt, Artificial Sequence
  Description of Artificial Sequence Synthetic oligonucleotide
acuuaaagcugguucauau

SEQ ID NO: 2 - RNA, 19 nt, Artificial Sequence
  Description of Artificial Sequence Synthetic oligonucleotide
gcaugaaugccucucgacu

SEQ ID NO: 3 - RNA, 19 nt, Artificial Sequence
  Description of Artificial Sequence Synthetic oligonucleotide
gguggugagcuuaaugaau

SEQ ID NO: 4 - DNA, 19 bp, Artificial Sequence
  Description of Artificial Sequence Synthetic oligonucleotide
tguggugagcuuaaugaau

SEQ ID NO: 5 - RNA, 19 nt, Artificial Sequence
  Description of Artificial Sequence Synthetic oligonucleotide
gcaagggagcuguacagga

SEQ ID NO: 6 - DNA, 19 bp, Artificial Sequence
  Description of Artificial Sequence Synthetic oligonucleotide
tcaugaaugccucucgacc

SEQ ID NO: 7 - DNA, 19 bp, Artificial Sequence
  Description of Artificial Sequence Synthetic oligonucleotide
tguggugagcuuaaugaac

SEQ ID NO: 8 - RNA, 19 nt, Artificial Sequence
  Description of Artificial Sequence Synthetic oligonucleotide
aguacacagugaccgucga

SEQ ID NO: 9 - DNA, 19 bp, Artificial Sequence
  Description of Artificial Sequence Synthetic oligonucleotide
tguacacagugaccgucgc

SEQ ID NO: 10 - DNA, 19 bp, Artificial Sequence
  Description of Artificial Sequence Synthetic oligonucleotide
tcuuaaagcugguucauac

SEQ ID NO: 11 - DNA, 19 bp, Artificial Sequence
  Description of Artificial Sequence Synthetic oligonucleotide
tcaagggagcuguacaggc

SEQ ID NO: 12 - DNA, 19 bp, Artificial Sequence
  Description of Artificial Sequence Synthetic oligonucleotide
tguacacagugaccgucga

SEQ ID NO: 13 - DNA, 19 bp, Artificial Sequence
  Description of Artificial Sequence Synthetic oligonucleotide
tcaagggagcuguacagga

SEQ ID NO: 14 - DNA, 19 bp, Artificial Sequence
  Description of Artificial Sequence Synthetic oligonucleotide
tcaugaaugccucucgacu

SEQ ID NO: 15 - RNA, 19 nt, Artificial Sequence
  Description of Artificial Sequence Synthetic oligonucleotide
ggaugagaagacuguuguc

SEQ ID NO: 16 - RNA, 19 nt, Artificial Sequence
  Description of Artificial Sequence Synthetic oligonucleotide
gcuagaaaaugccauacag

SEQ ID NO: 17 - RNA, 19 nt, Artificial Sequence
  Description of Artificial Sequence Synthetic oligonucleotide
cugaagauugcuuguagca

SEQ ID NO: 18 - RNA, 19 nt, Artificial Sequence
  Description of Artificial Sequence Synthetic oligonucleotide
ccgacagaguuagcacgac

SEQ ID NO: 19 - RNA, 19 nt, Artificial Sequence
  Description of Artificial Sequence Synthetic oligonucleotide
agugucagcuguauccuuc

SEQ ID NO: 20 - RNA, 19 nt, Artificial Sequence
  Description of Artificial Sequence Synthetic oligonucleotide
aggcaauucagagcuucga

SEQ ID NO: 21 - DNA, 19 bp, Artificial Sequence
  Description of Artificial Sequence Synthetic oligonucleotide
tcuuaaagcugguucauau

SEQ ID NO: 22 - RNA, 19 nt, Artificial Sequence
  Description of Artificial Sequence Synthetic oligonucleotide
gaucagggcagucuuugca

SEQ ID NO: 23 - RNA, 19 nt, Artificial Sequence
  Description of Artificial Sequence Synthetic oligonucleotide
ucaggaaaugguacggcug

SEQ ID NO: 24 - RNA, 19 nt, Artificial Sequence
  Description of Artificial Sequence Synthetic oligonucleotide
ugguucauaucuugaacau

SEQ ID NO: 25 - RNA, 19 nt, Artificial Sequence
  Description of Artificial Sequence Synthetic oligonucleotide
acagaaucauccaucggga

SEQ ID NO: 26 - RNA, 19 nt, Artificial Sequence
  Description of Artificial Sequence Synthetic oligonucleotide
guacacagugaccgucgac

SEQ ID NO: 27 - RNA, 19 nt, Artificial Sequence
  Description of Artificial Sequence Synthetic oligonucleotide
uugcuuguagcaagguccg

SEQ ID NO: 28 - RNA, 19 nt, Artificial Sequence
  Description of Artificial Sequence Synthetic oligonucleotide
cugccgacagaguuagcac

SEQ ID NO: 29 - RNA, 19 nt, Artificial Sequence
  Description of Artificial Sequence Synthetic oligonucleotide
gaaagugcgagugaucuau

SEQ ID NO: 30 - RNA, 19 nt, Artificial Sequence
  Description of Artificial Sequence Synthetic oligonucleotide
ccugaagauugcuuguagc

SEQ ID NO: 31 - RNA, 19 nt, Artificial Sequence
  Description of Artificial Sequence Synthetic oligonucleotide
cgacagaguuagcacgaca

SEQ ID NO: 32 - RNA, 19 nt, Artificial Sequence
  Description of Artificial Sequence Synthetic oligonucleotide
cuagaaaaugccauacagg

SEQ ID NO: 33 - RNA, 19 nt, Artificial Sequence
  Description of Artificial Sequence Synthetic oligonucleotide
cugcccgcguuaagauucc

SEQ ID NO: 34 - RNA, 19 nt, Artificial Sequence
  Description of Artificial Sequence Synthetic oligonucleotide
cagaaucauccaucgggau

SEQ ID NO: 35 - RNA, 19 nt, Artificial Sequence
  Description of Artificial Sequence Synthetic oligonucleotide
gccagaaaacaucguccug

SEQ ID NO: 36 - RNA, 19 nt, Artificial Sequence
  Description of Artificial Sequence Synthetic oligonucleotide
guuugcaagcagaaggcgc

SEQ ID NO: 37 - RNA, 19 nt, Artificial Sequence
  Description of Artificial Sequence Synthetic oligonucleotide
ugcuagaaaaugccauaca

SEQ ID NO: 38 - RNA, 19 nt, Artificial Sequence
  Description of Artificial Sequence Synthetic oligonucleotide
agguggugagcuuaaugaa

SEQ ID NO: 39 - RNA, 19 nt, Artificial Sequence
  Description of Artificial Sequence Synthetic oligonucleotide
gacagaguuagcacgacau

SEQ ID NO: 40 - RNA, 19 nt, Artificial Sequence
  Description of Artificial Sequence Synthetic oligonucleotide
gcgggagaacgaagugaaa

SEQ ID NO: 41 - RNA, 19 nt, Artificial Sequence
  Description of Artificial Sequence Synthetic oligonucleotide
gagcuguacaggagacuaa

SEQ ID NO: 42 - RNA, 19 nt, Artificial Sequence
  Description of Artificial Sequence Synthetic oligonucleotide
ccucgagaccagcgaacug

SEQ ID NO: 43 - RNA, 19 nt, Artificial Sequence
  Description of Artificial Sequence Synthetic oligonucleotide
agccagaaaacaucguccu

SEQ ID NO: 44 - RNA, 19 nt, Artificial Sequence
  Description of Artificial Sequence Synthetic oligonucleotide
cugguuacagacggaagaa

SEQ ID NO: 45 - RNA, 19 nt, Artificial Sequence
  Description of Artificial Sequence Synthetic oligonucleotide
agaguuucacggcccuaga

SEQ ID NO: 46 - RNA, 19 nt, Artificial Sequence
  Description of Artificial Sequence Synthetic oligonucleotide
uacacagugaccgucgacu

SEQ ID NO: 47 - RNA, 19 nt, Artificial Sequence
  Description of Artificial Sequence Synthetic oligonucleotide
aaagugcgagugaucuaua

SEQ ID NO: 48 - RNA, 19 nt, Artificial Sequence
  Description of Artificial Sequence Synthetic oligonucleotide
cagcgaacugagggugaca

SEQ ID NO: 49 - RNA, 19 nt, Artificial Sequence
  Description of Artificial Sequence Synthetic oligonucleotide
ccgcguuaagauucccgca

SEQ ID NO: 50 - RNA, 19 nt, Artificial Sequence
  Description of Artificial Sequence Synthetic oligonucleotide
acuccugguagaacggaug

SEQ ID NO: 51 - RNA, 19 nt, Artificial Sequence
  Description of Artificial Sequence Synthetic oligonucleotide
gcguuaagauucccgcauu

SEQ ID NO: 52 - RNA, 19 nt, Artificial Sequence
  Description of Artificial Sequence Synthetic oligonucleotide
aagcccggauagcaugaau

SEQ ID NO: 53 - RNA, 19 nt, Artificial Sequence
  Description of Artificial Sequence Synthetic oligonucleotide
uucccgcauuuuaauguuu

SEQ ID NO: 54 - RNA, 19 nt, Artificial Sequence
  Description of Artificial Sequence Synthetic oligonucleotide
aacuccugguagaacggau

SEQ ID NO: 55 - RNA, 19 nt, Artificial Sequence
  Description of Artificial Sequence Synthetic oligonucleotide
uuguagcaagguccguggu

SEQ ID NO: 56 - RNA, 19 nt, Artificial Sequence
  Description of Artificial Sequence Synthetic oligonucleotide
cguuaagauucccgcauuu

SEQ ID NO: 57 - RNA, 19 nt, Artificial Sequence
  Description of Artificial Sequence Synthetic oligonucleotide
acaaaauuauugaccuagg

SEQ ID NO: 58 - RNA, 19 nt, Artificial Sequence
  Description of Artificial Sequence Synthetic oligonucleotide
gcuuguagcaagguccgug

SEQ ID NO: 59 - RNA, 19 nt, Artificial Sequence
  Description of Artificial Sequence Synthetic oligonucleotide
aacuuaaagcugguucaua

SEQ ID NO: 60 - RNA, 19 nt, Artificial Sequence
  Description of Artificial Sequence Synthetic oligonucleotide
ugaccgucgacuacuggag

SEQ ID NO: 61 - RNA, 19 nt, Artificial Sequence
  Description of Artificial Sequence Synthetic oligonucleotide
auucccgcauuuuaauguu

SEQ ID NO: 62 - RNA, 19 nt, Artificial Sequence
  Description of Artificial Sequence Synthetic oligonucleotide
aaugugguggcugcccgag

SEQ ID NO: 63 - RNA, 19 nt, Artificial Sequence
  Description of Artificial Sequence Synthetic oligonucleotide
gccucucgacuuagccagc

SEQ ID NO: 64 - RNA, 19 nt, Artificial Sequence
  Description of Artificial Sequence Synthetic oligonucleotide
acgacaucaguaugagcug

SEQ ID NO: 65 - RNA, 19 nt, Artificial Sequence
  Description of Artificial Sequence Synthetic oligonucleotide
cuuguagcaagguccgugg

SEQ ID NO: 66 - RNA, 19 nt, Artificial Sequence
  Description of Artificial Sequence Synthetic oligonucleotide
gccaugaugaaucuccucc

SEQ ID NO: 67 - RNA, 19 nt, Artificial Sequence
  Description of Artificial Sequence Synthetic oligonucleotide
ucauccgauggcacaauca

SEQ ID NO: 68 - RNA, 19 nt, Artificial Sequence
  Description of Artificial Sequence Synthetic oligonucleotide
ggaaaugucauccgauggc

SEQ ID NO: 69 - RNA, 19 nt, Artificial Sequence
  Description of Artificial Sequence Synthetic oligonucleotide
cgugguccugucaguggaa

SEQ ID NO: 70 - RNA, 19 nt, Artificial Sequence
  Description of Artificial Sequence Synthetic oligonucleotide
aauuauugaccuaggauau

SEQ ID NO: 71 - RNA, 19 nt, Artificial Sequence
  Description of Artificial Sequence Synthetic oligonucleotide
gccgacagaguuagcacga

SEQ ID NO: 72 - RNA, 19 nt, Artificial Sequence
  Description of Artificial Sequence Synthetic oligonucleotide
ugcuuguagcaagguccgu

SEQ ID NO: 73 - RNA, 19 nt, Artificial Sequence
  Description of Artificial Sequence Synthetic oligonucleotide
aguggaagcccggauagca

SEQ ID NO: 74 - RNA, 19 nt, Artificial Sequence
  Description of Artificial Sequence Synthetic oligonucleotide
aagugucagcuguauccuu

SEQ ID NO: 75 - RNA, 19 nt, Artificial Sequence
  Description of Artificial Sequence Synthetic oligonucleotide
caggaaaugguacggcugc

SEQ ID NO: 76 - RNA, 19 nt, Artificial Sequence
  Description of Artificial Sequence Synthetic oligonucleotide
gucccugccgacagaguua

SEQ ID NO: 77 - RNA, 19 nt, Artificial Sequence
  Description of Artificial Sequence Synthetic oligonucleotide
cgcguuaagauucccgcau

SEQ ID NO: 78 - RNA, 19 nt, Artificial Sequence
  Description of Artificial Sequence Synthetic oligonucleotide
aagugcgagugaucuauac

SEQ ID NO: 79 - RNA, 19 nt, Artificial Sequence
  Description of Artificial Sequence Synthetic oligonucleotide
gaaugccucucgacuuagc

SEQ ID NO: 80 - RNA, 19 nt, Artificial Sequence
  Description of Artificial Sequence Synthetic oligonucleotide
cgagaccagcgaacugagg

SEQ ID NO: 81 - RNA, 19 nt, Artificial Sequence
  Description of Artificial Sequence Synthetic oligonucleotide
uguagcaagguccgugguc

SEQ ID NO: 82 - RNA, 19 nt, Artificial Sequence
  Description of Artificial Sequence Synthetic oligonucleotide
uuagcacgacaucaguaug

SEQ ID NO: 83 - RNA, 19 nt, Artificial Sequence
  Description of Artificial Sequence Synthetic oligonucleotide
cuguacaggagacuaaggg

SEQ ID NO: 84 - RNA, 19 nt, Artificial Sequence
  Description of Artificial Sequence Synthetic oligonucleotide
gagaacgaagugaaacucc

SEQ ID NO: 85 - RNA, 19 nt, Artificial Sequence
  Description of Artificial Sequence Synthetic oligonucleotide
gaacuuggcgcccaaugac

SEQ ID NO: 86 - RNA, 19 nt, Artificial Sequence
  Description of Artificial Sequence Synthetic oligonucleotide
gcugcccgcguuaagauuc

SEQ ID NO: 87 - RNA, 19 nt, Artificial Sequence
  Description of Artificial Sequence Synthetic oligonucleotide
aagcugguucauaucuuga

SEQ ID NO: 88 - RNA, 19 nt, Artificial Sequence
  Description of Artificial Sequence Synthetic oligonucleotide
gcccgcguuaagauucccg

SEQ ID NO: 89 - RNA, 19 nt, Artificial Sequence
  Description of Artificial Sequence Synthetic oligonucleotide
gaggaagucgcgccgcgcu

SEQ ID NO: 90 - RNA, 19 nt, Artificial Sequence
  Description of Artificial Sequence Synthetic oligonucleotide
cucgagaccagcgaacuga

SEQ ID NO: 91 - RNA, 19 nt, Artificial Sequence
  Description of Artificial Sequence Synthetic oligonucleotide
agagguggugagcuuaaug

SEQ ID NO: 92 - RNA, 19 nt, Artificial Sequence
  Description of Artificial Sequence Synthetic oligonucleotide
gagguggugagcuuaauga

SEQ ID NO: 93 - RNA, 19 nt, Artificial Sequence
  Description of Artificial Sequence Synthetic oligonucleotide
gaguuucacggcccuagac

SEQ ID NO: 94 - RNA, 19 nt, Artificial Sequence
  Description of Artificial Sequence Synthetic oligonucleotide
cggccuccaacagcuuacc

SEQ ID NO: 95 - RNA, 19 nt, Artificial Sequence
  Description of Artificial Sequence Synthetic oligonucleotide
agcccggauagcaugaaug

SEQ ID NO: 96 - RNA, 19 nt, Artificial Sequence
  Description of Artificial Sequence Synthetic oligonucleotide
gcgggccuggcguugaucc

SEQ ID NO: 97 - RNA, 19 nt, Artificial Sequence
  Description of Artificial Sequence Synthetic oligonucleotide
ugcccgcguuaagauuccc

SEQ ID NO: 98 - RNA, 19 nt, Artificial Sequence
  Description of Artificial Sequence Synthetic oligonucleotide
agauucccgcauuuuaaug

SEQ ID NO: 99 - RNA, 19 nt, Artificial Sequence
  Description of Artificial Sequence Synthetic oligonucleotide
ucucgacuuagccagccug

SEQ ID NO: 100 - RNA, 19 nt, Artificial Sequence
  Description of Artificial Sequence Synthetic oligonucleotide
caaugugguggcugcccga

SEQ ID NO: 101 - RNA, 19 nt, Artificial Sequence
  Description of Artificial Sequence Synthetic oligonucleotide
aaacugugguuugcaagca

SEQ ID NO: 102 - RNA, 19 nt, Artificial Sequence
  Description of Artificial Sequence Synthetic oligonucleotide
ccgcgucccugccgacaga

SEQ ID NO: 103 - RNA, 19 nt, Artificial Sequence
  Description of Artificial Sequence Synthetic oligonucleotide
ugggaucacaucagauaaa

SEQ ID NO: 104 - RNA, 19 nt, Artificial Sequence
  Description of Artificial Sequence Synthetic oligonucleotide
aacagaaucauccaucggg

SEQ ID NO: 105 - RNA, 19 nt, Artificial Sequence
  Description of Artificial Sequence Synthetic oligonucleotide
aaugccucucgacuuagcc

SEQ ID NO: 106 - RNA, 19 nt, Artificial Sequence
  Description of Artificial Sequence Synthetic oligonucleotide
uguacaggagacuaaggga

SEQ ID NO: 107 - RNA, 19 nt, Artificial Sequence
  Description of Artificial Sequence Synthetic oligonucleotide
ugugggcgggagaacgaag

SEQ ID NO: 108 - RNA, 19 nt, Artificial Sequence
  Description of Artificial Sequence Synthetic oligonucleotide
ucugugggcgggagaacga

SEQ ID NO: 109 - RNA, 19 nt, Artificial Sequence
  Description of Artificial Sequence Synthetic oligonucleotide
agauugcuuguagcaaggu

SEQ ID NO: 110 - RNA, 19 nt, Artificial Sequence
  Description of Artificial Sequence Synthetic oligonucleotide
auaugaaccagcuuuaagu

SEQ ID NO: 111 - RNA, 19 nt, Artificial Sequence
  Description of Artificial Sequence Synthetic oligonucleotide
agucgagaggcauucaugc

SEQ ID NO: 112 - RNA, 19 nt, Artificial Sequence
  Description of Artificial Sequence Synthetic oligonucleotide
auucauuaagcucaccacc

SEQ ID NO: 113 - RNA, 19 nt, Artificial Sequence
  Description of Artificial Sequence Synthetic oligonucleotide
uccuguacagcucccuugc

SEQ ID NO: 114 - RNA, 19 nt, Artificial Sequence
  Description of Artificial Sequence Synthetic oligonucleotide
ucgacggucacuguguacu

SEQ ID NO: 115 - RNA, 19 nt, Artificial Sequence
  Description of Artificial Sequence Synthetic oligonucleotide
gacaacagucuucucaucc

SEQ ID NO: 116 - RNA, 19 nt, Artificial Sequence
  Description of Artificial Sequence Synthetic oligonucleotide
cuguauggcauuuucuagc

SEQ ID NO: 117 - RNA, 19 nt, Artificial Sequence
  Description of Artificial Sequence Synthetic oligonucleotide
ugcuacaagcaaucuucag

SEQ ID NO: 118 - RNA, 19 nt, Artificial Sequence
  Description of Artificial Sequence Synthetic oligonucleotide
ggucgagaggcauucaugc

SEQ ID NO: 119 - RNA, 19 nt, Artificial Sequence
  Description of Artificial Sequence Synthetic oligonucleotide
gucgugcuaacucugucgg

SEQ ID NO: 120 - RNA, 19 nt, Artificial Sequence
  Description of Artificial Sequence Synthetic oligonucleotide
gaaggauacagcugacacu

SEQ ID NO: 121 - RNA, 19 nt, Artificial Sequence
  Description of Artificial Sequence Synthetic oligonucleotide
ucgaagcucugaauugccu

SEQ ID NO: 122 - RNA, 19 nt, Artificial Sequence
  Description of Artificial Sequence Synthetic oligonucleotide
ugcaaagacugcccugauc

SEQ ID NO: 123 - RNA, 19 nt, Artificial Sequence
  Description of Artificial Sequence Synthetic oligonucleotide
cagccguaccauuuccuga

SEQ ID NO: 124 - RNA, 19 nt, Artificial Sequence
  Description of Artificial Sequence Synthetic oligonucleotide
auguucaagauaugaacca

SEQ ID NO: 125 - RNA, 19 nt, Artificial Sequence
  Description of Artificial Sequence Synthetic oligonucleotide
ucccgauggaugauucugu

SEQ ID NO: 126 - RNA, 19 nt, Artificial Sequence
  Description of Artificial Sequence Synthetic oligonucleotide
gucgacggucacuguguac

SEQ ID NO: 127 - RNA, 19 nt, Artificial Sequence
  Description of Artificial Sequence Synthetic oligonucleotide
cggaccuugcuacaagcaa

SEQ ID NO: 128 - RNA, 19 nt, Artificial Sequence
  Description of Artificial Sequence Synthetic oligonucleotide
gugcuaacucugucggcag

SEQ ID NO: 129 - RNA, 19 nt, Artificial Sequence
  Description of Artificial Sequence Synthetic oligonucleotide
auagaucacucgcacuuuc

SEQ ID NO: 130 - RNA, 19 nt, Artificial Sequence
  Description of Artificial Sequence Synthetic oligonucleotide
gcuacaagcaaucuucagg

SEQ ID NO: 131 - RNA, 19 nt, Artificial Sequence
  Description of Artificial Sequence Synthetic oligonucleotide
ugucgugcuaacucugucg

SEQ ID NO: 132 - RNA, 19 nt, Artificial Sequence
  Description of Artificial Sequence Synthetic oligonucleotide
ccuguauggcauuuucuag

SEQ ID NO: 133 - RNA, 19 nt, Artificial Sequence
  Description of Artificial Sequence Synthetic oligonucleotide
ggaaucuuaacgcgggcag

SEQ ID NO: 134 - RNA, 19 nt, Artificial Sequence
  Description of Artificial Sequence Synthetic oligonucleotide
aucccgauggaugauucug

SEQ ID NO: 135 - RNA, 19 nt, Artificial Sequence
  Description of Artificial Sequence Synthetic oligonucleotide
caggacgauguuuucuggc

SEQ ID NO: 136 - RNA, 19 nt, Artificial Sequence
  Description of Artificial Sequence Synthetic oligonucleotide
gcgccuucugcuugcaaac

SEQ ID NO: 137 - RNA, 19 nt, Artificial Sequence
  Description of Artificial Sequence Synthetic oligonucleotide
uguauggcauuuucuagca

SEQ ID NO: 138 - RNA, 19 nt, Artificial Sequence
  Description of Artificial Sequence Synthetic oligonucleotide
uucauuaagcucaccaccu

SEQ ID NO: 139 - RNA, 19 nt, Artificial Sequence
  Description of Artificial Sequence Synthetic oligonucleotide
augucgugcuaacucuguc

SEQ ID NO: 140 - RNA, 19 nt, Artificial Sequence
  Description of Artificial Sequence Synthetic oligonucleotide
uuucacuucguucucccgc

SEQ ID NO: 141 - RNA, 19 nt, Artificial Sequence
  Description of Artificial Sequence Synthetic oligonucleotide
uuagucuccuguacagcuc

SEQ ID NO: 142 - RNA, 19 nt, Artificial Sequence
  Description of Artificial Sequence Synthetic oligonucleotide
caguucgcuggucucgagg

SEQ ID NO: 143 - RNA, 19 nt, Artificial Sequence
  Description of Artificial Sequence Synthetic oligonucleotide
guaugaaccagcuuuaagu

SEQ ID NO: 144 - RNA, 19 nt, Artificial Sequence
  Description of Artificial Sequence Synthetic oligonucleotide
aggacgauguuuucuggcu

SEQ ID NO: 145 - RNA, 19 nt, Artificial Sequence
  Description of Artificial Sequence Synthetic oligonucleotide
uucuuccgucuguaaccag

SEQ ID NO: 146 - RNA, 19 nt, Artificial Sequence
  Description of Artificial Sequence Synthetic oligonucleotide
ucuagggccgugaaacucu

SEQ ID NO: 147 - RNA, 19 nt, Artificial Sequence
  Description of Artificial Sequence Synthetic oligonucleotide
agucgacggucacugugua

SEQ ID NO: 148 - RNA, 19 nt, Artificial Sequence
  Description of Artificial Sequence Synthetic oligonucleotide
uauagaucacucgcacuuu

SEQ ID NO: 149 - RNA, 19 nt, Artificial Sequence
  Description of Artificial Sequence Synthetic oligonucleotide
ugucacccucaguucgcug

SEQ ID NO: 150 - RNA, 19 nt, Artificial Sequence
  Description of Artificial Sequence Synthetic oligonucleotide
ugcgggaaucuuaacgcgg

SEQ ID NO: 151 - RNA, 19 nt, Artificial Sequence
  Description of Artificial Sequence Synthetic oligonucleotide
cauccguucuaccaggagu

SEQ ID NO: 152 - RNA, 19 nt, Artificial Sequence
  Description of Artificial Sequence Synthetic oligonucleotide
aaugcgggaaucuuaacgc

SEQ ID NO: 153 - RNA, 19 nt, Artificial Sequence
  Description of Artificial Sequence Synthetic oligonucleotide
auucaugcuauccgggcuu

SEQ ID NO: 154 - RNA, 19 nt, Artificial Sequence
  Description of Artificial Sequence Synthetic oligonucleotide
aaacauuaaaaugcgggaa

SEQ ID NO: 155 - RNA, 19 nt, Artificial Sequence
  Description of Artificial Sequence Synthetic oligonucleotide
auccguucuaccaggaguu

SEQ ID NO: 156 - RNA, 19 nt, Artificial Sequence
  Description of Artificial Sequence Synthetic oligonucleotide
accacggaccuugcuacaa

SEQ ID NO: 157 - RNA, 19 nt, Artificial Sequence
  Description of Artificial Sequence Synthetic oligonucleotide
aaaugcgggaaucuuaacg

SEQ ID NO: 158 - RNA, 19 nt, Artificial Sequence
  Description of Artificial Sequence Synthetic oligonucleotide
ccuaggucaauaauuuugu

SEQ ID NO: 159 - RNA, 19 nt, Artificial Sequence
  Description of Artificial Sequence Synthetic oligonucleotide
cacggaccuugcuacaagc

SEQ ID NO: 160 - RNA, 19 nt, Artificial Sequence
  Description of Artificial Sequence Synthetic oligonucleotide
uaugaaccagcuuuaaguu

SEQ ID NO: 161 - RNA, 19 nt, Artificial Sequence
  Description of Artificial Sequence Synthetic oligonucleotide
cuccaguagucgacgguca

SEQ ID NO: 162 - RNA, 19 nt, Artificial Sequence
  Description of Artificial Sequence Synthetic oligonucleotide
aacauuaaaaugcgggaau

SEQ ID NO: 163 - RNA, 19 nt, Artificial Sequence
  Description of Artificial Sequence Synthetic oligonucleotide
cucgggcagccaccacauu

SEQ ID NO: 164 - RNA, 19 nt, Artificial Sequence
  Description of Artificial Sequence Synthetic oligonucleotide
gcuggcuaagucgagaggc

SEQ ID NO: 165 - RNA, 19 nt, Artificial Sequence
  Description of Artificial Sequence Synthetic oligonucleotide
cagcucauacugaugucgu

SEQ ID NO: 166 - RNA, 19 nt, Artificial Sequence
  Description of Artificial Sequence Synthetic oligonucleotide
ccacggaccuugcuacaag

SEQ ID NO: 167 - RNA, 19 nt, Artificial Sequence
  Description of Artificial Sequence Synthetic oligonucleotide
ggaggagauucaucauggc

SEQ ID NO: 168 - RNA, 19 nt, Artificial Sequence
  Description of Artificial Sequence Synthetic oligonucleotide
ugauugugccaucggauga

SEQ ID NO: 169 - RNA, 19 nt, Artificial Sequence
  Description of Artificial Sequence Synthetic oligonucleotide
gccaucggaugacauuucc

SEQ ID NO: 170 - RNA, 19 nt, Artificial Sequence
  Description of Artificial Sequence Synthetic oligonucleotide
uuccacugacaggaccacg

SEQ ID NO: 171 - RNA, 19 nt, Artificial Sequence
  Description of Artificial Sequence Synthetic oligonucleotide
auauccuaggucaauaauu

SEQ ID NO: 172 - RNA, 19 nt, Artificial Sequence
  Description of Artificial Sequence Synthetic oligonucleotide
ucgugcuaacucugucggc

SEQ ID NO: 173 - RNA, 19 nt, Artificial Sequence
  Description of Artificial Sequence Synthetic oligonucleotide
acggaccuugcuacaagca

SEQ ID NO: 174 - RNA, 19 nt, Artificial Sequence
  Description of Artificial Sequence Synthetic oligonucleotide
ugcuauccgggcuuccacu

SEQ ID NO: 175 - RNA, 19 nt, Artificial Sequence
  Description of Artificial Sequence Synthetic oligonucleotide
aaggauacagcugacacuu

SEQ ID NO: 176 - RNA, 19 nt, Artificial Sequence
  Description of Artificial Sequence Synthetic oligonucleotide
gcagccguaccauuuccug

SEQ ID NO: 177 - RNA, 19 nt, Artificial Sequence
  Description of Artificial Sequence Synthetic oligonucleotide
uaacucugucggcagggac

SEQ ID NO: 178 - RNA, 19 nt, Artificial Sequence
  Description of Artificial Sequence Synthetic oligonucleotide
augcgggaaucuuaacgcg

SEQ ID NO: 179 - RNA, 19 nt, Artificial Sequence
  Description of Artificial Sequence Synthetic oligonucleotide
guauagaucacucgcacuu

SEQ ID NO: 180 - RNA, 19 nt, Artificial Sequence
  Description of Artificial Sequence Synthetic oligonucleotide
gcuaagucgagaggcauuc

SEQ ID NO: 181 - RNA, 19 nt, Artificial Sequence
  Description of Artificial Sequence Synthetic oligonucleotide
ccucaguucgcuggucucg

SEQ ID NO: 182 - RNA, 19 nt, Artificial Sequence
  Description of Artificial Sequence Synthetic oligonucleotide
gaccacggaccuugcuaca

SEQ ID NO: 183 - RNA, 19 nt, Artificial Sequence
  Description of Artificial Sequence Synthetic oligonucleotide
cauacugaugucgugcuaa

SEQ ID NO: 184 - RNA, 19 nt, Artificial Sequence
  Description of Artificial Sequence Synthetic oligonucleotide
cccuuagucuccuguacag

SEQ ID NO: 185 - RNA, 19 nt, Artificial Sequence
  Description of Artificial Sequence Synthetic oligonucleotide
ggaguuucacuucguucuc

SEQ ID NO: 186 - RNA, 19 nt, Artificial Sequence
  Description of Artificial Sequence Synthetic oligonucleotide
gucauugggcgccaaguuc

SEQ ID NO: 187 - RNA, 19 nt, Artificial Sequence
  Description of Artificial Sequence Synthetic oligonucleotide
gaaucuuaacgcgggcagc

SEQ ID NO: 188 - RNA, 19 nt, Artificial Sequence
  Description of Artificial Sequence Synthetic oligonucleotide
ucaagauaugaaccagcuu

SEQ ID NO: 189 - RNA, 19 nt, Artificial Sequence
  Description of Artificial Sequence Synthetic oligonucleotide
cgggaaucuuaacgcgggc

SEQ ID NO: 190 - RNA, 19 nt, Artificial Sequence
  Description of Artificial Sequence Synthetic oligonucleotide
agcgcggcgcgacuuccuc

SEQ ID NO: 191 - RNA, 19 nt, Artificial Sequence
  Description of Artificial Sequence Synthetic oligonucleotide
ucaguucgcuggucucgag

SEQ ID NO: 192 - RNA, 19 nt, Artificial Sequence
  Description of Artificial Sequence Synthetic oligonucleotide
cauuaagcucaccaccucu

SEQ ID NO: 193 - RNA, 19 nt, Artificial Sequence
  Description of Artificial Sequence Synthetic oligonucleotide
ucauuaagcucaccaccuc

SEQ ID NO: 194 - RNA, 19 nt, Artificial Sequence
  Description of Artificial Sequence Synthetic oligonucleotide
gucuagggccgugaaacuc

SEQ ID NO: 195 - RNA, 19 nt, Artificial Sequence
  Description of Artificial Sequence Synthetic oligonucleotide
gguaagcuguuggaggccg

SEQ ID NO: 196 - RNA, 19 nt, Artificial Sequence
  Description of Artificial Sequence Synthetic oligonucleotide
cauucaugcuauccgggcu

SEQ ID NO: 197 - RNA, 19 nt, Artificial Sequence
  Description of Artificial Sequence Synthetic oligonucleotide
ggaucaacgccaggcccgc

SEQ ID NO: 198 - RNA, 19 nt, Artificial Sequence
  Description of Artificial Sequence Synthetic oligonucleotide
gggaaucuuaacgcgggca

SEQ ID NO: 199 - RNA, 19 nt, Artificial Sequence
  Description of Artificial Sequence Synthetic oligonucleotide
cauuaaaaugcgggaaucu

SEQ ID NO: 200 - RNA, 19 nt, Artificial Sequence
  Description of Artificial Sequence Synthetic oligonucleotide
caggcuggcuaagucgaga

SEQ ID NO: 201 - RNA, 19 nt, Artificial Sequence
  Description of Artificial Sequence Synthetic oligonucleotide
ucgggcagccaccacauug

SEQ ID NO: 202 - RNA, 19 nt, Artificial Sequence
  Description of Artificial Sequence Synthetic oligonucleotide
ugcuugcaaaccacaguuu

SEQ ID NO: 203 - RNA, 19 nt, Artificial Sequence
  Description of Artificial Sequence Synthetic oligonucleotide
ucugucggcagggacgcgg

SEQ ID NO: 204 - RNA, 19 nt, Artificial Sequence
  Description of Artificial Sequence Synthetic oligonucleotide
uuuaucugaugugauccca

SEQ ID NO: 205 - RNA, 19 nt, Artificial Sequence
  Description of Artificial Sequence Synthetic oligonucleotide
cccgauggaugauucuguu

SEQ ID NO: 206 - RNA, 19 nt, Artificial Sequence
  Description of Artificial Sequence Synthetic oligonucleotide
ggcuaagucgagaggcauu

SEQ ID NO: 207 - RNA, 19 nt, Artificial Sequence
  Description of Artificial Sequence Synthetic oligonucleotide
ucccuuagucuccuguaca

SEQ ID NO: 208 - RNA, 19 nt, Artificial Sequence
  Description of Artificial Sequence Synthetic oligonucleotide
cuucguucucccgcccaca

SEQ ID NO: 209 - RNA, 19 nt, Artificial Sequence
  Description of Artificial Sequence Synthetic oligonucleotide
ucguucucccgcccacaga

SEQ ID NO: 210 - RNA, 19 nt, Artificial Sequence
  Description of Artificial Sequence Synthetic oligonucleotide
accuugcuacaagcaaucu

SEQ ID NO: 211 - DNA, 21 bp, Artificial Sequence
  Description of Artificial Sequence Synthetic oligonucleotide
acuuaaagcugguucauautt

SEQ ID NO: 212 - DNA, 21 bp, Artificial Sequence
  Description of Artificial Sequence Synthetic oligonucleotide
auaugaaccagcuuuaagutt

SEQ ID NO: 213 - DNA, 21 bp, Artificial Sequence
  Description of Artificial Sequence Synthetic oligonucleotide
gcaugaaugccucucgacutt

SEQ ID NO: 214 - DNA, 21 bp, Artificial Sequence
  Description of Artificial Sequence Synthetic oligonucleotide
agucgagaggcauucaugctt

SEQ ID NO: 215 - DNA, 21 bp, Artificial Sequence
  Description of Artificial Sequence Synthetic oligonucleotide
gguggugagcuuaaugaautt

SEQ ID NO: 216 - DNA, 21 bp, Artificial Sequence
  Description of Artificial Sequence Synthetic oligonucleotide
auucauuaagcucaccacctt

SEQ ID NO: 217 - DNA, 21 bp, Artificial Sequence
  Description of Artificial Sequence Synthetic oligonucleotide
gguggugagcuuaaugaautt

SEQ ID NO: 218 - DNA, 21 bp, Artificial Sequence
  Description of Artificial Sequence Synthetic oligonucleotide
auucauuaagcucaccacctt

SEQ ID NO: 219 - DNA, 21 bp, Artificial Sequence
  Description of Artificial Sequence Synthetic oligonucleotide
acuuaaagcugguucauautt

SEQ ID NO: 220 - DNA, 21 bp, Artificial Sequence
  Description of Artificial Sequence Synthetic oligonucleotide
auaugaaccagcuuuaagutt

SEQ ID NO: 221 - DNA, 21 bp, Artificial Sequence
  Description of Artificial Sequence Synthetic oligonucleotide
tguggugagcuuaaugaautt

SEQ ID NO: 222 - DNA, 21 bp, Artificial Sequence
  Description of Artificial Sequence Synthetic oligonucleotide
auucauuaagcucaccacctt

SEQ ID NO: 223 - DNA, 21 bp, Artificial Sequence
  Description of Artificial Sequence Synthetic oligonucleotide
gcaagggagcuguacaggatt

SEQ ID NO: 224 - DNA, 21 bp, Artificial Sequence
  Description of Artificial Sequence Synthetic oligonucleotide
uccuguacagcucccuugctt

SEQ ID NO: 225 - DNA, 21 bp, Artificial Sequence
  Description of Artificial Sequence Synthetic oligonucleotide
tcaugaaugccucucgacctt

SEQ ID NO: 226 - DNA, 21 bp, Artificial Sequence
  Description of Artificial Sequence Synthetic oligonucleotide
agucgagaggcauucaugctt

SEQ ID NO: 227 - DNA, 21 bp, Artificial Sequence
  Description of Artificial Sequence Synthetic oligonucleotide
tguggugagcuuaaugaactt

SEQ ID NO: 228 - DNA, 21 bp, Artificial Sequence
  Description of Artificial Sequence Synthetic oligonucleotide
auucauuaagcucaccacctt

SEQ ID NO: 229 - DNA, 21 bp, Artificial Sequence
  Description of Artificial Sequence Synthetic oligonucleotide
aguacacagugaccgucgatt

SEQ ID NO: 230 - DNA, 21 bp, Artificial Sequence
  Description of Artificial Sequence Synthetic oligonucleotide
ucgacggucacuguguacutt

SEQ ID NO: 231 - DNA, 21 bp, Artificial Sequence
  Description of Artificial Sequence Synthetic oligonucleotide
tguacacagugaccgucgctt

SEQ ID NO: 232 - DNA, 21 bp, Artificial Sequence
  Description of Artificial Sequence Synthetic oligonucleotide
ucgacggucacuguguacutt

SEQ ID NO: 233 - DNA, 21 bp, Artificial Sequence
  Description of Artificial Sequence Synthetic oligonucleotide
gcaagggagcuguacaggatt

SEQ ID NO: 234 - DNA, 21 bp, Artificial Sequence
  Description of Artificial Sequence Synthetic oligonucleotide
uccuguacagcucccuugctt

SEQ ID NO: 235 - DNA, 21 bp, Artificial Sequence
  Description of Artificial Sequence Synthetic oligonucleotide
tcuuaaagcugguucauactt

SEQ ID NO: 236 - DNA, 21 bp, Artificial Sequence
  Description of Artificial Sequence Synthetic oligonucleotide
auaugaaccagcuuuaagutt

SEQ ID NO: 237 - DNA, 21 bp, Artificial Sequence
  Description of Artificial Sequence Synthetic oligonucleotide
gcaagggagcuguacaggatt

SEQ ID NO: 238 - DNA, 21 bp, Artificial Sequence
  Description of Artificial Sequence Synthetic oligonucleotide
uccuguacagcucccuugctt

SEQ ID NO: 239 - DNA, 21 bp, Artificial Sequence
  Description of Artificial Sequence Synthetic oligonucleotide
tguacacagugaccgucgctt

SEQ ID NO: 240 - DNA, 21 bp, Artificial Sequence
  Description of Artificial Sequence Synthetic oligonucleotide
ucgacggucacuguguacutt

SEQ ID NO: 241 - DNA, 21 bp, Artificial Sequence
  Description of Artificial Sequence Synthetic oligonucleotide
aguacacagugaccgucgatt

SEQ ID NO: 242 - DNA, 21 bp, Artificial Sequence
  Description of Artificial Sequence Synthetic oligonucleotide
ucgacggucacuguguacutt

SEQ ID NO: 243 - DNA, 21 bp, Artificial Sequence
  Description of Artificial Sequence Synthetic oligonucleotide
tcaagggagcuguacaggctt

SEQ ID NO: 244 - DNA, 21 bp, Artificial Sequence
  Description of Artificial Sequence Synthetic oligonucleotide
uccuguacagcucccuugctt

SEQ ID NO: 245 - DNA, 21 bp, Artificial Sequence
  Description of Artificial Sequence Synthetic oligonucleotide
tguacacagugaccgucgatt

SEQ ID NO: 246 - DNA, 21 bp, Artificial Sequence
  Description of Artificial Sequence Synthetic oligonucleotide
ucgacggucacuguguacutt

SEQ ID NO: 247 - DNA, 21 bp, Artificial Sequence
  Description of Artificial Sequence Synthetic oligonucleotide
tcaagggagcuguacaggatt

SEQ ID NO: 248 - DNA, 21 bp, Artificial Sequence
  Description of Artificial Sequence Synthetic oligonucleotide
uccuguacagcucccuugctt

SEQ ID NO: 249 - DNA, 21 bp, Artificial Sequence
  Description of Artificial Sequence Synthetic oligonucleotide
aguacacagugaccgucgatt

SEQ ID NO: 250 - DNA, 21 bp, Artificial Sequence
  Description of Artificial Sequence Synthetic oligonucleotide
ucgacggucacuguguacutt

SEQ ID NO: 251 - DNA, 21 bp, Artificial Sequence
  Description of Artificial Sequence Synthetic oligonucleotide
tguacacagugaccgucgctt

SEQ ID NO: 252 - DNA, 21 bp, Artificial Sequence
  Description of Artificial Sequence Synthetic oligonucleotide
ucgacggucacuguguacutt

SEQ ID NO: 253 - DNA, 21 bp, Artificial Sequence
  Description of Artificial Sequence Synthetic oligonucleotide
gcaugaaugccucucgacutt

SEQ ID NO: 254 - DNA, 21 bp, Artificial Sequence
  Description of Artificial Sequence Synthetic oligonucleotide
agucgagaggcauucaugctt

SEQ ID NO: 255 - DNA, 21 bp, Artificial Sequence
  Description of Artificial Sequence Synthetic oligonucleotide
tcaagggagcuguacaggatt

SEQ ID NO: 256 - DNA, 21 bp, Artificial Sequence
  Description of Artificial Sequence Synthetic oligonucleotide
uccuguacagcucccuugctt

SEQ ID NO: 257 - DNA, 21 bp, Artificial Sequence
  Description of Artificial Sequence Synthetic oligonucleotide
tcaagggagcuguacaggatt

SEQ ID NO: 258 - DNA, 21 bp, Artificial Sequence
  Description of Artificial Sequence Synthetic oligonucleotide
uccuguacagcucccuugctt

SEQ ID NO: 259 - DNA, 21 bp, Artificial Sequence
  Description of Artificial Sequence Synthetic oligonucleotide
tcaagggagcuguacaggctt

SEQ ID NO: 260 - DNA, 21 bp, Artificial Sequence
  Description of Artificial Sequence Synthetic oligonucleotide
uccuguacagcucccuugctt

SEQ ID NO: 261 - DNA, 21 bp, Artificial Sequence
  Description of Artificial Sequence Synthetic oligonucleotide
tguacacagugaccgucgatt

SEQ ID NO: 262 - DNA, 21 bp, Artificial Sequence
  Description of Artificial Sequence Synthetic oligonucleotide
ucgacggucacuguguacutt

SEQ ID NO: 263 - DNA, 21 bp, Artificial Sequence
  Description of Artificial Sequence Synthetic oligonucleotide
acuuaaagcugguucauautt

SEQ ID NO: 264 - DNA, 21 bp, Artificial Sequence
  Description of Artificial Sequence Synthetic oligonucleotide
auaugaaccagcuuuaagutt

SEQ ID NO: 265 - DNA, 21 bp, Artificial Sequence
  Description of Artificial Sequence Synthetic oligonucleotide
tguacacagugaccgucgctt

SEQ ID NO: 266 - DNA, 21 bp, Artificial Sequence
  Description of Artificial Sequence Synthetic oligonucleotide
ucgacggucacuguguacutt

SEQ ID NO: 267 - DNA, 21 bp, Artificial Sequence
  Description of Artificial Sequence Synthetic oligonucleotide
tcaugaaugccucucgacutt

SEQ ID NO: 268 - DNA, 21 bp, Artificial Sequence
  Description of Artificial Sequence Synthetic oligonucleotide
ggucgagaggcauucaugctt

SEQ ID NO: 269 - DNA, 21 bp, Artificial Sequence
  Description of Artificial Sequence Synthetic oligonucleotide
acuuaaagcugguucauautt

SEQ ID NO: 270 - DNA, 21 bp, Artificial Sequence
  Description of Artificial Sequence Synthetic oligonucleotide
auaugaaccagcuuuaagutt

SEQ ID NO: 271 - DNA, 21 bp, Artificial Sequence
  Description of Artificial Sequence Synthetic oligonucleotide
tcaagggagcuguacaggatt

SEQ ID NO: 272 - DNA, 21 bp, Artificial Sequence
  Description of Artificial Sequence Synthetic oligonucleotide
uccuguacagcucccuugctt

SEQ ID NO: 273 - DNA, 21 bp, Artificial Sequence
  Description of Artificial Sequence Synthetic oligonucleotide
tcaagggagcuguacaggctt

SEQ ID NO: 274 - DNA, 21 bp, Artificial Sequence
  Description of Artificial Sequence Synthetic oligonucleotide
uccuguacagcucccuugctt

SEQ ID NO: 275 - DNA, 21 bp, Artificial Sequence
  Description of Artificial Sequence Synthetic oligonucleotide
aguacacagugaccgucgatt

SEQ ID NO: 276 - DNA, 21 bp, Artificial Sequence
  Description of Artificial Sequence Synthetic oligonucleotide
ucgacggucacuguguacutt

SEQ ID NO: 277 - DNA, 21 bp, Artificial Sequence
  Description of Artificial Sequence Synthetic oligonucleotide
tcaugaaugccucucgacctt

SEQ ID NO: 278 - DNA, 21 bp, Artificial Sequence
  Description of Artificial Sequence Synthetic oligonucleotide
agucgagaggcauucaugctt

SEQ ID NO: 279 - DNA, 21 bp, Artificial Sequence
  Description of Artificial Sequence Synthetic oligonucleotide
aguacacagugaccgucgatt

SEQ ID NO: 280 - DNA, 21 bp, Artificial Sequence
  Description of Artificial Sequence Synthetic oligonucleotide
ucgacggucacuguguacutt

SEQ ID NO: 281 - DNA, 21 bp, Artificial Sequence
  Description of Artificial Sequence Synthetic oligonucleotide
tguacacagugaccgucgatt

SEQ ID NO: 282 - DNA, 21 bp, Artificial Sequence
  Description of Artificial Sequence Synthetic oligonucleotide
ucgacggucacuguguacutt

SEQ ID NO: 283 - DNA, 21 bp, Artificial Sequence
  Description of Artificial Sequence Synthetic oligonucleotide
gcaugaaugccucucgacutt

SEQ ID NO: 284 - DNA, 21 bp, Artificial Sequence
  Description of Artificial Sequence Synthetic oligonucleotide
agucgagaggcauucaugctt

SEQ ID NO: 285 - DNA, 21 bp, Artificial Sequence
  Description of Artificial Sequence Synthetic oligonucleotide
acuuaaagcugguucauautt

SEQ ID NO: 286 - DNA, 21 bp, Artificial Sequence
  Description of Artificial Sequence Synthetic oligonucleotide
auaugaaccagcuuuaagutt

SEQ ID NO: 287 - DNA, 21 bp, Artificial Sequence
  Description of Artificial Sequence Synthetic oligonucleotide
acuuaaagcugguucauautt

SEQ ID NO: 288 - DNA, 21 bp, Artificial Sequence
  Description of Artificial Sequence Synthetic oligonucleotide
auaugaaccagcuuuaagutt

SEQ ID NO: 289 - DNA, 21 bp, Artificial Sequence
  Description of Artificial Sequence Synthetic oligonucleotide
tcuuaaagcugguucauactt

SEQ ID NO: 290 - DNA, 21 bp, Artificial Sequence
  Description of Artificial Sequence Synthetic oligonucleotide
auaugaaccagcuuuaagutt

SEQ ID NO: 291 - DNA, 21 bp, Artificial Sequence
  Description of Artificial Sequence Synthetic oligonucleotide
ggaugagaagacuguuguctt

SEQ ID NO: 292 - DNA, 21 bp, Artificial Sequence
  Description of Artificial Sequence Synthetic oligonucleotide
gacaacagucuucucaucctt

SEQ ID NO: 293 - DNA, 21 bp, Artificial Sequence
  Description of Artificial Sequence Synthetic oligonucleotide
tcaagggagcuguacaggctt

SEQ ID NO: 294 - DNA, 21 bp, Artificial Sequence
  Description of Artificial Sequence Synthetic oligonucleotide
uccuguacagcucccuugctt

SEQ ID NO: 295 - DNA, 21 bp, Artificial Sequence
  Description of Artificial Sequence Synthetic oligonucleotide
gcaugaaugccucucgacutt

SEQ ID NO: 296 - DNA, 21 bp, Artificial Sequence
  Description of Artificial Sequence Synthetic oligonucleotide
agucgagaggcauucaugctt

SEQ ID NO: 297 - DNA, 21 bp, Artificial Sequence
  Description of Artificial Sequence Synthetic oligonucleotide
gguggugagcuuaaugaautt

SEQ ID NO: 298 - DNA, 21 bp, Artificial Sequence
  Description of Artificial Sequence Synthetic oligonucleotide
auucauuaagcucaccacctt

SEQ ID NO: 299 - DNA, 21 bp, Artificial Sequence
  Description of Artificial Sequence Synthetic oligonucleotide
gcuagaaaaugccauacagtt

SEQ ID NO: 300 - DNA, 21 bp, Artificial Sequence
  Description of Artificial Sequence Synthetic oligonucleotide
cuguauggcauuuucuagctt

SEQ ID NO: 301 - DNA, 21 bp, Artificial Sequence
  Description of Artificial Sequence Synthetic oligonucleotide
cugaagauugcuuguagcatt

SEQ ID NO: 302 - DNA, 21 bp, Artificial Sequence
  Description of Artificial Sequence Synthetic oligonucleotide
ugcuacaagcaaucuucagtt

SEQ ID NO: 303 - DNA, 21 bp, Artificial Sequence
  Description of Artificial Sequence Synthetic oligonucleotide
aguacacagugaccgucgatt

SEQ ID NO: 304 - DNA, 21 bp, Artificial Sequence
  Description of Artificial Sequence Synthetic oligonucleotide
ucgacggucacuguguacutt

SEQ ID NO: 305 - DNA, 21 bp, Artificial Sequence
  Description of Artificial Sequence Synthetic oligonucleotide
tcaugaaugccucucgacutt

SEQ ID NO: 306 - DNA, 21 bp, Artificial Sequence
  Description of Artificial Sequence Synthetic oligonucleotide
agucgagaggcauucaugctt

SEQ ID NO: 307 - DNA, 21 bp, Artificial Sequence
  Description of Artificial Sequence Synthetic oligonucleotide
tcaugaaugccucucgacutt

SEQ ID NO: 308 - DNA, 21 bp, Artificial Sequence
  Description of Artificial Sequence Synthetic oligonucleotide
agucgagaggcauucaugctt

SEQ ID NO: 309 - DNA, 21 bp, Artificial Sequence
  Description of Artificial Sequence Synthetic oligonucleotide
tcaugaaugccucucgacctt

SEQ ID NO: 310 - DNA, 21 bp, Artificial Sequence
  Description of Artificial Sequence Synthetic oligonucleotide
agucgagaggcauucaugctt

SEQ ID NO: 311 - DNA, 21 bp, Artificial Sequence
  Description of Artificial Sequence Synthetic oligonucleotide
tguacacagugaccgucgctt

SEQ ID NO: 312 - DNA, 21 bp, Artificial Sequence
  Description of Artificial Sequence Synthetic oligonucleotide
ucgacggucacuguguacutt

SEQ ID NO: 313 - DNA, 21 bp, Artificial Sequence
  Description of Artificial Sequence Synthetic oligonucleotide
gcaugaaugccucucgacutt

SEQ ID NO: 314 - DNA, 21 bp, Artificial Sequence
  Description of Artificial Sequence Synthetic oligonucleotide
ggucgagaggcauucaugctt

SEQ ID NO: 315 - DNA, 21 bp, Artificial Sequence
  Description of Artificial Sequence Synthetic oligonucleotide
tcaugaaugccucucgacutt

SEQ ID NO: 316 - DNA, 21 bp, Artificial Sequence
  Description of Artificial Sequence Synthetic oligonucleotide
agucgagaggcauucaugctt

SEQ ID NO: 317 - DNA, 21 bp, Artificial Sequence
  Description of Artificial Sequence Synthetic oligonucleotide
ccgacagaguuagcacgactt

SEQ ID NO: 318 - DNA, 21 bp, Artificial Sequence
  Description of Artificial Sequence Synthetic oligonucleotide
gucgugcuaacucugucggtt

SEQ ID NO: 319 - DNA, 21 bp, Artificial Sequence
  Description of Artificial Sequence Synthetic oligonucleotide
aguacacagugaccgucgatt

SEQ ID NO: 320 - DNA, 21 bp, Artificial Sequence
  Description of Artificial Sequence Synthetic oligonucleotide
ucgacggucacuguguacutt

SEQ ID NO: 321 - DNA, 21 bp, Artificial Sequence
  Description of Artificial Sequence Synthetic oligonucleotide
tguggugagcuuaaugaautt

SEQ ID NO: 322 - DNA, 21 bp, Artificial Sequence
  Description of Artificial Sequence Synthetic oligonucleotide
auucauuaagcucaccacctt

SEQ ID NO: 323 - DNA, 21 bp, Artificial Sequence
  Description of Artificial Sequence Synthetic oligonucleotide
gguggugagcuuaaugaautt

SEQ ID NO: 324 - DNA, 21 bp, Artificial Sequence
  Description of Artificial Sequence Synthetic oligonucleotide
auucauuaagcucaccacctt

SEQ ID NO: 325 - DNA, 21 bp, Artificial Sequence
  Description of Artificial Sequence Synthetic oligonucleotide
aguacacagugaccgucgatt

SEQ ID NO: 326 - DNA, 21 bp, Artificial Sequence
  Description of Artificial Sequence Synthetic oligonucleotide
ucgacggucacuguguacutt

SEQ ID NO: 327 - DNA, 21 bp, Artificial Sequence
  Description of Artificial Sequence Synthetic oligonucleotide
gcaugaaugccucucgacutt

SEQ ID NO: 328 - DNA, 21 bp, Artificial Sequence
  Description of Artificial Sequence Synthetic oligonucleotide
agucgagaggcauucaugctt

SEQ ID NO: 329 - DNA, 21 bp, Artificial Sequence
  Description of Artificial Sequence Synthetic oligonucleotide
tcaugaaugccucucgacutt

SEQ ID NO: 330 - DNA, 21 bp, Artificial Sequence
  Description of Artificial Sequence Synthetic oligonucleotide
agucgagaggcauucaugctt

SEQ ID NO: 331 - DNA, 21 bp, Artificial Sequence
  Description of Artificial Sequence Synthetic oligonucleotide
tguggugagcuuaaugaactt

SEQ ID NO: 332 - DNA, 21 bp, Artificial Sequence
  Description of Artificial Sequence Synthetic oligonucleotide
auucauuaagcucaccacctt

SEQ ID NO: 333 - DNA, 21 bp, Artificial Sequence
  Description of Artificial Sequence Synthetic oligonucleotide
agugucagcuguauccuuctt

SEQ ID NO: 334 - DNA, 21 bp, Artificial Sequence
  Description of Artificial Sequence Synthetic oligonucleotide
gaaggauacagcugacacutt

SEQ ID NO: 335 - DNA, 21 bp, Artificial Sequence
  Description of Artificial Sequence Synthetic oligonucleotide
aggcaauucagagcuucgatt

SEQ ID NO: 336 - DNA, 21 bp, Artificial Sequence
  Description of Artificial Sequence Synthetic oligonucleotide
ucgaagcucugaauugccutt

SEQ ID NO: 337 - DNA, 21 bp, Artificial Sequence
  Description of Artificial Sequence Synthetic oligonucleotide
gguggugagcuuaaugaautt

SEQ ID NO: 338 - DNA, 21 bp, Artificial Sequence
  Description of Artificial Sequence Synthetic oligonucleotide
auucauuaagcucaccacctt

SEQ ID NO: 339 - DNA, 21 bp, Artificial Sequence
  Description of Artificial Sequence Synthetic oligonucleotide
tguacacagugaccgucgatt

SEQ ID NO: 340 - DNA, 21 bp, Artificial Sequence
  Description of Artificial Sequence Synthetic oligonucleotide
ucgacggucacuguguacutt

SEQ ID NO: 341 - DNA, 21 bp, Artificial Sequence
  Description of Artificial Sequence Synthetic oligonucleotide
tcaugaaugccucucgacutt

SEQ ID NO: 342 - DNA, 21 bp, Artificial Sequence
  Description of Artificial Sequence Synthetic oligonucleotide
agucgagaggcauucaugctt

SEQ ID NO: 343 - DNA, 21 bp, Artificial Sequence
  Description of Artificial Sequence Synthetic oligonucleotide
gguggugagcuuaaugaautt

SEQ ID NO: 344 - DNA, 21 bp, Artificial Sequence
  Description of Artificial Sequence Synthetic oligonucleotide
auucauuaagcucaccacctt

SEQ ID NO: 345 - DNA, 21 bp, Artificial Sequence
  Description of Artificial Sequence Synthetic oligonucleotide
tguggugagcuuaaugaautt

SEQ ID NO: 346 - DNA, 21 bp, Artificial Sequence
  Description of Artificial Sequence Synthetic oligonucleotide
auucauuaagcucaccacctt

SEQ ID NO: 347 - DNA, 21 bp, Artificial Sequence
  Description of Artificial Sequence Synthetic oligonucleotide
tguggugagcuuaaugaautt

SEQ ID NO: 348 - DNA, 21 bp, Artificial Sequence
  Description of Artificial Sequence Synthetic oligonucleotide
auucauuaagcucaccacctt

SEQ ID NO: 349 - DNA, 21 bp, Artificial Sequence
  Description of Artificial Sequence Synthetic oligonucleotide
tguggugagcuuaaugaactt

SEQ ID NO: 350 - DNA, 21 bp, Artificial Sequence
  Description of Artificial Sequence Synthetic oligonucleotide
auucauuaagcucaccacctt

SEQ ID NO: 351 - DNA, 21 bp, Artificial Sequence
  Description of Artificial Sequence Synthetic oligonucleotide
tcuuaaagcugguucauautt

SEQ ID NO: 352 - DNA, 21 bp, Artificial Sequence
  Description of Artificial Sequence Synthetic oligonucleotide
auaugaaccagcuuuaagutt

SEQ ID NO: 353 - DNA, 21 bp, Artificial Sequence
  Description of Artificial Sequence Synthetic oligonucleotide
tcuuaaagcugguucauautt

SEQ ID NO: 354 - DNA, 21 bp, Artificial Sequence
  Description of Artificial Sequence Synthetic oligonucleotide
auaugaaccagcuuuaagutt

SEQ ID NO: 355 - DNA, 21 bp, Artificial Sequence
  Description of Artificial Sequence Synthetic oligonucleotide
gaucagggcagucuuugcatt

SEQ ID NO: 356 - DNA, 21 bp, Artificial Sequence
  Description of Artificial Sequence Synthetic oligonucleotide
ugcaaagacugcccugauctt

SEQ ID NO: 357 - DNA, 21 bp, Artificial Sequence
  Description of Artificial Sequence Synthetic oligonucleotide
ucaggaaaugguacggcugtt

SEQ ID NO: 358 - DNA, 21 bp, Artificial Sequence
  Description of Artificial Sequence Synthetic oligonucleotide
cagccguaccauuuccugatt

SEQ ID NO: 359 - DNA, 21 bp, Artificial Sequence
  Description of Artificial Sequence Synthetic oligonucleotide
gcaagggagcuguacaggatt

SEQ ID NO: 360 - DNA, 21 bp, Artificial Sequence
  Description of Artificial Sequence Synthetic oligonucleotide
uccuguacagcucccuugctt

SEQ ID NO: 361 - DNA, 21 bp, Artificial Sequence
  Description of Artificial Sequence Synthetic oligonucleotide
gcaagggagcuguacaggatt

SEQ ID NO: 362 - DNA, 21 bp, Artificial Sequence
  Description of Artificial Sequence Synthetic oligonucleotide
uccuguacagcucccuugctt

SEQ ID NO: 363 - DNA, 21 bp, Artificial Sequence
  Description of Artificial Sequence Synthetic oligonucleotide
tcaugaaugccucucgacctt

SEQ ID NO: 364 - DNA, 21 bp, Artificial Sequence
  Description of Artificial Sequence Synthetic oligonucleotide
ggucgagaggcauucaugctt

SEQ ID NO: 365 - DNA, 21 bp, Artificial Sequence
  Description of Artificial Sequence Synthetic oligonucleotide
gguggugagcuuaaugaautt

SEQ ID NO: 366 - DNA, 21 bp, Artificial Sequence
  Description of Artificial Sequence Synthetic oligonucleotide
auucauuaagcucaccacctt

SEQ ID NO: 367 - DNA, 21 bp, Artificial Sequence
  Description of Artificial Sequence Synthetic oligonucleotide
tguacacagugaccgucgatt

SEQ ID NO: 368 - DNA, 21 bp, Artificial Sequence
  Description of Artificial Sequence Synthetic oligonucleotide
ucgacggucacuguguacutt

SEQ ID NO: 369 - DNA, 21 bp, Artificial Sequence
  Description of Artificial Sequence Synthetic oligonucleotide
tcaugaaugccucucgacctt

SEQ ID NO: 370 - DNA, 21 bp, Artificial Sequence
  Description of Artificial Sequence Synthetic oligonucleotide
agucgagaggcauucaugctt

SEQ ID NO: 371 - DNA, 21 bp, Artificial Sequence
  Description of Artificial Sequence Synthetic oligonucleotide
gguggugagcuuaaugaautt

SEQ ID NO: 372 - DNA, 21 bp, Artificial Sequence
  Description of Artificial Sequence Synthetic oligonucleotide
auucauuaagcucaccacctt

SEQ ID NO: 373 - DNA, 21 bp, Artificial Sequence
  Description of Artificial Sequence Synthetic oligonucleotide
tcuuaaagcugguucauautt

SEQ ID NO: 374 - DNA, 21 bp, Artificial Sequence
  Description of Artificial Sequence Synthetic oligonucleotide
auaugaaccagcuuuaagutt

SEQ ID NO: 375 - DNA, 21 bp, Artificial Sequence
  Description of Artificial Sequence Synthetic oligonucleotide
tcuuaaagcugguucauactt

SEQ ID NO: 376 - DNA, 21 bp, Artificial Sequence
  Description of Artificial Sequence Synthetic oligonucleotide
auaugaaccagcuuuaagutt

SEQ ID NO: 377 - DNA, 21 bp, Artificial Sequence
  Description of Artificial Sequence Synthetic oligonucleotide
ugguucauaucuugaacautt

SEQ ID NO: 378 - DNA, 21 bp, Artificial Sequence
  Description of Artificial Sequence Synthetic oligonucleotide
auguucaagauaugaaccatt

SEQ ID NO: 379 - DNA, 21 bp, Artificial Sequence
  Description of Artificial Sequence Synthetic oligonucleotide
acagaaucauccaucgggatt

SEQ ID NO: 380 - DNA, 21 bp, Artificial Sequence
  Description of Artificial Sequence Synthetic oligonucleotide
ucccgauggaugauucugutt

SEQ ID NO: 381 - DNA, 21 bp, Artificial Sequence
  Description of Artificial Sequence Synthetic oligonucleotide
guacacagugaccgucgactt

SEQ ID NO: 382 - DNA, 21 bp, Artificial Sequence
  Description of Artificial Sequence Synthetic oligonucleotide
gucgacggucacuguguactt

SEQ ID NO: 383 - DNA, 21 bp, Artificial Sequence
  Description of Artificial Sequence Synthetic oligonucleotide
uugcuuguagcaagguccgtt

SEQ ID NO: 384 - DNA, 21 bp, Artificial Sequence
  Description of Artificial Sequence Synthetic oligonucleotide
cggaccuugcuacaagcaatt

SEQ ID NO: 385 - DNA, 21 bp, Artificial Sequence
  Description of Artificial Sequence Synthetic oligonucleotide
gguggugagcuuaaugaautt

SEQ ID NO: 386 - DNA, 21 bp, Artificial Sequence
  Description of Artificial Sequence Synthetic oligonucleotide
auucauuaagcucaccacctt

SEQ ID NO: 387 - DNA, 21 bp, Artificial Sequence
  Description of Artificial Sequence Synthetic oligonucleotide
tcaugaaugccucucgacctt

SEQ ID NO: 388 - DNA, 21 bp, Artificial Sequence
  Description of Artificial Sequence Synthetic oligonucleotide
agucgagaggcauucaugctt

SEQ ID NO: 389 - DNA, 21 bp, Artificial Sequence
  Description of Artificial Sequence Synthetic oligonucleotide
gguggugagcuuaaugaautt

SEQ ID NO: 390 - DNA, 21 bp, Artificial Sequence
  Description of Artificial Sequence Synthetic oligonucleotide
auucauuaagcucaccacctt

SEQ ID NO: 391 - DNA, 21 bp, Artificial Sequence
  Description of Artificial Sequence Synthetic oligonucleotide
gguggugagcuuaaugaautt

SEQ ID NO: 392 - DNA, 21 bp, Artificial Sequence
  Description of Artificial Sequence Synthetic oligonucleotide
auucauuaagcucaccacctt

SEQ ID NO: 393 - DNA, 21 bp, Artificial Sequence
  Description of Artificial Sequence Synthetic oligonucleotide
cugccgacagaguuagcactt

SEQ ID NO: 394 - DNA, 21 bp, Artificial Sequence
  Description of Artificial Sequence Synthetic oligonucleotide
gugcuaacucugucggcagtt

SEQ ID NO: 395 - DNA, 21 bp, Artificial Sequence
  Description of Artificial Sequence Synthetic oligonucleotide
gaaagugcgagugaucuautt

SEQ ID NO: 396 - DNA, 21 bp, Artificial Sequence
  Description of Artificial Sequence Synthetic oligonucleotide
auagaucacucgcacuuuctt

SEQ ID NO: 397 - DNA, 21 bp, Artificial Sequence
  Description of Artificial Sequence Synthetic oligonucleotide
gcaagggagcuguacaggatt

SEQ ID NO: 398 - DNA, 21 bp, Artificial Sequence
  Description of Artificial Sequence Synthetic oligonucleotide
uccuguacagcucccuugctt

SEQ ID NO: 399 - DNA, 21 bp, Artificial Sequence
  Description of Artificial Sequence Synthetic oligonucleotide
aguacacagugaccgucgatt

SEQ ID NO: 400 - DNA, 21 bp, Artificial Sequence
  Description of Artificial Sequence Synthetic oligonucleotide
ucgacggucacuguguacutt

SEQ ID NO: 401 - DNA, 21 bp, Artificial Sequence
  Description of Artificial Sequence Synthetic oligonucleotide
aguacacagugaccgucgatt

SEQ ID NO: 402 - DNA, 21 bp, Artificial Sequence
  Description of Artificial Sequence Synthetic oligonucleotide
ucgacggucacuguguacutt

SEQ ID NO: 403 - DNA, 21 bp, Artificial Sequence
  Description of Artificial Sequence Synthetic oligonucleotide
tguacacagugaccgucgctt

SEQ ID NO: 404 - DNA, 21 bp, Artificial Sequence
  Description of Artificial Sequence Synthetic oligonucleotide
ucgacggucacuguguacutt

SEQ ID NO: 405 - DNA, 21 bp, Artificial Sequence
  Description of Artificial Sequence Synthetic oligonucleotide
tguacacagugaccgucgctt

SEQ ID NO: 406 - DNA, 21 bp, Artificial Sequence
  Description of Artificial Sequence Synthetic oligonucleotide
ucgacggucacuguguacutt

SEQ ID NO: 407 - DNA, 21 bp, Artificial Sequence
  Description of Artificial Sequence Synthetic oligonucleotide
gcaugaaugccucucgacutt

SEQ ID NO: 408 - DNA, 21 bp, Artificial Sequence
  Description of Artificial Sequence Synthetic oligonucleotide
agucgagaggcauucaugctt

SEQ ID NO: 409 - DNA, 21 bp, Artificial Sequence
  Description of Artificial Sequence Synthetic oligonucleotide
tcaugaaugccucucgacctt

SEQ ID NO: 410 - DNA, 21 bp, Artificial Sequence
  Description of Artificial Sequence Synthetic oligonucleotide
agucgagaggcauucaugctt

SEQ ID NO: 411 - DNA, 21 bp, Artificial Sequence
  Description of Artificial Sequence Synthetic oligonucleotide
tcaugaaugccucucgacctt

SEQ ID NO: 412 - DNA, 21 bp, Artificial Sequence
  Description of Artificial Sequence Synthetic oligonucleotide
agucgagaggcauucaugctt

SEQ ID NO: 413 - DNA, 21 bp, Artificial Sequence
  Description of Artificial Sequence Synthetic oligonucleotide
tcuuaaagcugguucauactt

SEQ ID NO: 414 - DNA, 21 bp, Artificial Sequence
  Description of Artificial Sequence Synthetic oligonucleotide
auaugaaccagcuuuaagutt

SEQ ID NO: 415 - DNA, 21 bp, Artificial Sequence
  Description of Artificial Sequence Synthetic oligonucleotide
ccugaagauugcuuguagctt

SEQ ID NO: 416 - DNA, 21 bp, Artificial Sequence
  Description of Artificial Sequence Synthetic oligonucleotide
gcuacaagcaaucuucaggtt

SEQ ID NO: 417 - DNA, 21 bp, Artificial Sequence
  Description of Artificial Sequence Synthetic oligonucleotide
cgacagaguuagcacgacatt

SEQ ID NO: 418 - DNA, 21 bp, Artificial Sequence
  Description of Artificial Sequence Synthetic oligonucleotide
ugucgugcuaacucugucgtt

SEQ ID NO: 419 - DNA, 21 bp, Artificial Sequence
  Description of Artificial Sequence Synthetic oligonucleotide
aguacacagugaccgucgatt

SEQ ID NO: 420 - DNA, 21 bp, Artificial Sequence
  Description of Artificial Sequence Synthetic oligonucleotide
ucgacggucacuguguacutt

SEQ ID NO: 421 - DNA, 21 bp, Artificial Sequence
  Description of Artificial Sequence Synthetic oligonucleotide
gcaugaaugccucucgacutt

SEQ ID NO: 422 - DNA, 21 bp, Artificial Sequence
  Description of Artificial Sequence Synthetic oligonucleotide
agucgagaggcauucaugctt

SEQ ID NO: 423 - DNA, 21 bp, Artificial Sequence
  Description of Artificial Sequence Synthetic oligonucleotide
tguggugagcuuaaugaactt

SEQ ID NO: 424 - DNA, 21 bp, Artificial Sequence
  Description of Artificial Sequence Synthetic oligonucleotide
auucauuaagcucaccacctt

SEQ ID NO: 425 - DNA, 21 bp, Artificial Sequence
  Description of Artificial Sequence Synthetic oligonucleotide
cuagaaaaugccauacaggtt

SEQ ID NO: 426 - DNA, 21 bp, Artificial Sequence
  Description of Artificial Sequence Synthetic oligonucleotide
ccuguauggcauuuucuagtt

SEQ ID NO: 427 - DNA, 21 bp, Artificial Sequence
  Description of Artificial Sequence Synthetic oligonucleotide
cugcccgcguuaagauucctt

SEQ ID NO: 428 - DNA, 21 bp, Artificial Sequence
  Description of Artificial Sequence Synthetic oligonucleotide
ggaaucuuaacgcgggcagtt

SEQ ID NO: 429 - DNA, 21 bp, Artificial Sequence
  Description of Artificial Sequence Synthetic oligonucleotide
tcaugaaugccucucgacutt

SEQ ID NO: 430 - DNA, 21 bp, Artificial Sequence
  Description of Artificial Sequence Synthetic oligonucleotide
agucgagaggcauucaugctt

SEQ ID NO: 431 - DNA, 21 bp, Artificial Sequence
  Description of Artificial Sequence Synthetic oligonucleotide
tguggugagcuuaaugaautt

SEQ ID NO: 432 - DNA, 21 bp, Artificial Sequence
  Description of Artificial Sequence Synthetic oligonucleotide
auucauuaagcucaccacctt

SEQ ID NO: 433 - DNA, 21 bp, Artificial Sequence
  Description of Artificial Sequence Synthetic oligonucleotide
tguggugagcuuaaugaactt

SEQ ID NO: 434 - DNA, 21 bp, Artificial Sequence
  Description of Artificial Sequence Synthetic oligonucleotide
auucauuaagcucaccacctt

SEQ ID NO: 435 - DNA, 21 bp, Artificial Sequence
  Description of Artificial Sequence Synthetic oligonucleotide
tguacacagugaccgucgatt

SEQ ID NO: 436 - DNA, 21 bp, Artificial Sequence
  Description of Artificial Sequence Synthetic oligonucleotide
ucgacggucacuguguacutt

SEQ ID NO: 437 - DNA, 21 bp, Artificial Sequence
  Description of Artificial Sequence Synthetic oligonucleotide
acuuaaagcugguucauautt

SEQ ID NO: 438 - DNA, 21 bp, Artificial Sequence
  Description of Artificial Sequence Synthetic oligonucleotide
auaugaaccagcuuuaagutt

SEQ ID NO: 439 - DNA, 21 bp, Artificial Sequence
  Description of Artificial Sequence Synthetic oligonucleotide
cagaaucauccaucgggautt

SEQ ID NO: 440 - DNA, 21 bp, Artificial Sequence
  Description of Artificial Sequence Synthetic oligonucleotide
aucccgauggaugauucugtt

SEQ ID NO: 441 - DNA, 21 bp, Artificial Sequence
  Description of Artificial Sequence Synthetic oligonucleotide
gccagaaaacaucguccugtt

SEQ ID NO: 442 - DNA, 21 bp, Artificial Sequence
  Description of Artificial Sequence Synthetic oligonucleotide
caggacgauguuuucuggctt

SEQ ID NO: 443 - DNA, 21 bp, Artificial Sequence
  Description of Artificial Sequence Synthetic oligonucleotide
guuugcaagcagaaggcgctt

SEQ ID NO: 444 - DNA, 21 bp, Artificial Sequence
  Description of Artificial Sequence Synthetic oligonucleotide
gcgccuucugcuugcaaactt

SEQ ID NO: 445 - DNA, 21 bp, Artificial Sequence
  Description of Artificial Sequence Synthetic oligonucleotide
ugcuagaaaaugccauacatt

SEQ ID NO: 446 - DNA, 21 bp, Artificial Sequence
  Description of Artificial Sequence Synthetic oligonucleotide
uguauggcauuuucuagcatt

SEQ ID NO: 447 - DNA, 21 bp, Artificial Sequence
  Description of Artificial Sequence Synthetic oligonucleotide
aguacacagugaccgucgatt

SEQ ID NO: 448 - DNA, 21 bp, Artificial Sequence
  Description of Artificial Sequence Synthetic oligonucleotide
ucgacggucacuguguacutt

SEQ ID NO: 449 - DNA, 21 bp, Artificial Sequence
  Description of Artificial Sequence Synthetic oligonucleotide
gguggugagcuuaaugaautt

SEQ ID NO: 450 - DNA, 21 bp, Artificial Sequence
  Description of Artificial Sequence Synthetic oligonucleotide
auucauuaagcucaccacctt

SEQ ID NO: 451 - DNA, 21 bp, Artificial Sequence
  Description of Artificial Sequence Synthetic oligonucleotide
agguggugagcuuaaugaatt

SEQ ID NO: 452 - DNA, 21 bp, Artificial Sequence
  Description of Artificial Sequence Synthetic oligonucleotide
uucauuaagcucaccaccutt

SEQ ID NO: 453 - DNA, 21 bp, Artificial Sequence
  Description of Artificial Sequence Synthetic oligonucleotide
gacagaguuagcacgacautt

SEQ ID NO: 454 - DNA, 21 bp, Artificial Sequence
  Description of Artificial Sequence Synthetic oligonucleotide
augucgugcuaacucuguctt

SEQ ID NO: 455 - DNA, 21 bp, Artificial Sequence
  Description of Artificial Sequence Synthetic oligonucleotide
aguacacagugaccgucgatt

SEQ ID NO: 456 - DNA, 21 bp, Artificial Sequence
  Description of Artificial Sequence Synthetic oligonucleotide
ucgacggucacuguguacutt

SEQ ID NO: 457 - DNA, 21 bp, Artificial Sequence
  Description of Artificial Sequence Synthetic oligonucleotide
gcaugaaugccucucgacutt

SEQ ID NO: 458 - DNA, 21 bp, Artificial Sequence
  Description of Artificial Sequence Synthetic oligonucleotide
agucgagaggcauucaugctt

SEQ ID NO: 459 - DNA, 21 bp, Artificial Sequence
  Description of Artificial Sequence Synthetic oligonucleotide
gcaugaaugccucucgacutt

SEQ ID NO: 460 - DNA, 21 bp, Artificial Sequence
  Description of Artificial Sequence Synthetic oligonucleotide
agucgagaggcauucaugctt

SEQ ID NO: 461 - DNA, 21 bp, Artificial Sequence
  Description of Artificial Sequence Synthetic oligonucleotide
gcgggagaacgaagugaaatt

SEQ ID NO: 462 - DNA, 21 bp, Artificial Sequence
  Description of Artificial Sequence Synthetic oligonucleotide
uuucacuucguucucccgctt

SEQ ID NO: 463 - DNA, 21 bp, Artificial Sequence
  Description of Artificial Sequence Synthetic oligonucleotide
aguacacagugaccgucgatt

SEQ ID NO: 464 - DNA, 21 bp, Artificial Sequence
  Description of Artificial Sequence Synthetic oligonucleotide
ucgacggucacuguguacutt

SEQ ID NO: 465 - DNA, 21 bp, Artificial Sequence
  Description of Artificial Sequence Synthetic oligonucleotide
tguacacagugaccgucgatt

SEQ ID NO: 466 - DNA, 21 bp, Artificial Sequence
  Description of Artificial Sequence Synthetic oligonucleotide
ucgacggucacuguguacutt

SEQ ID NO: 467 - DNA, 21 bp, Artificial Sequence
  Description of Artificial Sequence Synthetic oligonucleotide
gagcuguacaggagacuaatt

SEQ ID NO: 468 - DNA, 21 bp, Artificial Sequence
  Description of Artificial Sequence Synthetic oligonucleotide
uuagucuccuguacagcuctt

SEQ ID NO: 469 - DNA, 21 bp, Artificial Sequence
  Description of Artificial Sequence Synthetic oligonucleotide
ccucgagaccagcgaacugtt

SEQ ID NO: 470 - DNA, 21 bp, Artificial Sequence
  Description of Artificial Sequence Synthetic oligonucleotide
caguucgcuggucucgaggtt

SEQ ID NO: 471 - DNA, 21 bp, Artificial Sequence
  Description of Artificial Sequence Synthetic oligonucleotide
acuuaaagcugguucauautt

SEQ ID NO: 472 - DNA, 21 bp, Artificial Sequence
  Description of Artificial Sequence Synthetic oligonucleotide
guaugaaccagcuuuaagutt

SEQ ID NO: 473 - DNA, 21 bp, Artificial Sequence
  Description of Artificial Sequence Synthetic oligonucleotide
agccagaaaacaucguccutt

SEQ ID NO: 474 - DNA, 21 bp, Artificial Sequence
  Description of Artificial Sequence Synthetic oligonucleotide
aggacgauguuuucuggcutt

SEQ ID NO: 475 - DNA, 21 bp, Artificial Sequence
  Description of Artificial Sequence Synthetic oligonucleotide
cugguuacagacggaagaatt

SEQ ID NO: 476 - DNA, 21 bp, Artificial Sequence
  Description of Artificial Sequence Synthetic oligonucleotide
uucuuccgucuguaaccagtt

SEQ ID NO: 477 - DNA, 21 bp, Artificial Sequence
  Description of Artificial Sequence Synthetic oligonucleotide
agaguuucacggcccuagatt

SEQ ID NO: 478 - DNA, 21 bp, Artificial Sequence
  Description of Artificial Sequence Synthetic oligonucleotide
ucuagggccgugaaacucutt

SEQ ID NO: 479 - DNA, 21 bp, Artificial Sequence
  Description of Artificial Sequence Synthetic oligonucleotide
tcuuaaagcugguucauactt

SEQ ID NO: 480 - DNA, 21 bp, Artificial Sequence
  Description of Artificial Sequence Synthetic oligonucleotide
auaugaaccagcuuuaagutt

SEQ ID NO: 481 - DNA, 21 bp, Artificial Sequence
  Description of Artificial Sequence Synthetic oligonucleotide
uacacagugaccgucgacutt

SEQ ID NO: 482 - DNA, 21 bp, Artificial Sequence
  Description of Artificial Sequence Synthetic oligonucleotide
agucgacggucacuguguatt

SEQ ID NO: 483 - DNA, 21 bp, Artificial Sequence
  Description of Artificial Sequence Synthetic oligonucleotide
aaagugcgagugaucuauatt

SEQ ID NO: 484 - DNA, 21 bp, Artificial Sequence
  Description of Artificial Sequence Synthetic oligonucleotide
uauagaucacucgcacuuutt

SEQ ID NO: 485 - DNA, 21 bp, Artificial Sequence
  Description of Artificial Sequence Synthetic oligonucleotide
cagcgaacugagggugacatt

SEQ ID NO: 486 - DNA, 21 bp, Artificial Sequence
  Description of Artificial Sequence Synthetic oligonucleotide
ugucacccucaguucgcugtt

SEQ ID NO: 487 - DNA, 21 bp, Artificial Sequence
  Description of Artificial Sequence Synthetic oligonucleotide
tcaugaaugccucucgacutt

SEQ ID NO: 488 - DNA, 21 bp, Artificial Sequence
  Description of Artificial Sequence Synthetic oligonucleotide
agucgagaggcauucaugctt

SEQ ID NO: 489 - DNA, 21 bp, Artificial Sequence
  Description of Artificial Sequence Synthetic oligonucleotide
tcuuaaagcugguucauautt

SEQ ID NO: 490 - DNA, 21 bp, Artificial Sequence
  Description of Artificial Sequence Synthetic oligonucleotide
auaugaaccagcuuuaagutt

SEQ ID NO: 491 - DNA, 21 bp, Artificial Sequence
  Description of Artificial Sequence Synthetic oligonucleotide
ccgcguuaagauucccgcatt

SEQ ID NO: 492 - DNA, 21 bp, Artificial Sequence
  Description of Artificial Sequence Synthetic oligonucleotide
ugcgggaaucuuaacgcggtt

SEQ ID NO: 493 - DNA, 21 bp, Artificial Sequence
  Description of Artificial Sequence Synthetic oligonucleotide
acuccugguagaacggaugtt

SEQ ID NO: 494 - DNA, 21 bp, Artificial Sequence
  Description of Artificial Sequence Synthetic oligonucleotide
cauccguucuaccaggagutt

SEQ ID NO: 495 - DNA, 21 bp, Artificial Sequence
  Description of Artificial Sequence Synthetic oligonucleotide
gcguuaagauucccgcauutt

SEQ ID NO: 496 - DNA, 21 bp, Artificial Sequence
  Description of Artificial Sequence Synthetic oligonucleotide
aaugcgggaaucuuaacgctt

SEQ ID NO: 497 - DNA, 21 bp, Artificial Sequence
  Description of Artificial Sequence Synthetic oligonucleotide
aagcccggauagcaugaautt

SEQ ID NO: 498 - DNA, 21 bp, Artificial Sequence
  Description of Artificial Sequence Synthetic oligonucleotide
auucaugcuauccgggcuutt

SEQ ID NO: 499 - DNA, 21 bp, Artificial Sequence
  Description of Artificial Sequence Synthetic oligonucleotide
aguacacagugaccgucgatt

SEQ ID NO: 500 - DNA, 21 bp, Artificial Sequence
  Description of Artificial Sequence Synthetic oligonucleotide
ucgacggucacuguguacutt

SEQ ID NO: 501 - DNA, 21 bp, Artificial Sequence
  Description of Artificial Sequence Synthetic oligonucleotide
uucccgcauuuuaauguuutt

SEQ ID NO: 502 - DNA, 21 bp, Artificial Sequence
  Description of Artificial Sequence Synthetic oligonucleotide
aaacauuaaaaugcgggaatt

SEQ ID NO: 503 - DNA, 21 bp, Artificial Sequence
  Description of Artificial Sequence Synthetic oligonucleotide
aacuccugguagaacggautt

SEQ ID NO: 504 - DNA, 21 bp, Artificial Sequence
  Description of Artificial Sequence Synthetic oligonucleotide
auccguucuaccaggaguutt

SEQ ID NO: 505 - DNA, 21 bp, Artificial Sequence
  Description of Artificial Sequence Synthetic oligonucleotide
uuguagcaagguccguggutt

SEQ ID NO: 506 - DNA, 21 bp, Artificial Sequence
  Description of Artificial Sequence Synthetic oligonucleotide
accacggaccuugcuacaatt

SEQ ID NO: 507 - DNA, 21 bp, Artificial Sequence
  Description of Artificial Sequence Synthetic oligonucleotide
tcuuaaagcugguucauautt

SEQ ID NO: 508 - DNA, 21 bp, Artificial Sequence
  Description of Artificial Sequence Synthetic oligonucleotide
auaugaaccagcuuuaagutt

SEQ ID NO: 509 - DNA, 21 bp, Artificial Sequence
  Description of Artificial Sequence Synthetic oligonucleotide
aguacacagugaccgucgatt

SEQ ID NO: 510 - DNA, 21 bp, Artificial Sequence
  Description of Artificial Sequence Synthetic oligonucleotide
ucgacggucacuguguacutt

SEQ ID NO: 511 - DNA, 21 bp, Artificial Sequence
  Description of Artificial Sequence Synthetic oligonucleotide
cguuaagauucccgcauuutt

SEQ ID NO: 512 - DNA, 21 bp, Artificial Sequence
  Description of Artificial Sequence Synthetic oligonucleotide
aaaugcgggaaucuuaacgtt

SEQ ID NO: 513 - DNA, 21 bp, Artificial Sequence
  Description of Artificial Sequence Synthetic oligonucleotide
acaaaauuauugaccuaggtt

SEQ ID NO: 514 - DNA, 21 bp, Artificial Sequence
  Description of Artificial Sequence Synthetic oligonucleotide
ccuaggucaauaauuuugutt

SEQ ID NO: 515 - DNA, 21 bp, Artificial Sequence
  Description of Artificial Sequence Synthetic oligonucleotide
tcuuaaagcugguucauautt

SEQ ID NO: 516 - DNA, 21 bp, Artificial Sequence
  Description of Artificial Sequence Synthetic oligonucleotide
guaugaaccagcuuuaagutt

SEQ ID NO: 517 - DNA, 21 bp, Artificial Sequence
  Description of Artificial Sequence Synthetic oligonucleotide
gcuuguagcaagguccgugtt

SEQ ID NO: 518 - DNA, 21 bp, Artificial Sequence
  Description of Artificial Sequence Synthetic oligonucleotide
cacggaccuugcuacaagctt

SEQ ID NO: 519 - DNA, 21 bp, Artificial Sequence
  Description of Artificial Sequence Synthetic oligonucleotide
aacuuaaagcugguucauatt

SEQ ID NO: 520 - DNA, 21 bp, Artificial Sequence
  Description of Artificial Sequence Synthetic oligonucleotide
uaugaaccagcuuuaaguutt

SEQ ID NO: 521 - DNA, 21 bp, Artificial Sequence
  Description of Artificial Sequence Synthetic oligonucleotide
ugaccgucgacuacuggagtt

SEQ ID NO: 522 - DNA, 21 bp, Artificial Sequence
  Description of Artificial Sequence Synthetic oligonucleotide
cuccaguagucgacggucatt

SEQ ID NO: 523 - DNA, 21 bp, Artificial Sequence
  Description of Artificial Sequence Synthetic oligonucleotide
auucccgcauuuuaauguutt

SEQ ID NO: 524 - DNA, 21 bp, Artificial Sequence
  Description of Artificial Sequence Synthetic oligonucleotide
aacauuaaaaugcgggaautt

SEQ ID NO: 525 - DNA, 21 bp, Artificial Sequence
  Description of Artificial Sequence Synthetic oligonucleotide
aaugugguggcugcccgagtt

SEQ ID NO: 526 - DNA, 21 bp, Artificial Sequence
  Description of Artificial Sequence Synthetic oligonucleotide
cucgggcagccaccacauutt

SEQ ID NO: 527 - DNA, 21 bp, Artificial Sequence
  Description of Artificial Sequence Synthetic oligonucleotide
gccucucgacuuagccagctt

SEQ ID NO: 528 - DNA, 21 bp, Artificial Sequence
  Description of Artificial Sequence Synthetic oligonucleotide
gcuggcuaagucgagaggctt

SEQ ID NO: 529 - DNA, 21 bp, Artificial Sequence
  Description of Artificial Sequence Synthetic oligonucleotide
acgacaucaguaugagcugtt

SEQ ID NO: 530 - DNA, 21 bp, Artificial Sequence
  Description of Artificial Sequence Synthetic oligonucleotide
cagcucauacugaugucgutt

SEQ ID NO: 531 - DNA, 21 bp, Artificial Sequence
  Description of Artificial Sequence Synthetic oligonucleotide
cuuguagcaagguccguggtt

SEQ ID NO: 532 - DNA, 21 bp, Artificial Sequence
  Description of Artificial Sequence Synthetic oligonucleotide
ccacggaccuugcuacaagtt

SEQ ID NO: 533 - DNA, 21 bp, Artificial Sequence
  Description of Artificial Sequence Synthetic oligonucleotide
gccaugaugaaucuccucctt

SEQ ID NO: 534 - DNA, 21 bp, Artificial Sequence
  Description of Artificial Sequence Synthetic oligonucleotide
ggaggagauucaucauggctt

SEQ ID NO: 535 - DNA, 21 bp, Artificial Sequence
  Description of Artificial Sequence Synthetic oligonucleotide
ucauccgauggcacaaucatt

SEQ ID NO: 536 - DNA, 21 bp, Artificial Sequence
  Description of Artificial Sequence Synthetic oligonucleotide
ugauugugccaucggaugatt

SEQ ID NO: 537 - DNA, 21 bp, Artificial Sequence
  Description of Artificial Sequence Synthetic oligonucleotide
ggaaaugucauccgauggctt

SEQ ID NO: 538 - DNA, 21 bp, Artificial Sequence
  Description of Artificial Sequence Synthetic oligonucleotide
gccaucggaugacauuucctt

SEQ ID NO: 539 - DNA, 21 bp, Artificial Sequence
  Description of Artificial Sequence Synthetic oligonucleotide
cgugguccugucaguggaatt

SEQ ID NO: 540 - DNA, 21 bp, Artificial Sequence
  Description of Artificial Sequence Synthetic oligonucleotide
uuccacugacaggaccacgtt

SEQ ID NO: 541 - DNA, 21 bp, Artificial Sequence
  Description of Artificial Sequence Synthetic oligonucleotide
aauuauugaccuaggauautt

SEQ ID NO: 542 - DNA, 21 bp, Artificial Sequence
  Description of Artificial Sequence Synthetic oligonucleotide
auauccuaggucaauaauutt

SEQ ID NO: 543 - DNA, 21 bp, Artificial Sequence
  Description of Artificial Sequence Synthetic oligonucleotide
gccgacagaguuagcacgatt

SEQ ID NO: 544 - DNA, 21 bp, Artificial Sequence
  Description of Artificial Sequence Synthetic oligonucleotide
ucgugcuaacucugucggctt

SEQ ID NO: 545 - DNA, 21 bp, Artificial Sequence
  Description of Artificial Sequence Synthetic oligonucleotide
ugcuuguagcaagguccgutt

SEQ ID NO: 546 - DNA, 21 bp, Artificial Sequence
  Description of Artificial Sequence Synthetic oligonucleotide
acggaccuugcuacaagcatt

SEQ ID NO: 547 - DNA, 21 bp, Artificial Sequence
  Description of Artificial Sequence Synthetic oligonucleotide
aguggaagcccggauagcatt

SEQ ID NO: 548 - DNA, 21 bp, Artificial Sequence
  Description of Artificial Sequence Synthetic oligonucleotide
ugcuauccgggcuuccacutt

SEQ ID NO: 549 - DNA, 21 bp, Artificial Sequence
  Description of Artificial Sequence Synthetic oligonucleotide
aagugucagcuguauccuutt

SEQ ID NO: 550 - DNA, 21 bp, Artificial Sequence
  Description of Artificial Sequence Synthetic oligonucleotide
aaggauacagcugacacuutt

SEQ ID NO: 551 - DNA, 21 bp, Artificial Sequence
  Description of Artificial Sequence Synthetic oligonucleotide
caggaaaugguacggcugctt

SEQ ID NO: 552 - DNA, 21 bp, Artificial Sequence
  Description of Artificial Sequence Synthetic oligonucleotide
gcagccguaccauuuccugtt

SEQ ID NO: 553 - DNA, 21 bp, Artificial Sequence
  Description of Artificial Sequence Synthetic oligonucleotide
gucccugccgacagaguuatt

SEQ ID NO: 554 - DNA, 21 bp, Artificial Sequence
  Description of Artificial Sequence Synthetic oligonucleotide
uaacucugucggcagggactt

SEQ ID NO: 555 - DNA, 21 bp, Artificial Sequence
  Description of Artificial Sequence Synthetic oligonucleotide
cgcguuaagauucccgcautt

SEQ ID NO: 556 - DNA, 21 bp, Artificial Sequence
  Description of Artificial Sequence Synthetic oligonucleotide
augcgggaaucuuaacgcgtt

SEQ ID NO: 557 - DNA, 21 bp, Artificial Sequence
  Description of Artificial Sequence Synthetic oligonucleotide
aagugcgagugaucuauactt

SEQ ID NO: 558 - DNA, 21 bp, Artificial Sequence
  Description of Artificial Sequence Synthetic oligonucleotide
guauagaucacucgcacuutt

SEQ ID NO: 559 - DNA, 21 bp, Artificial Sequence
  Description of Artificial Sequence Synthetic oligonucleotide
gaaugccucucgacuuagctt

SEQ ID NO: 560 - DNA, 21 bp, Artificial Sequence
  Description of Artificial Sequence Synthetic oligonucleotide
gcuaagucgagaggcauuctt

SEQ ID NO: 561 - DNA, 21 bp, Artificial Sequence
  Description of Artificial Sequence Synthetic oligonucleotide
cgagaccagcgaacugaggtt

SEQ ID NO: 562 - DNA, 21 bp, Artificial Sequence
  Description of Artificial Sequence Synthetic oligonucleotide
ccucaguucgcuggucucgtt

SEQ ID NO: 563 - DNA, 21 bp, Artificial Sequence
  Description of Artificial Sequence Synthetic oligonucleotide
uguagcaagguccgugguctt

SEQ ID NO: 564 - DNA, 21 bp, Artificial Sequence
  Description of Artificial Sequence Synthetic oligonucleotide
gaccacggaccuugcuacatt

SEQ ID NO: 565 - DNA, 21 bp, Artificial Sequence
  Description of Artificial Sequence Synthetic oligonucleotide
uuagcacgacaucaguaugtt

SEQ ID NO: 566 - DNA, 21 bp, Artificial Sequence
  Description of Artificial Sequence Synthetic oligonucleotide
cauacugaugucgugcuaatt

SEQ ID NO: 567 - DNA, 21 bp, Artificial Sequence
  Description of Artificial Sequence Synthetic oligonucleotide
cuguacaggagacuaagggtt

SEQ ID NO: 568 - DNA, 21 bp, Artificial Sequence
  Description of Artificial Sequence Synthetic oligonucleotide
cccuuagucuccuguacagtt

SEQ ID NO: 569 - DNA, 21 bp, Artificial Sequence
  Description of Artificial Sequence Synthetic oligonucleotide
gagaacgaagugaaacucctt

SEQ ID NO: 570 - DNA, 21 bp, Artificial Sequence
  Description of Artificial Sequence Synthetic oligonucleotide
ggaguuucacuucguucuctt

SEQ ID NO: 571 - DNA, 21 bp, Artificial Sequence
  Description of Artificial Sequence Synthetic oligonucleotide
gaacuuggcgcccaaugactt

SEQ ID NO: 572 - DNA, 21 bp, Artificial Sequence
  Description of Artificial Sequence Synthetic oligonucleotide
gucauugggcgccaaguuctt

SEQ ID NO: 573 - DNA, 21 bp, Artificial Sequence
  Description of Artificial Sequence Synthetic oligonucleotide
gcugcccgcguuaagauuctt

SEQ ID NO: 574 - DNA, 21 bp, Artificial Sequence
  Description of Artificial Sequence Synthetic oligonucleotide
gaaucuuaacgcgggcagctt

SEQ ID NO: 575 - DNA, 21 bp, Artificial Sequence
  Description of Artificial Sequence Synthetic oligonucleotide
aagcugguucauaucuugatt

SEQ ID NO: 576 - DNA, 21 bp, Artificial Sequence
  Description of Artificial Sequence Synthetic oligonucleotide
ucaagauaugaaccagcuutt

SEQ ID NO: 577 - DNA, 21 bp, Artificial Sequence
  Description of Artificial Sequence Synthetic oligonucleotide
gcccgcguuaagauucccgtt

SEQ ID NO: 578 - DNA, 21 bp, Artificial Sequence
  Description of Artificial Sequence Synthetic oligonucleotide
cgggaaucuuaacgcgggctt

SEQ ID NO: 579 - DNA, 21 bp, Artificial Sequence
  Description of Artificial Sequence Synthetic oligonucleotide
gaggaagucgcgccgcgcutt

SEQ ID NO: 580 - DNA, 21 bp, Artificial Sequence
  Description of Artificial Sequence Synthetic oligonucleotide
agcgcggcgcgacuuccuctt

SEQ ID NO: 581 - DNA, 21 bp, Artificial Sequence
  Description of Artificial Sequence Synthetic oligonucleotide
cucgagaccagcgaacugatt

SEQ ID NO: 582 - DNA, 21 bp, Artificial Sequence
  Description of Artificial Sequence Synthetic oligonucleotide
ucaguucgcuggucucgagtt

SEQ ID NO: 583 - DNA, 21 bp, Artificial Sequence
  Description of Artificial Sequence Synthetic oligonucleotide
agagguggugagcuuaaugtt

SEQ ID NO: 584 - DNA, 21 bp, Artificial Sequence
  Description of Artificial Sequence Synthetic oligonucleotide
cauuaagcucaccaccucutt

SEQ ID NO: 585 - DNA, 21 bp, Artificial Sequence
  Description of Artificial Sequence Synthetic oligonucleotide
gagguggugagcuuaaugatt

SEQ ID NO: 586 - DNA, 21 bp, Artificial Sequence
  Description of Artificial Sequence Synthetic oligonucleotide
ucauuaagcucaccaccuctt

SEQ ID NO: 587 - DNA, 21 bp, Artificial Sequence
  Description of Artificial Sequence Synthetic oligonucleotide
gaguuucacggcccuagactt

SEQ ID NO: 588 - DNA, 21 bp, Artificial Sequence
  Description of Artificial Sequence Synthetic oligonucleotide
gucuagggccgugaaacuctt

SEQ ID NO: 589 - DNA, 21 bp, Artificial Sequence
  Description of Artificial Sequence Synthetic oligonucleotide
cggccuccaacagcuuacctt

SEQ ID NO: 590 - DNA, 21 bp, Artificial Sequence
  Description of Artificial Sequence Synthetic oligonucleotide
gguaagcuguuggaggccgtt

SEQ ID NO: 591 - DNA, 21 bp, Artificial Sequence
  Description of Artificial Sequence Synthetic oligonucleotide
agcccggauagcaugaaugtt

SEQ ID NO: 592 - DNA, 21 bp, Artificial Sequence
  Description of Artificial Sequence Synthetic oligonucleotide
cauucaugcuauccgggcutt

SEQ ID NO: 593 - DNA, 21 bp, Artificial Sequence
  Description of Artificial Sequence Synthetic oligonucleotide
tcuuaaagcugguucauactt

SEQ ID NO: 594 - DNA, 21 bp, Artificial Sequence
  Description of Artificial Sequence Synthetic oligonucleotide
guaugaaccagcuuuaagutt

SEQ ID NO: 595 - DNA, 21 bp, Artificial Sequence
  Description of Artificial Sequence Synthetic oligonucleotide
gcgggccuggcguugaucctt

SEQ ID NO: 596 - DNA, 21 bp, Artificial Sequence
  Description of Artificial Sequence Synthetic oligonucleotide
ggaucaacgccaggcccgctt

SEQ ID NO: 597 - DNA, 21 bp, Artificial Sequence
  Description of Artificial Sequence Synthetic oligonucleotide
ugcccgcguuaagauuccctt

SEQ ID NO: 598 - DNA, 21 bp, Artificial Sequence
  Description of Artificial Sequence Synthetic oligonucleotide
gggaaucuuaacgcgggcatt

SEQ ID NO: 599 - DNA, 21 bp, Artificial Sequence
  Description of Artificial Sequence Synthetic oligonucleotide
agauucccgcauuuuaaugtt

SEQ ID NO: 600 - DNA, 21 bp, Artificial Sequence
  Description of Artificial Sequence Synthetic oligonucleotide
cauuaaaaugcgggaaucutt

SEQ ID NO: 601 - DNA, 21 bp, Artificial Sequence
  Description of Artificial Sequence Synthetic oligonucleotide
ucucgacuuagccagccugtt

SEQ ID NO: 602 - DNA, 21 bp, Artificial Sequence
  Description of Artificial Sequence Synthetic oligonucleotide
caggcuggcuaagucgagatt

SEQ ID NO: 603 - DNA, 21 bp, Artificial Sequence
  Description of Artificial Sequence Synthetic oligonucleotide
caaugugguggcugcccgatt

SEQ ID NO: 604 - DNA, 21 bp, Artificial Sequence
  Description of Artificial Sequence Synthetic oligonucleotide
ucgggcagccaccacauugtt

SEQ ID NO: 605 - DNA, 21 bp, Artificial Sequence
  Description of Artificial Sequence Synthetic oligonucleotide
aaacugugguuugcaagcatt

SEQ ID NO: 606 - DNA, 21 bp, Artificial Sequence
  Description of Artificial Sequence Synthetic oligonucleotide
ugcuugcaaaccacaguuutt

SEQ ID NO: 607 - DNA, 21 bp, Artificial Sequence
  Description of Artificial Sequence Synthetic oligonucleotide
ccgcgucccugccgacagatt

SEQ ID NO: 608 - DNA, 21 bp, Artificial Sequence
  Description of Artificial Sequence Synthetic oligonucleotide
ucugucggcagggacgcggtt

SEQ ID NO: 609 - DNA, 21 bp, Artificial Sequence
  Description of Artificial Sequence Synthetic oligonucleotide
ugggaucacaucagauaaatt

SEQ ID NO: 610 - DNA, 21 bp, Artificial Sequence
  Description of Artificial Sequence Synthetic oligonucleotide
uuuaucugaugugaucccatt

SEQ ID NO: 611 - DNA, 21 bp, Artificial Sequence
  Description of Artificial Sequence Synthetic oligonucleotide
aacagaaucauccaucgggtt

SEQ ID NO: 612 - DNA, 21 bp, Artificial Sequence
  Description of Artificial Sequence Synthetic oligonucleotide
cccgauggaugauucuguutt

SEQ ID NO: 613 - DNA, 21 bp, Artificial Sequence
  Description of Artificial Sequence Synthetic oligonucleotide
aaugccucucgacuuagcctt

SEQ ID NO: 614 - DNA, 21 bp, Artificial Sequence
  Description of Artificial Sequence Synthetic oligonucleotide
ggcuaagucgagaggcauutt

SEQ ID NO: 615 - DNA, 21 bp, Artificial Sequence
  Description of Artificial Sequence Synthetic oligonucleotide
uguacaggagacuaagggatt

SEQ ID NO: 616 - DNA, 21 bp, Artificial Sequence
  Description of Artificial Sequence Synthetic oligonucleotide
ucccuuagucuccuguacatt

SEQ ID NO: 617 - DNA, 21 bp, Artificial Sequence
  Description of Artificial Sequence Synthetic oligonucleotide
ugugggcgggagaacgaagtt

SEQ ID NO: 618 - DNA, 21 bp, Artificial Sequence
  Description of Artificial Sequence Synthetic oligonucleotide
cuucguucucccgcccacatt

SEQ ID NO: 619 - DNA, 21 bp, Artificial Sequence
  Description of Artificial Sequence Synthetic oligonucleotide
ucugugggcgggagaacgatt

SEQ ID NO: 620 - DNA, 21 bp, Artificial Sequence
  Description of Artificial Sequence Synthetic oligonucleotide
ucguucucccgcccacagatt

SEQ ID NO: 621 - DNA, 21 bp, Artificial Sequence
  Description of Artificial Sequence Synthetic oligonucleotide
agauugcuuguagcaaggutt

SEQ ID NO: 622 - DNA, 21 bp, Artificial Sequence
  Description of Artificial Sequence Synthetic oligonucleotide
accuugcuacaagcaaucutt

SEQ ID NO: 623 - DNA, 21 bp, Artificial Sequence
  Description of Artificial Sequence Synthetic oligonucleotide
gcuagaaaaugccauacagtt

SEQ ID NO: 624 - DNA, 21 bp, Artificial Sequence
  Description of Artificial Sequence Synthetic oligonucleotide
cuguauggcauuuucuagctt

SEQ ID NO: 625 - DNA, 21 bp, Artificial Sequence
  Description of Artificial Sequence Synthetic oligonucleotide
cagaaucauccaucgggautt

SEQ ID NO: 626 - DNA, 21 bp, Artificial Sequence
  Description of Artificial Sequence Synthetic oligonucleotide
aucccgauggaugauucugtt

SEQ ID NO: 627 - DNA, 21 bp, Artificial Sequence
  Description of Artificial Sequence Synthetic oligonucleotide
cagaaucauccaucgggautt

SEQ ID NO: 628 - DNA, 21 bp, Artificial Sequence
  Description of Artificial Sequence Synthetic oligonucleotide
aucccgauggaugauucugtt

SEQ ID NO: 629 - DNA, 21 bp, Artificial Sequence
  Description of Artificial Sequence Synthetic oligonucleotide
ugguucauaucuugaacautt

SEQ ID NO: 630 - DNA, 21 bp, Artificial Sequence
  Description of Artificial Sequence Synthetic oligonucleotide
auguucaagauaugaaccatt

SEQ ID NO: 631 - DNA, 21 bp, Artificial Sequence
  Description of Artificial Sequence Synthetic oligonucleotide
cagaaucauccaucgggautt

SEQ ID NO: 632 - DNA, 21 bp, Artificial Sequence
  Description of Artificial Sequence Synthetic oligonucleotide
aucccgauggaugauucugtt

SEQ ID NO: 633 - DNA, 21 bp, Artificial Sequence
  Description of Artificial Sequence Synthetic oligonucleotide
cagaaucauccaucgggautt

SEQ ID NO: 634 - DNA, 21 bp, Artificial Sequence
  Description of Artificial Sequence Synthetic oligonucleotide
aucccgauggaugauucugtt

SEQ ID NO: 635 - DNA, 21 bp, Artificial Sequence
  Description of Artificial Sequence Synthetic oligonucleotide
gcaagggagcuguacaggatt

SEQ ID NO: 636 - DNA, 21 bp, Artificial Sequence
  Description of Artificial Sequence Synthetic oligonucleotide
uccuguacagcucccuugctt

SEQ ID NO: 637 - DNA, 21 bp, Artificial Sequence
  Description of Artificial Sequence Synthetic oligonucleotide
ugguucauaucuugaacautt

SEQ ID NO: 638 - DNA, 21 bp, Artificial Sequence
  Description of Artificial Sequence Synthetic oligonucleotide
auguucaagauaugaaccatt

SEQ ID NO: 639 - DNA, 21 bp, Artificial Sequence
  Description of Artificial Sequence Synthetic oligonucleotide
gcuagaaaaugccauacagtt

SEQ ID NO: 640 - DNA, 21 bp, Artificial Sequence
  Description of Artificial Sequence Synthetic oligonucleotide
cuguauggcauuuucuagctt

SEQ ID NO: 641 - DNA, 21 bp, Artificial Sequence
  Description of Artificial Sequence Synthetic oligonucleotide
cagaaucauccaucgggautt

SEQ ID NO: 642 - DNA, 21 bp, Artificial Sequence
  Description of Artificial Sequence Synthetic oligonucleotide
aucccgauggaugauucugtt

SEQ ID NO: 643 - DNA, 21 bp, Artificial Sequence
  Description of Artificial Sequence Synthetic oligonucleotide
cagaaucauccaucgggautt

SEQ ID NO: 644 - DNA, 21 bp, Artificial Sequence
  Description of Artificial Sequence Synthetic oligonucleotide
aucccgauggaugauucugtt

SEQ ID NO: 645 - RNA, 19 nt, Artificial Sequence
  Description of Artificial Sequence Synthetic oligonucleotide
ggcgggagaaugacgugaa

SEQ ID NO: 646 - RNA, 19 nt, Artificial Sequence
  Description of Artificial Sequence Synthetic oligonucleotide
ugggcgggagaaugacgug

SEQ ID NO: 647 - RNA, 19 nt, Artificial Sequence
  Description of Artificial Sequence Synthetic oligonucleotide
guagcaaaguccgaggucc

SEQ ID NO: 648 - RNA, 19 nt, Artificial Sequence
  Description of Artificial Sequence Synthetic oligonucleotide
gaccgugguuuguaagcag

SEQ ID NO: 649 - RNA, 19 nt, Artificial Sequence
  Description of Artificial Sequence Synthetic oligonucleotide
agaccgugguuuguaagca

SEQ ID NO: 650 - RNA, 19 nt, Artificial Sequence
  Description of Artificial Sequence Synthetic oligonucleotide
guaagaccgugguuuguaa

SEQ ID NO: 651 - RNA, 19 nt, Artificial Sequence
  Description of Artificial Sequence Synthetic oligonucleotide
caaaauuauugaucuagga

SEQ ID NO: 652 - RNA, 19 nt, Artificial Sequence
  Description of Artificial Sequence Synthetic oligonucleotide
cucaguaagaccgugguuu

SEQ ID NO: 653 - RNA, 19 nt, Artificial Sequence
  Description of Artificial Sequence Synthetic oligonucleotide
ucggcucuuagauaccuuc

SEQ ID NO: 654 - RNA, 19 nt, Artificial Sequence
  Description of Artificial Sequence Synthetic oligonucleotide
agcaugaaugugucucgac

SEQ ID NO: 655 - RNA, 19 nt, Artificial Sequence
  Description of Artificial Sequence Synthetic oligonucleotide
auugaucuaggauaugcca

SEQ ID NO: 656 - RNA, 19 nt, Artificial Sequence
  Description of Artificial Sequence Synthetic oligonucleotide
gaagaucgccuguagcaaa

SEQ ID NO: 657 - DNA, 20 bp, Artificial Sequence
  Description of Artificial Sequence Synthetic oligonucleotide
tggcgggagaaugacgugaa

SEQ ID NO: 658 - DNA, 20 bp, Artificial Sequence
  Description of Artificial Sequence Synthetic oligonucleotide
tguagcaaaguccgaggucc

SEQ ID NO: 659 - RNA, 19 nt, Artificial Sequence
  Description of Artificial Sequence Synthetic oligonucleotide
aucgauaugagcuggucac

SEQ ID NO: 660 - RNA, 19 nt, Artificial Sequence
  Description of Artificial Sequence Synthetic oligonucleotide
gauacuugaaccaguucga

SEQ ID NO: 661 - RNA, 19 nt, Artificial Sequence
  Description of Artificial Sequence Synthetic oligonucleotide
aucggcucuuagauaccuu

SEQ ID NO: 662 - RNA, 19 nt, Artificial Sequence
  Description of Artificial Sequence Synthetic oligonucleotide
gccaucaagcaaugccgac

SEQ ID NO: 663 - RNA, 19 nt, Artificial Sequence
  Description of Artificial Sequence Synthetic oligonucleotide
ucaguaagaccgugguuug

SEQ ID NO: 664 - RNA, 19 nt, Artificial Sequence
  Description of Artificial Sequence Synthetic oligonucleotide
ucgccuguagcaaaguccg

SEQ ID NO: 665 - RNA, 19 nt, Artificial Sequence
  Description of Artificial Sequence Synthetic oligonucleotide
gccuguagcaaaguccgag

SEQ ID NO: 666 - RNA, 19 nt, Artificial Sequence
  Description of Artificial Sequence Synthetic oligonucleotide
ggagcccgaugggucggaa

SEQ ID NO: 667 - RNA, 19 nt, Artificial Sequence
  Description of Artificial Sequence Synthetic oligonucleotide
ccaucaagcaaugccgaca

SEQ ID NO: 668 - RNA, 19 nt, Artificial Sequence
  Description of Artificial Sequence Synthetic oligonucleotide
cggcucuuagauaccuuca

SEQ ID NO: 669 - RNA, 19 nt, Artificial Sequence
  Description of Artificial Sequence Synthetic oligonucleotide
uauugaucuaggauaugcc

SEQ ID NO: 670 - RNA, 19 nt, Artificial Sequence
  Description of Artificial Sequence Synthetic oligonucleotide
uaagaccgugguuuguaag

SEQ ID NO: 671 - RNA, 19 nt, Artificial Sequence
  Description of Artificial Sequence Synthetic oligonucleotide
caucgauaugagcugguca

SEQ ID NO: 672 - RNA, 19 nt, Artificial Sequence
  Description of Artificial Sequence Synthetic oligonucleotide
uguagcaaaguccgagguc

SEQ ID NO: 673 - RNA, 19 nt, Artificial Sequence
  Description of Artificial Sequence Synthetic oligonucleotide
gggcgggagaaugacguga

SEQ ID NO: 674 - RNA, 19 nt, Artificial Sequence
  Description of Artificial Sequence Synthetic oligonucleotide
gaucgccuguagcaaaguc

SEQ ID NO: 675 - RNA, 19 nt, Artificial Sequence
  Description of Artificial Sequence Synthetic oligonucleotide
ucgauaugagcuggucacc

SEQ ID NO: 676 - RNA, 19 nt, Artificial Sequence
  Description of Artificial Sequence Synthetic oligonucleotide
aggagcccgaugggucgga

SEQ ID NO: 677 - RNA, 19 nt, Artificial Sequence
  Description of Artificial Sequence Synthetic oligonucleotide
ugggaaaugaaagaacgcc

SEQ ID NO: 678 - RNA, 19 nt, Artificial Sequence
  Description of Artificial Sequence Synthetic oligonucleotide
ggaagacuguaaccggcug

SEQ ID NO: 679 - RNA, 19 nt, Artificial Sequence
  Description of Artificial Sequence Synthetic oligonucleotide
aucgccuguagcaaagucc

SEQ ID NO: 680 - RNA, 19 nt, Artificial Sequence
  Description of Artificial Sequence Synthetic oligonucleotide
cccgaguuuucauguccuc

SEQ ID NO: 681 - RNA, 19 nt, Artificial Sequence
  Description of Artificial Sequence Synthetic oligonucleotide
ccgaugggucggaagcagg

SEQ ID NO: 682 - RNA, 19 nt, Artificial Sequence
  Description of Artificial Sequence Synthetic oligonucleotide
cgccuguagcaaaguccga

SEQ ID NO: 683 - RNA, 19 nt, Artificial Sequence
  Description of Artificial Sequence Synthetic oligonucleotide
gaagacuguaaccggcugc

SEQ ID NO: 684 - RNA, 19 nt, Artificial Sequence
  Description of Artificial Sequence Synthetic oligonucleotide
aagacuguaaccggcugca

SEQ ID NO: 685 - RNA, 19 nt, Artificial Sequence
  Description of Artificial Sequence Synthetic oligonucleotide
acgucauccgguggcacaa

SEQ ID NO: 686 - RNA, 19 nt, Artificial Sequence
  Description of Artificial Sequence Synthetic oligonucleotide
ggaaacgucauccgguggc

SEQ ID NO: 687 - RNA, 19 nt, Artificial Sequence
  Description of Artificial Sequence Synthetic oligonucleotide
gauuuggaaacgucauccg

SEQ ID NO: 688 - RNA, 19 nt, Artificial Sequence
  Description of Artificial Sequence Synthetic oligonucleotide
uguccucagcgggugucgc

SEQ ID NO: 689 - RNA, 19 nt, Artificial Sequence
  Description of Artificial Sequence Synthetic oligonucleotide
aaacgucauccgguggcac

SEQ ID NO: 690 - RNA, 19 nt, Artificial Sequence
  Description of Artificial Sequence Synthetic oligonucleotide
cauccgguggcacaaucag

SEQ ID NO: 691 - RNA, 19 nt, Artificial Sequence
  Description of Artificial Sequence Synthetic oligonucleotide
uggaaacgucauccggugg

SEQ ID NO: 692 - RNA, 19 nt, Artificial Sequence
  Description of Artificial Sequence Synthetic oligonucleotide
cauguccucagcggguguc

SEQ ID NO: 693 - RNA, 19 nt, Artificial Sequence
  Description of Artificial Sequence Synthetic oligonucleotide
uucacgucauucucccgcc

SEQ ID NO: 694 - RNA, 19 nt, Artificial Sequence
  Description of Artificial Sequence Synthetic oligonucleotide
cacgucauucucccgccca

SEQ ID NO: 695 - RNA, 19 nt, Artificial Sequence
  Description of Artificial Sequence Synthetic oligonucleotide
ggaccucggacuuugcuac

SEQ ID NO: 696 - RNA, 19 nt, Artificial Sequence
  Description of Artificial Sequence Synthetic oligonucleotide
cugcuuacaaaccacgguc

SEQ ID NO: 697 - RNA, 19 nt, Artificial Sequence
  Description of Artificial Sequence Synthetic oligonucleotide
ugcuuacaaaccacggucu

SEQ ID NO: 698 - RNA, 19 nt, Artificial Sequence
  Description of Artificial Sequence Synthetic oligonucleotide
uuacaaaccacggucuuac

SEQ ID NO: 699 - RNA, 19 nt, Artificial Sequence
  Description of Artificial Sequence Synthetic oligonucleotide
uccuagaucaauaauuuug

SEQ ID NO: 700 - RNA, 19 nt, Artificial Sequence
  Description of Artificial Sequence Synthetic oligonucleotide
aaaccacggucuuacugag

SEQ ID NO: 701 - RNA, 19 nt, Artificial Sequence
  Description of Artificial Sequence Synthetic oligonucleotide
gaagguaucuaagagccga

SEQ ID NO: 702 - RNA, 19 nt, Artificial Sequence
  Description of Artificial Sequence Synthetic oligonucleotide
gucgagacacauucaugcu

SEQ ID NO: 703 - RNA, 19 nt, Artificial Sequence
  Description of Artificial Sequence Synthetic oligonucleotide
uggcauauccuagaucaau

SEQ ID NO: 704 - RNA, 20 nt, Artificial Sequence
  Description of Artificial Sequence Synthetic oligonucleotide
uuucacgucauucucccgcc

SEQ ID NO: 705 - RNA, 19 nt, Artificial Sequence
  Description of Artificial Sequence Synthetic oligonucleotide
gugaccagcucauaucgau

SEQ ID NO: 706 - RNA, 19 nt, Artificial Sequence
  Description of Artificial Sequence Synthetic oligonucleotide
ucgaacugguucaaguauc

SEQ ID NO: 707 - RNA, 19 nt, Artificial Sequence
  Description of Artificial Sequence Synthetic oligonucleotide
aagguaucuaagagccgau

SEQ ID NO: 708 - RNA, 19 nt, Artificial Sequence
  Description of Artificial Sequence Synthetic oligonucleotide
gucggcauugcuugauggc

SEQ ID NO: 709 - RNA, 19 nt, Artificial Sequence
  Description of Artificial Sequence Synthetic oligonucleotide
caaaccacggucuuacuga

SEQ ID NO: 710 - RNA, 19 nt, Artificial Sequence
  Description of Artificial Sequence Synthetic oligonucleotide
cggacuuugcuacaggcga

SEQ ID NO: 711 - RNA, 19 nt, Artificial Sequence
  Description of Artificial Sequence Synthetic oligonucleotide
cucggacuuugcuacaggc

SEQ ID NO: 712 - RNA, 19 nt, Artificial Sequence
  Description of Artificial Sequence Synthetic oligonucleotide
uuccgacccaucgggcucc

SEQ ID NO: 713 - RNA, 19 nt, Artificial Sequence
  Description of Artificial Sequence Synthetic oligonucleotide
ugucggcauugcuugaugg

SEQ ID NO: 714 - RNA, 19 nt, Artificial Sequence
  Description of Artificial Sequence Synthetic oligonucleotide
ugaagguaucuaagagccg

SEQ ID NO: 715 - RNA, 19 nt, Artificial Sequence
  Description of Artificial Sequence Synthetic oligonucleotide
ggcauauccuagaucaaua

SEQ ID NO: 716 - RNA, 19 nt, Artificial Sequence
  Description of Artificial Sequence Synthetic oligonucleotide
cuuacaaaccacggucuua

SEQ ID NO: 717 - RNA, 19 nt, Artificial Sequence
  Description of Artificial Sequence Synthetic oligonucleotide
ugaccagcucauaucgaug

SEQ ID NO: 718 - RNA, 19 nt, Artificial Sequence
  Description of Artificial Sequence Synthetic oligonucleotide
gaccucggacuuugcuaca

SEQ ID NO: 719 - RNA, 19 nt, Artificial Sequence
  Description of Artificial Sequence Synthetic oligonucleotide
ucacgucauucucccgccc

SEQ ID NO: 720 - RNA, 19 nt, Artificial Sequence
  Description of Artificial Sequence Synthetic oligonucleotide
gacuuugcuacaggcgauc

SEQ ID NO: 721 - RNA, 19 nt, Artificial Sequence
  Description of Artificial Sequence Synthetic oligonucleotide
ggugaccagcucauaucga

SEQ ID NO: 722 - RNA, 19 nt, Artificial Sequence
  Description of Artificial Sequence Synthetic oligonucleotide
uccgacccaucgggcuccu

SEQ ID NO: 723 - RNA, 19 nt, Artificial Sequence
  Description of Artificial Sequence Synthetic oligonucleotide
ggcguucuuucauuuccca

SEQ ID NO: 724 - RNA, 19 nt, Artificial Sequence
  Description of Artificial Sequence Synthetic oligonucleotide
cagccgguuacagucuucc

SEQ ID NO: 725 - RNA, 19 nt, Artificial Sequence
  Description of Artificial Sequence Synthetic oligonucleotide
ggacuuugcuacaggcgau

SEQ ID NO: 726 - RNA, 19 nt, Artificial Sequence
  Description of Artificial Sequence Synthetic oligonucleotide
gaggacaugaaaacucggg

SEQ ID NO: 727 - RNA, 19 nt, Artificial Sequence
  Description of Artificial Sequence Synthetic oligonucleotide
ccugcuuccgacccaucgg

SEQ ID NO: 728 - RNA, 19 nt, Artificial Sequence
  Description of Artificial Sequence Synthetic oligonucleotide
ucggacuuugcuacaggcg

SEQ ID NO: 729 - RNA, 19 nt, Artificial Sequence
  Description of Artificial Sequence Synthetic oligonucleotide
gcagccgguuacagucuuc

SEQ ID NO: 730 - RNA, 19 nt, Artificial Sequence
  Description of Artificial Sequence Synthetic oligonucleotide
ugcagccgguuacagucuu

SEQ ID NO: 731 - RNA, 19 nt, Artificial Sequence
  Description of Artificial Sequence Synthetic oligonucleotide
uugugccaccggaugacgu

SEQ ID NO: 732 - RNA, 19 nt, Artificial Sequence
  Description of Artificial Sequence Synthetic oligonucleotide
gccaccggaugacguuucc

SEQ ID NO: 733 - RNA, 19 nt, Artificial Sequence
  Description of Artificial Sequence Synthetic oligonucleotide
cggaugacguuuccaaauc

SEQ ID NO: 734 - RNA, 19 nt, Artificial Sequence
  Description of Artificial Sequence Synthetic oligonucleotide
gcgacacccgcugaggaca

SEQ ID NO: 735 - RNA, 19 nt, Artificial Sequence
  Description of Artificial Sequence Synthetic oligonucleotide
gugccaccggaugacguuu

SEQ ID NO: 736 - RNA, 19 nt, Artificial Sequence
  Description of Artificial Sequence Synthetic oligonucleotide
cugauugugccaccggaug

SEQ ID NO: 737 - RNA, 19 nt, Artificial Sequence
  Description of Artificial Sequence Synthetic oligonucleotide
ccaccggaugacguuucca

SEQ ID NO: 738 - RNA, 19 nt, Artificial Sequence
  Description of Artificial Sequence Synthetic oligonucleotide
gacacccgcugaggacaug

SEQ ID NO: 739 - DNA, 21 bp, Artificial Sequence
  Description of Artificial Sequence Synthetic oligonucleotide
ggcgggagaaugacgugaatt

SEQ ID NO: 740 - DNA, 21 bp, Artificial Sequence
  Description of Artificial Sequence Synthetic oligonucleotide
uucacgucauucucccgcctt

SEQ ID NO: 741 - DNA, 21 bp, Artificial Sequence
  Description of Artificial Sequence Synthetic oligonucleotide
ugggcgggagaaugacgugtt

SEQ ID NO: 742 - DNA, 21 bp, Artificial Sequence
  Description of Artificial Sequence Synthetic oligonucleotide
cacgucauucucccgcccatt

SEQ ID NO: 743 - DNA, 21 bp, Artificial Sequence
  Description of Artificial Sequence Synthetic oligonucleotide
guagcaaaguccgaggucctt

SEQ ID NO: 744 - DNA, 21 bp, Artificial Sequence
  Description of Artificial Sequence Synthetic oligonucleotide
ggaccucggacuuugcuactt

SEQ ID NO: 745 - DNA, 21 bp, Artificial Sequence
  Description of Artificial Sequence Synthetic oligonucleotide
gaccgugguuuguaagcagtt

SEQ ID NO: 746 - DNA, 21 bp, Artificial Sequence
  Description of Artificial Sequence Synthetic oligonucleotide
cugcuuacaaaccacgguctt

SEQ ID NO: 747 - DNA, 21 bp, Artificial Sequence
  Description of Artificial Sequence Synthetic oligonucleotide
agaccgugguuuguaagcatt

SEQ ID NO: 748 - DNA, 21 bp, Artificial Sequence
  Description of Artificial Sequence Synthetic oligonucleotide
ugcuuacaaaccacggucutt

SEQ ID NO: 749 - DNA, 21 bp, Artificial Sequence
  Description of Artificial Sequence Synthetic oligonucleotide
guaagaccgugguuuguaatt

SEQ ID NO: 750 - DNA, 21 bp, Artificial Sequence
  Description of Artificial Sequence Synthetic oligonucleotide
uuacaaaccacggucuuactt

SEQ ID NO: 751 - DNA, 21 bp, Artificial Sequence
  Description of Artificial Sequence Synthetic oligonucleotide
caaaauuauugaucuaggatt

SEQ ID NO: 752 - DNA, 21 bp, Artificial Sequence
  Description of Artificial Sequence Synthetic oligonucleotide
uccuagaucaauaauuuugtt

SEQ ID NO: 753 - DNA, 21 bp, Artificial Sequence
  Description of Artificial Sequence Synthetic oligonucleotide
cucaguaagaccgugguuutt

SEQ ID NO: 754 - DNA, 21 bp, Artificial Sequence
  Description of Artificial Sequence Synthetic oligonucleotide
aaaccacggucuuacugagtt

SEQ ID NO: 755 - DNA, 21 bp, Artificial Sequence
  Description of Artificial Sequence Synthetic oligonucleotide
ucggcucuuagauaccuuctt

SEQ ID NO: 756 - DNA, 21 bp, Artificial Sequence
  Description of Artificial Sequence Synthetic oligonucleotide
gaagguaucuaagagccgatt

SEQ ID NO: 757 - DNA, 21 bp, Artificial Sequence
  Description of Artificial Sequence Synthetic oligonucleotide
agcaugaaugugucucgactt

SEQ ID NO: 758 - DNA, 21 bp, Artificial Sequence
  Description of Artificial Sequence Synthetic oligonucleotide
gucgagacacauucaugcutt

SEQ ID NO: 759 - DNA, 21 bp, Artificial Sequence
  Description of Artificial Sequence Synthetic oligonucleotide
auugaucuaggauaugccatt

SEQ ID NO: 760 - DNA, 21 bp, Artificial Sequence
  Description of Artificial Sequence Synthetic oligonucleotide
uggcauauccuagaucaautt

SEQ ID NO: 761 - DNA, 21 bp, Artificial Sequence
  Description of Artificial Sequence Synthetic oligonucleotide
gaagaucgccuguagcaaatt

SEQ ID NO: 762 - DNA, 21 bp, Artificial Sequence
  Description of Artificial Sequence Synthetic oligonucleotide
uuugcuacaggcgaucuuctt

SEQ ID NO: 763 - DNA, 21 bp, Artificial Sequence
  Description of Artificial Sequence Synthetic oligonucleotide
guagcaaaguccgaggucctt

SEQ ID NO: 764 - DNA, 21 bp, Artificial Sequence
  Description of Artificial Sequence Synthetic oligonucleotide
ggaccucggacuuugcuactt

SEQ ID NO: 765 - DNA, 21 bp, Artificial Sequence
  Description of Artificial Sequence Synthetic oligonucleotide
ggcgggagaaugacgugaatt

SEQ ID NO: 766 - DNA, 21 bp, Artificial Sequence
  Description of Artificial Sequence Synthetic oligonucleotide
uucacgucauucucccgcctt

SEQ ID NO: 767 - DNA, 21 bp, Artificial Sequence
  Description of Artificial Sequence Synthetic oligonucleotide
ggcgggagaaugacgugaatt

SEQ ID NO: 768 - DNA, 21 bp, Artificial Sequence
  Description of Artificial Sequence Synthetic oligonucleotide
uucacgucauucucccgcctt

SEQ ID NO: 769 - DNA, 21 bp, Artificial Sequence
  Description of Artificial Sequence Synthetic oligonucleotide
guagcaaaguccgaggucctt

SEQ ID NO: 770 - DNA, 21 bp, Artificial Sequence
  Description of Artificial Sequence Synthetic oligonucleotide
ggaccucggacuuugcuactt

SEQ ID NO: 771 - DNA, 21 bp, Artificial Sequence
  Description of Artificial Sequence Synthetic oligonucleotide
guagcaaaguccgaggucctt

SEQ ID NO: 772 - DNA, 21 bp, Artificial Sequence
  Description of Artificial Sequence Synthetic oligonucleotide
ggaccucggacuuugcuactt

SEQ ID NO: 773 - DNA, 21 bp, Artificial Sequence
  Description of Artificial Sequence Synthetic oligonucleotide
ggcgggagaaugacgugaatt

SEQ ID NO: 774 - DNA, 21 bp, Artificial Sequence
  Description of Artificial Sequence Synthetic oligonucleotide
uucacgucauucucccgcctt

SEQ ID NO: 775 - DNA, 21 bp, Artificial Sequence
  Description of Artificial Sequence Synthetic oligonucleotide
guagcaaaguccgaggucctt

SEQ ID NO: 776 - DNA, 21 bp, Artificial Sequence
  Description of Artificial Sequence Synthetic oligonucleotide
ggaccucggacuuugcuactt

SEQ ID NO: 777 - DNA, 22 bp, Artificial Sequence
  Description of Artificial Sequence Synthetic oligonucleotide
tggcgggagaaugacgugaatt

SEQ ID NO: 778 - DNA, 21 bp, Artificial Sequence
  Description of Artificial Sequence Synthetic oligonucleotide
uucacgucauucucccgcctt

SEQ ID NO: 779 - DNA, 21 bp, Artificial Sequence
  Description of Artificial Sequence Synthetic oligonucleotide
ggcgggagaaugacgugaatt

SEQ ID NO: 780 - DNA, 21 bp, Artificial Sequence
  Description of Artificial Sequence Synthetic oligonucleotide
uucacgucauucucccgcctt

SEQ ID NO: 781 - DNA, 22 bp, Artificial Sequence
  Description of Artificial Sequence Synthetic oligonucleotide
tggcgggagaaugacgugaatt

SEQ ID NO: 782 - DNA, 21 bp, Artificial Sequence
  Description of Artificial Sequence Synthetic oligonucleotide
uucacgucauucucccgcctt

SEQ ID NO: 783 - DNA, 21 bp, Artificial Sequence
  Description of Artificial Sequence Synthetic oligonucleotide
ggcgggagaaugacgugaatt

SEQ ID NO: 784 - DNA, 21 bp, Artificial Sequence
  Description of Artificial Sequence Synthetic oligonucleotide
uucacgucauucucccgcctt

SEQ ID NO: 785 - DNA, 22 bp, Artificial Sequence
  Description of Artificial Sequence Synthetic oligonucleotide
tguagcaaaguccgaggucctt

SEQ ID NO: 786 - DNA, 21 bp, Artificial Sequence
  Description of Artificial Sequence Synthetic oligonucleotide
ggaccucggacuuugcuactt

SEQ ID NO: 787 - DNA, 21 bp, Artificial Sequence
  Description of Artificial Sequence Synthetic oligonucleotide
guagcaaaguccgaggucctt

SEQ ID NO: 788 - DNA, 21 bp, Artificial Sequence
  Description of Artificial Sequence Synthetic oligonucleotide
ggaccucggacuuugcuactt

SEQ ID NO: 789 - DNA, 21 bp, Artificial Sequence
  Description of Artificial Sequence Synthetic oligonucleotide
aucgauaugagcuggucactt

SEQ ID NO: 790 - DNA, 21 bp, Artificial Sequence
  Description of Artificial Sequence Synthetic oligonucleotide
gugaccagcucauaucgautt

SEQ ID NO: 791 - DNA, 21 bp, Artificial Sequence
  Description of Artificial Sequence Synthetic oligonucleotide
gauacuugaaccaguucgatt

SEQ ID NO: 792 - DNA, 21 bp, Artificial Sequence
  Description of Artificial Sequence Synthetic oligonucleotide
ucgaacugguucaaguauctt

SEQ ID NO: 793 - DNA, 21 bp, Artificial Sequence
  Description of Artificial Sequence Synthetic oligonucleotide
aucggcucuuagauaccuutt

SEQ ID NO: 794 - DNA, 21 bp, Artificial Sequence
  Description of Artificial Sequence Synthetic oligonucleotide
aagguaucuaagagccgautt

SEQ ID NO: 795 - DNA, 21 bp, Artificial Sequence
  Description of Artificial Sequence Synthetic oligonucleotide
gccaucaagcaaugccgactt

SEQ ID NO: 796 - DNA, 21 bp, Artificial Sequence
  Description of Artificial Sequence Synthetic oligonucleotide
gucggcauugcuugauggctt

SEQ ID NO: 797 - DNA, 21 bp, Artificial Sequence
  Description of Artificial Sequence Synthetic oligonucleotide
ucaguaagaccgugguuugtt

SEQ ID NO: 798 - DNA, 21 bp, Artificial Sequence
  Description of Artificial Sequence Synthetic oligonucleotide
caaaccacggucuuacugatt

SEQ ID NO: 799 - DNA, 21 bp, Artificial Sequence
  Description of Artificial Sequence Synthetic oligonucleotide
ucgccuguagcaaaguccgtt

SEQ ID NO: 800 - DNA, 21 bp, Artificial Sequence
  Description of Artificial Sequence Synthetic oligonucleotide
cggacuuugcuacaggcgatt

SEQ ID NO: 801 - DNA, 21 bp, Artificial Sequence
  Description of Artificial Sequence Synthetic oligonucleotide
gccuguagcaaaguccgagtt

SEQ ID NO: 802 - DNA, 21 bp, Artificial Sequence
  Description of Artificial Sequence Synthetic oligonucleotide
cucggacuuugcuacaggctt

SEQ ID NO: 803 - DNA, 21 bp, Artificial Sequence
  Description of Artificial Sequence Synthetic oligonucleotide
ggagcccgaugggucggaatt

SEQ ID NO: 804 - DNA, 21 bp, Artificial Sequence
  Description of Artificial Sequence Synthetic oligonucleotide
uuccgacccaucgggcucctt

SEQ ID NO: 805 - DNA, 21 bp, Artificial Sequence
  Description of Artificial Sequence Synthetic oligonucleotide
ccaucaagcaaugccgacatt

SEQ ID NO: 806 - DNA, 21 bp, Artificial Sequence
  Description of Artificial Sequence Synthetic oligonucleotide
ugucggcauugcuugauggtt

SEQ ID NO: 807 - DNA, 21 bp, Artificial Sequence
  Description of Artificial Sequence Synthetic oligonucleotide
cggcucuuagauaccuucatt

SEQ ID NO: 808 - DNA, 21 bp, Artificial Sequence
  Description of Artificial Sequence Synthetic oligonucleotide
ugaagguaucuaagagccgtt

SEQ ID NO: 809 - DNA, 21 bp, Artificial Sequence
  Description of Artificial Sequence Synthetic oligonucleotide
uauugaucuaggauaugcctt

SEQ ID NO: 810 - DNA, 21 bp, Artificial Sequence
  Description of Artificial Sequence Synthetic oligonucleotide
ggcauauccuagaucaauatt

SEQ ID NO: 811 - DNA, 21 bp, Artificial Sequence
  Description of Artificial Sequence Synthetic oligonucleotide
uaagaccgugguuuguaagtt

SEQ ID NO: 812 - DNA, 21 bp, Artificial Sequence
  Description of Artificial Sequence Synthetic oligonucleotide
cuuacaaaccacggucuuatt

SEQ ID NO: 813 - DNA, 21 bp, Artificial Sequence
  Description of Artificial Sequence Synthetic oligonucleotide
caucgauaugagcuggucatt

SEQ ID NO: 814 - DNA, 21 bp, Artificial Sequence
  Description of Artificial Sequence Synthetic oligonucleotide
ugaccagcucauaucgaugtt

SEQ ID NO: 815 - DNA, 21 bp, Artificial Sequence
  Description of Artificial Sequence Synthetic oligonucleotide
uguagcaaaguccgagguctt

SEQ ID NO: 816 - DNA, 21 bp, Artificial Sequence
  Description of Artificial Sequence Synthetic oligonucleotide
gaccucggacuuugcuacatt

SEQ ID NO: 817 - DNA, 21 bp, Artificial Sequence
  Description of Artificial Sequence Synthetic oligonucleotide
gggcgggagaaugacgugatt

SEQ ID NO: 818 - DNA, 21 bp, Artificial Sequence
  Description of Artificial Sequence Synthetic oligonucleotide
ucacgucauucucccgccctt

SEQ ID NO: 819 - DNA, 21 bp, Artificial Sequence
  Description of Artificial Sequence Synthetic oligonucleotide
gaucgccuguagcaaaguctt

SEQ ID NO: 820 - DNA, 21 bp, Artificial Sequence
  Description of Artificial Sequence Synthetic oligonucleotide
gacuuugcuacaggcgauctt

SEQ ID NO: 821 - DNA, 21 bp, Artificial Sequence
  Description of Artificial Sequence Synthetic oligonucleotide
ucgauaugagcuggucacctt

SEQ ID NO: 822 - DNA, 21 bp, Artificial Sequence
  Description of Artificial Sequence Synthetic oligonucleotide
ggugaccagcucauaucgatt

SEQ ID NO: 823 - DNA, 21 bp, Artificial Sequence
  Description of Artificial Sequence Synthetic oligonucleotide
aggagcccgaugggucggatt

SEQ ID NO: 824 - DNA, 21 bp, Artificial Sequence
  Description of Artificial Sequence Synthetic oligonucleotide
uccgacccaucgggcuccutt

SEQ ID NO: 825 - DNA, 21 bp, Artificial Sequence
  Description of Artificial Sequence Synthetic oligonucleotide
ugggaaaugaaagaacgcctt

SEQ ID NO: 826 - DNA, 21 bp, Artificial Sequence
  Description of Artificial Sequence Synthetic oligonucleotide
ggcguucuuucauuucccatt

SEQ ID NO: 827 - DNA, 21 bp, Artificial Sequence
  Description of Artificial Sequence Synthetic oligonucleotide
ggaagacuguaaccggcugtt

SEQ ID NO: 828 - DNA, 21 bp, Artificial Sequence
  Description of Artificial Sequence Synthetic oligonucleotide
cagccgguuacagucuucctt

SEQ ID NO: 829 - DNA, 21 bp, Artificial Sequence
  Description of Artificial Sequence Synthetic oligonucleotide
aucgccuguagcaaagucctt

SEQ ID NO: 830 - DNA, 21 bp, Artificial Sequence
  Description of Artificial Sequence Synthetic oligonucleotide
ggacuuugcuacaggcgautt

SEQ ID NO: 831 - DNA, 21 bp, Artificial Sequence
  Description of Artificial Sequence Synthetic oligonucleotide
cccgaguuuucauguccuctt

SEQ ID NO: 832 - DNA, 21 bp, Artificial Sequence
  Description of Artificial Sequence Synthetic oligonucleotide
gaggacaugaaaacucgggtt

SEQ ID NO: 833 - DNA, 21 bp, Artificial Sequence
  Description of Artificial Sequence Synthetic oligonucleotide
ccgaugggucggaagcaggtt

SEQ ID NO: 834 - DNA, 21 bp, Artificial Sequence
  Description of Artificial Sequence Synthetic oligonucleotide
ccugcuuccgacccaucggtt

SEQ ID NO: 835 - DNA, 21 bp, Artificial Sequence
  Description of Artificial Sequence Synthetic oligonucleotide
cgccuguagcaaaguccgatt

SEQ ID NO: 836 - DNA, 21 bp, Artificial Sequence
  Description of Artificial Sequence Synthetic oligonucleotide
ucggacuuugcuacaggcgtt

SEQ ID NO: 837 - DNA, 21 bp, Artificial Sequence
  Description of Artificial Sequence Synthetic oligonucleotide
gaagacuguaaccggcugctt

SEQ ID NO: 838 - DNA, 21 bp, Artificial Sequence
  Description of Artificial Sequence Synthetic oligonucleotide
gcagccgguuacagucuuctt

SEQ ID NO: 839 - DNA, 21 bp, Artificial Sequence
  Description of Artificial Sequence Synthetic oligonucleotide
aagacuguaaccggcugcatt

SEQ ID NO: 840 - DNA, 21 bp, Artificial Sequence
  Description of Artificial Sequence Synthetic oligonucleotide
ugcagccgguuacagucuutt

SEQ ID NO: 841 - DNA, 21 bp, Artificial Sequence
  Description of Artificial Sequence Synthetic oligonucleotide
acgucauccgguggcacaatt

SEQ ID NO: 842 - DNA, 21 bp, Artificial Sequence
  Description of Artificial Sequence Synthetic oligonucleotide
uugugccaccggaugacgutt

SEQ ID NO: 843 - DNA, 21 bp, Artificial Sequence
  Description of Artificial Sequence Synthetic oligonucleotide
ggaaacgucauccgguggctt

SEQ ID NO: 844 - DNA, 21 bp, Artificial Sequence
  Description of Artificial Sequence Synthetic oligonucleotide
gccaccggaugacguuucctt

SEQ ID NO: 845 - DNA, 21 bp, Artificial Sequence
  Description of Artificial Sequence Synthetic oligonucleotide
gauuuggaaacgucauccgtt

SEQ ID NO: 846 - DNA, 21 bp, Artificial Sequence
  Description of Artificial Sequence Synthetic oligonucleotide
cggaugacguuuccaaauctt

SEQ ID NO: 847 - DNA, 21 bp, Artificial Sequence
  Description of Artificial Sequence Synthetic oligonucleotide
uguccucagcgggugucgctt

SEQ ID NO: 848 - DNA, 21 bp, Artificial Sequence
  Description of Artificial Sequence Synthetic oligonucleotide
gcgacacccgcugaggacatt

SEQ ID NO: 849 - DNA, 21 bp, Artificial Sequence
  Description of Artificial Sequence Synthetic oligonucleotide
aaacgucauccgguggcactt

SEQ ID NO: 850 - DNA, 21 bp, Artificial Sequence
  Description of Artificial Sequence Synthetic oligonucleotide
gugccaccggaugacguuutt

SEQ ID NO: 851 - DNA, 21 bp, Artificial Sequence
  Description of Artificial Sequence Synthetic oligonucleotide
cauccgguggcacaaucagtt

SEQ ID NO: 852 - DNA, 21 bp, Artificial Sequence
  Description of Artificial Sequence Synthetic oligonucleotide
cugauugugccaccggaugtt

SEQ ID NO: 853 - DNA, 21 bp, Artificial Sequence
  Description of Artificial Sequence Synthetic oligonucleotide
uggaaacgucauccgguggtt

SEQ ID NO: 854 - DNA, 21 bp, Artificial Sequence
  Description of Artificial Sequence Synthetic oligonucleotide
ccaccggaugacguuuccatt

SEQ ID NO: 855 - DNA, 21 bp, Artificial Sequence
  Description of Artificial Sequence Synthetic oligonucleotide
cauguccucagcggguguctt

SEQ ID NO: 856 - DNA, 21 bp, Artificial Sequence
  Description of Artificial Sequence Synthetic oligonucleotide
gacacccgcugaggacaugtt

SEQ ID NO: 857 - DNA, 42 bp, Artificial Sequence
  Description of Artificial Sequence Synthetic oligonucleotide
gatgttttctggctttagatccctttttgaagttaccgtttt

SEQ ID NO: 858 - DNA, 40 bp, Artificial Sequence
  Description of Artificial Sequence Synthetic oligonucleotide
ctgttctccttgctgcaggactttttctgagtcaaagcat

SEQ ID NO: 859 - DNA, 50 bp, Artificial Sequence
  Description of Artificial Sequence Synthetic oligonucleotide
cctaggtcaataattttgtgtattaaccttttttctcttggaaagaaagt

SEQ ID NO: 860 - DNA, 42 bp, Artificial Sequence
  Description of Artificial Sequence Synthetic oligonucleotide
tgatccagctccttggcatattttttctcttggaaagaaagt

SEQ ID NO: 861 - DNA, 40 bp, Artificial Sequence
  Description of Artificial Sequence Synthetic oligonucleotide
aatgatgtgcaaagactgccctttttgaagttaccgtttt

SEQ ID NO: 862 - DNA, 37 bp, Artificial Sequence
  Description of Artificial Sequence Synthetic oligonucleotide
tactgcagggtccccacgtttttctgagtcaaagcat

SEQ ID NO: 863 - DNA, 40 bp, Artificial Sequence
  Description of Artificial Sequence Synthetic oligonucleotide
cagtagctctggggccaggtttttctcttggaaagaaagt

SEQ ID NO: 864 - DNA, 43 bp, Artificial Sequence
  Description of Artificial Sequence Synthetic oligonucleotide
ggtcactgtgtacttctgctgctctttttgaagttaccgtttt

SEQ ID NO: 865 - DNA, 40 bp, Artificial Sequence
  Description of Artificial Sequence Synthetic oligonucleotide
gccgaagctccagtagtcgactttttctgagtcaaagcat

SEQ ID NO: 866 - DNA, 40 bp, Artificial Sequence
  Description of Artificial Sequence Synthetic oligonucleotide
tgcactcaaaggccagggttttttctcttggaaagaaagt

SEQ ID NO: 867 - DNA, 35 bp, Artificial Sequence
  Description of Artificial Sequence Synthetic oligonucleotide
ggccggaagcccgtgatttttgaagttaccgtttt

SEQ ID NO: 868 - DNA, 38 bp, Artificial Sequence
  Description of Artificial Sequence Synthetic oligonucleotide
ctgccagttggggaggaagtttttctgagtcaaagcat

SEQ ID NO: 869 - DNA, 39 bp, Artificial Sequence
  Description of Artificial Sequence Synthetic oligonucleotide
tgaatgccactgcacgggtttttctcttggaaagaaagt

SEQ ID NO: 870 - DNA, 40 bp, Artificial Sequence
  Description of Artificial Sequence Synthetic oligonucleotide
ctcactcttctgccgcacttttttttgaagttaccgtttt

SEQ ID NO: 871 - DNA, 42 bp, Artificial Sequence
  Description of Artificial Sequence Synthetic oligonucleotide
tcttcgctaacaacaatgtccactttttctgagtcaaagcat

SEQ ID NO: 872 - DNA, 23 bp, Artificial Sequence
  Description of Artificial Sequence Synthetic oligonucleotide
aaaacttcaccgttccattcaag

SEQ ID NO: 873 - DNA, 42 bp, Artificial Sequence
  Description of Artificial Sequence Synthetic oligonucleotide
tggggtagggtaaagagcttgtttttctcttggaaagaaagt

SEQ ID NO: 874 - DNA, 44 bp, Artificial Sequence
  Description of Artificial Sequence Synthetic oligonucleotide
tcagccaggacactgttaagattattttttgaagttaccgtttt

SEQ ID NO: 875 - DNA, 39 bp, Artificial Sequence
  Description of Artificial Sequence Synthetic oligonucleotide
gcagccacttctccagtcgctttttctgagtcaaagcat

SEQ ID NO: 876 - DNA, 41 bp, Artificial Sequence
  Description of Artificial Sequence Synthetic oligonucleotide
gaatttgccatgggtggaattttttctcttggaaagaaagt

SEQ ID NO: 877 - DNA, 41 bp, Artificial Sequence
  Description of Artificial Sequence Synthetic oligonucleotide
ggagggatctcgctcctggatttttctcttggaaagaaagt

SEQ ID NO: 878 - DNA, 40 bp, Artificial Sequence
  Description of Artificial Sequence Synthetic oligonucleotide
ccccagccttctccatggttttttctcttggaaagaaagt

SEQ ID NO: 879 - DNA, 40 bp, Artificial Sequence
  Description of Artificial Sequence Synthetic oligonucleotide
gctcccccctgcaaatgagtttttctcttggaaagaaagt

SEQ ID NO: 880 - DNA, 42 bp, Artificial Sequence
  Description of Artificial Sequence Synthetic oligonucleotide
agccttgacggtgccatgtttttaggcataggacccgtgtct

SEQ ID NO: 881 - DNA, 45 bp, Artificial Sequence
  Description of Artificial Sequence Synthetic oligonucleotide
gatgacaagcttcccgttctctttttaggcataggacccgtgtct

SEQ ID NO: 882 - DNA, 46 bp, Artificial Sequence
  Description of Artificial Sequence Synthetic oligonucleotide
agatggtgatgggatttccatttttttaggcataggacccgtgtct

SEQ ID NO: 883 - DNA, 44 bp, Artificial Sequence
  Description of Artificial Sequence Synthetic oligonucleotide
gcatcgccccacttgatttttttttaggcataggacccgtgtct

SEQ ID NO: 884 - DNA, 43 bp, Artificial Sequence
  Description of Artificial Sequence Synthetic oligonucleotide
cacgacgtactcagcgccatttttaggcataggacccgtgtct

SEQ ID NO: 885 - DNA, 46 bp, Artificial Sequence
  Description of Artificial Sequence Synthetic oligonucleotide
ggcagagatgatgacccttttgtttttaggcataggacccgtgtct

SEQ ID NO: 886 - DNA, 21 bp, Artificial Sequence
  Description of Artificial Sequence Synthetic oligonucleotide
ggtgaagacgccagtggactc

SEQ ID NO: 887 - DNA, 37 bp, Artificial Sequence
  Description of Artificial Sequence Synthetic oligonucleotide
agcctgctccatctcccgtttttgaagttaccgtttt

SEQ ID NO: 888 - DNA, 37 bp, Artificial Sequence
  Description of Artificial Sequence Synthetic oligonucleotide
ccgcccacactgctccactttttctgagtcaaagcat

SEQ ID NO: 889 - DNA, 45 bp, Artificial Sequence
  Description of Artificial Sequence Synthetic oligonucleotide
tctactagatgcttcacgtcattctctttttgaagttaccgtttt

SEQ ID NO: 890 - DNA, 37 bp, Artificial Sequence
  Description of Artificial Sequence Synthetic oligonucleotide
tgcagtgccatcatccgctttttctgagtcaaagcat

SEQ ID NO: 891 - DNA, 41 bp, Artificial Sequence
  Description of Artificial Sequence Synthetic oligonucleotide
ctgcaggtccacaatgtcagtctttttgaagttaccgtttt

SEQ ID NO: 892 - DNA, 36 bp, Artificial Sequence
  Description of Artificial Sequence Synthetic oligonucleotide
cgacccatcgggctccttttttctgagtcaaagcat

SEQ ID NO: 893 - DNA, 17 bp, Artificial Sequence
  Description of Artificial Sequence Synthetic oligonucleotide
gggtgcccccctgcttc

SEQ ID NO: 894 - DNA, 43 bp, Artificial Sequence
  Description of Artificial Sequence Synthetic oligonucleotide
gcttgttcctctaggtcatccatttttctcttggaaagaaagt

SEQ ID NO: 895 - DNA, 41 bp, Artificial Sequence
  Description of Artificial Sequence Synthetic oligonucleotide
gagtcttcggtagagctccctctttttgaagttaccgtttt

SEQ ID NO: 896 - DNA, 40 bp, Artificial Sequence
  Description of Artificial Sequence Synthetic oligonucleotide
ttggtctcttggcttctcccttttttctgagtcaaagcat

SEQ ID NO: 897 - DNA, 43 bp, Artificial Sequence
  Description of Artificial Sequence Synthetic oligonucleotide
cctggctgtcaccttctgtccttttttctcttggaaagaaagt

SEQ ID NO: 898 - DNA, 39 bp, Artificial Sequence
  Description of Artificial Sequence Synthetic oligonucleotide
aagcagcagccgtaccatcttttttgaagttaccgtttt

SEQ ID NO: 899 - DNA, 40 bp, Artificial Sequence
  Description of Artificial Sequence Synthetic oligonucleotide
ctcaaagctttggattgcctgtttttctgagtcaaagcat

SEQ ID NO: 900 - DNA, 46 bp, Artificial Sequence
  Description of Artificial Sequence Synthetic oligonucleotide
gtgtataaatcacccgaactttctttttttctcttggaaagaaagt

SEQ ID NO: 901 - DNA, 22 bp, Artificial Sequence
  Description of Artificial Sequence Synthetic oligonucleotide
caaaccacggtcttactgagct

SEQ ID NO: 902 - DNA, 43 bp, Artificial Sequence
  Description of Artificial Sequence Synthetic oligonucleotide
caactccagtgccttctgcttatttttctcttggaaagaaagt

SEQ ID NO: 903 - DNA, 41 bp, Artificial Sequence
  Description of Artificial Sequence Synthetic oligonucleotide
cttcaccattttgtctacgggatttttgaagttaccgtttt

SEQ ID NO: 904 - DNA, 39 bp, Artificial Sequence
  Description of Artificial Sequence Synthetic oligonucleotide
ccaaatccgttcacaccgactttttctgagtcaaagcat

SEQ ID NO: 905 - DNA, 38 bp, Artificial Sequence
  Description of Artificial Sequence Synthetic oligonucleotide
ccaggcgcccaatacggtttttctcttggaaagaaagt

SEQ ID NO: 906 - DNA, 38 bp, Artificial Sequence
  Description of Artificial Sequence Synthetic oligonucleotide
caaatggcagccctggtgatttttgaagttaccgtttt

SEQ ID NO: 907 - DNA, 41 bp, Artificial Sequence
  Description of Artificial Sequence Synthetic oligonucleotide
aacaatctccactttgccactgtttttctgagtcaaagcat

SEQ ID NO: 908 - DNA, 19 bp, Artificial Sequence
  Description of Artificial Sequence Synthetic oligonucleotide
tgaaggggtcgttgatggc

SEQ ID NO: 909 - DNA, 26 bp, Artificial Sequence
  Description of Artificial Sequence Synthetic oligonucleotide
catgtagaccatgtagttgaggtcaa

SEQ ID NO: 910 - DNA, 44 bp, Artificial Sequence
  Description of Artificial Sequence Synthetic oligonucleotide
ccgtgagtggagtcatactggaatttttctcttggaaagaaagt

SEQ ID NO: 911 - DNA, 42 bp, Artificial Sequence
  Description of Artificial Sequence Synthetic oligonucleotide
ttgactgtgccgttgaatttgtttttctcttggaaagaaagt

SEQ ID NO: 912 - DNA, 37 bp, Artificial Sequence
  Description of Artificial Sequence Synthetic oligonucleotide
agcttcccattctcggcctttttgaagttaccgtttt

SEQ ID NO: 913 - DNA, 38 bp, Artificial Sequence
  Description of Artificial Sequence Synthetic oligonucleotide
gggcttcccgttgatgacatttttctgagtcaaagcat

SEQ ID NO: 914 - DNA, 41 bp, Artificial Sequence
  Description of Artificial Sequence Synthetic oligonucleotide
cgctcctggaagatggtgattttttctcttggaaagaaagt

SEQ ID NO: 915 - DNA, 23 bp, Artificial Sequence
  Description of Artificial Sequence Synthetic oligonucleotide
cccatttgatgttagtggggtct

SEQ ID NO: 916 - DNA, 41 bp, Artificial Sequence
  Description of Artificial Sequence Synthetic oligonucleotide
atactcagcaccggcctcactttttctcttggaaagaaagt

SEQ ID NO: 917 - DNA, 3856 bp, Homo sapiens
atgttttcaggggggtgtcatagccccgggtttggccgccccagccccgccttccccgccccggggagcccgccccctgccccgcgtccctgccgacagagttagcacgacatcagtatgagctggtcaccttccctgacaacgcagacatgtggggcctgggaaatgaaagagcgccttgggacagggggatttggaaatgtcatccgatggcacaatcaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatgatgtggggggactcgaccccctgacatggggcagcccatagcaggccttgtgcagtggggggactcgaccccctgacatggggctgcctggagcaggccgcgtgacgtggggctgcctggccgcggctctcacatggtggttcctgctgcactgatggcccaggggtctctggtatccagatggagctctcgcttcctcagcagctgtgactttcacccaggacccaggacgcagccctccgtgggcactgccggcgccttgtctgcacactggaggtcctccattacagaggcccagcgcacatcgctggccccacaaacgttcaggggtacagccatggcagctccttcctctgccgtgagaaaagtgcttggagtacggtttgccacacacgtgactggacagtgtccaattcaaatctttcagggcagagtccgagcagcgcttggtgacagcctgtcctctcctgctctccaaaggccctgctccctgtcctctctcactttacagcttgtgtttcttctggattcagcttctcctaaacagacagtttaattatagttgcggcctggccccatcctcacttcctctttttatttcactgctgctaaaattgtgtttttacctactactttggtggttgtcctcttttcggcaaagttggagcgagtgccaagctctccatctgtggtcctttctgccaagagcgactcatagtaaccaggatgggagagcagctgccttattctgaatcccaaaaattacttgggggtgattgtcacagaggagggacagaaagggtatctgctgaccaccagcctgcctacccatgcccatgtctccattcctgctcaagcgtgtgtgctgggccggggagtccctgtctctcacagcatctagcagtattattaaatggattcattttaaaaatagctcctatattttgtaacatgtctcaaacactcatactgggttccacaatccactgttagaatacctatggttagggcttctgaactaaaataatggaaaattttaacaatttgtatagtgcctggatcattactagtgccataaccctgcttcttcaacatttcacagaacttctcttttatataaaggcaagagcacaaaatgagttcagatgatcacaaacaggtgagttttgttggagaagaaagttggagtaggagactttcacaagtggtttccatggagatagaatgaagcattctgtggtcaagtaagtttagggagctattcatgtttcacttgctttgtggagattcacactatgcactgggaaagtatctgaaaagtcttataataaagaaacaggcttaactttgtgtaagaacactgtttatcaatgtcatttggctatagaaacattttctcctgctgattgtgtgtgtgaaacatgtattaacattccaatgaactagcatttaataaagcacaattttgg

SEQ ID NO: 918 - DNA, 5002 bp, Homo sapiens
catagccccgggtttggccgccccagccccgccttccccgccccggggagcccgccccctgccccgcgtccctgccgacagagttagcacgacatcagtatgagctggtcaccttccctgacaacgcagacatgtggggcctgggaaatgaaagagcgccttgggacagggggatttggaaatgtcatccgatggcacaatcaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcagtgagtgtgcactttgcaatgagtttaaagacaccatttttttttctttttctctttctaaggtttcttttgtcctattataattgagttgcttatttctgtttcagttttgtggtgtttttattttgttttgttttgtttttccttctcaattttttttcagcatcagataaactgctgctggcctggagggaaatggagcaggctgtggagctctgtgggcgggtaggagactcattttgggtttcggaacttaccaagggggtgagattttgtgctcccacttttcactttctcatcaaacactgggtcgatctctgtcaaaaatcaggtgttcctcaccatgctttgagcttaactggaccagctgaaaagaggccaagagagtagccagtgaagggagcccctctgtggccagatgcaagagctgcatgaatggacagagatggtccaggtttaaataagaagcaagggttaggttcccttggggaaaagccaagaccatttcctgttaggaagggcattggatggatgatcaacactccctcttgtcgaaggtttttgactaattttgttgggatgctaaatccataggaaataaaaggtctaggcagaaagtctcagggcagcgtaaatcagatgtcaccaaaaaaagttaattatgaaggcaaaggaaatacatgtgacttaatgaacctcacacaaactgaaggttgaagtcaggctccggggctttttcctggacttatcctctgttttcccctgtgtacttgggcaaggatcttcacctgtgtcttagtatcgatttttatgaatcagtactgatggagtatatgtgtgtttaatggtgaatattaaccatagaagggctgagtgctcctccccatcttgggactcataatctgtgaaataaaacagtctgggcctccctgcccgggtagcatcaggatagacagatgaagagaagagaaagtaatgtgtcttgggcatcttctgtatgccaggcaccatgccagaggctttaagtacttcatcttacttgactcctcagatggccctgttagaagcctattttatgcaaaaggaaactgtagctgggggtaagtaacttgccaaggggtcacacagctagaaagcggtggaccctagatgcaggcgcagtcattcagaccccacagtccacattcctttgagccagtccattgagggtcctcagggaatgtggcgggtcccctggtctcgctcccccgcagatcttgcatctcagcatgcgcctaccacatcagttgacattagcacagcttttccattaggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatgatgtggggggactcgaccccctgacatggggcagcccatagcaggccttgtgcggtggggggactcgaccccctgacatggggctgcctggagcaggccgcgtgacgtggggctgcctggccgcggctctcacatggtggttcctgctgcactgatggcccaggggtctctggtatccagatggagctctcgcttcctcagcagctgtgactttcacccaggacccaggacgcagccctccgtgggcactgccggcgccttgtctgcacactggaggtcctccattacagaggcccagcgcacatcgctggccccacaaacgttcaggggtacagccatggcagctccttcctctgccgtgagaaaagtgcttggagtacggtttgccacacacgtgactggacagtgtccaattcaaatctttcagggcagagtccgagcagcgcttggtgacagcctgtcctctcctgctctccaaaggccctgctccctgtcctctctcactttacagcttgtgtttcttctggattcagcttctcctaaacagacagtttaattatagttgcggcctggccccatcctcacttcctctttttatttcactgctgctaaaattgtgtttttacctactactttggtggttgtcctcttttcggcaaagttggagcgagtgccaagctctccatctgtggtcctttctgccaagagcgactcatagtaaccaggatgggagagcagctgccttattctgaatcccaaaaattacttgggggtgattgtcacagaggagggacagaaagggtatctgctgaccaccagcctgcctacccatgcccatgtctccattcctgctcaagcgtgtgtgctgggccggggagtccctgtctctcacagcatctagcagtattattaaatggattcattttaaaaatagctcctatattttgtaacatgtctcaaacactcatactgggttccacaatccactgttagaatacctatggttagggcttctgaactaaaataatggaaaattttaacaatttgtatagtgcctggatcattactagtgccataaccctgcttcttcaacatttcacagaacttctcttttatataaaggcaagagcacaaaatgagttcagatgatcacaaacaggtgagttttgttggagaagaaagttggagtaggagactttcacaagtggtttccatggagatagaatgaagcattctgtggtcaagtaagtttagggagctattcatgtttcacttgctttgtggagattcacactatgcactgggaaagtatctgaaaagtcttataataaagaaacaggcttaactttgtgtaagaacactgtttatcaatgtcatttggctatagaaacattttctcctgctgattgtgtgtgtgaaacatgtattaacattccaatgaactagcatttaataaagcacaattttggaaaccctg

SEQ ID NO: 919 - DNA, 784 bp, Macaca fascicularis
tcctcgagcactgttggcctactgggatgtcagagcaggaagtgttagaggaagtcgcgccgcgctgcccgcgttaagattcccgcattttaatgttttcagaggggtatcagagcccagggtccggccgccccagccccgcctcccccgccccggggagccctccccctgccccgcgtccctgccgacagagttagcacgacatcagtatgagctggtcaccttccctgacaacccagacatgtggggcctgggaaatgaaagagcgccttgggacagggggatttggaaatgtcatccgatggcacaatcaggctgacgcaccccaatgtggaggctgcccgagatgtccctgaggggatgcagaacttggcgcccaatgacttgcccctgctggccatggagcattgccaaggaggagatctacggaagtacctagaccagattgagaattgatgcggtctgcaggaaggtgccatactcacacttgcagaagtgacaatagcctctgaacttatataccctgatgataacagaattatccatggggatcaatggcaaaaaactttatacagtattaaggaaacagatgatatttcacgatatatatagactataatatgccagtaagttggaataaggaagttattgctcattattaagtgtgaaaccacatacacgggacagatacgttagagatgtataaataacaatgacattacatagtagtatagaaggacgaatgctatacaagcataatagaattatatcaaaaatataatat

SEQ ID NO: 920 - DNA, 2250 bp, Macaca fascicularis
  modified_base (878)..(878): a, c, t, g, unknown or other
atgagctggtcaccttccctgacaacccagacatgtggggcctgggaaatgaaagagcgccttgggacagggggatttggaaatgtcatccgatggcacaatcaggaaacaggtgagcagattgccatcaagcagtgccggcaggagctcagcccccggaatcgagagcggtggtgcctggagatccagatcatgagaaggctgacgcaccccaatgtggtggctgcccgagatgtccctgaggggatgcagaacttggcgcccaatgacttgcccctgctggccatggagtattgccaaggaggagatctccggaagtacctgaaccagtttgagaattgctgtggtctgcgggaaggtgccatcctcaccttgctgagtgacattgcctctgcacttagataccttcatgaaaacagaatcatccatcgggatctaaagccagaaaacatcgtcctgcagcaaggagaacagaggttaatacacaaaattattgacctaggatatgccaaggagctggatcagggcagtctttgcacgtcattcgtggggaccctgcagtacctggccccagagctgctggagcagcagaagtacacagtgaccgtcgactactggagctttggcaccctggccttcgagtgcatcacgggcttccggcccttcctccccaactggcagcccgtgcagtggcattcgaaagtccggcagaagagtgaggtggatattgttgtcagcgaagacttgaatggaacagtgaagttttcaagctctttaccctaccccaataatcttaacagcgtcctggctgagcgactggagaagtggctgcaactgatgctgatgtggcatccccgacagaggggcacagatcccangtatgggcccaatggctgcttcaaggccctggatgacatcttaaacttaaagctggttcatatcttgaacatggtcacaggcaccatccacacctaccctgtgacagaggacgagagtctgcagagcttgaaggccagaatccagcaggacacgggcatcccagaggaggaccaggagctgctgcaggaagcgggcctggcgttgatccccgataaacctgccactcagtgtatttcagatggcaagttaaatgagggccgcacattggacatggatcttgtttttctctttgacaacagtaaaatcacctatgagactcagatctccccacggccccaacctgaaagtgtcagctgtatccttcaagagcccaagaggaacctcgccttcttccagctgaggaaggtgtggggccaggtctggcacagcatccagaccctaaaggaggattgcaaccggctgcagcagggacagcgagccgccatgatgaatctcctccggaacaacagctgcctctccaagatgaagaattccatggcttccatgtctcagcagctcaaggccaagttggatttcttcaaaaccagcatccagattgacctggagaagtacagcgagcaaactgagtttgggatcacatcagataaactgctgctggcctggagggaaatggagcaggccgtggagctctgtgggcgggagaacgaagtgaaactcctggtagaacggatgatggctctgcagacggacatcgtggacttacagaggagccccatgggccggaagcaggggggaacgttggacgacctagaggagcaagcaagggagctgtacaggagactaagggaaaagcctcgagaccagcgaactgagggtgacagtcaggaaatggtacggctgctgcttcaggcaattcagagcttcgagaagaaagtgcgagtgatctatacacagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacccgaaccagccaagaagagtgaagaactggtggccgaagcacataacctgtgcaccctgctagaaaatgccatacaggacaccgtgagggagcaagaccagagtttcacggccctagactggagctggttacagacggaagaagaagagcacagc

SEQ ID NO: 921 - DNA, 3973 bp, Mus musculus
gcggccgcgtcgacgtttgaggaagtggcgctgggctggctgcgtggggaccccgagttttcatgtcctcagcgggtgtcgctggcggccccgcggcccccggggcagagttagcagggcatcgatatgagctggtcaccgtccctcccaacccagacatgtggagcctgggaaatgaaagaacgcctggggaccgggggatttggaaacgtcatccggtggcacaatcaggcgacaggtgaacagatcgccatcaagcaatgccgacaggagctcagcccaaagaacagagaccgctggtgcctcgaaatccagatcatgagaaggctgaaccatcccaatgtggtggctgcccgggatgtcccagaggggatgcagaacctggcacccaatgatttgccactgctggccatggagtactgccaaggaggagatctccgaagatacttgaaccagttcgagaactgctgtggcctgcgggaaggagctgtccttaccctgctgagtgacatcgcatcggctcttagataccttcacgaaaacagaatcatccatcgagacctgaagccagaaaacatcgttctgcagcaaggagagaaaagattaatacacaaaattattgatctaggatatgccaaggagctggatcagggcagtctgtgcacgtcatttgtggggactctgcaatacctggcgccagagcttctggagcagcagaagtacaccgtgaccgttgactactggagcttcggcaccctggccttcgagtgcatcactggcttccggcccttcctccctaactggcagcctgtgcagtggcactccaaagtccggcagaagagcgaagtggacatcgttgttagtgaagacttgaatggagcagtgaagttttcaagttcgctacccttccccaataatcttaacagtgtcttggctgaacggctggagaagtggctgcagctgatgcttatgtggcaccctcggcaaaggggcacggatccccagtatggccccaacggctgcttcagagccctggatgacatcttgaacttgaagctggttcatgtcttgaacatggtcacaggcaccgttcacacataccccgtgacggaggatgagagtctgcagagcttaaaaaccagaatccaggaagacacggggatcctggagacagaccaggagctgctgcaagaggcagggctggtgctgctccctgacaagcctgctactcagtgcatctcagacagcaagacaaacgagggcctcacgttggacatggatcttgtttttctctttgacaacagtaaaatcaactatgagactcagatcaccccccgaccccaaccggaaagtgtcagctgtatccttcaggagcccaagcggaacctctccttcttccagctgaggaaagtgtggggccaagtctggcacagcatccagacgctgaaggaagactgtaaccggctgcagcagggacagcgagcagccatgatgagtctcctccggaataacagctgcctctctaagatgaagaacgccatggcctccacggcccagcagctcaaggccaagctggacttcttcaaaaccagcatccagatcgacctggagaagtataaagagcagaccgagtttgggatcacctcagataaattgctgctggcttggcgggagatggagcaggctgtggagcagtgtgggcgggagaatgacgtgaagcatctagtagagcggatgatggcactgcagactgacattgtggacctgcagaggagcccgatgggtcggaagcaggggggcaccctggatgacctagaggaacaagcgagggagctctaccgaagactcagggagaagccaagagaccaaaggacagaaggtgacagccaggagatggtacggctgctgcttcaggcaatccaaagctttgagaagaaagttcgggtgatttatacacagctcagtaagaccgtggtttgtaagcagaaggcactggagttgctgcccaaggtagaagaggtagtgagccttatgaacgaggacgagaggaccgtggtccggcttcaggagaagcggcagaaggaactctggaacctcctgaagatcgcctgtagcaaagtccgaggtcccgtgagtggaagcccagacagcatgaatgtgtctcgactcagtcaccctggtcagctaatgtcccagccttccagtgcctgtgacagcttacctgaatcagacaagaaaagtgaagaactggtggccgaagcccacgccctctgctcccggctagaaagtgcgctgcaggacactgtgaaggagcaagacagaagcttcacggtaaccgcctgagggagcccggcttgtccgtcccttcctctctgcagcaaaagtgcaaacgcagtttggcatcagccttggctcagcagagactcatggttggccctgcggttttcaggtttttgtatttatttcatgcttaggactgccaggtgcattcattatcttaatcccttatgacacgtaatcctaaggaggtgacagcagtaccaaaagccccttctttctctgtgggaccattgcagaacatctgtatgtgctgggcactgtgctgggctctgtgacaaaagagaaacctgtgtgtgtctcaaggactcagtttagccacaagcagtatcagtgtcagagaaactgacctgaaggaaatctagccacgatccaggcactttcagacagagcttaaacagttgttggtgtctaaatggccttttcctgctaatgcaagcatccactaactactcttgcatctaactttggggagtcagaagaaaccccattcagttgccgtcctcgctaacctgctcactggtgagcctcctaactcctgctgcttctcacttgtctcctggtgctgtttcttccatctcactacttgcttgcactttagccagagaaaagggcagccttctggttggcatggaaacggataactgagatgattcccacctatctgcagtgagtatgtaaatgagagctctatgctacaggccgtactaaaatgaccatccacattccacaggcacagagttccaggaacttcagctagtccaaaggaaatctgctgcacatttgaatctgtaactcacttggctcaagggtgccctgtagtagcaggactgacgctgataaggcttaaacgtacttctagattgtgttgcttgcctcctgaagaggaacgatggccccgccacttgagtatttaatgtgcctgttgactttcagactctagactggagctggttacagatggaggatgaagaaaggtgtagcctggagcaggcctgtgactgaggtgcccatgagctggcccgtggctctgcatgtgacgatgttctggtatctccaggtggacatctcactctgccagcagctgtgatacttgcccgcagcagcctaggctggccctgtccacacactggattctcgcccacagaagcacaacaatgcaggtacaagcacttgcagcagcgacgcttcatggctgctgtacaaagaaagcgacacagagaaatgacaagtgtctatcttgagtgtttccagacagcagacagcctgtctgctttggactcctgggggaggaagctctgccctgcccacccctcctctccttacagctggacgtcatccggatccagctctcttagacagttaatcctaagtttgactatcctatatctctcttcctcttcacttctcccctggtaaatgtttcaaccttctgtgctggtcgctgtggcaaacgacctttgactatgctgtagtgatggcgatgccaaactccctgctgtttctggtcctttaagccacaaagtgacttacaaagccaggcagaacagaaatgcagctacatatcttgagtcccaaagattacctagaatcaattgtcacagagaaaaaaggaagatgaaggtgttctcgtgcc

SEQ ID NO: 922 - DNA, 3557 bp, Rattus norvegicus
gtggggtggcgctgagctggctgcgtggggaccccgagttttcatgtcctcagcgggtgtcgctagcggccccgcggcccccggggcagagttagcagggagggcatcgatatgagctggtcaccttctctcccaacccagacatgtggggcctgggaaatgaaagaacgcctcggaactgggggatttggaaacgtcatccggtggcacaatcaggtgacaggtgaacaaattgccatcaagcaatgccgacaggagctcagccccaagaaccgcgatcgctggtgcctagagatccagatcatgagaaggctgaaccatcccaacgtggtggccgcccgggatgtcccagaggggatgcagaacttggcacccaatgatttgcctctgctggccatggagtactgccaaggaggagacctccggagatacttgaaccagttcgaaaactgctgtggcctccgggaaggagccatcctcaccctgctgagtgacatagcatcggctcttagataccttcatgaaaacagaatcatccaccgggacctgaagccagagaacattgtcctacagcaaggagagaaaagattaatacacaaaattattgatctaggatatgccaaggagctggatcagggcagtctgtgcacgtcatttgtggggaccctgcaatacctggccccagagcttctggagcagcagaagtacaccgtgactgttgactactggagcttcggcaccctggcctttgaatgcatcaccggcttccggccctttctccctaactggcagcctgtgcagtggcactctaaagtccggcagaagagcgaagtggacattgttgttagcgaggacttgaacggaacagtgaagttctcaagttccttacccttccccaataatctcaacagtgtcctggctgagcgtctggagaagtggcttcagctgatgctcacgtggcaacctcggcaaaggggcgtggacccccagtacggtcccaatggctgtttcagggccctcgatgacatcttgaacttaaagctggttcatatcttgaacatggtcacaggcaccattcacacataccctgtgatggaggatgaaagtctgcagagcttaaaaaccagaatccgggaagacacagggatcctggagacagaccaggaactgctgcaggaggcagggctggtgctgctccctgacaagcctgctactcagtgcatctcagacagcaagacaaatgagggcctcacactggacatggatctcgtctttctctttgacaacagtaaaatgtcctatgagactcagatcaccccccgaccccaacctgagagcgtcagctgtgtccttcaggagcccaagcggaacctctccttcttccagatgaggaaagtgtggggccaagtctggcacagcatccaaacgctgaaggaagactgtaaccggctgcagcagggacagcgagctgccatgatgaacctccttcggaataacagctgcctctccaagatgaagaatgccatggcctccacggcgcagcagctcaaggccaagttggacttcttcaaaaccagcatccagattgacctggagaagtacagggagcagacggagtttggcatcacatcggacaaactgctgctggcctggcgggaaatggagcaggccgtggagcagtgtgggcgggagaatgacgtgaaggtcctggtagaacggatgatggcactgcagaccgacattgtggacctacagaggagcccgatgggtcggaagcaggggggcaccttggatgacctagaggaacaagcaagagaactctacagaagactcagggagaagccaagagaccaaaggacagaaggtgacagccaggatatggtacggctgctgctgcaagccatccagagcttcgagaagaaagtgcgggtgatttactctcagctcagtaagaccgtggtttgtaagcagaaggccctggagctgctgcccaaagtagaagaggtggtgaggctcatgaacgaggatgagaagactgtggtccggctccaggagaagcggcagaaggagctctggaacctcctgaagatcgcctgtagcaaagtccgaggtccggtgagtggaagcccagatagcatgaatgtgtctcgacttagtcaccctggtcatctaatgtcccagccttccagtgcctgtgacagcttacctgattcagacaagaaaagtgaagaactggtggccgaagcacacgccctctgctcccggctggaaagcgcgctgcaggacacggtgaagcagcaggaccgaagcttcacgaccctagactggagctggttacagatggaggatgaagaaaggtgtggcctggagcaggcctgtgactgaggtgcccgtgaactggcccgcggctcggcatgtgaggatgctctggtacctccagggggacatcccgctctcctggcagctgcgatccttgcccacagcagcctgtgctggcccagtctacacactggacgtcctccaccacagaagcacaacaatgtcagttacaagcccgtgcggcaacaagactgcagtgctgctacacaaagagcttacgacagacgtctaccttgagtgtttccagacagcagccagcctgtcccctttggactgctgcgggggagggagccctgccctcctcgcctttcagctggggtgtcatctggatccagcttccttagacatttaatcagaagcctgatgattctacgtctttcttcctcttcacttccccctggtaaatgtttctaccttctgtgccggtcgttgtggcaaacggcctttgactaagttgtaatgacggtgataccaagctccctgatgtcccggctcctttaagccacagagcgacttacacagccaggcagaacagaaatgcagctacacatcctgagtcccaaagattacctggagtcaactgtcatggagagaaatggaagatgaaggtcctacaccagcccctcccctgctgagcactgtgtgactctgccacctgtcaggtgtgtgaggacgttgctgcctctcacataagctagcagcgttattaaactggttcgttttataaatagtttctattttttgtaacattactcagatatgtatcctaggctcaagggtgtatttccacagtaatactaccactagtacttctgagctcagagggcaacatcatgggaaatttcgtaagattgaacagcgtatctagatcatgtcatttttacatcacacaaaacttgtgtgtttgcgtgcatatgtgtgtatgaaagcgaagcaggctcagattgtcataaacaggtttatttgattagagaataaagttggaacagagaaatgtcagagcggccaccgcagacacagtatgaagcgtcactggtcacgtgagctcagcggtgctgcatgctttctttcccagagattgatgctgtgcactgggaacctgtttgaaaactccgacaataaagatgctggcttaagctcagcgtaaattcaaaaaaaaaaaaaaaaaaaaaaaaaaa

SEQ ID NO: 923 - DNA, 44 bp, Artificial Sequence
  Description of Artificial Sequence Synthetic oligonucleotide
tctgctctttatacttctccaggtctttttctgagtcaaagcat

SEQ ID NO: 924 - DNA, 41 bp, Artificial Sequence
  Description of Artificial Sequence Synthetic oligonucleotide
tgaggtgatcccaaactcggtttttctcttggaaagaaagt

SEQ ID NO: 925 - DNA, 42 bp, Artificial Sequence
  Description of Artificial Sequence Synthetic oligonucleotide
ccaagccagcagcaatttatctttttctcttggaaagaaagt

SEQ ID NO: 926 - DNA, 23 bp, Artificial Sequence
  Description of Artificial Sequence Synthetic oligonucleotide
ggcaacaatatccactttaccag

SEQ ID NO: 927 - DNA, 20 bp, Artificial Sequence
  Description of Artificial Sequence Synthetic oligonucleotide
gacgtactcagcgccagcat

SEQ ID NO: 928 - DNA, 39 bp, Artificial Sequence
  Description of Artificial Sequence Synthetic oligonucleotide
gaggctgcgggctcaatttttttctcttggaaagaaagt

SEQ ID NO: 929 - DNA, 41 bp, Artificial Sequence
  Description of Artificial Sequence Synthetic oligonucleotide
tggcgacgcaaaagaagatgtttttctcttggaaagaaagt

SEQ ID NO: 930 - DNA, 42 bp, Artificial Sequence
  Description of Artificial Sequence Synthetic oligonucleotide
caaatccgttgactccgaccttttttctcttggaaagaaagt

SEQ ID NO: 931 - DNA, 43 bp, Artificial Sequence
  Description of Artificial Sequence Synthetic oligonucleotide
ggtcaatgaaggggtcattgattttttctcttggaaagaaagt

SEQ ID NO: 932 - DNA, 41 bp, Artificial Sequence
  Description of Artificial Sequence Synthetic oligonucleotide
ccttgacggtgccatggaattttttctcttggaaagaaagt

SEQ ID NO: 933 - DNA, 41 bp, Artificial Sequence
  Description of Artificial Sequence Synthetic oligonucleotide
aagacgccagtggactccactttttctcttggaaagaaagt

SEQ ID NO: 934 - DNA, 38 bp, Artificial Sequence
  Description of Artificial Sequence Synthetic oligonucleotide
ggagcagagagcgaagcggtttttgaagttaccgtttt

SEQ ID NO: 935 - DNA, 39 bp, Artificial Sequence
  Description of Artificial Sequence Synthetic oligonucleotide
cggctgactgtcgaacaggatttttctgagtcaaagcat

SEQ ID NO: 936 - DNA, 36 bp, Artificial Sequence
  Description of Artificial Sequence Synthetic oligonucleotide
gagcgatgtggctcggctttttgaagttaccgtttt

SEQ ID NO: 937 - DNA, 39 bp, Artificial Sequence
  Description of Artificial Sequence Synthetic oligonucleotide
tcaccttccccatggtgtcttttttctgagtcaaagcat

SEQ ID NO: 938 - DNA, 37 bp, Artificial Sequence
  Description of Artificial Sequence Synthetic oligonucleotide
ccaggcgcccaatacgactttttgaagttaccgtttt

SEQ ID NO: 939 - DNA, 40 bp, Artificial Sequence
  Description of Artificial Sequence Synthetic oligonucleotide
agttaaaagcagccctggtgatttttctgagtcaaagcat

SEQ ID NO: 940 - DNA, 44 bp, Artificial Sequence
  Description of Artificial Sequence Synthetic oligonucleotide
tggaacatgtaaaccatgtagttgatttttgaagttaccgtttt

SEQ ID NO: 941 - DNA, 40 bp, Artificial Sequence
  Description of Artificial Sequence Synthetic oligonucleotide
ttgccatgggtggaatcatattttttctgagtcaaagcat

SEQ ID NO: 942 - DNA, 40 bp, Artificial Sequence
  Description of Artificial Sequence Synthetic oligonucleotide
tgacaagcttcccgttctcagtttttgaagttaccgtttt

SEQ ID NO: 943 - DNA, 40 bp, Artificial Sequence
  Description of Artificial Sequence Synthetic oligonucleotide
tggtgatgggatttccattgatttttctgagtcaaagcat

SEQ ID NO: 944 - DNA, 39 bp, Artificial Sequence
  Description of Artificial Sequence Synthetic oligonucleotide
gggatctcgctcctggaagatttttgaagttaccgtttt

SEQ ID NO: 945 - DNA, 38 bp, Artificial Sequence
  Description of Artificial Sequence Synthetic oligonucleotide
cgccccacttgattttggatttttctgagtcaaagcat

SEQ ID NO: 946 - DNA, 13 bp, Artificial Sequence
  Description of Artificial Sequence Synthetic oligonucleotide
ccgccgccccccc

SEQ ID NO: 947 - DNA, 21 bp, Artificial Sequence
  Description of Artificial Sequence Synthetic oligonucleotide
aagcctgccagtgatggtcta

SEQ ID NO: 948 - DNA, 17 bp, Artificial Sequence
  Description of Artificial Sequence Synthetic oligonucleotide
aatatgggggccgccag

SEQ ID NO: 949 - DNA, 21 bp, Artificial Sequence
  Description of Artificial Sequence Synthetic oligonucleotide
ttttgggtggtctccttagca

SEQ ID NO: 950 - DNA, 35 bp, Artificial Sequence
  Description of Artificial Sequence Synthetic oligonucleotide
cgggtccgccgggttttttctcttggaaagaaagt

SEQ ID NO: 951 - DNA, 37 bp, Artificial Sequence
  Description of Artificial Sequence Synthetic oligonucleotide
ggagcaccgcccaagctttttctcttggaaagaaagt

SEQ ID NO: 952 - DNA, 38 bp, Artificial Sequence
  Description of Artificial Sequence Synthetic oligonucleotide
caggcccagcacctgcttttttctcttggaaagaaagt

SEQ ID NO: 953 - DNA, 42 bp, Artificial Sequence
  Description of Artificial Sequence Synthetic oligonucleotide
agggcacacttctgtccagtgtttttctcttggaaagaaagt

SEQ ID NO: 954 - DNA, 42 bp, Artificial Sequence
  Description of Artificial Sequence Synthetic oligonucleotide
cgctcctgaatcctgctgaactttttctcttggaaagaaagt

SEQ ID NO: 955 - DNA, 40 bp, Artificial Sequence
  Description of Artificial Sequence Synthetic oligonucleotide
tggcagtgccaatatcccgtttttctcttggaaagaaagt

SEQ ID NO: 956 - DNA, 41 bp, Artificial Sequence
  Description of Artificial Sequence Synthetic oligonucleotide
aagccaaaatcggtgagctttttttctcttggaaagaaagt

SEQ ID NO: 957 - DNA, 39 bp, Artificial Sequence
  Description of Artificial Sequence Synthetic oligonucleotide
cagggtgtctgcagggcatttttctcttggaaagaaagt

SEQ ID NO: 958 - DNA, 40 bp, Artificial Sequence
  Description of Artificial Sequence Synthetic oligonucleotide
cactgcgtacttcttgggctctttttgaagttaccgtttt

SEQ ID NO: 959 - DNA, 41 bp, Artificial Sequence
  Description of Artificial Sequence Synthetic oligonucleotide
tggacaactggtagtcgtcggttttttctgagtcaaagcat

SEQ ID NO: 960 - DNA, 39 bp, Artificial Sequence
  Description of Artificial Sequence Synthetic oligonucleotide
agcactttgccgttcacacctttttgaagttaccgtttt

SEQ ID NO: 961 - DNA, 37 bp, Artificial Sequence
  Description of Artificial Sequence Synthetic oligonucleotide
cgccgatggaagcactcctttttctgagtcaaagcat

SEQ ID NO: 962 - DNA, 41 bp, Artificial Sequence
  Description of Artificial Sequence Synthetic oligonucleotide
ggggctgtcatacaggagcttctttttgaagttaccgtttt

SEQ ID NO: 963 - DNA, 35 bp, Artificial Sequence
  Description of Artificial Sequence Synthetic oligonucleotide
cctcctgccgggcctttttttctgagtcaaagcat

SEQ ID NO: 964 - DNA, 42 bp, Artificial Sequence
  Description of Artificial Sequence Synthetic oligonucleotide
tcatacacatccaggatgcagactttttgaagttaccgtttt

SEQ ID NO: 965 - DNA, 39 bp, Artificial Sequence
  Description of Artificial Sequence Synthetic oligonucleotide
gcttgccatggtgcatgttctttttctgagtcaaagcat

SEQ ID NO: 966 - DNA, 41 bp, Artificial Sequence
  Description of Artificial Sequence Synthetic oligonucleotide
tccatgatgatgaggagacagctttttgaagttaccgtttt

SEQ ID NO: 967 - DNA, 40 bp, Artificial Sequence
  Description of Artificial Sequence Synthetic oligonucleotide
aactcaccaccttccatgcattttttctgagtcaaagcat

SEQ ID NO: 968 - DNA, 37 bp, Artificial Sequence
  Description of Artificial Sequence Synthetic oligonucleotide
gtgaaagcctggtcgccatttttgaagttaccgtttt

SEQ ID NO: 969 - DNA, 44 bp, Artificial Sequence
  Description of Artificial Sequence Synthetic oligonucleotide
cattatctctgcagcttctctctcatttttctgagtcaaagcat

SEQ ID NO: 970 - DNA, 40 bp, Artificial Sequence
  Description of Artificial Sequence Synthetic oligonucleotide
tatggctgtgcagaaactggatttttgaagttaccgtttt

SEQ ID NO: 971 - DNA, 39 bp, Artificial Sequence
  Description of Artificial Sequence Synthetic oligonucleotide
gacatctcggtgggcaatgttttttctgagtcaaagcat

SEQ ID NO: 972 - DNA, 44 bp, Artificial Sequence
  Description of Artificial Sequence Synthetic oligonucleotide
atgtgtagagtaggttttcaggctttttttgaagttaccgtttt

SEQ ID NO: 973 - DNA, 42 bp, Artificial Sequence
  Description of Artificial Sequence Synthetic oligonucleotide
aagcactgcgtctttctccttagtttttctgagtcaaagcat

SEQ ID NO: 974 - DNA, 26 bp, Artificial Sequence
  Description of Artificial Sequence Synthetic oligonucleotide
ctcagtctctatggcatgattcatat

SEQ ID NO: 975 - DNA, 17 bp, Artificial Sequence
  Description of Artificial Sequence Synthetic oligonucleotide
ggctgaacccccagggc

SEQ ID NO: 976 - DNA, 19 bp, Artificial Sequence
  Description of Artificial Sequence Synthetic oligonucleotide
acttggttccgctacgggt

SEQ ID NO: 977 - DNA, 22 bp, Artificial Sequence
  Description of Artificial Sequence Synthetic oligonucleotide
ccccaaacttctcattgcagag

SEQ ID NO: 978 - DNA, 26 bp, Artificial Sequence
  Description of Artificial Sequence Synthetic oligonucleotide
cttgacttcatcattctctgctaaga

SEQ ID NO: 979 - DNA, 45 bp, Artificial Sequence
  Description of Artificial Sequence Synthetic oligonucleotide
tggtgtattcatctagttcaggcatttttctcttggaaagaaagt

SEQ ID NO: 980 - DNA, 39 bp, Artificial Sequence
  Description of Artificial Sequence Synthetic oligonucleotide
ttcgatccccaggccttctttttctcttggaaagaaagt

SEQ ID NO: 981 - DNA, 42 bp, Artificial Sequence
  Description of Artificial Sequence Synthetic oligonucleotide
gattccataacaggcctggtgtttttctcttggaaagaaagt

SEQ ID NO: 982 - DNA, 41 bp, Artificial Sequence
  Description of Artificial Sequence Synthetic oligonucleotide
agcacagctaacgtggaccctttttctcttggaaagaaagt

SEQ ID NO: 983 - DNA, 41 bp, Artificial Sequence
  Description of Artificial Sequence Synthetic oligonucleotide
gacaccttggtgatgggctctttttctcttggaaagaaagt

SEQ ID NO: 984 - DNA, 47 bp, Artificial Sequence
  Description of Artificial Sequence Synthetic oligonucleotide
gttcttcacagagcactgtatagaggtttttctcttggaaagaaagt

SEQ ID NO: 985 - DNA, 38 bp, Artificial Sequence
  Description of Artificial Sequence Synthetic oligonucleotide
tggaaggctgtgcggcatttttctcttggaaagaaagt

SEQ ID NO: 986 - DNA, 43 bp, Artificial Sequence
  Description of Artificial Sequence Synthetic oligonucleotide
gctttgggcaataggacttgaatttttctcttggaaagaaagt

SEQ ID NO: 987 - DNA, 39 bp, Artificial Sequence
  Description of Artificial Sequence Synthetic oligonucleotide
tggtcagttccagccatgcatttttgaagttaccgtttt

SEQ ID NO: 988 - DNA, 43 bp, Artificial Sequence
  Description of Artificial Sequence Synthetic oligonucleotide
ttcccatctccttaaattcttcattttttctgagtcaaagcat

SEQ ID NO: 989 - DNA, 41 bp, Artificial Sequence
  Description of Artificial Sequence Synthetic oligonucleotide
aattcctctaggaccctctccatttttgaagttaccgtttt

SEQ ID NO: 990 - DNA, 39 bp, Artificial Sequence
  Description of Artificial Sequence Synthetic oligonucleotide
tgtcgtagcatcgctgctcatttttctgagtcaaagcat

SEQ ID NO: 991 - DNA, 46 bp, Artificial Sequence
  Description of Artificial Sequence Synthetic oligonucleotide
gacatcacagacaacatcttcatcatatttttgaagttaccgtttt

SEQ ID NO: 992 - DNA, 40 bp, Artificial Sequence
  Description of Artificial Sequence Synthetic oligonucleotide
ctcaccatcaggagactggcatttttctgagtcaaagcat

SEQ ID NO: 993 - DNA, 40 bp, Artificial Sequence
  Description of Artificial Sequence Synthetic oligonucleotide
gaacaccatctcattgccgtctttttgaagttaccgtttt

SEQ ID NO: 994 - DNA, 43 bp, Artificial Sequence
  Description of Artificial Sequence Synthetic oligonucleotide
cacacagatgttgcatttgtcacatttttctgagtcaaagcat

SEQ ID NO: 995 - DNA, 40 bp, Artificial Sequence
  Description of Artificial Sequence Synthetic oligonucleotide
gctgccctctggtaccttgagtttttgaagttaccgtttt

SEQ ID NO: 996 - DNA, 37 bp, Artificial Sequence
  Description of Artificial Sequence Synthetic oligonucleotide
acatgtccggcacagccatttttctgagtcaaagcat

SEQ ID NO: 997 - DNA, 41 bp, Artificial Sequence
  Description of Artificial Sequence Synthetic oligonucleotide
cttcggacacagcagacatttttttttgaagttaccgtttt

SEQ ID NO: 998 - DNA, 40 bp, Artificial Sequence
  Description of Artificial Sequence Synthetic oligonucleotide
gggcttcatagctccaccctttttttctgagtcaaagcat

SEQ ID NO: 999 - DNA, 40 bp, Artificial Sequence
  Description of Artificial Sequence Synthetic oligonucleotide
gctcacctcagggatccacagtttttgaagttaccgtttt

SEQ ID NO: 1000 - DNA, 40 bp, Artificial Sequence
  Description of Artificial Sequence Synthetic oligonucleotide
catcttctctgggctgccaattttttctgagtcaaagcat

SEQ ID NO: 1001 - DNA, 37 bp, Artificial Sequence
  Description of Artificial Sequence Synthetic oligonucleotide
cggctgctgggaatgtgttttttgaagttaccgtttt

SEQ ID NO: 1002 - DNA, 38 bp, Artificial Sequence
  Description of Artificial Sequence Synthetic oligonucleotide
gctgcacactagcgcccactttttctgagtcaaagcat

SEQ ID NO: 1003 - DNA, 40 bp, Artificial Sequence
  Description of Artificial Sequence Synthetic oligonucleotide
cggtcaaaagcacaggtcacatttttgaagttaccgtttt

SEQ ID NO: 1004 - DNA, 39 bp, Artificial Sequence
  Description of Artificial Sequence Synthetic oligonucleotide
tggtcttcatctccaggccctttttctgagtcaaagcat

SEQ ID NO: 1005 - DNA, 23 bp, Artificial Sequence
  Description of Artificial Sequence Synthetic oligonucleotide
aaaacttcaccgttccattcaag

SEQ ID NO: 1006 - DNA, 50 bp, Artificial Sequence; identical to SEQ ID NO: 859
  Description of Artificial Sequence Synthetic oligonucleotide

SEQ ID NO: 1007 - DNA, 42 bp, Artificial Sequence; identical to SEQ ID NO: 860
  Description of Artificial Sequence Synthetic oligonucleotide

SEQ ID NO: 1008 - DNA, 40 bp, Artificial Sequence; identical to SEQ ID NO: 863
  Description of Artificial Sequence Synthetic oligonucleotide

SEQ ID NO: 1009 - DNA, 40 bp, Artificial Sequence; identical to SEQ ID NO: 866
  Description of Artificial Sequence Synthetic oligonucleotide

SEQ ID NO: 1010 - DNA, 39 bp, Artificial Sequence; identical to SEQ ID NO: 869
  Description of Artificial Sequence Synthetic oligonucleotide

SEQ ID NO: 1011 - DNA, 42 bp, Artificial Sequence; identical to SEQ ID NO: 873
  Description of Artificial Sequence Synthetic oligonucleotide

SEQ ID NO: 1012 - DNA, 42 bp, Artificial Sequence; identical to SEQ ID NO: 857
  Description of Artificial Sequence Synthetic oligonucleotide

SEQ ID NO: 1013 - DNA, 40 bp, Artificial Sequence; identical to SEQ ID NO: 858
  Description of Artificial Sequence Synthetic oligonucleotide

SEQ ID NO: 1014 - DNA, 40 bp, Artificial Sequence; identical to SEQ ID NO: 861
  Description of Artificial Sequence Synthetic oligonucleotide

SEQ ID NO: 1015 - DNA, 37 bp, Artificial Sequence; identical to SEQ ID NO: 862
  Description of Artificial Sequence Synthetic oligonucleotide

SEQ ID NO: 1016 - DNA, 43 bp, Artificial Sequence; identical to SEQ ID NO: 864
  Description of Artificial Sequence Synthetic oligonucleotide

SEQ ID NO: 1017 - DNA, 40 bp, Artificial Sequence; identical to SEQ ID NO: 865
  Description of Artificial Sequence Synthetic oligonucleotide

SEQ ID NO: 1018 - DNA, 35 bp, Artificial Sequence; identical to SEQ ID NO: 867
  Description of Artificial Sequence Synthetic oligonucleotide

SEQ ID NO: 1019 - DNA, 38 bp, Artificial Sequence; identical to SEQ ID NO: 868
  Description of Artificial Sequence Synthetic oligonucleotide

SEQ ID NO: 1020 - DNA, 40 bp, Artificial Sequence; identical to SEQ ID NO: 870
  Description of Artificial Sequence Synthetic oligonucleotide

SEQ ID NO: 1021 - DNA, 42 bp, Artificial Sequence; identical to SEQ ID NO: 871
  Description of Artificial Sequence Synthetic oligonucleotide

SEQ ID NO: 1022 - DNA, 44 bp, Artificial Sequence; identical to SEQ ID NO: 874
  Description of Artificial Sequence Synthetic oligonucleotide

SEQ ID NO: 1023 - DNA, 39 bp, Artificial Sequence; identical to SEQ ID NO: 875
  Description of Artificial Sequence Synthetic oligonucleotide

SEQ ID NO: 1024 - DNA, 19 bp, Artificial Sequence
  Description of Artificial Sequence Synthetic oligonucleotide
gcaagggagctgtacagga

SEQ ID NO: 1025 - DNA, 19 bp, Artificial Sequence
  Description of Artificial Sequence Synthetic oligonucleotide
gaaatgaagctgtacagga

SEQ ID NO: 1026 - DNA, 19 bp, Artificial Sequence
  Description of Artificial Sequence Synthetic oligonucleotide
tcaatccagctgtacaggg

SEQ ID NO: 1027 - DNA, 19 bp, Artificial Sequence
  Description of Artificial Sequence Synthetic oligonucleotide
ccaagagagttttacaggc

SEQ ID NO: 1028 - DNA, 19 bp, Artificial Sequence
  Description of Artificial Sequence Synthetic oligonucleotide
ccaaggtacctttacagga

SEQ ID NO: 1029 - DNA, 19 bp, Artificial Sequence
  Description of Artificial Sequence Synthetic oligonucleotide
acacgggagctgtagaggg

SEQ ID NO: 1030 - DNA, 19 bp, Artificial Sequence
  Description of Artificial Sequence Synthetic oligonucleotide
ccaaggaagctgtacatgc

SEQ ID NO: 1031 - DNA, 19 bp, Artificial Sequence
  Description of Artificial Sequence Synthetic oligonucleotide
gcaaggaagctggacaggg

SEQ ID NO: 1032 - DNA, 19 bp, Artificial Sequence
  Description of Artificial Sequence Synthetic oligonucleotide
gcaagggggctgcacagga

SEQ ID NO: 1033 - DNA, 19 bp, Artificial Sequence
  Description of Artificial Sequence Synthetic oligonucleotide
tcaaggaagctgtgcaggg

SEQ ID NO: 1034 - DNA, 19 bp, Artificial Sequence
  Description of Artificial Sequence Synthetic oligonucleotide
gcaagggacctgaacagga

SEQ ID NO: 1035 - DNA, 19 bp, Artificial Sequence
  Description of Artificial Sequence Synthetic oligonucleotide
tcaagggagcagtactggt

SEQ ID NO: 1036 - DNA, 19 bp, Artificial Sequence
  Description of Artificial Sequence Synthetic oligonucleotide
gctggggagctgaacaggg

SEQ ID NO: 1037 - DNA, 19 bp, Artificial Sequence
  Description of Artificial Sequence Synthetic oligonucleotide
ttctgtccagctcccttgc

SEQ ID NO: 1038 - DNA, 19 bp, Artificial Sequence
  Description of Artificial Sequence Synthetic oligonucleotide
tcgtgtacagctcccttcc

SEQ ID NO: 1039 - DNA, 19 bp, Artificial Sequence
  Description of Artificial Sequence Synthetic oligonucleotide
tcttaaagctggttcatac

SEQ ID NO: 1040 - DNA, 19 bp, Artificial Sequence
  Description of Artificial Sequence Synthetic oligonucleotide
tctcagaactggttcatag

SEQ ID NO: 1041 - DNA, 19 bp, Artificial Sequence
  Description of Artificial Sequence Synthetic oligonucleotide
atataaagttggttcatag

SEQ ID NO: 1042 - DNA, 19 bp, Artificial Sequence
  Description of Artificial Sequence Synthetic oligonucleotide
tcttgatgttggttcatag

SEQ ID NO: 1043 - DNA, 19 bp, Artificial Sequence
  Description of Artificial Sequence Synthetic oligonucleotide
gttcaaagctgcttcatac

SEQ ID NO: 1044 - DNA, 19 bp, Artificial Sequence
  Description of Artificial Sequence Synthetic oligonucleotide
gtttagagctgcttcatat

SEQ ID NO: 1045 - DNA, 19 bp, Artificial Sequence
  Description of Artificial Sequence Synthetic oligonucleotide
tttttaagctgtttcatat

SEQ ID NO: 1046 - DNA, 19 bp, Artificial Sequence
  Description of Artificial Sequence Synthetic oligonucleotide
gcttaaaactggatcatat

SEQ ID NO: 1047 - DNA, 19 bp, Artificial Sequence
  Description of Artificial Sequence Synthetic oligonucleotide
cctaacagctggttcctac

SEQ ID NO: 1048 - DNA, 19 bp, Artificial Sequence
  Description of Artificial Sequence Synthetic oligonucleotide
actttaagcaggttcaaag

SEQ ID NO: 1049 - DNA, 19 bp, Artificial Sequence
  Description of Artificial Sequence Synthetic oligonucleotide
cctcaaagttgggtcatac

SEQ ID NO: 1050 - DNA, 19 bp, Artificial Sequence
  Description of Artificial Sequence Synthetic oligonucleotide
ctttaaagatgggtcatat

SEQ ID NO: 1051 - DNA, 19 bp, Artificial Sequence
  Description of Artificial Sequence Synthetic oligonucleotide
gcttatagctgcttcattc

SEQ ID NO: 1052 - DNA, 19 bp, Artificial Sequence
  Description of Artificial Sequence Synthetic oligonucleotide
tgatgaaccagatttaagc

SEQ ID NO: 1053 - DNA, 19 bp, Artificial Sequence
  Description of Artificial Sequence Synthetic oligonucleotide
ttatgaaccagatttcagg

SEQ ID NO: 1054 - DNA, 19 bp, Artificial Sequence
  Description of Artificial Sequence Synthetic oligonucleotide
acttaaagctggttcatat

SEQ ID NO: 1055 - DNA, 19 bp, Artificial Sequence
  Description of Artificial Sequence Synthetic oligonucleotide
tctcagaactggttcatag

SEQ ID NO: 1056 - DNA, 19 bp, Artificial Sequence
  Description of Artificial Sequence Synthetic oligonucleotide
atataaagttggttcatag

SEQ ID NO: 1057 - DNA, 19 bp, Artificial Sequence
  Description of Artificial Sequence Synthetic oligonucleotide
tcttgatgttggttcatag

SEQ ID NO: 1058 - DNA, 19 bp, Artificial Sequence
  Description of Artificial Sequence Synthetic oligonucleotide
gttcaaagctgcttcatac

SEQ ID NO: 1059 - DNA, 19 bp, Artificial Sequence
  Description of Artificial Sequence Synthetic oligonucleotide
gtttagagctgcttcatat

SEQ ID NO: 1060 - DNA, 19 bp, Artificial Sequence
  Description of Artificial Sequence Synthetic oligonucleotide
tttttaagctgtttcatat

SEQ ID NO: 1061 - DNA, 19 bp, Artificial Sequence
  Description of Artificial Sequence Synthetic oligonucleotide
gcttaaaactggatcatat

SEQ ID NO: 1062 - DNA, 19 bp, Artificial Sequence
  Description of Artificial Sequence Synthetic oligonucleotide
cctaacagctggttcctac

SEQ ID NO: 1063 - DNA, 19 bp, Artificial Sequence
  Description of Artificial Sequence Synthetic oligonucleotide
actttaagcaggttcaaag

SEQ ID NO: 1064 - DNA, 19 bp, Artificial Sequence
  Description of Artificial Sequence Synthetic oligonucleotide
cctcaaagttgggtcatac

SEQ ID NO: 1065 - DNA, 19 bp, Artificial Sequence
  Description of Artificial Sequence Synthetic oligonucleotide
ctttaaagatgggtcatat

SEQ ID NO: 1066 - DNA, 19 bp, Artificial Sequence
  Description of Artificial Sequence Synthetic oligonucleotide
gcttatagctgcttcattc

SEQ ID NO: 1067 - DNA, 19 bp, Artificial Sequence
  Description of Artificial Sequence Synthetic oligonucleotide
tgatgaaccagatttaagc

SEQ ID NO: 1068 - DNA, 19 bp, Artificial Sequence
  Description of Artificial Sequence Synthetic oligonucleotide
ttatgaaccagatttcagg

SEQ ID NO: 1069 - DNA, 19 bp, Artificial Sequence
  Description of Artificial Sequence Synthetic oligonucleotide
agtacacagtgaccgtcga

SEQ ID NO: 1070 - DNA, 19 bp, Artificial Sequence
  Description of Artificial Sequence Synthetic oligonucleotide
tgtacaccgcggccgtcgc

SEQ ID NO: 1071 - DNA, 19 bp, Artificial Sequence
  Description of Artificial Sequence Synthetic oligonucleotide
agtacgcagtgaccgacga

SEQ ID NO: 1072 - DNA, 19 bp, Artificial Sequence
  Description of Artificial Sequence Synthetic oligonucleotide
tggacacagtgaccgtctt

SEQ ID NO: 1073 - DNA, 19 bp, Artificial Sequence
  Description of Artificial Sequence Synthetic oligonucleotide
ggtacacagtgtccgccga

SEQ ID NO: 1074 - DNA, 19 bp, Artificial Sequence
  Description of Artificial Sequence Synthetic oligonucleotide
agaccacagtgaccgtgga

SEQ ID NO: 1075 - DNA, 19 bp, Artificial Sequence
  Description of Artificial Sequence Synthetic oligonucleotide
agtacaacgtgaccatcgt

SEQ ID NO: 1076 - DNA, 19 bp, Artificial Sequence
  Description of Artificial Sequence Synthetic oligonucleotide
cctacacagagaccgtgga

SEQ ID NO: 1077 - DNA, 19 bp, Artificial Sequence
  Description of Artificial Sequence Synthetic oligonucleotide
tgtacaaagagaccgtcta

SEQ ID NO: 1078 - DNA, 19 bp, Artificial Sequence
  Description of Artificial Sequence Synthetic oligonucleotide
tatacacagtcaccgtccc

SEQ ID NO: 1079 - DNA, 19 bp, Artificial Sequence
  Description of Artificial Sequence Synthetic oligonucleotide
tgtacacaaagaccatcga

SEQ ID NO: 1080 - DNA, 19 bp, Artificial Sequence
  Description of Artificial Sequence Synthetic oligonucleotide
agaacacagtggccgtctc

SEQ ID NO: 1081 - DNA, 19 bp, Artificial Sequence
  Description of Artificial Sequence Synthetic oligonucleotide
agtacagagtgcccgtggg

SEQ ID NO: 1082 - DNA, 19 bp, Artificial Sequence
  Description of Artificial Sequence Synthetic oligonucleotide
ccggcgggcactgtgtaca

SEQ ID NO: 1083 - DNA, 19 bp, Artificial Sequence
  Description of Artificial Sequence Synthetic oligonucleotide
tccccggtcactgtgtacc

(19) United States
(12) Patent Application Publication
(10) Pub. No.: US 2011/0196016 A1
(43) Pub. Date: Aug. 11, 2011

(54) Compositions and Methods for Inhibiting Expression of IKK2 Genes

(76) Inventors: Anke Geick, Bayreuth (DE); Markus Hossbach, Kulmbach (DE); Grace Ju, Montclair, NJ (US); Ingo Roehl, Memmelsdorf (DE)

(21) Appl. No.: 13/018,471

(22) Filed: Feb. 1, 2011

(30) Foreign Application Priority Data
Feb. 5, 2010 (EP) 10152801.6

(51) Int. Cl. A61K 31/713; C07H 21/02; C12N 15/63; C12N 5/10; A61P 11/00; A61P 9/00; A61P 19/02; A61P 37/02

(52) U.S. Cl. 514/44 A; 536/24.5; 435/320.1; 435/325

(57) ABSTRACT

The invention relates to a double-stranded ribonucleic acid (dsRNA) for inhibiting the expression of an IKK2 gene. The invention also relates to a pharmaceutical composition comprising the dsRNA or nucleic acid molecules or vectors encoding the same together with a pharmaceutically acceptable carrier; methods for treating diseases caused by the expression of an IKK2 gene using said pharmaceutical composition; and methods for inhibiting the expression of IKK2 in a cell.

### PRIORITY TO RELATED APPLICATION(S)

This application claims the benefit of European Patent Application No. 10152801.6, filed Feb. 5, 2010, which is hereby incorporated by reference in its entirety.

### SEQUENCE LISTING

The instant application contains a Sequence Listing which has been submitted in ASCII format via EFS-Web and is hereby incorporated by reference in its entirety. Said ASCII copy, created on Jan. 13, 2011, is named 26557.txt and is 333,512 bytes in size.

### BACKGROUND OF THE INVENTION

This invention relates to double-stranded ribonucleic acids (dsRNAs), and their use in mediating RNA interference to inhibit the expression of Inhibitor of kappa B kinase 2 (IKK2). Furthermore, the use of said dsRNA to treat autoimmune and inflammatory diseases including but not limited to respiratory diseases/disorders (e.g. asthma and chronic obstructive pulmonary diseases (COPD)) and rheumatoid arthritis is part of this invention. Inhibition of expression of IKK2 by dsRNA is also of use for the treatment of additional diseases such as cancer, type 2 diabetes, non-alcoholic steatohepatitis (NASH), and chronic heart failure.

The transcription factor Nuclear Factor kappa-B (NF-κB) is expressed in numerous cell types in which it functions as a master regulator of immune and inflammatory responses. In unstimulated cells, NF-κB is complexed with Inhibitor of kappa-B (IκB) proteins in an inactive state. Upon stimulation, IκB is phosphorylated by IKK2 which leads to ubiquitination and subsequent degradation of IκB by the proteasome pathway. Loss of IκB exposes a nuclear translocation signal on NF-κB, allowing the transcription factor to enter the nucleus and bind to promoter response elements of NF-κB responsive genes. These NF-κB activated genes include a wide array of inflammatory mediators such as cytokines, chemokines, adhesion molecules, growth factors, and inflammatory enzymes.

There are two main pathways by which NF-κB can be activated: the canonical pathway and the non-canonical pathway. IKK2 is the key regulatory enzyme in the canonical or classical pathway. IKK2 is activated by proinflammatory stimuli which leads to phosphorylation of IκB and its subsequent degradation. It is widely accepted that the canonical pathway is responsible for NF-κB activation in inflammatory states and that inhibition of IKK2 is sufficient to block the majority of NF-κB induced inflammation. The non-canonical or alternative pathway involves activation of IκB kinase 1 (IKK1), which phosphorylates p100 (NF-κB2), subsequently releasing RelB to form transcriptionally active p52-RelB heterodimers. IKK1 is required for secondary lymphoid organogenesis and B cell maturation and survival. IKK1 does also contribute to inflammatory resolution. Thus, a method to inhibit selectively IKK2 rather than IKK1 has distinct advantages for the treatment of inflammatory diseases.

The importance of IKK2 and its role in NF-κB activation in respiratory disorders such as asthma and COPD has been highlighted by numerous studies in mice and in humans. The NF-κB pathway is known to be hyperactivated in uncontrolled severe and moderate asthmatics, who have higher levels of IKK2 protein in peripheral blood mononuclear cells compared to normal individuals. NF-κB activation has been shown to be essential for the inflammatory response in rodent models of allergic asthma in rats and mice. In COPD patients, evidence for activation of the NF-κB pathway has been obtained through analysis of bronchial biopsies and sputum macrophages. In rodent models of cigarette smoking, NF-κB activation has been implicated in disease pathogenesis. Exposure of human bronchial epithelial cells to cigarette smoke extract was demonstrated to stimulate NF-κB activity.

The rationale for treatment of respiratory disorders through suppression of IKK2 is based on studies using chemical or genetic inhibition. Selective small molecule inhibitors of IKK2 have been reported to block ovalbumin-induced lung inflammation and airway hyperresponsiveness in rats (Birrell et al., Am J Respir Crit Care Med 2005; 172: 962-971) and to reduce pulmonary inflammation in LPS or antigen treated mice (Birrell et al., Molec Pharmacol 2006; 69: 1791-1800). Transgenic mice expressing a dominant negative mutant form of IKK2 in airway epithelium were resistant to ovalbumin-induced lung eosinophilia, bronchial fibrosis, and airway mucus production (Broide et al., Proc Natl Acad Sci 2005; 102: 17723-17728). Mice in which IKK2 has been deleted specifically in lung epithelial cells were reported to have reduced numbers of bronchoalveolar lavage neutrophils in response to LPS or cigarette smoke exposure (Lamb et al., Am Thoracic Soc 2008; A12). Human A549 pulmonary cells and primary human bronchial epithelial cells pretreated with IKK2-selective small molecule inhibitors showed profound decreases in the production of inflammatory mediators and inhibition of adhesion molecule expression induced by proinflammatory cytokines IL-1β and TNFα (Newton et al., J Pharmacol Exper Ther 2007; 321: 734-742).

IKK2 plays a role in autoimmune diseases such as rheumatoid arthritis (RA), multiple sclerosis, and Crohn's disease (Ahn et al., Curr Mol Med 2007; 7: 619-637). For example, macrophages from RA synovium have nuclear NF-κB expression, consistent with activation of the NF-κB pathway. In rodent models, increased NF-κB activity has been demonstrated in the synovium of mice and rats following development of collagen- or adjuvant-induced arthritis. Transfer of a dominant-negative IKK2 gene resulted in a decrease in severity of adjuvant arthritis in rats. Several distinct small molecule IKK2 inhibitors have shown significant efficacy in preclinical models of arthritis and inflammatory bowel disease (Strnad and Burke, Trends Pharmacol Sci 2007; 28: 142-148; Bamborough et al., Curr Top Med Chem 2009; 9: 623-639). Administration of an IKK2 inhibitory compound during the induction phase reduced clinical signs of experimental autoimmune encephalomyelitis (EAE), a rodent model of multiple sclerosis (Greve et al., J Immunol 2007; 179: 179-185).

IKK2 has been implicated in other diseases associated with hyperactivation of the NF-κB pathway and underlying chronic inflammatory conditions (Sethi and Tergaonkar, Trends Pharmacol Sci 2009; 30: 313-321). These diseases include cancer (Karin, Cell Res 2008; 18: 334-342; Lee and Hung, Clin Cancer Res 2008; 14: 5656-5662), cardiovascular disease (Li et al., J Mol Med 2008; 86: 1113-1126), and Type 2 diabetes (Bhatt and O'Doherty, Adv Mol Cell Endocrinol 2006; 5: 279-302). IKK inhibitors have shown in vivo activity in preclinical models of melanoma (Yang et al., Clin Cancer Res 2006; 12: 950-960; Hideshima et al., Clin Cancer Res 2006; 12: 5887-5894) and ovarian cancer (Mabuchi et al., Clin Cancer Res 2004; 10: 7645-7654). Transgenic mice with a deletion of the IKK2 gene specifically in hepatocytes retain liver insulin responsiveness on a high-fat diet, while deletion of IKK2 in myeloid cells resulted in systemic insulin responsiveness, suggesting a strong causal relationship between IKK2-regulated inflammation, and obesity-induced insulin resistance (Arkan et al., Nature Med 2005; 11: 191-198). Treatment with an IKK inhibitor significantly reduced plasma glucose levels during insulin resistance tests in mice fed a high-fat diet (Kamon et al., Biochem Biophys Res Commun 2004; 323: 242-248). In a rodent model of NASH, pharmacological inhibition of IKK2 in liver non-parenchymal cells prevented liver steatosis and inflammation, as well as attenuation of liver fibrosis (Beraza et al., Gut 2008; 57: 655-663).

Data from patients and from rodent models clearly indicate that IKK2 is an important mediator of the inflammatory response in respiratory diseases like COPD and asthma, rheumatoid arthritis, NASH and other diseases involving inappropriate NF-κB activation and chronic inflammation. These afore-named diseases represent diseases of significant unmet medical need. COPD is responsible for about 100,000 cases of death per year in the US with increasing prevalence. Statistical extrapolations predict that COPD will be the third leading cause of death worldwide by 2020. Current therapies do not treat underlying inflammation and tissue damage, which is considered to be steroid resistant. About 46 million patients worldwide suffer from asthma. The symptoms of the disease are transiently reversible by treatment with inhaled glucocorticoids. Treatment of severe, steroid resistant asthma and exacerbations remains an unmet medical need. Despite medical advances in the treatment of RA, significant number of patients remain resistant to conventional therapies. The growing prevalence of Type 2 diabetes and NASH is associated with the increase in obesity, thus representing diseases of rising morbidity worldwide.

Double-stranded RNA molecules (dsRNA) have been shown to block gene expression in a highly conserved regulatory mechanism known as RNA interference (RNAi). Downregulation of IKK2 by an IKK2 specific siRNA is expected to inhibit this essential inflammatory regulator and thus ameliorate diseases in which the NF-κB pathway plays an important role in pathogenesis. Thus, an inhibitor of IKK2 expression, and specifically of the expression of IKK2 with the dsRNA molecules of this invention, may be used in the treatment of autoimmune and inflammatory diseases including but not limited to respiratory diseases/disorders (e.g. asthma and chronic obstructive pulmonary diseases (COPD) and rheumatoid arthritis, as well as cancer, type 2 diabetes, non-alcoholic steatohepatitis (NASH), and chronic heart failure.

### SUMMARY OF THE INVENTION

The present invention relates to double-stranded ribonucleic acid molecules (dsRNAs), as well as compositions and methods for inhibiting the expression of the IKK2 gene, and in particular the expression of the IKK2 gene in a cell, tissue or mammal using such dsRNA. The invention also provides compositions and methods for treating pathological conditions and diseases caused by the expression of the IKK2 gene such as autoimmune and inflammatory diseases including but not limited to respiratory diseases/disorders (e.g. asthma and chronic obstructive pulmonary diseases (COPD) and rheumatoid arthritis, as well as cancer, type 2 diabetes, non-alcoholic steatohepatitis (NASH), and chronic heart failure.

In one preferred embodiment the described dsRNA molecule is capable of inhibiting the expression of an IKK2 gene by at least 60%, preferably by at least 70%, and most preferably by at least 80%. The invention also provides compositions and methods for specifically targeting cells in which the NF-kappaB pathway is activated in pathological conditions by expression of the IKK2 gene. These cells include but are not limited to lung epithelial cells, macrophages, T cells, neutrophils, hepatocytes, and tumor cells.

### DETAILED DESCRIPTION OF THE INVENTION

The invention provides double-stranded ribonucleic acid (dsRNA) molecules able to selectively and efficiently decrease the expression of IKK2. The use of IKK2 RNAi provides a method for the therapeutic and/or prophylactic treatment of diseases/disorders which are associated with autoimmune and inflammatory diseases including but not limited to respiratory diseases/disorders (e.g. asthma and chronic obstructive pulmonary diseases (COPD) and rheumatoid arthritis, as well as cancer, type 2 diabetes, non-alcoholic steatohepatitis (NASH), and chronic heart failure.

Particular disease/disorder states include the therapeutic and/or prophylactic treatment of autoimmune and inflammatory diseases including but not limited to respiratory diseases/disorders (e.g. asthma and chronic obstructive pulmonary diseases (COPD) and rheumatoid arthritis, as well as cancer, type 2 diabetes, non-alcoholic steatohepatitis (NASH), and chronic heart failure, which method comprises administration of dsRNA targeting IKK2 to a human being or animal.

In one embodiment, the invention provides double-stranded ribonucleic acid (dsRNA) molecules for inhibiting the expression of an IKK2 gene, in particular the expression of the mammalian or human IKK2 gene. The dsRNA comprises at least two sequences that are complementary to each other. The dsRNA comprises a sense strand comprising a first sequence and an antisense strand comprising a second sequence, see sequences provided in the sequence listing and also the specific dsRNA pairs in the appended tables 1 and 2. In one embodiment the sense strand comprises a sequence which has an identity of at least 90% to at least a portion of an mRNA encoding IKK2. Said sequence is located in a region of complementarity of the sense strand to the antisense strand, preferably within nucleotides 2-7 of the 5′ terminus of the antisense strand. In one preferred embodiment the dsRNA targets particularly the human IKK2 gene. In another embodiment the dsRNA targets the mouse (Mus musculus) and rat (Rattus norvegicus) IKK2 gene.

In one embodiment, the antisense strand comprises a nucleotide sequence which is substantially complementary to at least part of an mRNA encoding said IKK2 gene, and the region of complementarity is most preferably less than 30 nucleotides in length. Furthermore, it is preferred that the length of the herein described inventive dsRNA molecules (duplex length) is in the range of about 16 to 30 nucleotides, in particular in the range of about 18 to 28 nucleotides. Particularly useful in context of this invention are duplex lengths of about 19, 20, 21, 22, 23 or 24 nucleotides. Most preferred are duplex stretches of 19, 21 or 23 nucleotides. The dsRNA, upon contacting with a cell expressing an IKK2 gene, inhibits the expression of an IKK2 gene in vitro by at least 60%, preferably by at least 70%, and most preferably by 80%.

Appended Table 1 relates to preferred molecules to be used as dsRNA in accordance with this invention. Also modified dsRNA molecules are provided herein and are in particular disclosed in appended table 2, providing illustrative examples of modified dsRNA molecules of the present invention. As pointed out herein above, Table 2 provides for illustrative examples of modified dsRNAs of this invention (whereby the corresponding sense strand and antisense strand is provided in this table). The relation of the unmodified preferred molecules shown in Table 1 to the modified dsRNAs of Table 2 is illustrated in Table 13. Yet, the illustrative modifications of these constituents of the inventive dsRNAs are provided herein as examples of modifications.

Tables 3 and 4 provide for selective biological, clinically and pharmaceutical relevant parameters of certain dsRNA molecules of this invention.

Particularly useful with respect to the assessment of therapeutic dsRNAs is the set of dsRNAs targeting mouse and rat IKK2 which can be used to estimate toxicity, therapeutic efficacy, and effective dosages and in vivo half-lives for the individual dsRNAs in an animal or cell culture model. Appended Tables 5 and 6 relate to preferred molecules targeting murine IKK2. Table 6 provides illustrative examples of modified dsRNAs targeting murine IKK2 (whereby the corresponding sense strand and antisense strand is provided in this table). Tables 7 and 8 provide for selective biological, clinically and pharmaceutical relevant parameters of certain dsRNA molecules of this invention. The relation of the unmodified preferred molecules shown in Table 5 to the modified dsRNAs of Table 6 is illustrated in Table 14.

Most preferred dsRNA molecules are provided in the appended table 1 and, inter alia preferably, wherein the sense strand is selected from the group consisting of the nucleic acid sequences depicted in SEQ ID NOs: 1, 2, 3, 5, 6, 8, 9, and 10 and the antisense strand is selected from the from the group consisting of the nucleic acid sequences depicted in SEQ ID NOs: 110, 111, 112, 113 and 114. Accordingly, the inventive dsRNA molecule may, inter alia, comprise the sequence pairs selected from the group consisting of SEQ ID NOs: 1/110, 2/111, 3/112, 5/113, 6/111, 8/114, 9/114 and 10/110. In the context of specific dsRNA molecules provided herein, pairs of SEQ ID NOs relate to corresponding sense and antisense strands sequences (5′ to 3′) as also shown in the appended and included tables.

In one embodiment said dsRNA molecules comprise an antisense strand with a 3′ overhang of 1-5 nucleotides length, preferably of 1-2 nucleotides length. Preferably said overhang of the antisense strand comprises uracil or nucleotides which are complementary to the mRNA encoding IKK2.

In another preferred embodiment, said dsRNA molecules comprise a sense strand with a 3′ overhang of 1-5 nucleotides length, preferably of 1-2 nucleotides length. Preferably said overhang of the sense strand comprises uracil or nucleotides which are identical to the mRNA encoding IKK2.

In another preferred embodiment, said dsRNA molecules comprise a sense strand with a 3′ overhang of 1-5 nucleotides length, preferably of 1-2 nucleotides length, and an antisense strand with a 3′ overhang of 1-5 nucleotides length, preferably of 1-2 nucleotides length. Preferably said overhang of the sense strand comprises uracil or nucleotides which are at least 90% identical to the mRNA encoding IKK2 and said overhang of the antisense strand comprises uracil or nucleotides which are at least 90% complementary to the mRNA encoding IKK2.

The dsRNA molecules of the invention may be comprised of naturally occurring nucleotides or may be comprised of at least one modified nucleotide, such as a 2′-O-methyl modified nucleotide, a nucleotide comprising a 5′-phosphorothioate group, and a terminal nucleotide linked to a cholesteryl derivative or dodecanoic acid bisdecylamide group. 2′ modified nucleotides may have the additional advantage that certain immunostimulatory factors or cytokines are suppressed when the inventive dsRNA molecules are employed in vivo, for example in a medical setting. Alternatively and non-limiting, the modified nucleotide may be chosen from the group of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide. In one preferred embodiment the dsRNA molecules comprise at least one of the following modified nucleotides: a 2′-O-methyl modified nucleotide, a 5′ O-methyl modified nucleotide, a 2′ deoxy-fluoro modification, a nucleotide comprising a 5′-phosphorothioate group, inverted deoxythymidine, a deoxythymidine and 5′ phosphate group at the 5′ end of the antisense strand. Preferred dsRNA molecules comprising modified nucleotides are given in table 2.

In a preferred embodiment the inventive dsRNA molecules comprise modified nucleotides as detailed in the sequences given in table 2. In one preferred embodiment the inventive dsRNA molecule comprises sequence pairs selected from the group consisting of SEQ ID NOs: 1/110, 2/111, 3/112, 5/113, 6/111, 8/114, 9/114 and 10/110, and comprises overhangs at the antisense and/or sense strand of 1-2 deoxythymidines. In one preferred embodiment the inventive dsRNA molecule comprises sequence pairs selected from the group consisting of SEQ ID NOs: 1/110, 2/111, 3/112, 5/113, 6/111, 8/114, 9/114 and 10/110, and comprise modifications as detailed in table 2. Preferred dsRNA molecules comprising modified nucleotides are listed in table 4, with most preferred /dsRNA molecules depicted in SEQ ID Nos: 211/212, 213/214, 215/216, 217/218, 219/220, 223/224, 225/226, 229/230, 231/232, 233/234, 235/236 and 241/242. The relation between the core sequences and their modified counterparts is shown in table 13.

In another embodiment the inventive dsRNAs comprise modified nucleotides on positions different from those disclosed in tables 2. In one preferred embodiment two deoxythymidine nucleotides are found at the 3′ of both strands of the dsRNA molecule.

In one embodiment the dsRNA molecules of the invention comprise a sense and an antisense strand wherein both strands have a half-life of at least 0.4 hours. In one preferred embodiment the dsRNA molecules of the invention comprise a sense and an antisense strand wherein both strands have a half-life of at least 8.6 hours in human ARDS bronchoalveolar lavage (BAL) fluid (BAL fluid from patients suffering from acute respiratory distress syndrome (ARDS)). In another embodiment the dsRNA molecules of the invention are non-immunostimulatory, e.g. do not stimulate IFN-alpha and TNF-alpha in vitro. In another embodiment the dsRNA molecules of the invention do stimulate IFN-alpha and TNF-alpha in vitro to a very minor degree.

The invention also provides for cells comprising at least one of the dsRNAs of the invention. The cell is preferably a mammalian cell, such as a human cell. Furthermore, tissues and/or non-human organisms comprising the herein defined dsRNA molecules are also contemplated, whereby said non-human organisms are particularly useful for research purposes, as research tools, or in drug testing.

Furthermore, the invention relates to a method for inhibiting the expression of an IKK2 gene, in particular a mammalian or human IKK2 gene, in a cell, tissue or organism comprising the following steps:

(a) introducing into the cell, tissue or organism a double-stranded ribonucleic acid (dsRNA) as defined herein;
(b) maintaining said cell, tissue or organism produced in step (a) for a time sufficient to obtain degradation of the mRNA transcript of an IKK2 gene, thereby inhibiting expression of an IKK2 gene in a given cell.

The invention also relates to pharmaceutical compositions comprising the inventive dsRNAs of this invention. These pharmaceutical compositions are particularly useful in the inhibition of the expression of an IKK2 gene in a cell, a tissue or an organism. The pharmaceutical composition comprising one or more of the dsRNAs of the invention may also comprise (a) pharmaceutically acceptable carrier(s), diluent(s) and/or excipient(s).

In another embodiment, the invention provides methods for treating, preventing or managing autoimmune and inflammatory diseases including but not limited to respiratory diseases/disorders (e.g. asthma and chronic obstructive pulmonary diseases (COPD) and rheumatoid arthritis, as well as cancer, type 2 diabetes, non-alcoholic steatohepatitis (NASH), and chronic heart failure which are associated with IKK2, said method comprising administering to a subject in need of such treatment, prevention or management a therapeutically or prophylactically effective amount of one or more of the dsRNAs of the invention. Preferably, said subject is a mammal, most preferably a human patient.

In one embodiment, the invention provides a method for treating a subject having a pathological condition mediated by the expression of an IKK2 gene. Such conditions comprise disorders associated with autoimmune and inflammatory diseases including but not limited to respiratory diseases/disorders (e.g. asthma and chronic obstructive pulmonary diseases (COPD) and rheumatoid arthritis, as well as cancer, type 2 diabetes, non-alcoholic steatohepatitis (NASH), and chronic heart failure.

In this embodiment, the dsRNA acts as a therapeutic agent for controlling the expression of an IKK2 gene. The method comprises administering a pharmaceutical composition of the invention to the patient (e.g., human), such that expression of an IKK2 gene is silenced. Because of their high specificity, the dsRNAs of the invention specifically target mRNAs of an IKK2 gene. In one preferred embodiment the described dsRNAs specifically decrease IKK2 mRNA levels and do not directly affect the expression and/or mRNA levels of off-target genes in the cell.

In one embodiment the described dsRNAs decrease IKK2 mRNA levels in vivo for at least 4 days. In another embodiment, the invention provides vectors for inhibiting the expression of an IKK2 gene in a cell, in particular an IKK2 gene comprising a regulatory sequence operably linked to a nucleotide sequence that encodes at least one strand of one of the dsRNA of the invention.

In another embodiment, the invention provides a cell comprising a vector for inhibiting the expression of an IKK2 gene in a cell. Said vector comprises a regulatory sequence operably linked to a nucleotide sequence that encodes at least one strand of one of the dsRNAs of the invention. Yet, it is preferred that said vector comprises, besides said regulatory sequence a sequence that encodes at least one “sense strand” of the inventive dsRNA and at least one “anti sense strand” of said dsRNA. It is also envisaged that the claimed cell comprises two or more vectors comprising, besides said regulatory sequences, the herein defined sequence(s) that encode(s) at least one strand of one of the dsRNAs of the invention.

In one embodiment, the method comprises administering a composition comprising a dsRNA, wherein the dsRNA comprises a nucleotide sequence which is complementary to at least a part of an RNA transcript of an IKK2 gene of the mammal to be treated. As pointed out above, also vectors and cells comprising nucleic acid molecules that encode for at least one strand of the herein defined dsRNA molecules can be used as pharmaceutical compositions and may, therefore, also be employed in the herein disclosed methods of treating a subject in need of medical intervention. It is also of note that these embodiments relating to pharmaceutical compositions and to corresponding methods of treating a (human) subject also relate to approaches like gene therapy approaches. IKK2 specific dsRNA molecules as provided herein or nucleic acid molecules encoding individual strands of these inventive dsRNA molecules may also be inserted into vectors and used as gene therapy vectors for human patients. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In another aspect of the invention, IKK2 specific dsRNA molecules that modulate IKK2 gene expression activity are expressed from transcription units inserted into DNA or RNA vectors (see, e.g., Skillern, A., et al., International PCT Publication No. WO 00/22113). These transgenes can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann, et al., Proc. Natl. Acad. Sci. USA (1995) 92:1292).

The individual strands of a dsRNA can be transcribed by promoters on two separate expression vectors and co-transfected into a target cell. Alternatively each individual strand of the dsRNA can be transcribed by promoters both of which are located on the same expression plasmid. In a preferred embodiment, a dsRNA is expressed as an inverted repeat joined by a linker polynucleotide sequence such that the dsRNA has a stem and loop structure.

The recombinant dsRNA expression vectors are preferably DNA plasmids or viral vectors. dsRNA expressing viral vectors can be constructed based on, but not limited to, adeno-associated virus (for a review, see Muzyczka, et al., Curr. Topics Micro. Immunol. (1992) 158:97-129)); adenovirus (see, for example, Berkner, et al., BioTechniques (1998) 6:616), Rosenfeld et al. (1991, Science 252:431-434), and Rosenfeld et al. (1992), Cell 68:143-155)); or alphavirus as well as others known in the art. Retroviruses have been used to introduce a variety of genes into many different cell types, including epithelial cells, in vitro and/or in vivo (see, e.g., Danos and Mulligan, Proc. Natl. Acad. Sci. USA (1998) 85:6460-6464). Recombinant retroviral vectors capable of transducing and expressing genes inserted into the genome of a cell can be produced by transfecting the recombinant retroviral genome into suitable packaging cell lines such as PA317 and Psi-CRIP (Comette et al., 1991, Human Gene Therapy 2:5-10; Cone et al., 1984, Proc. Natl. Acad. Sci. USA 81:6349). Recombinant adenoviral vectors can be used to infect a wide variety of cells and tissues in susceptible hosts (e.g., rat, hamster, dog, and chimpanzee) (Hsu et al., 1992, J. Infectious Disease, 166:769), and also have the advantage of not requiring mitotically active cells for infection.

The promoter driving dsRNA expression in either a DNA plasmid or viral vector of the invention may be a eukaryotic RNA polymerase I (e.g. ribosomal RNA promoter), RNA polymerase II (e.g. CMV early promoter or actin promoter or Ul snRNA promoter) or preferably RNA polymerase III promoter (e.g. U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to the pancreas (see, e.g. the insulin regulatory sequence for pancreas (Bucchini et al., 1986, Proc. Natl. Acad. Sci. USA 83:2511-2515)).

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals include regulation by ecdysone, by estrogen, progesterone, tetracycline, chemical inducers of dimerization, and isopropyl-beta-D1-thiogalactopyranoside (EPTG). A person skilled in the art would be able to choose the appropriate regulatory/promoter sequence based on the intended use of the dsRNA transgene.

Preferably, recombinant vectors capable of expressing dsRNA molecules are delivered as described below, and persist in target cells. Alternatively, viral vectors can be used that provide for transient expression of dsRNA molecules. Such vectors can be repeatedly administered as necessary. Once expressed, the dsRNAs bind to target RNA and modulate its function or expression. Delivery of dsRNA expressing vectors can be systemic, such as by intravenous or intramuscular administration, by administration to target cells ex-planted from the patient followed by reintroduction into the patient, or by any other means that allows for introduction into a desired target cell.

dsRNA expression DNA plasmids are typically transfected into target cells as a complex with cationic lipid carriers (e.g. Oligofectamine) or non-cationic lipid-based carriers (e.g. Transit-TKO™). Multiple lipid transfections for dsRNA-mediated knockdowns targeting different regions of a single IKK2 gene or multiple IKK2 genes over a period of a week or more are also contemplated by the invention. Successful introduction of the vectors of the invention into host cells can be monitored using various known methods. For example, transient transfection can be signaled with a reporter, such as a fluorescent marker, such as Green Fluorescent Protein (GFP). Stable transfection of ex vivo cells can be ensured using markers that provide the transfected cell with resistance to specific environmental factors (e.g., antibiotics and drugs), such as hygromycin B resistance.

The following detailed description discloses how to make and use the dsRNA and compositions containing dsRNA to inhibit the expression of a target IKK2 gene, as well as compositions and methods for treating diseases and disorders caused by the expression of said IKK2 gene.

### DEFINITIONS

For convenience, the meaning of certain terms and phrases used in the specification, examples, and appended claims, are provided below. If there is an apparent discrepancy between the usage of a term in other parts of this specification and its definition provided in this section, the definition in this section shall prevail.

“G,” “C,” “A”, “U” and “T” or “dT” respectively, each generally stand for a nucleotide that contains guanine, cytosine, adenine, uracil and deoxythymidine as a base, respectively. However, the term “ribonucleotide” or “nucleotide” can also refer to a modified nucleotide, as further detailed below, or a surrogate replacement moiety. Sequences comprising such replacement moieties are embodiments of the invention. As detailed below, the herein described dsRNA molecules may also comprise “overhangs”, i.e. unpaired, overhanging nucleotides which are not directly involved in the RNA double helical structure normally formed by the herein defined pair of “sense strand” and “anti sense strand”. Often, such an overhanging stretch comprises the deoxythymidine nucleotide, in most embodiments, 2 deoxythymidines in the 3′ end. Such overhangs will be described and illustrated below.

The term “IKK2” as used herein relates in particular to the Inhibitor of kappa B kinase 2 also known as inhibitor of kappa light polypeptide gene enhancer in B-cells, kinase beta inhibitor of nuclear factor kappa B kinase beta subunit, nuclear factor NF-kappa-B inhibitor, kinase beta IKK2, IKBKB, IKK-beta, F1140509, IKKB, MGC131801, NFKBIKB, gxHOMSA22818 and said term relates to the corresponding gene, encoded mRNA, encoded protein/polypeptide as well as functional fragments of the same. Preferred is the human IKK2 gene. In other preferred embodiments the dsRNAs of the invention target the IKK2 gene of human (H. sapiens) and cynomolgous monkey (Macaca fascicularis) IKK2 gene. Also dsRNAs targeting the rat (Rattus norvegicus) and mouse (Mus musculus) IKK2 gene are part of this invention. The term “IKK2 gene/sequence” does not only relate to (the) wild-type sequence(s) but also to mutations and alterations which may be comprised in said gene/sequence. Accordingly, the present invention is not limited to the specific dsRNA molecules provided herein. The invention also relates to dsRNA molecules that comprise an antisense strand that is at least 85% complementary to the corresponding nucleotide stretch of an RNA transcript of an IKK2 gene that comprises such mutations/alterations.

As used herein, “target sequence” refers to a contiguous portion of the nucleotide sequence of an mRNA molecule formed during the transcription of an IKK2 gene, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature. However, as detailed herein, such a “strand comprising a sequence” may also comprise modifications, like modified nucleotides.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence. “Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled.

Sequences referred to as “fully complementary” comprise base-pairing of the oligonucleotide or polynucleotide comprising the first nucleotide sequence to the oligonucleotide or polynucleotide comprising the second nucleotide sequence over the entire length of the first and second nucleotide sequence.

However, where a first sequence is referred to as “substantially complementary” with respect to a second sequence herein, the two sequences can be fully complementary, or they may form one or more, but preferably not more than 13 mismatched base pairs upon hybridization.

The terms “complementary”, “fully complementary” and “substantially complementary” herein may be used with respect to the base matching between the sense strand and the antisense strand of a dsRNA, or between the antisense strand of a dsRNA and a target sequence, as will be understood from the context of their use.

The term “double-stranded RNA”, “dsRNA molecule”, or “dsRNA”, as used herein, refers to a ribonucleic acid molecule, or complex of ribonucleic acid molecules, having a duplex structure comprising two anti-parallel and substantially complementary nucleic acid strands. The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop”. Where the two strands are connected covalently by means other than an uninterrupted chain of nucleotides between the 3′-end of one strand and the 5′ end of the respective other strand forming the duplex structure, the connecting structure is referred to as a “linker”. The RNA strands may have the same or a different number of nucleotides. In addition to the duplex structure, a dsRNA may comprise one or more nucleotide overhangs. The nucleotides in said “overhangs” may comprise between 0 and 5 nucleotides, whereby “0” means no additional nucleotide(s) that form(s) an “overhang” and whereas “5” means five additional nucleotides on the individual strands of the dsRNA duplex. These optional “overhangs” are located in the 3′ end of the individual strands. As will be detailed below, also dsRNA molecules which comprise only an “overhang” in one of the two strands may be useful and even advantageous in context of this invention. The “overhang” comprises preferably between 0 and 2 nucleotides. Most preferably 2 “dT” (deoxythymidine) nucleotides are found at the 3′ end of both strands of the dsRNA. Also 2 “U” (uracil) nucleotides can be used as overhangs at the 3′ end of both strands of the dsRNA. Accordingly, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. For example the antisense strand comprises 23 nucleotides and the sense strand comprises 21 nucleotides, forming a 2 nucleotide overhang at the 3′ end of the antisense strand. Preferably, the 2 nucleotide overhang is fully complementary to the mRNA of the target gene. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

The term “antisense strand” refers to the strand of a dsRNA which includes a region that is substantially complementary to a target sequence. As used herein, the term “region of complementarity” refers to the region on the antisense strand that is substantially complementary to a sequence, for example a target sequence. Where the region of complementarity is not fully complementary to the target sequence, the mismatches are most tolerated outside nucleotides 2-7 of the 5′ terminus of the antisense strand

The term “sense strand,” as used herein, refers to the strand of a dsRNA that includes a region that is substantially complementary to a region of the antisense strand. “Substantially complementary” means preferably at least 85% of the overlapping nucleotides in sense and antisense strand are complementary.

“Introducing into a cell”, when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a dsRNA may also be “introduced into a cell”, wherein the cell is part of a living organism. In such instance, introduction into the cell will include the delivery to the organism. For example, for in vivo delivery, dsRNA can be injected into a tissue site or administered systemically. It is, for example envisaged that the dsRNA molecules of this invention be administered to a subject in need of medical intervention. Such an administration may comprise the injection of the dsRNA, the vector or a cell of this invention into a diseased side in said subject. In addition, the injection is preferably in close proximity of the diseased tissue is envisaged. In vitro introduction into a cell includes methods known in the art such as electroporation and lipofection.

The term “inflammation” as used herein refers to the biologic response of body tissue to injury, irritation, or disease which can be caused by harmful stimuli, for example, pathogens, damaged cells, or irritants. Inflammation is typically characterized by pain and swelling. Inflammation is intended to encompass both acute responses, in which inflammatory processes are active (e.g., neutrophils and leukocytes), and chronic responses, which are marked by slow progress, a shift in the type of cell present at the site of inflammation, and the formation of connective tissue.

Cancers to be treated comprise, but are again not limited to leukemia, solid tumors, liver cancer, brain cancer, breast cancer, lung cancer and prostate cancer.

The terms “silence”, “inhibit the expression of” and “knock down”, in as far as they refer to an IKK2 gene, herein refer to the at least partial suppression of the expression of an IKK2 gene, as manifested by a reduction of the amount of mRNA transcribed from an IKK2 gene which may be isolated from a first cell or group of cells in which an IKK2 gene is transcribed and which has or have been treated such that the expression of an IKK2 gene is inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to the IKK2 gene transcription, e.g. the amount of protein encoded by an IKK2 gene which is secreted by a cell, or the number of cells displaying a certain phenotype.

As illustrated in the appended examples and in the appended tables provided herein, the inventive dsRNA molecules are capable of inhibiting the expression of a human IKK2 by at least about 60%, preferably by at least 70%, and most preferably by at least 80%, i.e. in vitro. The term “in vitro” as used herein includes but is not limited to cell culture assays. In another embodiment the inventive dsRNA molecules are capable of inhibiting the expression of a mouse or rat IKK2 by at least 60% preferably by at least 70%, and most preferably by at least 80%. The person skilled in the art can readily determine such an inhibition rate and related effects, in particular in light of the assays provided herein.

The term “off target” as used herein refers to all non-target mRNAs of the transcriptome that are predicted by in silico methods to hybridize to the described dsRNAs based on sequence complementarity. The dsRNAs of the present invention preferably do specifically inhibit the expression of IKK2, i.e. do not inhibit the expression of any off-target.

The term “half-life” as used herein is a measure of stability of a compound or molecule and can be assessed by methods known to a person skilled in the art, especially in light of the assays provided herein.

The term “non-immunostimulatory” as used herein refers to the absence of any induction of a immune response by the invented dsRNA molecules. Methods to determine immune responses are well known to a person skilled in the art, for example by assessing the release of cytokines, as described in the examples section.

The terms “treat”, “treatment”, and the like, mean in context of this invention the relief from or alleviation of a disorder related to IKK2 expression, like inflammation and proliferative disorders, like cancers.

As used herein, a “pharmaceutical composition” comprises a pharmacologically effective amount of a dsRNA and a pharmaceutically acceptable carrier. However, such a “pharmaceutical composition” may also comprise individual strands of such a dsRNA molecule or the herein described vector(s) comprising a regulatory sequence operably linked to a nucleotide sequence that encodes at least one strand of a sense or an antisense strand comprised in the dsRNAs of this invention. It is also envisaged that cells, tissues or isolated organs that express or comprise the herein defined dsRNAs may be used as “pharmaceutical compositions”. As used herein, “pharmacologically effective amount,” “therapeutically effective amount” or simply “effective amount” refers to that amount of an RNA effective to produce the intended pharmacological, therapeutic or preventive result.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives as known to persons skilled in the art.

It is in particular envisaged that the pharmaceutically acceptable carrier allows for the systemic administration of the dsRNAs, vectors or cells of this invention. Whereas also the enteric administration is envisaged the parenteral administration and also transdermal or transmucosal (e.g. insufflation, buccal, vaginal, anal) administration as well as inhalation of the drug are feasible ways of administering to a patient in need of medical intervention the compounds of this invention. When parenteral administration is employed, this can comprise the direct injection of the compounds of this invention into the diseased tissue or at least in close proximity. However, also intravenous, intraarterial, subcutaneous, intramuscular, intraperitoneal, intradermal, intrathecal and other administrations of the compounds of this invention are within the skill of the artisan, for example the attending physician.

For intramuscular, subcutaneous and intravenous use, the pharmaceutical compositions of the invention will generally be provided in sterile aqueous solutions or suspensions, buffered to an appropriate pH and isotonicity. In a preferred embodiment, the carrier consists exclusively of an aqueous buffer. In this context, “exclusively” means no auxiliary agents or encapsulating substances are present which might affect or mediate uptake of dsRNA in the cells that express an IKK2 gene. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate. The pharmaceutical compositions useful according to the invention also include encapsulated formulations to protect the dsRNA against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in PCT publication WO 91/06309 which is incorporated by reference herein.

As used herein, a “transformed cell” is a cell into which at least one vector has been introduced from which a dsRNA molecule or at least one strand of such a dsRNA molecule may be expressed. Such a vector is preferably a vector comprising a regulatory sequence operably linked to nucleotide sequence that encodes at least one sense strand or antisense strand of a dsRNA of the present invention.

It can be reasonably expected that shorter dsRNAs comprising one of the sequences in Table 1 and 2 minus only a few nucleotides on one or both ends may be similarly effective as compared to the dsRNAs described above.

As pointed out above, in most embodiments of this invention, the dsRNA molecules provided herein comprise a duplex length (i.e. without “overhangs”) of about 16 to about 30 nucleotides. Particular useful dsRNA duplex lengths are about 19 to about 25 nucleotides. Most preferred are duplex structures with a length of 19 nucleotides. In the inventive dsRNA molecules, the antisense strand is at least partially complementary to the sense strand.

The dsRNA of the invention can contain one or more mismatches to the target sequence. In a preferred embodiment, the dsRNA of the invention contains no more than 13 mismatches. If the antisense strand of the dsRNA contains mismatches to a target sequence, it is preferable that the area of mismatch not be located within nucleotides 2-7 of the 5′ terminus of the antisense strand. In another embodiment it is preferable that the area of mismatch not be located within nucleotides 2-9 of the 5′ terminus of the antisense strand.

As mentioned above, at least one end/strand of the dsRNA may have a single-stranded nucleotide overhang of 1 to 5, preferably 1 or 2 nucleotides. dsRNAs having at least one nucleotide overhang have unexpectedly superior inhibitory properties than their blunt-ended counterparts. Moreover, the present inventors have discovered that the presence of only one nucleotide overhang strengthens the interference activity of the dsRNA, without affecting its overall stability. dsRNA having only one overhang has proven particularly stable and effective in vivo, as well as in a variety of cells, cell culture mediums, blood, and serum. Preferably, the single-stranded overhang is located at the 3′-terminal end of the antisense strand or, alternatively, at the 3′-terminal end of the sense strand. The dsRNA may also have a blunt end, preferably located at the 5′-end of the antisense strand. Preferably, the antisense strand of the dsRNA has a nucleotide overhang at the 3′-end, and the 5′-end is blunt. In another embodiment, one or more of the nucleotides in the overhang is replaced with a nucleoside thiophosphate.

The dsRNA of the present invention may also be chemically modified to enhance stability. The nucleic acids of the invention may be synthesized and/or modified by methods well established in the art, such as those described in “Current protocols in nucleic acid chemistry”, Beaucage, S. L. et al. (Edrs.), John Wiley & Sons, Inc., New York, N.Y., USA, which is hereby incorporated herein by reference. Chemical modifications may include, but are not limited to 2′ modifications, introduction of non-natural bases, covalent attachment to a ligand, and replacement of phosphate linkages with thiophosphate linkages. In this embodiment, the integrity of the duplex structure is strengthened by at least one, and preferably two, chemical linkages. Chemical linking may be achieved by any of a variety of well-known techniques, for example by introducing covalent, ionic or hydrogen bonds; hydrophobic interactions, van der Waals or stacking interactions; by means of metal-ion coordination, or through use of purine analogues. Preferably, the chemical groups that can be used to modify the dsRNA include, without limitation, methylene blue; bifunctional groups, preferably bis-(2-chloroethyl)amine; N-acetyl-N′-(p-glyoxylbenzoyl)cystamine; 4-thiouracil; and psoralen. In one preferred embodiment, the linker is a hexa-ethylene glycol linker. In this case, the dsRNA are produced by solid phase synthesis and the hexa-ethylene glycol linker is incorporated according to standard methods (e.g., Williams, D. J., and K. B. Hall, Biochem. (1996) 35:14665-14670). In a particular embodiment, the 5′-end of the antisense strand and the 3′-end of the sense strand are chemically linked via a hexaethylene glycol linker. In another embodiment, at least one nucleotide of the dsRNA comprises a phosphorothioate or phosphorodithioate groups. The chemical bond at the ends of the dsRNA is preferably formed by triple-helix bonds.

In certain embodiments, a chemical bond may be formed by means of one or several bonding groups, wherein such bonding groups are preferably poly-(oxyphosphinicooxy-1,3-propandiol)- and/or polyethylene glycol chains. In other embodiments, a chemical bond may also be formed by means of purine analogs introduced into the double-stranded structure instead of purines. In further embodiments, a chemical bond may be formed by azabenzene units introduced into the double-stranded structure. In still further embodiments, a chemical bond may be formed by branched nucleotide analogs instead of nucleotides introduced into the double-stranded structure. In certain embodiments, a chemical bond may be induced by ultraviolet light.

In yet another embodiment, the nucleotides at one or both of the two single strands may be modified to prevent or inhibit the activation of cellular enzymes, for example certain nucleases. Techniques for inhibiting the activation of cellular enzymes are known in the art including, but not limited to, 2′-amino modifications, 2′-amino sugar modifications, 2′-F sugar modifications, 2′-F modifications, 2′-alkyl sugar modifications, uncharged backbone modifications, morpholino modifications, 2′-O-methyl modifications, and phosphoramidate (see, e.g., Wagner, Nat. Med. (1995) 1:1116-8). Thus, at least one 2′-hydroxyl group of the nucleotides on a dsRNA is replaced by a chemical group, preferably by a 2′-amino or a 2′-methyl group. Also, at least one nucleotide may be modified to form a locked nucleotide. Such locked nucleotide contains a methylene bridge that connects the 2′-oxygen of ribose with the 4′-carbon of ribose. Introduction of a locked nucleotide into an oligonucleotide improves the affinity for complementary sequences and increases the melting temperature by several degrees.

Modifications of dsRNA molecules provided herein may positively influence their stability in vivo as well as in vitro and also improve their delivery to the (diseased) target side. Furthermore, such structural and chemical modifications may positively influence physiological reactions towards the dsRNA molecules upon administration, e.g. the cytokine release which is preferably suppressed. Such chemical and structural modifications are known in the art and are, inter alia, illustrated in Nawrot (2006) Current Topics in Med Chem, 6, 913-925.

Conjugating a ligand to a dsRNA can enhance its cellular absorption as well as targeting to a particular tissue. In certain instances, a hydrophobic ligand is conjugated to the dsRNA to facilitate direct permeation of the cellular membrane. Alternatively, the ligand conjugated to the dsRNA is a substrate for receptor-mediated endocytosis. These approaches have been used to facilitate cell permeation of antisense oligonucleotides. For example, cholesterol has been conjugated to various antisense oligonucleotides resulting in compounds that are substantially more active compared to their non-conjugated analogs. See M. Manoharan Antisense & Nucleic Acid Drug Development 2002, 12, 103. Other lipophilic compounds that have been conjugated to oligonucleotides include 1-pyrene butyric acid, 1,3-bis-O-(hexadecyl)glycerol, and menthol. One example of a ligand for receptor-mediated endocytosis is folic acid. Folic acid enters the cell by folate-receptor-mediated endocytosis. dsRNA compounds bearing folic acid would be efficiently transported into the cell via the folate-receptor-mediated endocytosis. Attachment of folic acid to the 3′-terminus of an oligonucleotide results in increased cellular uptake of the oligonucleotide (Li, S.; Deshmukh, H. M.; Huang, L. Pharm. Res. 1998, 15, 1540). Other ligands that have been conjugated to oligonucleotides include polyethylene glycols, carbohydrate clusters, cross-linking agents, porphyrin conjugates, and delivery peptides.

In certain instances, conjugation of a cationic ligand to oligonucleotides often results in improved resistance to nucleases. Representative examples of cationic ligands are propylammonium and dimethylpropylammonium. Interestingly, antisense oligonucleotides were reported to retain their high binding affinity to mRNA when the cationic ligand was dispersed throughout the oligonucleotide. See M. Manoharan Antisense & Nucleic Acid Drug Development 2002, 12, 103 and references therein.

The ligand-conjugated dsRNA of the invention may be synthesized by the use of a dsRNA that bears a pendant reactive functionality, such as that derived from the attachment of a linking molecule onto the dsRNA. This reactive oligonucleotide may be reacted directly with commercially-available ligands, ligands that are synthesized bearing any of a variety of protecting groups, or ligands that have a linking moiety attached thereto. The methods of the invention facilitate the synthesis of ligand-conjugated dsRNA by the use of, in some preferred embodiments, nucleoside monomers that have been appropriately conjugated with ligands and that may further be attached to a solid-support material. Such ligand-nucleoside conjugates, optionally attached to a solid-support material, are prepared according to some preferred embodiments of the methods of the invention via reaction of a selected serum-binding ligand with a linking moiety located on the 5′ position of a nucleoside or oligonucleotide. In certain instances, an dsRNA bearing an aralkyl ligand attached to the 3′-terminus of the dsRNA is prepared by first covalently attaching a monomer building block to a controlled-pore-glass support via a long-chain aminoalkyl group. Then, nucleotides are bonded via standard solid-phase synthesis techniques to the monomer building-block bound to the solid support. The monomer building block may be a nucleoside or other organic compound that is compatible with solid-phase synthesis.

The dsRNA used in the conjugates of the invention may be conveniently and routinely made through the well-known technique of solid-phase synthesis. It is also known to use similar techniques to prepare other oligonucleotides, such as the phosphorothioates and alkylated derivatives.

Teachings regarding the synthesis of particular modified oligonucleotides may be found in the following U.S. patents: U.S. Pat. No. 5,218,105, drawn to polyamine conjugated oligonucleotides; U.S. Pat. No. 5,541,307, drawn to oligonucleotides having modified backbones; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having β-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups may be used as linkers to other moieties attached at any of a variety of positions of the nucleoside; U.S. Pat. No. 5,587,361 drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,608,046, both drawn to conjugated 4′-desmethyl nucleoside analogs; U.S. Pat. No. 5,610,289, drawn to backbone-modified oligonucleotide analogs; U.S. Pat. No. 6,262,241 drawn to, inter alia, methods of synthesizing 2′-fluoro-oligonucleotides.

In the ligand-conjugated dsRNA and ligand-molecule bearing sequence-specific linked nucleosides of the invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. Oligonucleotide conjugates bearing a variety of molecules such as steroids, vitamins, lipids and reporter molecules, has previously been described (see Manoharan et al., PCT Application WO 93/07883). In a preferred embodiment, the oligonucleotides or linked nucleosides of the invention are synthesized by an automated synthesizer using phosphoramidites derived from ligand-nucleoside conjugates in addition to commercially available phosphoramidites.

The incorporation of a 2′-O-methyl, 2′-O-ethyl, 2′-O-propyl, 2′-O-allyl, 2′-O-aminoalkyl or 2′-deoxy-2′-fluoro group in nucleosides of an oligonucleotide confers enhanced hybridization properties to the oligonucleotide. Further, oligonucleotides containing phosphorothioate backbones have enhanced nuclease stability. Thus, functionalized, linked nucleosides of the invention can be augmented to include either or both a phosphorothioate backbone or a 2′-β-methyl, 2′-O-ethyl, 2′-O-propyl, 2′-O-aminoalkyl, 2′-O-allyl or 2′-deoxy-2′-fluoro group.

In some preferred embodiments, functionalized nucleoside sequences of the invention possessing an amino group at the 5′-terminus are prepared using a DNA synthesizer, and then reacted with an active ester derivative of a selected ligand. Active ester derivatives are well known to those skilled in the art. Representative active esters include N-hydrosuccinimide esters, tetrafluorophenolic esters, pentafluorophenolic esters and pentachlorophenolic esters. The reaction of the amino group and the active ester produces an oligonucleotide in which the selected ligand is attached to the 5′-position through a linking group. The amino group at the 5′-terminus can be prepared utilizing a 5′-Amino-Modifier C6 reagent. In a preferred embodiment, ligand molecules may be conjugated to oligonucleotides at the 5′-position by the use of a ligand-nucleoside phosphoramidite wherein the ligand is linked to the 5′-hydroxy group directly or indirectly via a linker. Such ligand-nucleoside phosphoramidites are typically used at the end of an automated synthesis procedure to provide a ligand-conjugated oligonucleotide bearing the ligand at the 5′-terminus.

In one preferred embodiment of the methods of the invention, the preparation of ligand conjugated oligonucleotides commences with the selection of appropriate precursor molecules upon which to construct the ligand molecule. Typically, the precursor is an appropriately-protected derivative of the commonly-used nucleosides. For example, the synthetic precursors for the synthesis of the ligand-conjugated oligonucleotides of the invention include, but are not limited to, 2′-aminoalkoxy-5′-ODMT-nucleosides, 2′-6-aminoalkylamino-5′-ODMT-nucleosides, 5′-6-aminoalkoxy-2′-deoxy-nucleosides, 5′-6-aminoalkoxy-2-protected-nucleosides, 3′-6-aminoalkoxy-5′-ODMT-nucleosides, and 3′-aminoalkylamino-5′-ODMT-nucleosides that may be protected in the nucleobase portion of the molecule. Methods for the synthesis of such amino-linked protected nucleoside precursors are known to those of ordinary skill in the art.

In many cases, protecting groups are used during the preparation of the compounds of the invention. As used herein, the term “protected” means that the indicated moiety has a protecting group appended thereon. In some preferred embodiments of the invention, compounds contain one or more protecting groups. A wide variety of protecting groups can be employed in the methods of the invention. In general, protecting groups render chemical functionalities inert to specific reaction conditions, and can be appended to and removed from such functionalities in a molecule without substantially damaging the remainder of the molecule.

Representative hydroxylprotecting groups, as well as other representative protecting groups, are disclosed in Greene and Wuts, Protective Groups in Organic Synthesis, Chapter 2, 2d ed., John Wiley & Sons, New York, 1991, and Oligonucleotides And Analogues A Practical Approach, Ekstein, F. Ed., IRL Press, N.Y, 1991.

Amino-protecting groups stable to acid treatment are selectively removed with base treatment, and are used to make reactive amino groups selectively available for substitution. Examples of such groups are the Fmoc (E. Atherton and R. C. Sheppard in The Peptides, S. Udenfriend, J. Meienhofer, Eds., Academic Press, Orlando, 1987, volume 9, p. 1) and various substituted sulfonylethyl carbamates exemplified by the Nsc group (Samukov et al., Tetrahedron Lett., 1994, 35:7821.

Additional amino-protecting groups include, but are not limited to, carbamate protecting groups, such as 2-trimethylsilylethoxycarbonyl (Teoc), 1-methyl-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide protecting groups, such as formyl, acetyl, trihaloacetyl, benzoyl, and nitrophenylacetyl; sulfonamide protecting groups, such as 2-nitrobenzenesulfonyl; and imine and cyclic imide protecting groups, such as phthalimido and dithiasuccinoyl. Equivalents of these amino-protecting groups are also encompassed by the compounds and methods of the invention.

Many solid supports are commercially available and one of ordinary skill in the art can readily select a solid support to be used in the solid-phase synthesis steps. In certain embodiments, a universal support is used. A universal support allows for the preparation of oligonucleotides having unusual or modified nucleotides located at the 3′-terminus of the oligonucleotide. For further details about universal supports see Scott et al., Innovations and Perspectives in solid-phase Synthesis, 3rd International Symposium, 1994, Ed. Roger Epton, Mayflower Worldwide, 115-124]. In addition, it has been reported that the oligonucleotide can be cleaved from the universal support under milder reaction conditions when the oligonucleotide is bonded to the solid support via a syn-1,2-acetoxyphosphate group which more readily undergoes basic hydrolysis. See Guzaev, A. I.; Manoharan, M. J. Am. Chem. Soc. 2003, 125, 2380.

The nucleosides are linked by phosphorus-containing or non-phosphorus-containing covalent internucleoside linkages. For the purposes of identification, such conjugated nucleosides can be characterized as ligand-bearing nucleosides or ligand-nucleoside conjugates. The linked nucleosides having an aralkyl ligand conjugated to a nucleoside within their sequence will demonstrate enhanced dsRNA activity when compared to like dsRNA compounds that are not conjugated.

The aralkyl-ligand-conjugated oligonucleotides of the invention also include conjugates of oligonucleotides and linked nucleosides wherein the ligand is attached directly to the nucleoside or nucleotide without the intermediacy of a linker group. The ligand may preferably be attached, via linking groups, at a carboxyl, amino or oxo group of the ligand. Typical linking groups may be ester, amide or carbamate groups.

Specific examples of preferred modified oligonucleotides envisioned for use in the ligand-conjugated oligonucleotides of the invention include oligonucleotides containing modified backbones or non-natural internucleoside linkages. As defined here, oligonucleotides having modified backbones or internucleoside linkages include those that retain a phosphorus atom in the backbone and those that do not have a phosphorus atom in the backbone. For the purposes of the invention, modified oligonucleotides that do not have a phosphorus atom in their intersugar backbone can also be considered to be oligonucleosides.

Specific oligonucleotide chemical modifications are described below. It is not necessary for all positions in a given compound to be uniformly modified. Conversely, more than one modifications may be incorporated in a single dsRNA compound or even in a single nucleotide thereof.

Preferred modified internucleoside linkages or backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free-acid forms are also included. Teachings relating to the preparation of the above phosphorus-atom-containing linkages are well known in the art.

Preferred modified internucleoside linkages or backbones that do not include a phosphorus atom therein (i.e., oligonucleosides) have backbones that are formed by short chain alkyl or cycloalkyl intersugar linkages, mixed heteroatom and alkyl or cycloalkyl intersugar linkages, or one or more short chain heteroatomic or heterocyclic intersugar linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents relating to the preparation of the above oligonucleosides include, but are not limited to, U.S. Pat. Nos. 5,034,506; 5,214,134; 5,216,141; 5,264,562; 5,466,677; 5,470,967; 5,489,677; 5,602,240 and 5,663,312, each of which is herein incorporated by reference in their entirety.

In other preferred oligonucleotide mimetics, both the sugar and the internucleoside linkage, i.e., the backbone, of the nucleoside units are replaced with novel groups. The nucleobase units are maintained for hybridization with an appropriate nucleic acid target compound. One such oligonucleotide, an oligonucleotide mimetic, that has been shown to have excellent hybridization properties, is referred to as a peptide nucleic acid (PNA). In PNA compounds, the sugar-backbone of an oligonucleotide is replaced with an amide-containing backbone, in particular an aminoethylglycine backbone. The nucleobases are retained and are bound directly or indirectly to atoms of the amide portion of the backbone. Teaching of PNA compounds can be found for example in U.S. Pat. No. 5,539,082.

Some preferred embodiments of the invention employ oligonucleotides with phosphorothioate linkages and oligonucleosides with heteroatom backbones, and in particular—CH₂—NH—O—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂—[wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

The oligonucleotides employed in the ligand-conjugated oligonucleotides of the invention may additionally or alternatively comprise nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

Further nucleobases include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligonucleotides of the invention. These include 5-substituted pyrimidines, 6-azapyrimidines and N-2, N-6 and O-6 substituted purines, including 2-aminopropyladenine, 5-propynyluracil and 5-propynylcytosine. 5-Methylcytosine substitutions have been shown to increase nucleic acid duplex stability by 0.6-1.2° C. (Id., pages 276-278) and are presently preferred base substitutions, even more particularly when combined with 2′-methoxyethyl sugar modifications.

Representative United States patents relating to the preparation of certain of the above-noted modified nucleobases as well as other modified nucleobases include, but are not limited to, the above noted U.S. Pat. No. 3,687,808, as well as U.S. Pat. Nos. 5,134,066; 5,459,255; 5,552,540; 5,594,121 and 5,596,091 all of which are hereby incorporated by reference.

In certain embodiments, the oligonucleotides employed in the ligand-conjugated oligonucleotides of the invention may additionally or alternatively comprise one or more substituted sugar moieties. Preferred oligonucleotides comprise one of the following at the 2′ position: OH; F; O-, S-, or N-alkyl, O-, S-, or N-alkenyl, or O, S- or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy [2′-O—CH₂CH₂OCH₃, also known as 2′-O-(2-methoxyethyl) or 2′-MOE], i.e., an alkoxyalkoxy group. A further preferred modification includes 2′-dimethylaminooxyethoxy, i.e., a O(CH₂)₂ON(CH₃)₂ group, also known as 2′-DMAOE, as described in U.S. Pat. No. 6,127,533, filed on Jan. 30, 1998, the contents of which are incorporated by reference.

Other preferred modifications include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides.

As used herein, the term “sugar substituent group” or “2′-substituent group” includes groups attached to the 2′-position of the ribofuranosyl moiety with or without an oxygen atom. Sugar substituent groups include, but are not limited to, fluoro, O-alkyl, O-alkylamino, O-alkylalkoxy, protected O-alkylamino, O-alkylaminoalkyl, O-alkyl imidazole and polyethers of the formula (O-alkyl)ₘ, wherein m is 1 to about 10. Preferred among these polyethers are linear and cyclic polyethylene glycols (PEGs), and (PEG)-containing groups, such as crown ethers and, inter alia, those which are disclosed by Delgardo et. al. (Critical Reviews in Therapeutic Drug Carrier Systems 1992, 9:249), which is hereby incorporated by reference in its entirety. Further sugar modifications are disclosed by Cook (Anti-fibrosis Drug Design, 1991, 6:585-607). Fluoro, O-alkyl, O-alkylamino, O-alkyl imidazole, O-alkylaminoalkyl, and alkyl amino substitution is described in U.S. Pat. No. 6,166,197, entitled “Oligomeric Compounds having Pyrimidine Nucleotide(s) with 2′ and 5′ Substitutions,” hereby incorporated by reference in its entirety.

Additional sugar substituent groups amenable to the invention include 2′-SR and 2′-NR₂ groups, wherein each R is, independently, hydrogen, a protecting group or substituted or unsubstituted alkyl, alkenyl, or alkynyl. 2′-SR Nucleosides are disclosed in U.S. Pat. No. 5,670,633, hereby incorporated by reference in its entirety. The incorporation of 2′-SR monomer synthons is disclosed by Hamm et al. (J. Org. Chem., 1997, 62:3415-3420). 2′-NR nucleosides are disclosed by Goettingen, M., J. Org. Chem., 1996, 61, 6273-6281; and Polushin et al., Tetrahedron Lett., 1996, 37, 3227-3230. Further representative 2′-substituent groups amenable to the invention include those having one of formula I or II:

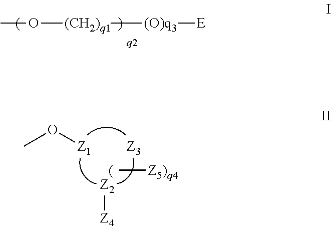

wherein,

E is C₁-C₁₀ alkyl, N(Q₃)(Q₄) or N═C(Q₃)(Q₄); each Q₃ and Q₄ is, independently, H, C₁-C₁₀ alkyl, dialkylaminoalkyl, a nitrogen protecting group, a tethered or untethered conjugate group, a linker to a solid support; or Q₃ and Q₄, together, form a nitrogen protecting group or a ring structure optionally including at least one additional heteroatom selected from N and O;

q1 is an integer from 1 to 10;
q2 is an integer from 1 to 10;
q3 is 0 or 1;
q4 is 0, 1 or 2;
each Z₁, Z₂ and Z₃ is, independently, C₄-C₇ cycloalkyl, C₅-C₁₄ aryl or C₃-Cᵢₛ heterocyclyl, wherein the heteroatom in said heterocyclyl group is selected from oxygen, nitrogen and sulfur; Z₄ is OM₁, SM₁, or N(M₁)₂; each M₁ is, independently, H, C₁-C₈ alkyl, C₁-C₈ haloalkyl, C(═NH)N(H)M₂, C(═O)N(H)M₂ or OC(═O)N(H)M₂; M₂ is H or C₁-C₈ alkyl; and Z₅ is C₁-C₁₀ alkyl, C₁-C₁₀ haloalkyl, C₂-C₁₀ alkenyl, C₂-C₁₀ alkynyl, C₆-C₁₄ aryl, N(Q₃)(Q₄), OQ₃, halo, SQ3 or CN.
Representative 2′-O-sugar substituent groups of formula I are disclosed in U.S. Pat. No. 6,172,209, entitled “Capped 2′-Oxyethoxy Oligonucleotides,” hereby incorporated by reference in its entirety. Representative cyclic 2′-O-sugar substituent groups of formula II are disclosed in U.S. Pat. No. 6,271,358, entitled “RNA Targeted 2′-Modified Oligonucleotides that are Conformationally Preorganized,” hereby incorporated by reference in its entirety.

Sugars having O-substitutions on the ribosyl ring are also amenable to the invention. Representative substitutions for ring O include, but are not limited to, S, CH₂, CHF, and CF₂.

Oligonucleotides may also have sugar mimetics, such as cyclobutyl moieties, in place of the pentofuranosyl sugar. Representative United States patents relating to the preparation of such modified sugars include, but are not limited to, U.S. Pat. Nos. 5,359,044; 5,466,786; 5,519,134; 5,591,722; 5,597,909; 5,646,265 and 5,700,920, all of which are hereby incorporated by reference.

Additional modifications may also be made at other positions on the oligonucleotide, particularly the 3′ position of the sugar on the 3′ terminal nucleotide. For example, one additional modification of the ligand-conjugated oligonucleotides of the invention involves chemically linking to the oligonucleotide one or more additional non-ligand moieties or conjugates which enhance the activity, cellular distribution or cellular uptake of the oligonucleotide. Such moieties include but are not limited to lipid moieties, such as a cholesterol moiety (Letsinger et al., Proc. Natl. Acad. Sci. USA, 1989, 86, 6553), cholic acid (Manoharan et al., Bioorg. Med. Chem. Lett., 1994, 4, 1053), a thioether, e.g., hexyl-5-tritylthiol (Manoharan et al., Ann. N.Y. Acad. Sci., 1992, 660, 306; Manoharan et al., Bioorg. Med. Chem. Let., 1993, 3, 2765), a thiocholesterol (Oberhauser et al., Nucl. Acids Res., 1992, 20, 533), an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), a polyamine or a polyethylene glycol chain (Manoharan et al., Nucleosides & Nucleotides, 1995, 14, 969), or adamantane acetic acid (Manoharan et al., Tetrahedron Lett., 1995, 36, 3651), a palmityl moiety (Mishra et al., Biochim. Biophys. Acta, 1995, 1264, 229), or an octadecylamine or hexylamino-carbonyl-oxycholesterol moiety (Crooke et al., J. Pharmacol. Exp. Ther., 1996, 277, 923).

The invention also includes compositions employing oligonucleotides that are substantially chirally pure with regard to particular positions within the oligonucleotides. Examples of substantially chirally pure oligonucleotides include, but are not limited to, those having phosphorothioate linkages that are at least 75% Sp or Rp (Cook et al., U.S. Pat. No. 5,587,361) and those having substantially chirally pure (Sp or Rp) alkylphosphonate, phosphoramidate or phosphotriester linkages (Cook, U.S. Pat. Nos. 5,212,295 and 5,521,302).

In certain instances, the oligonucleotide may be modified by a non-ligand group. A number of non-ligand molecules have been conjugated to oligonucleotides in order to enhance the activity, cellular distribution or cellular uptake of the oligonucleotide, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-5-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Typical conjugation protocols involve the synthesis of oligonucleotides bearing an aminolinker at one or more positions of the sequence. The amino group is then reacted with the molecule being conjugated using appropriate coupling or activating reagents. The conjugation reaction may be performed either with the oligonucleotide still bound to the solid support or following cleavage of the oligonucleotide in solution phase. Purification of the oligonucleotide conjugate by HPLC typically affords the pure conjugate.

Alternatively, the molecule being conjugated may be converted into a building block, such as a phosphoramidite, via an alcohol group present in the molecule or by attachment of a linker bearing an alcohol group that may be phosphorylated.

Importantly, each of these approaches may be used for the synthesis of ligand conjugated oligonucleotides. Amino linked oligonucleotides may be coupled directly with ligand via the use of coupling reagents or following activation of the ligand as an NHS or pentfluorophenolate ester. Ligand phosphoramidites may be synthesized via the attachment of an aminohexanol linker to one of the carboxyl groups followed by phosphitylation of the terminal alcohol functionality. Other linkers, such as cysteamine, may also be utilized for conjugation to a chloroacetyl linker present on a synthesized oligonucleotide.

The person skilled in the art is readily aware of methods to introduce the molecules of this invention into cells, tissues or organisms. Corresponding examples have also been provided in the detailed description of the invention above. For example, the nucleic acid molecules or the vectors of this invention, encoding for at least one strand of the inventive dsRNAs may be introduced into cells or tissues by methods known in the art, like transfections etc.

Also for the introduction of dsRNA molecules, means and methods have been provided. For example, targeted delivery by glycosylated and folate-modified molecules, including the use of polymeric carriers with ligands, such as galactose and lactose or the attachment of folic acid to various macromolecules allows the binding of molecules to be delivered to folate receptors. Targeted delivery by peptides and proteins other than antibodies, for example, including RGD-modified nanoparticles to deliver siRNA in vivo or multicomponent (nonviral) delivery systems including short cyclodextrins, adamantine-PEG are known. Yet, also the targeted delivery using antibodies or antibody fragments, including (monovalent) Fab-fragments of an antibody (or other fragments of such an antibody) or single-chain antibodies are envisaged. Injection approaches for target directed delivery comprise, inter alia, hydrodynamic i.v. injection. Also cholesterol conjugates of dsRNA may be used for targeted delivery, whereby the conjugation to lipohilic groups enhances cell uptake and improve pharmacokinetics and tissue biodistribution of oligonucleotides. Also cationic delivery systems are known, whereby synthetic vectors with net positive (cationic) charge to facilitate the complex formation with the polyanionic nucleic acid and interaction with the negatively charged cell membrane. Such cationic delivery systems comprise also cationic liposomal delivery systems, cationic polymer and peptide delivery systems. Other delivery systems for the cellular uptake of dsRNA/siRNA are aptamer-ds/siRNA. Also gene therapy approaches can be used to deliver the inventive dsRNA molecules or nucleic acid molecules encoding the same. Such systems comprise the use of non-pathogenic virus, modified viral vectors, as well as deliveries with nanoparticles or liposomes. Other delivery methods for the cellular uptake of dsRNA are extracorporeal, for example ex vivo treatments of cells, organs or tissues. Certain of these technologies are described and summarized in publications, like Akhtar (2007), Journal of Clinical Investigation 117, 3623-3632, Nguyen et al. (2008), Current Opinion in Moleculare Therapeutics 10, 158-167, Zamboni (2005), Clin Cancer Res 11, 8230-8234 or Ikeda et al. (2006), Pharmaceutical Research 23, 1631-1640

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The above provided embodiments and items of the present invention are now illustrated with the following, non-limiting examples.

### DESCRIPTION OF APPENDED TABLES

Table 1—Core sequences of dsRNA molecules targeting human IKK2 gene. Letters in capitals represent RNA nucleotides.

Table 2—dsRNA targeting human IKK2 gene with modifications. Letters in capitals represent RNA nucleotides, (i.e. a 2′-hydroxy corresponding nucleoside), lower case letters “c”, “g”, “a” and “u” represent 2′ O-methyl-modified nucleotides, “s” represents phosphorothioate, “dT” represents deoxythymidine, “OMedT” represents 5′-O-methyl-thymidine, “f” represents a 2′-deoxy-2′-fluoro corresponding nucleoside. Nucleotides at position 1, excluding those previously designated as “OMedT” and “2′ O-methyl-modified nucleotides,” lack a phosphate group on the 5′ nucleoside.

Table 3—Characterization of dsRNAs targeting human IKK2: Activity testing for dose response in HCT-116 cells. IC 50: 50% inhibitory concentration, IC 80: 80% inhibitory concentration, IC 20: 20% inhibitory concentration.

Table 4—Characterization of dsRNAs targeting human IKK2: Stability and Cytokine Induction. t ½: half-life of a strand as defined in examples, PBMC: Human peripheral blood mononuclear cells., cyno BAL: bronchoalveolar lavage fluid from cynomolgous monkey, human ARDS BAL: human bronchoalveolar lavage fluid from patients suffering from acute respiratory distress syndrome (ARDS).

Table 5—Core sequences of dsRNA molecules targeting murine IKK2 gene. Letters in capitals represent RNA nucleotides.

Table 6—dsRNA targeting murine IKK2 gene with modifications. Letters in capitals represent RNA nucleotides (i.e. a 2′-hydroxy corresponding nucleoside), lower case letters “c”, “g”, “a” and “u” represent 2′ O-methyl-modified nucleotides, “s” represents phosphorothioate, “dT” represents deoxythymidine, “OMedT” represents 5′-O-methyl-thymidine, “f” represents a 2′-deoxy-2′-fluoro corresponding nucleoside. Nucleotides at position 1, excluding those previously designated as “OMedT” and “2′ O-methyl-modified nucleotides,” lack a phosphate group on the 5′ nucleoside.

Table 7—Characterization of dsRNAs targeting murine IKK2: Activity testing for dose response in P388D1 cells. IC 50: 50% inhibitory concentration, IC 80: 80% inhibitory concentration, IC 20: 20% inhibitory concentration.

Table 8—Characterization of dsRNAs targeting murine IKK2: Stability and Cytokine Induction. t ½: half-life of a strand as defined in examples, PBMC: Human peripheral blood mononuclear cells.

Table 9—Sequences of bDNA probes for determination of human IKK2. LE=label extender, CE=capture extender, BL=blocking probe.

Table 10—Sequences of bDNA probes for determination of human GAPDH. LE=label extender, CE=capture extender, BL=blocking probe.

Table 11—Sequences of bDNA probes for determination of murine IKK2. LE=label extender, CE=capture extender, BL=blocking probe.

Table 12—Sequences of bDNA probes for determination of murine GAPDH. LE=label extender, CE=capture extender, BL=blocking probe.

Table 13—dsRNA targeting human IKK2 gene without modifications (“core sequences”) and their modified counterparts. Letters in capitals represent RNA nucleotides (in the “modified sequences” capital letters represent a 2′-hydroxy corresponding nucleoside), lower case letters “c”, “g”, “a” and “u” represent 2′ O-methyl-modified nucleotides, “s” represents phosphorothioate, “dT” represents deoxythymidine, “OMedT” represents 5′-O-methyl-thymidine and “f” represents a 2′-deoxy-2′-fluoro corresponding nucleoside. Nucleotides at position 1, excluding those previously designated as “OMedT” and “2′ O-methyl-modified nucleotides,” lack a phosphate group on the 5′ nucleoside.

Table 14—dsRNA targeting murine IKK2 gene without modifications (“core sequences”) and their modified counterparts. Letters in capitals represent RNA nucleotides (in the “modified sequences” capital letters represent a 2′-hydroxy corresponding nucleoside), lower case letters “c”, “g”, “a” and “u” represent 2′ O-methyl-modified nucleotides, “s” represents phosphorothioate, “dT” represents deoxythymidine, “OMedT” represents 5′-O-methyl-thymidine and “f” represents a 2′-deoxy-2′-fluoro corresponding nucleoside. Nucleotides at position 1, excluding those previously designated as “OMedT” and “2′ O-methyl-modified nucleotides,” lack a phosphate group on the 5′ nucleoside.

Table 15—Potential off-target targets, mismatch locations and (on)off-target activity of dsRNA targeting human IKK2 comprising sequence ID pair 223/224.

Table 16—Potential off-target targets, mismatch locations and (on)off-target activity of dsRNAs targeting human IKK2 comprising sequence ID pair 235/236.

Table 17—Potential off-target targets, mismatch locations and (on)off-target activity of dsRNAs targeting human IKK2 comprising sequence ID pair 219/220.

Table 18—Potential off-target targets, mismatch locations and (on)off-target activity of dsRNAs targeting human IKK2 comprising sequence ID pair 229/230.

Table 19—Sequences of bDNA probes for determination of human GAPDH during in vitro off-target analysis. LE=label extender, CE=capture extender, BL=blocking probe.

Table 20—Sequences of bDNA probes for determination of human MAPKAPK3 during in vitro off-target analysis. LE=label extender, CE=capture extender, BL=blocking probe.

Table 21—Sequences of bDNA probes for determination of human PHF17 during in vitro off-target analysis. LE=label extender, CE=capture extender, BL=blocking probe.

Table 22—Sequences of bDNA probes for determination of human IKK2 during in vitro off-target analysis. LE=label extender, CE=capture extender, BL=blocking probe.

Table 23—Activity testing for dose response in A549 cells, SEQ ID pair 223/224.

Table 24—Activity testing for dose response in A549 cells, SEQ ID pair 229/230.

### EXAMPLES

### Identification of dsRNAs for Therapeutic Use

dsRNA design was carried out to identify dsRNAs specifically targeting human IKK2 for therapeutic use. First, known mRNA sequences of human (Homo sapiens) IKK2 (NM_{—}001556.1 listed as SEQ ID NO. 917, AB209090.1 listed as SEQ ID NO. 918), and an EST for the cynomolgous monkey (Macaca fascicularis) IKK2 (CJ452271.1 listed as SEQ ID NO. 919) were downloaded from NCBI Genbank.

The coding sequence of cynomolgous monkey (Macaca fascicularis) IKK2 gene was sequenced (see SEQ ID NO. 920).

The human IKK2 mRNA sequences (SEQ ID NO. 917 and SEQ ID NO. 918) were examined together with the cynomolgous monkey sequences (SEQ ID NO. 919 and SEQ ID NO. 920) by computer analysis to identify homologous sequences of 19 nucleotides that yield RNA interference (RNAi) agents cross-reactive to both sequences.

In identifying RNAi agents, the selection was limited to 19mer antisense sequences having at least 2 mismatches to any other sequence and to 19mer sense sequences having at least 1 mismatch to any other sequence in the human RefSeq database (release 27), which we assumed to represent the comprehensive human transcriptome, by using a proprietary algorithm.

All sequences containing 4 or more consecutive G's (poly-G sequences) were excluded from the synthesis.

The sequences thus identified formed the basis for the synthesis of the RNAi agents in appended Tables 1 and 2. dsRNAs cross-reactive to human as well as cynomolgous monkey IKK2 were defined as most preferable for therapeutic use.

Identification of Murine dsRNAs targeting IKK2

dsRNA design was carried out to identify dsRNAs specifically targeting murine IKK2 for prove-of-concept. The known mRNA sequence of mouse (Mus musculus) IKK2 (NM_{—}010546.1 listed as SEQ ID NO. 921) and the rat (Rattus norvegicus) IKK2 mRNA (NM_{—}053355.2 listed as SEQ ID NO. 922) were downloaded from NCBI Genbank.

The mouse IKK2 mRNA sequence (SEQ ID NO. 921) was examined together with the rat sequence (SEQ ID NO. 922) by computer analysis to identify homologous sequences of 19 nucleotides that yield RNA interference (RNAi) agents cross-reactive to both sequences.

In identifying RNAi agents, the selection was limited to 19mer antisense sequences having at least 2 mismatches to any other sequence in the mouse RefSeq database (release 27), which we assumed to represent the comprehensive mouse transcriptome, by using a proprietary algorithm.

All sequences containing 4 or more consecutive G's (poly-G sequences) were excluded from the synthesis.

The sequences thus identified formed the basis for the synthesis of the RNAi agents in appended Table 5 and 6. dsRNAs cross-reactive to mouse and rat IKK2.

dsRNA Synthesis

Where the source of a reagent is not specifically given herein, such reagent may be obtained from any supplier of reagents for molecular biology at a quality/purity standard for application in molecular biology.

Single-stranded RNAs were produced by solid phase synthesis on a scale of 1 μmole using an Expedite 8909 synthesizer (Applied Biosystems, Applera Deutschland GmbH, Darmstadt, Germany) and controlled pore glass (CPG, 500A, Proligo Biochemie GmbH, Hamburg, Germany) as solid support. RNA and RNA containing 2′-O-methyl nucleotides were generated by solid phase synthesis employing the corresponding phosphoramidites and 2′-O-methyl phosphoramidites, respectively (Proligo Biochemie GmbH, Hamburg, Germany). These building blocks were incorporated at selected sites within the sequence of the oligoribonucleotide chain using standard nucleoside phosphoramidite chemistry such as described in Current protocols in nucleic acid chemistry, Beaucage, S. L. et al. (Edrs.), John Wiley & Sons, Inc., New York, N.Y., USA. Phosphorothioate linkages were introduced by replacement of the iodine oxidizer solution with a solution of the Beaucage reagent (Chruachem Ltd, Glasgow, UK) in acetonitrile (1%). Further ancillary reagents were obtained from Mallinckrodt Baker (Griesheim, Germany).

Deprotection and purification of the crude oligoribonucleotides by anion exchange HPLC were carried out according to established procedures. Yields and concentrations were determined by UV absorption of a solution of the respective RNA at a wavelength of 260 nm using a spectral photometer (DU 640B, Beckman Coulter GmbH, Unterschleiβheim, Germany). Double stranded RNA was generated by mixing an equimolar solution of complementary strands in annealing buffer (20 mM sodium phosphate, pH 6.8; 100 mM sodium chloride), heated in a water bath at 85-90° C. for 3 minutes and cooled to room temperature over a period of 3-4 hours. The annealed RNA solution was stored at −20° C. until use.

Activity Testing of Therapeutic dsRNAs Targeting IKK2

HCT-116 cells and A549 cells in culture were used for quantitation of IKK2 mRNA by branched DNA in total mRNA isolated from cells incubated with IKK2 specific siRNAs assay.

HCT-116 cells were obtained from American Type Culture Collection (Rockville, Md., cat. No. CCL-247) and cultured in McCoy's 5a medium (Biochrom AG, Berlin, Germany, cat. No. F 1015) supplemented to contain 10% fetal calf serum (FCS) (Biochrom AG, Berlin, Germany, cat. No. 50115), 2 mM L-Glutamin (Biochrom AG, Berlin, Germany, cat. No. K0283) and Penicillin 100 U/ml, Streptomycin 100 mg/ml (Biochrom AG, Berlin, Germany, cat. No. A2213) at 37° C. in an atmosphere with 5% CO₂ in a humidified incubator (Heraeus HERAcell, Kendro Laboratory Products, Langenselbold, Germany).

A549 cells were obtained from American Type Culture Collection (Rockville, Md., cat. No. CCL-185) and cultured in RPMI 1640 medium (Biochrom AG, Berlin, Germany, cat. No. FG1215) supplemented to contain 10% fetal calf serum (FCS) (Biochrom AG, Berlin, Germany, cat. No. 50115), 2 mM L-Glutamin (Biochrom AG, Berlin, Germany, cat. No. K0283) and Penicillin 100 U/ml, Streptomycin 100 mg/ml (Biochrom AG, Berlin, Germany, cat. No. A2213) at 37° C. in an atmosphere with 5% CO₂ in a humidified incubator (Heraeus HERAcell, Kendro Laboratory Products, Langenselbold, Germany).

Transfection of siRNA in HCT-116 cells was performed directly after seeding 20,000 cells/well on a 96-well plate, and was carried out with Lipofectamine 2000 (Invitrogen GmbH, Karlsruhe, Germany, cat. No. 11668-019) as described by the manufacturer.

Transfection of siRNA in A549 cells was performed directly after seeding 15,000 cells/well on a 96-well plate, and was carried out with Interferin (Polyplus transfection, Peqlab, Erlangen, Germany, cat. No. 409-10) as described by the manufacturer.

In two independent single dose experiments performed in quadruplicates siRNAs were transfected at a concentration of 50 nM. Most effective siRNAs against IKK2 from the single dose screens were further characterized by dose response curves. For dose response curves, transfections were performed as for the single dose screen above, but with concentrations starting with 100 nM and decreasing in 6-fold dilutions down to 10 fM. After transfection cells were incubated for 24 h at 37° C. and 5% CO₂ in a humidified incubator (Heraeus GmbH, Hanau, Germany). For measurement of IKK2 mRNA cells were harvested and lysed at 53° C. following procedures recommended by the manufacturer for GAPDH of the Quantigene Explore Kit (Panomics, Fremont, Calif., USA, cat. No. QG0004) or for IKK2 of the Quantigene II Explore Kit (Panomics, Fremont, Calif., USA, cat. No. QS9900) for bDNA. Afterwards, 50 μl of the lysates were incubated with probesets specific to human IKK2 and 10 μl of the lysates for human GAPDH (sequences of probesets see below) and processed according to the manufacturer's protocol for the respective QuantiGene kit. Chemoluminescence was measured in a Victor2-Light (Perkin Elmer, Wiesbaden, Germany) as RLUs (relative light units) and values obtained with the human IKK2 probeset were normalized to the respective human GAPDH values for each well and in then related to the mean of three unrelated control siRNAs in the single dose experiments, whereas in the dose response experiments the values obtained with the specific siRNAs where related to the mock transfection (=respective transfection reagent without siRNA).

Inhibition data are given in appended tables 2 and 3.

Activity Testing of dsRNAs Targeting Murine IKK2

P388D1 cells in culture were used for quantitation of murine IKK2 mRNA by branched DNA in total mRNA isolated from cells incubated with murine IKK2 specific siRNAs assay. P388D1 cells were obtained from American Type Culture Collection (Rockville, Md., cat. No. CCL-46) and cultured in RPMI1640 (Biochrom AG, Berlin, Germany, cat. No. FG1215) supplemented to contain 20% donor horse serum (Biochrom AG, Berlin, Germany, cat. No. S9135), 2 mM L-Glutamin (Biochrom AG, Berlin, Germany, cat. No. K0283), 2 mM L-Glutamin (Biochrom AG, Berlin, Germany, cat. No. K0283) and Penicillin 100 U/ml, Streptomycin 100 mg/ml (Biochrom AG, Berlin, Germany, cat. No. A2213) at 37° C. in an atmosphere with 5% CO2 in a humidified incubator (Heraeus HERAcell, Kendro Laboratory Products, Langenselbold, Germany).

Transfection of siRNA was performed directly after seeding 25,000 cells/well on a 96-well plate, and was carried out with Lipofectamine 2000 (Invitrogen GmbH, Karlsruhe, Germany, cat. No. 11668-019) as described by the manufacturer. In two independent single dose experiments performed in quadruplicates siRNAs were transfected at a concentration of 50 nM. Most effective siRNAs against murine IKK2 from the single dose screens were further characterized by dose response curves. For dose response curves, transfections were performed as for the single dose screen above, but with concentrations starting with 100 nM and decreasing in 6-fold dilutions down to 10 fM. After transfection cells were incubated for 24 h at 37° C. and 5% CO2 in a humidified incubator (Heraeus GmbH, Hanau, Germany). For measurement of murine IKK2 mRNA cells were harvested and lysed at 53° C. following procedures recommended by the manufacturer of the Quantigene II Explore Kit (Panomics, Fremont, Calif., USA, cat. No. QS9900) for bDNA. Afterwards, 50 μl of the lysates were incubated with probesets specific to murine IKK2 and 10 μl of the lysates for murine GAPDH (sequences of probesets see appended tables 9-12) and processed according to the manufacturer's protocol for QuantiGene. Chemoluminescence was measured in a Victor2-Light (Perkin Elmer, Wiesbaden, Germany) as RLUs (relative light units) and values obtained with the human murine IKK2 probeset were normalized to the respective human GAPDH values for each well and then related to the mean of three unrelated control siRNAs in the single dose experiments, whereas in the dose response experiments the values obtained with the specific siRNAs where related to the mock transfection (=Lipofectamine 2000 without siRNA).

Inhibition data are given in tables 6 and 7.

Stability of dsRNAs

Stability of dsRNAs targeting human IKK2 was determined in vitro from various biological fluids (e.g. human serum, human bronchoalveolar lavage (BAL) fluid, cynomolgous serum etc.) by measuring the half-life of each single strand.

Measurements were carried out in triplicates for each time point, using 3 μl 50 μM dsRNA sample mixed with 30 μl of the biological fluid. Mixtures were incubated for either 0 min, 30 min, 1 h, 3 h, 6 h, 24 h, or 48 h at 37° C. As control for unspecific degradation dsRNA was incubated with 30 μl 1×PBS pH 6.8 for 48 h. Reactions were stopped by the addition of 4 μl proteinase K (20 mg/ml), 25 μl of “Tissue and Cell Lysis Solution” (Epicentre) and 138 μl Millipore water for 30 min at 65° C.

For separation of single strands and analysis of remaining full length product (FLP), samples were run through an ion exchange Dionex Summit HPLC under denaturing conditions-t A. The following gradient was applied:

| Time | % A | % B |
|---|---|---|
| −1.0 min | 75 | 25 |
| 1.00 min | 75 | 25 |
| 19.0 min | 38 | 62 |
| 19.5 min | 0 | 100 |
| 21.5 min | 0 | 100 |
| 22.0 min | 75 | 25 |
| 24.0 min | 75 | 25 |

For every injection, the chromatograms were integrated automatically by the Dionex Chromeleon 6.60 HPLC software, and were adjusted manually if necessary. All peak areas were corrected to the internal standard (IS) peak and normalized to the incubation at t=0 min. The area under the peak and resulting remaining FLP was calculated for each single strand and triplicate separately. Half-life (t1/2) of a strand was defined by the average time point [h] for triplicates at which half of the FLP was degraded. Results are given in appended tables 4 and 8.

### Cytokine Induction

Potential cytokine induction of dsRNAs was determined by measuring the release of IFN-a and TNF-a in an in vitro PBMC assay.

Human peripheral blood mononuclear cells (PBMC) were isolated from buffy coat blood of two donors by Ficoll centrifugation at the day of transfection. Cells were transfected in quadruplicates with dsRNA and cultured for 24 h at 37° C. at a final concentration of 130 nM in Opti-MEM, using either Gene Porter 2 (GP2) or DOTAP. dsRNA sequences that were known to induce IFN-a and TNF-a in this assay, as well as a CpG oligo, were used as positive controls. Chemical conjugated dsRNA or CpG oligonucleotides that did not need a transfection reagent for cytokine induction, were incubated at a concentration of 500 nM in culture medium. At the end of incubation, the quadruplicate culture supernatant were pooled.

IFN-a and TNF-a was then measured in these pooled supernatants by standard sandwich ELISA with two data points per pool. The degree of cytokine induction was expressed relative to positive controls using a score from 0 to 5, with 5 indicating maximum induction. Results are given in appended tables 4 and 8.

### IKK2 In Vitro Analysis of Putative Off Targets

The psiCHECK™—vector (Promega) contains two reporter genes for monitoring RNAi-activity: a synthetic version of the Renilla luciferase (hRluc) gene and a synthetic firefly luciferase gene (hluc+). The firefly luciferase gene permits normalization of changes in Renilla luciferase expression to firefly luciferase expression. Renilla and firefly luciferase activities were measured using the Dual-Glo® Luciferase Assay System (Promega). To use the psiCHECK™ vectors for analyzing off-target effects of the inventive dsRNAs, the predicted off-target sequence was cloned into the multiple cloning region located 3′ to the synthetic Renilla luciferase gene and its translational stop codon. After cloning, the vector is transfected into a mammalian cell line, and subsequently cotransfected with dsRNAs targeting IKK2. If the dsRNA effectively initiates the RNAi process on the target RNA of the predicted off-target, the fused Renilla target gene mRNA sequence will be degraded, resulting in reduced Renilla luciferase activity.

### In Silico Off-Target Prediction

The human genome was searched by computer analysis for sequences homologous to the inventive dsRNAs. Homologous sequences that displayed less than 6 mismatches with the inventive dsRNAs were defined as a possible off-targets. Off-targets selected for in vitro off target analysis are given in appended tables 15-18.

Generation of psiCHECK Vectors Containing Predicted Off-Target Sequences

The strategy for analyzing potential off-target effects for an siRNA lead candidate includes the cloning of the predicted off-target sites into the psiCHECK2 Vector system (Dual Glo®-system, Promega, Braunschweig, Germany cat. No C8021) via XhoI and NotI restriction sites. Therefore the off-target site is extended with 10 nucleotides upstream and downstream of the dsRNA target site followed by the sequence for cloning. Additionally a NheI restriction site is integrated to prove insertion of the fragment by restriction analysis. The single-stranded oligonucleotides were annealed according to a standard protocol (e.g. protocol by Metabion) in a Mastercycler (Eppendorf) and then cloned into psiCHECK (Promega) previously digested with XhoI and NotI restriction enzymes (e.g. New England Biolabs). Successful insertion was verified by restriction analysis with NheI and subsequent sequencing of the positive clones. After clonal production the correct plasmids were used in cell culture experiments.

Analysis of dsRNA Off-Target Effects

Cell Culture: Cos7 cells were obtained from Deutsche Sammlung für Mikroorganismen and Zellkulturen (DSMZ, Braunschweig, Germany, cat. No. ACC-60) and cultured in DMEM (Biochrom AG, Berlin, Germany, cat. No. F0435) supplemented to contain 10% fetal calf serum (FCS) (Biochrom AG, Berlin, Germany, cat. No. 50115), Penicillin 100 U/ml, and Streptomycin 100 μg/ml (Biochrom AG, Berlin, Germany, cat. No. A2213) and 2 mM L-Glutamine (Biochrom AG, Berlin, Germany, cat. No. K0283) as well as 12 μg/ml Natrium-bicarbonate at 37° C. in an atmosphere with 5% CO₂ in a humidified incubator (Heraeus HERAcell, Kendro Laboratory Products, Langenselbold, Germany).

Transfection and Luciferase quantification: For transfection with plasmids, Cos-7 cells were seeded at a density of 2.25×10⁴ cells/well in 96-well plates and transfected directly. Transfection of plasmids was carried out with lipofectamine 2000 (Invitrogen GmbH, Karlsruhe, Germany, cat. No. 11668-019) as described by the manufacturer at a concentration of 50 ng/well. 4 h after transfection, the medium was discarded and fresh medium was added. Now the siRNAs were transfected in a concentration at 50 nM using lipofectamine 2000 as described above. 24 h after siRNA transfection the cells were lysed using Luciferase reagent described by the manufacturer (Dual-Glo™ Luciferase Assay system, Promega, Mannheim, Germany, cat. No. E2980) and Firefly and Renilla Luciferase were quantified according to the manufacturer's protocol. Renilla Luciferase protein levels were normalized to Firefly Luciferase levels. For each siRNA eight individual data points were collected in two independent experiments. A siRNA unrelated to all target sites was used as a control to determine the relative Renilla Luciferase protein levels in siRNA treated cells.

Endogenous analysis was performed with off targets showing a Renilla Luciferase knockdown of more than 25% from single dose screen at 50 nM. Those were further characterized in dose response curves in concentrations ranging from 100 nM down to 10 fM in 6-fold dilutions. The transfection was performed as described above using Lipofectamine 2000 in human A431 cells. A431 cells were obtained from Deutsche Sammlung für Mikroorganismen and Zellkulturen (DSMZ, Braunschweig, Germany, cat. No. ACC-91) and cultured in RPMI (Biochrom AG, Berlin, Germany, cat. No. FG 1215) supplemented to contain 10% fetal calf serum (FCS) (Biochrom AG, Berlin, Germany, cat. No. S0115), Penicillin 100 U/ml, and Streptomycin 100 μg/ml (Biochrom AG, Berlin, Germany, cat. No. A2213) at 37° C. in an atmosphere with 5% CO2 in a humidified incubator (Heraeus HERAcell, Kendro Laboratory Products, Langenselbold, Germany). After transfection cells were incubated for 24 h at 37° C. and 5% CO2 in a humidified incubator (Heraeus GmbH, Hanau, Germany). For measurement of IKK2 mRNA and all off target mRNAs as well as GAPDH mRNA the QuantiGene 2.0 Assay Kit (Panomics, Fremont, Calif., USA) for bDNA quantitation of mRNA was used. Transfected A431 cells were harvested and lysed at 53° C. following procedures recommended by the manufacturer. 50 μl of the lysates were incubated with probesets specific for human IKK2 (table 22) or the specific off target mRNA (sequence of probesets see Table 20 and 21) and processed according to the manufacturer's protocol for QuantiGene. For measurement of GAPDH mRNA 10 μl of the cell lysate was analyzed with the GAPDH specific probeset (table 19). Chemoluminescence was measured in a Victor2-Light (Perkin Elmer, Wiesbaden, Germany) as RLUs (relative light units) and values obtained with the human IKK2 or off target probeset were normalized to the respective human GAPDH values for each well. Unrelated control siRNAs were used as a negative control.

All ranges recited herein encompass all combinations and subcombinations included within that range limit. All patents and publications cited herein are hereby incorporated by reference in their entirety.

TABLE 1

| SEQ | sense strand | SEQ | antisense strand |
|---|---|---|---|
| ID NO | sequence (5′-3′) | ID NO | sequence (5′-3′) |
| 1 | ACUUAAAGCUGGUUCAUAU | 110 | AUAUGAACCAGCUUUAAGU |
| 1 | ACUUAAAGCUGGUUCAUAU | 143 | GUAUGAACCAGCUUUAAGU |
| 2 | GCAUGAAUGCCUCUCGACU | 111 | AGUCGAGAGGCAUUCAUGC |
| 2 | GCAUGAAUGCCUCUCGACU | 118 | GGUCGAGAGGCAUUCAUGC |
| 3 | GGUGGUGAGCUUAAUGAAU | 112 | AUUCAUUAAGCUCACCACC |
| 4 | TGUGGUGAGCUUAAUGAAU | 112 | AUUCAUUAAGCUCACCACC |
| 5 | GCAAGGGAGCUGUACAGGA | 113 | UCCUGUACAGCUCCCUUGC |
| 6 | TCAUGAAUGCCUCUCGACC | 111 | AGUCGAGAGGCAUUCAUGC |
| 7 | TGUGGUGAGCUUAAUGAAC | 112 | AUUCAUUAAGCUCACCACC |
| 8 | AGUACACAGUGACCGUCGA | 114 | UCGACGGUCACUGUGUACU |
| 9 | TGUACACAGUGACCGUCGC | 114 | UCGACGGUCACUGUGUACU |
| 10 | TCUUAAAGCUGGUUCAUAC | 110 | AUAUGAACCAGCUUUAAGU |
| 11 | TCAAGGGAGCUGUACAGGC | 113 | UCCUGUACAGCUCCCUUGC |
| 12 | TGUACACAGUGACCGUCGA | 114 | UCGACGGUCACUGUGUACU |
| 13 | TCAAGGGAGCUGUACAGGA | 113 | UCCUGUACAGCUCCCUUGC |
| 14 | TCAUGAAUGCCUCUCGACU | 118 | GGUCGAGAGGCAUUCAUGC |
| 15 | GGAUGAGAAGACUGUUGUC | 115 | GACAACAGUCUUCUCAUCC |
| 16 | GCUAGAAAAUGCCAUACAG | 116 | CUGUAUGGCAUUUUCUAGC |
| 17 | CUGAAGAUUGCUUGUAGCA | 117 | UGCUACAAGCAAUCUUCAG |
| 18 | CCGACAGAGUUAGCACGAC | 119 | GUCGUGCUAACUCUGUCGG |
| 19 | AGUGUCAGCUGUAUCCUUC | 120 | GAAGGAUACAGCUGACACU |
| 20 | AGGCAAUUCAGAGCUUCGA | 121 | UCGAAGCUCUGAAUUGCCU |
| 21 | TCUUAAAGCUGGUUCAUAU | 110 | AUAUGAACCAGCUUUAAGU |
| 22 | GAUCAGGGCAGUCUUUGCA | 122 | UGCAAAGACUGCCCUGAUC |
| 23 | UCAGGAAAUGGUACGGCUG | 123 | CAGCCGUACCAUUUCCUGA |
| 24 | UGGUUCAUAUCUUGAACAU | 124 | AUGUUCAAGAUAUGAACCA |
| 25 | ACAGAAUCAUCCAUCGGGA | 125 | UCCCGAUGGAUGAUUCUGU |
| 26 | GUACACAGUGACCGUCGAC | 126 | GUCGACGGUCACUGUGUAC |
| 27 | UUGCUUGUAGCAAGGUCCG | 127 | CGGACCUUGCUACAAGCAA |
| 28 | CUGCCGACAGAGUUAGCAC | 128 | GUGCUAACUCUGUCGGCAG |
| 29 | GAAAGUGCGAGUGAUCUAU | 129 | AUAGAUCACUCGCACUUUC |
| 30 | CCUGAAGAUUGCUUGUAGC | 130 | GCUACAAGCAAUCUUCAGG |
| 31 | CGACAGAGUUAGCACGACA | 131 | UGUCGUGCUAACUCUGUCG |
| 32 | CUAGAAAAUGCCAUACAGG | 132 | CCUGUAUGGCAUUUUCUAG |
| 33 | CUGCCCGCGUUAAGAUUCC | 133 | GGAAUCUUAACGCGGGCAG |
| 34 | CAGAAUCAUCCAUCGGGAU | 134 | AUCCCGAUGGAUGAUUCUG |
| 35 | GCCAGAAAACAUCGUCCUG | 135 | CAGGACGAUGUUUUCUGGC |
| 36 | GUUUGCAAGCAGAAGGCGC | 136 | GCGCCUUCUGCUUGCAAAC |
| 37 | UGCUAGAAAAUGCCAUACA | 137 | UGUAUGGCAUUUUCUAGCA |
| 38 | AGGUGGUGAGCUUAAUGAA | 138 | UUCAUUAAGCUCACCACCU |
| 39 | GACAGAGUUAGCACGACAU | 139 | AUGUCGUGCUAACUCUGUC |
| 40 | GCGGGAGAACGAAGUGAAA | 140 | UUUCACUUCGUUCUCCCGC |
| 41 | GAGCUGUACAGGAGACUAA | 141 | UUAGUCUCCUGUACAGCUC |
| 42 | CCUCGAGACCAGCGAACUG | 142 | CAGUUCGCUGGUCUCGAGG |
| 43 | AGCCAGAAAACAUCGUCCU | 144 | AGGACGAUGUUUUCUGGCU |
| 44 | CUGGUUACAGACGGAAGAA | 145 | UUCUUCCGUCUGUAACCAG |
| 45 | AGAGUUUCACGGCCCUAGA | 146 | UCUAGGGCCGUGAAACUCU |
| 46 | UACACAGUGACCGUCGACU | 147 | AGUCGACGGUCACUGUGUA |
| 47 | AAAGUGCGAGUGAUCUAUA | 148 | UAUAGAUCACUCGCACUUU |
| 48 | CAGCGAACUGAGGGUGACA | 149 | UGUCACCCUCAGUUCGCUG |
| 49 | CCGCGUUAAGAUUCCCGCA | 150 | UGCGGGAAUCUUAACGCGG |
| 50 | ACUCCUGGUAGAACGGAUG | 151 | CAUCCGUUCUACCAGGAGU |
| 51 | GCGUUAAGAUUCCCGCAUU | 152 | AAUGCGGGAAUCUUAACGC |
| 52 | AAGCCCGGAUAGCAUGAAU | 153 | AUUCAUGCUAUCCGGGCUU |
| 53 | UUCCCGCAUUUUAAUGUUU | 154 | AAACAUUAAAAUGCGGGAA |
| 54 | AACUCCUGGUAGAACGGAU | 155 | AUCCGUUCUACCAGGAGUU |
| 55 | UUGUAGCAAGGUCCGUGGU | 156 | ACCACGGACCUUGCUACAA |
| 56 | CGUUAAGAUUCCCGCAUUU | 157 | AAAUGCGGGAAUCUUAACG |
| 57 | ACAAAAUUAUUGACCUAGG | 158 | CCUAGGUCAAUAAUUUUGU |
| 58 | GCUUGUAGCAAGGUCCGUG | 159 | CACGGACCUUGCUACAAGC |
| 59 | AACUUAAAGCUGGUUCAUA | 160 | UAUGAACCAGCUUUAAGUU |
| 60 | UGACCGUCGACUACUGGAG | 161 | CUCCAGUAGUCGACGGUCA |
| 61 | AUUCCCGCAUUUUAAUGUU | 162 | AACAUUAAAAUGCGGGAAU |
| 62 | AAUGUGGUGGCUGCCCGAG | 163 | CUCGGGCAGCCACCACAUU |
| 63 | GCCUCUCGACUUAGCCAGC | 164 | GCUGGCUAAGUCGAGAGGC |
| 64 | ACGACAUCAGUAUGAGCUG | 165 | CAGCUCAUACUGAUGUCGU |
| 65 | CUUGUAGCAAGGUCCGUGG | 166 | CCACGGACCUUGCUACAAG |
| 66 | GCCAUGAUGAAUCUCCUCC | 167 | GGAGGAGAUUCAUCAUGGC |
| 67 | UCAUCCGAUGGCACAAUCA | 168 | UGAUUGUGCCAUCGGAUGA |
| 68 | GGAAAUGUCAUCCGAUGGC | 169 | GCCAUCGGAUGACAUUUCC |
| 69 | CGUGGUCCUGUCAGUGGAA | 170 | UUCCACUGACAGGACCACG |
| 70 | AAUUAUUGACCUAGGAUAU | 171 | AUAUCCUAGGUCAAUAAUU |
| 71 | GCCGACAGAGUUAGCACGA | 172 | UCGUGCUAACUCUGUCGGC |
| 72 | UGCUUGUAGCAAGGUCCGU | 173 | ACGGACCUUGCUACAAGCA |
| 73 | AGUGGAAGCCCGGAUAGCA | 174 | UGCUAUCCGGGCUUCCACU |
| 74 | AAGUGUCAGCUGUAUCCUU | 175 | AAGGAUACAGCUGACACUU |
| 75 | CAGGAAAUGGUACGGCUGC | 176 | GCAGCCGUACCAUUUCCUG |
| 76 | GUCCCUGCCGACAGAGUUA | 177 | UAACUCUGUCGGCAGGGAC |
| 77 | CGCGUUAAGAUUCCCGCAU | 178 | AUGCGGGAAUCUUAACGCG |
| 78 | AAGUGCGAGUGAUCUAUAC | 179 | GUAUAGAUCACUCGCACUU |
| 79 | GAAUGCCUCUCGACUUAGC | 180 | GCUAAGUCGAGAGGCAUUC |
| 80 | CGAGACCAGCGAACUGAGG | 181 | CCUCAGUUCGCUGGUCUCG |
| 81 | UGUAGCAAGGUCCGUGGUC | 182 | GACCACGGACCUUGCUACA |
| 82 | UUAGCACGACAUCAGUAUG | 183 | CAUACUGAUGUCGUGCUAA |
| 83 | CUGUACAGGAGACUAAGGG | 184 | CCCUUAGUCUCCUGUACAG |
| 84 | GAGAACGAAGUGAAACUCC | 185 | GGAGUUUCACUUCGUUCUC |
| 85 | GAACUUGGCGCCCAAUGAC | 186 | GUCAUUGGGCGCCAAGUUC |
| 86 | GCUGCCCGCGUUAAGAUUC | 187 | GAAUCUUAACGCGGGCAGC |
| 87 | AAGCUGGUUCAUAUCUUGA | 188 | UCAAGAUAUGAACCAGCUU |
| 88 | GCCCGCGUUAAGAUUCCCG | 189 | CGGGAAUCUUAACGCGGGC |
| 89 | GAGGAAGUCGCGCCGCGCU | 190 | AGCGCGGCGCGACUUCCUC |
| 90 | CUCGAGACCAGCGAACUGA | 191 | UCAGUUCGCUGGUCUCGAG |
| 91 | AGAGGUGGUGAGCUUAAUG | 192 | CAUUAAGCUCACCACCUCU |
| 92 | GAGGUGGUGAGCUUAAUGA | 193 | UCAUUAAGCUCACCACCUC |
| 93 | GAGUUUCACGGCCCUAGAC | 194 | GUCUAGGGCCGUGAAACUC |
| 94 | CGGCCUCCAACAGCUUACC | 195 | GGUAAGCUGUUGGAGGCCG |
| 95 | AGCCCGGAUAGCAUGAAUG | 196 | CAUUCAUGCUAUCCGGGCU |
| 96 | GCGGGCCUGGCGUUGAUCC | 197 | GGAUCAACGCCAGGCCCGC |
| 97 | UGCCCGCGUUAAGAUUCCC | 198 | GGGAAUCUUAACGCGGGCA |
| 98 | AGAUUCCCGCAUUUUAAUG | 199 | CAUUAAAAUGCGGGAAUCU |
| 99 | UCUCGACUUAGCCAGCCUG | 200 | CAGGCUGGCUAAGUCGAGA |
| 100 | CAAUGUGGUGGCUGCCCGA | 201 | UCGGGCAGCCACCACAUUG |
| 101 | AAACUGUGGUUUGCAAGCA | 202 | UGCUUGCAAACCACAGUUU |
| 102 | CCGCGUCCCUGCCGACAGA | 203 | UCUGUCGGCAGGGACGCGG |
| 103 | UGGGAUCACAUCAGAUAAA | 204 | UUUAUCUGAUGUGAUCCCA |
| 104 | AACAGAAUCAUCCAUCGGG | 205 | CCCGAUGGAUGAUUCUGUU |
| 105 | AAUGCCUCUCGACUUAGCC | 206 | GGCUAAGUCGAGAGGCAUU |
| 106 | UGUACAGGAGACUAAGGGA | 207 | UCCCUUAGUCUCCUGUACA |
| 107 | UGUGGGCGGGAGAACGAAG | 208 | CUUCGUUCUCCCGCCCACA |
| 108 | UCUGUGGGCGGGAGAACGA | 209 | UCGUUCUCCCGCCCACAGA |
| 109 | AGAUUGCUUGUAGCAAGGU | 210 | ACCUUGCUACAAGCAAUCU |

TABLE 2

| | | |
|---|---|---|
| Activity | Activity | Activity |
| testing | testing | testing |
| with 50 nM | with 50 nM | with 0.5 nM |
| siRNA in | siRNA | siRNA in A549 |
| HCT-116 cells | in A549 cells | cells |

| | | | | | | | | | |
|---|---|---|---|---|---|---|---|---|---|
| | | | | mean | | mean | | mean | |
| | | | | re- | stand- | re- | stand- | re- | stand- |
| | | | | main- | ard | main- | ard | main- | ard |
| SEQ | | SEQ | | ing | devia- | ing | devia- | ing | devia- |
| ID | | ID | antisense strand | mRNA | tion | mRNA | tion | mRNA | tion |
| NO | sense strand sequence (5′-3′) | NO | sequence (5′-3′) | [%] | [%] | [%] | [%] | [%] | [%] |

| | | | | | | | | | |
|---|---|---|---|---|---|---|---|---|---|
| 211 | AcuuAAAGcuGGuucAuAudTsdT | 212 | AuAUGAACcAGCUUuAAGUdTsdT | 43 | 2 | 36 | 7 | 42 | 9 |
| 213 | GcAuGAAuGccucucGAcudTsdT | 214 | AGUCGAGAGGcAUUcAUGCdTsdT | 46 | 8 | 38 | 2 | 36 | 4 |
| 215 | GGuGGuGAGcuuAAuGAAudTs | 216 | AUUcAUuAAGCUcACcACCdTsdT | 43 | 2 | 30 | 8 | 34 | 5 |
| | dT | | | | | | | | |
| 217 | GguGGuGAGcuuAAuGAAudTsdT | 218 | AUUcAUuAAGCUcACcACCdTsdT | n.d. | n.d. | 29 | 6 | 42 | 7 |
| 219 | ACfUfUfAAAGCfUfGGUfUfCf | 220 | pAUfAUfGAACfCfAGCfUfUfUfAAG | n.d. | n.d. | 29 | 7 | 34 | 2 |
| | AUfAUfdTsdT | | UfdTsdT | | | | | | |
| 221 | (OMedT)guGGuGAGcuuAAuGA | 222 | AUUcAUuAAGCUcACcACCdTsdT | n.d. | n.d. | 25 | 12 | 49 | 9 |
| | AudTsdT | | | | | | | | |
| 223 | GcAAGGGAGcuGuAcAGGAdTs | 224 | puCCUGuAcAGCUCCCUUGcdTsdT | 50 | 3 | 26 | 4 | 36 | 5 |
| | dT | | | | | | | | |
| 225 | (OMedT)cAuGAAuGccucucGAc | 226 | pAGuCGAGAGGcAUUcAUGcdTsdT | n.d. | n.d. | 31 | 4 | 41 | 7 |
| | cdTsdT | | | | | | | | |
| 227 | (OMedT)guGGuGAGcuuAAuGA | 228 | AUUcAUuAAGCUcACcACCdTsdT | n.d. | n.d. | 30 | 7 | 41 | 5 |
| | AcdTsdT | | | | | | | | |
| 229 | AguAcAcAGuGAccGucGAdTsdT | 230 | puCGACGGUcACUGUGuACudTsdT | n.d. | n.d. | 29 | 5 | 26 | 2 |
| 231 | (OMedT)guAcAcAGuGAccGucG | 232 | puCGACGGUcACUGUGuACUdTsdT | n.d. | n.d. | 28 | 9 | 36 | 5 |
| | cdTsdT | | | | | | | | |
| 233 | GcAAGGGAGcuGuAcAGGAdTs | 234 | UCCUGuAcAGCUCCCUUGCdTsdT | 40 | 3 | 31 | 6 | 33 | 5 |
| | dT | | | | | | | | |
| 235 | (OMedT)cuuAAAGcuGGuucAuA | 236 | pAuAUGAACcAGCUUuAAGudTsdT | n.d. | n.d. | 31 | 5 | 42 | 4 |
| | cdTsdT | | | | | | | | |
| 237 | GcAAGGGAGcuGuAcAGGAdTs | 238 | puCCuGuAcAGCUCCCUuGcdTsdT | 49 | 2 | n.d. | n.d. | n.d. | n.d. |
| | dT | | | | | | | | |
| 239 | (OMedT)guAcAcAGuGAccGucG | 240 | puCGACGGUcACUGUGuACudTsdT | n.d. | n.d. | 29 | 3 | 36 | 2 |
| | cdTsdT | | | | | | | | |
| 241 | AGuAcAcAGuGAccGucGAdTsdT | 242 | UCGACGGUcACUGUGuACUdTsdT | 42 | 7 | 29 | 5 | 34 | 2 |
| 243 | (OMedT)cAAGGGAGcuGuAcA | 244 | puCCUGuAcAGCUCCCUUGcdTsdT | n.d. | n.d. | 30 | 13 | 39 | 5 |
| | GGcdTsdT | | | | | | | | |
| 245 | (OMedT)guAcAcAGuGAccGucG | 246 | puCGACGGUcACUGUGuACUdTsdT | n.d. | n.d. | 35 | 2 | 39 | 4 |
| | AdTsdT | | | | | | | | |
| 247 | (OMedT)cAAGGGAGcuGuAcA | 248 | puCCuGuAcAGCUCCCUuGcdTsdT | n.d. | n.d. | 29 | 11 | 39 | 7 |
| | GGAdTsdT | | | | | | | | |
| 249 | AGuAcAcAGuGAccGucGAdTsdT | 250 | uCGACGGUcACUGUGuACudTsdT | n.d. | n.d. | 23 | 3 | 40 | 6 |
| 251 | (OMedT)guAcAcAGuGAccGucG | 252 | uCGACGGUcACUGUGuACudTsdT | n.d. | n.d. | 24 | 4 | 39 | 7 |
| | cdTsdT | | | | | | | | |
| 253 | GcAuGAAuGccucucGAcudTsdT | 254 | AGuCGAGAGGcAUUcAUGCdTsdT | n.d. | n.d. | 24 | 8 | 36 | 4 |
| 255 | (OMedT)cAAGGGAGcuGuAcA | 256 | UCCUGuAcAGCUCCCUUGCdTsdT | n.d. | n.d. | 25 | 3 | 43 | 9 |
| | GGAdTsdT | | | | | | | | |
| 257 | (OMedT)cAAGGGAGcuGuAcA | 258 | uCCuGuAcAGCUCCCUuGcdTsdT | n.d. | n.d. | 25 | 9 | 61 | 17 |
| | GGAdTsdT | | | | | | | | |
| 259 | (OMedT)cAAGGGAGcuGuAcA | 260 | puCCuGuAcAGCUCCCUuGcdTsdT | n.d. | n.d. | 25 | 7 | 46 | 5 |
| | GGcdTsdT | | | | | | | | |
| 261 | (OMedT)guAcAcAGuGAccGucG | 262 | UCGACGGUcACUGUGuACUdTsdT | n.d. | n.d. | 25 | 5 | 37 | 5 |
| | AdTsdT | | | | | | | | |
| 263 | AcuuAAAGcuGGuucAuAudTsdT | 264 | AuAUGAACcAGCUUuAAGudTsdT | n.d. | n.d. | 25 | 8 | 35 | 8 |
| 265 | (OMedT)guAcAcAGuGAccGucG | 266 | UCGACGGUcACUGUGuACUdTsdT | n.d. | n.d. | 26 | 6 | 34 | 4 |
| | cdTsdT | | | | | | | | |
| 267 | (OMedT)cAuGAAuGccucucGAc | 268 | GGuCGAGAGGcAUUcAUGcdTsdT | n.d. | n.d. | 26 | 9 | 39 | 5 |
| | udTsdT | | | | | | | | |
| 269 | AcuuAAAGcuGGuucAuAudTsdT | 270 | pAuAUGAACcAGCUUuAAGudTsdT | n.d. | n.d. | 26 | 7 | 41 | 13 |
| 271 | (OMedT)cAAGGGAGcuGuAcA | 272 | puCCUGuAcAGCUCCCUUGcdTsdT | n.d. | n.d. | 27 | 2 | 46 | 12 |
| | GGAdTsdT | | | | | | | | |
| 273 | (OMedT)cAAGGGAGcuGuAcA | 274 | UCCUGuAcAGCUCCCUUGCdTsdT | n.d. | n.d. | 27 | 8 | 36 | 4 |
| | GGcdTsdT | | | | | | | | |
| 275 | AguAcAcAGuGAccGucGAdTsdT | 276 | puCGACGGUcACUGUGuACUdTsdT | n.d. | n.d. | 27 | 7 | 32 | 4 |
| 277 | (OMedT)cAuGAAuGccucucGAc | 278 | AGuCGAGAGGcAUUcAuGCdTsdT | n.d. | n.d. | 27 | 4 | 42 | 7 |
| | cdTsdT | | | | | | | | |
| 279 | AguAcAcAGuGAccGucGAdTsdT | 280 | uCGACGGUcACUGUGuACudTsdT | n.d. | n.d. | 28 | 7 | 46 | 4 |
| 281 | (OMedT)guAcAcAGuGAccGucG | 282 | uCGACGGUcACUGUGuACudTsdT | n.d. | n.d. | 28 | 5 | 51 | 14 |
| | AdTsdT | | | | | | | | |
| 283 | GcAuGAAuGccucucGAcudTsdT | 284 | pAGuCGAGAGGcAUUcAUGCdTsdT | n.d. | n.d. | 28 | 5 | 38 | 6 |
| 285 | AcuuAAAGcuGGuucAuAudTsdT | 286 | pAuAUGAACcAGCUuuAAGudTsdT | n.d. | n.d. | 28 | 10 | 57 | 11 |
| 287 | ACfUfUfAAAGCfUfGGUfUfCf | 288 | AUfAUfGAACfCfAGCfUfUfUfAAGU | n.d. | n.d. | 28 | 6 | 35 | 4 |
| | AUfAUfdTsdT | | fdTsdT | | | | | | |
| 289 | (OMedT)cuuAAAGcuGGuucAuA | 290 | AuAUGAACcAGCUUuAAGUdTsdT | n.d. | n.d. | 28 | 2 | 41 | 3 |
| | cdTsdT | | | | | | | | |
| 291 | GGAuGAGAAGAcuGuuGucdTs | 292 | GAcAAcAGUCUUCUcAUCCdTsdT | 42 | 5 | 29 | 4 | n.d. | n.d. |
| | dT | | | | | | | | |
| 293 | (OMedT)cAAGGGAGcuGuAcA | 294 | uCCuGuAcAGCUCCCUuGcdTsdT | n.d. | n.d. | 29 | 11 | 80 | 13 |
| | GGcdTsdT | | | | | | | | |
| 295 | GcAuGAAuGccucucGAcudTsdT | 296 | pAGuCGAGAGGcAUUcAuGCdTsdT | n.d. | n.d. | 29 | 5 | 38 | 6 |
| 297 | GGUfGGUfGAGCfUfUfAAUfG | 298 | pAUfUfCfAUfUfAAGCfUfCfACf | n.d. | n.d. | 29 | 7 | 67 | 20 |
| | AAUfdTsdT | | CfACfCfdTsdT | | | | | | |
| 299 | GcuAGAAAAuGccAuAcAGdTsdT | 300 | CUGuAUGGcAUUUUCuAGCdTsdT | 28 | 2 | 30 | 6 | n.d. | n.d. |
| 301 | cuGAAGAuuGcuuGuAGcAdTsdT | 302 | UGCuAcAAGcAAUCUUcAGdTsdT | 42 | 7 | 30 | 3 | n.d. | n.d. |
| 303 | AguAcAcAGuGAccGucGAdTsdT | 304 | uCGACGGUcACUGUGuACUdTsdT | n.d. | n.d. | 30 | 13 | 46 | 6 |
| 305 | (OMedT)cAuGAAuGccucucGAc | 306 | AGuCGAGAGGcAUUcAUGCdTsdT | n.d. | n.d. | 30 | 8 | 35 | 1 |
| | udTsdT | | | | | | | | |
| 307 | (OMedT)cAuGAAuGccucucGAc | 308 | pAGuCGAGAGGcAUUcAUGcdTsdT | n.d. | n.d. | 30 | 8 | 39 | 3 |
| | udTsdT | | | | | | | | |
| 309 | (OMedT)cAuGAAuGccucucGAc | 310 | AGuCGAGAGGcAUUcAUGcdTsdT | n.d. | n.d. | 30 | 6 | 40 | 8 |
| | cdTsdT | | | | | | | | |
| 311 | (OMedT)guAcAcAGuGAccGucG | 312 | uCGACGGUcACUGUGuACUdTsdT | n.d. | n.d. | 31 | 5 | 48 | 10 |
| | cdTsdT | | | | | | | | |
| 313 | GcAuGAAuGccucucGAcudTsdT | 314 | GGuCGAGAGGcAUUcAUGcdTsdT | n.d. | n.d. | 31 | 10 | 38 | 2 |
| 315 | (OMedT)cAuGAAuGccucucGAc | 316 | AGuCGAGAGGcAUUcAUGcdTsdT | n.d. | n.d. | 31 | 5 | 41 | 3 |
| | udTsdT | | | | | | | | |
| 317 | ccGAcAGAGuuAGcAcGAcdTsdT | 318 | GUCGUGCuAACUCUGUCGGdTsdT | 47 | 8 | 32 | 6 | n.d. | n.d. |
| 319 | AguAcAcAGuGAccGucGAdTsdT | 320 | UCGACGGUcACUGUGuACUdTsdT | n.d. | n.d. | 32 | 14 | 33 | 7 |
| 321 | (OMedT)guGGuGAGcuuAAuGA | 322 | AuucAUuAAGCUcACcACcdTsdT | n.d. | n.d. | 32 | 4 | 76 | 12 |
| | AudTsdT | | | | | | | | |
| 323 | GGuGGuGAGcuuAAuGAAudTs | 324 | pAuucAUuAAGCUcACcACcdTsdT | 56 | 5 | 33 | 7 | 68 | 6 |
| | dT | | | | | | | | |
| 325 | AGuAcAcAGuGAccGucGAdTsdT | 326 | puCGACGGUcACUGUGuACUdTsdT | n.d. | n.d. | 33 | 4 | 35 | 3 |
| 327 | GCfAUfGAAUfGCfCfUfCfUfCf | 328 | pAGUfCfGAGAGGCfAUfUfCfAUfG | n.d. | n.d. | 33 | 9 | 35 | 5 |
| | GACfUfdTsdT | | CfdTsdT | | | | | | |
| 329 | (OMedT)cAuGAAuGccucucGAc | 330 | pAGuCGAGAGGcAUUcAuGCdTsdT | n.d. | n.d. | 33 | 6 | 36 | 2 |
| | udTsdT | | | | | | | | |
| 331 | (OMedT)guGGuGAGcuuAAuGA | 332 | pAuucAUuAAGCUcACcACcdTsdT | n.d. | n.d. | 33 | 15 | 65 | 11 |
| | AcdTsdT | | | | | | | | |
| 333 | AGuGucAGcuGuAuccuucdTsdT | 334 | GAAGGAuAcAGCUGAcACUdTsdT | 44 | 12 | 34 | 6 | n.d. | n.d. |
| 335 | AGGcAAuucAGAGcuucGAdTsdT | 336 | UCGAAGCUCUGAAUUGCCUdTsdT | 59 | 9 | 34 | 3 | n.d. | n.d. |
| 337 | GGuGGuGAGcuuAAuGAAudTs | 338 | AuucAUuAAGCUcACcACcdTsdT | 62 | 10 | 34 | 5 | 63 | 15 |
| | dT | | | | | | | | |
| 339 | (OMedT)guAcAcAGuGAccGucG | 340 | puCGACGGUcACUGUGuACudTsdT | n.d. | n.d. | 34 | 2 | 42 | 12 |
| | AdTsdT | | | | | | | | |
| 341 | (OMedT)cAuGAAuGccucucGAc | 342 | AGuCGAGAGGcAUUcAuGCdTsdT | n.d. | n.d. | 34 | 12 | 36 | 4 |
| | udTsdT | | | | | | | | |
| 343 | GguGGuGAGcuuAAuGAAudTsdT | 344 | AuucAUuAAGCUcACcACcdTsdT | n.d. | n.d. | 34 | 4 | 65 | 8 |
| 345 | (OMedT)guGGuGAGcuuAAuGA | 346 | AuUcAUuAAGCUcACcACcdTsdT | n.d. | n.d. | 34 | 10 | 71 | 13 |
| | AudTsdT | | | | | | | | |
| 347 | (OMedT)guGGuGAGcuuAAuGA | 348 | pAuUcAUuAAGCUcACcACcdTsdT | n.d. | n.d. | 34 | 11 | 66 | 20 |
| | AudTsdT | | | | | | | | |
| 349 | (OMedT)guGGuGAGcuuAAuGA | 350 | AuucAUuAAGCUcACcACcdTsdT | n.d. | n.d. | 34 | 5 | 70 | 12 |
| | AcdTsdT | | | | | | | | |
| 351 | (OMedT)cuuAAAGcuGGuucAuA | 352 | pAuAUGAACcAGCUUuAAGudTsdT | n.d. | n.d. | 34 | 5 | 54 | 2 |
| | udTsdT | | | | | | | | |
| 353 | (OMedT)cuuAAAGcuGGuucAuA | 354 | AuAUGAACcAGCUUuAAGudTsdT | n.d. | n.d. | 34 | 3 | 54 | 3 |
| | udTsdT | | | | | | | | |
| 355 | GAucAGGGcAGucuuuGcAdTsdT | 356 | UGcAAAGACUGCCCUGAUCdTsdT | 38 | 5 | 35 | 5 | n.d. | n.d. |
| 357 | ucAGGAAAuGGuAcGGcuGdTs | 358 | cAGCCGuACcAUUUCCUGAdTsdT | 50 | 7 | 35 | 5 | n.d. | n.d. |
| | dT | | | | | | | | |
| 359 | GcAAGGGAGcuGuAcAGGAdTs | 360 | uCCuGuAcAGCUCCCUuGcdTsdT | 53 | 5 | 35 | 7 | 74 | 23 |
| | dT | | | | | | | | |
| 361 | GCfAAGGGAGCfUfGUfACfAG | 362 | pUfCfCfUfGUfACfAGCfUfCfCf | n.d. | n.d. | 35 | 5 | 67 | 1 |
| | GAdTsdT | | CfUfUfGCfdTsdT | | | | | | |
| 363 | (OMedT)cAuGAAuGccucucGAc | 364 | GGuCGAGAGGcAUUcAUGcdTsdT | n.d. | n.d. | 35 | 5 | 43 | 10 |
| | cdTsdT | | | | | | | | |
| 365 | GGuGGuGAGcuuAAuGAAudTs | 366 | AuUcAUuAAGCUcACcACcdTsdT | 69 | 12 | 36 | 2 | 53 | 8 |
| | dT | | | | | | | | |
| 367 | (OMedT)guAcAcAGuGAccGucG | 368 | uCGACGGUcACUGUGuACUdTsdT | n.d. | n.d. | 36 | 3 | 50 | 12 |
| | AdTsdT | | | | | | | | |
| 369 | (OMedT)cAuGAAuGccucucGAc | 370 | AGUCGAGAGGcAUUcAUGCdTsdT | n.d. | n.d. | 36 | 5 | 37 | 5 |
| | cdTsdT | | | | | | | | |
| 371 | GguGGuGAGcuuAAuGAAudTsdT | 372 | AuUcAUuAAGCUcACcACcdTsdT | n.d. | n.d. | 36 | 6 | 55 | 12 |
| 373 | (OMedT)cuuAAAGcuGGuucAuA | 374 | AuAUGAACcAGCUUuAAGUdTsdT | n.d. | n.d. | 36 | 8 | 58 | 5 |
| | udTsdT | | | | | | | | |
| 375 | (OMedT)cuuAAAGcuGGuucAuA | 376 | AuAUGAACcAGCUUuAAGudTsdT | n.d. | n.d. | 36 | 5 | 36 | 4 |
| | cdTsdT | | | | | | | | |
| 377 | uGGuucAuAucuuGAAcAudTsdT | 378 | AUGUUcAAGAuAUGAACcAdTsdT | 39 | 3 | 37 | 4 | n.d. | n.d. |
| 379 | AcAGAAucAuccAucGGGAdTsdT | 380 | UCCCGAUGGAUGAUUCUGUdTsdT | 40 | 4 | 37 | 6 | n.d. | n.d. |
| 381 | GuAcAcAGuGAccGucGAcdTsdT | 382 | GUCGACGGUcACUGUGuACdTsdT | 51 | 6 | 37 | 11 | n.d. | n.d. |
| 383 | uuGcuuGuAGcAAGGuccGdTsdT | 384 | CGGACCUUGCuAcAAGcAAdTsdT | 51 | 9 | 37 | 11 | n.d. | n.d. |
| 385 | GGuGGuGAGcuuAAuGAAudTs | 386 | pAuUcAUuAAGCUcACcACcdTsdT | 59 | 5 | 37 | 5 | 59 | 3 |
| | dT | | | | | | | | |
| 387 | (OMedT)cAuGAAuGccucucGAc | 388 | pAGuCGAGAGGcAUUcAUGCdTsdT | n.d. | n.d. | 37 | 4 | 35 | 4 |
| | cdTsdT | | | | | | | | |
| 389 | GguGGuGAGcuuAAuGAAudTsdT | 390 | pAuUcAUuAAGCUcACcACcdTsdT | n.d. | n.d. | 37 | 6 | 61 | 9 |
| 391 | GguGGuGAGcuuAAuGAAudTsdT | 392 | pAuucAUuAAGCUcACcACcdTsdT | n.d. | n.d. | 37 | 5 | 58 | 3 |
| 393 | cuGccGAcAGAGuuAGcAcdTsdT | 394 | GUGCuAACUCUGUCGGcAGdTsdT | 52 | 4 | 38 | 6 | n.d. | n.d. |
| 395 | GAAAGuGcGAGuGAucuAudTs | 396 | AuAGAUcACUCGcACUUUCdTsdT | 55 | 6 | 38 | 2 | n.d. | n.d. |
| | dT | | | | | | | | |
| 397 | GCfAAGGGAGCfUfGUfACfAG | 398 | UfCfCfUfGUfACfAGCfUfCfCf | n.d. | n.d. | 38 | 3 | 62 | 10 |
| | GAdTsdT | | CfUfUfGCfdTsdT | | | | | | |
| 399 | AGuAcAcAGuGAccGucGAdTsdT | 400 | puCGACGGUcACUGUGuACudTsdT | n.d. | n.d. | 38 | 18 | 29 | 4 |
| 401 | AGUfACfACfAGUfGACfCfGUf | 402 | UfCfGACfGGUfCfACfUfGUfGUf | n.d. | n.d. | 38 | 7 | 63 | 13 |
| | CfGAdTsdT | | ACfUfdTsdT | | | | | | |
| 403 | (OMedT)guAcAcAGuGAccGucG | 404 | puCGACGGUcACUGuGuACudTsdT | n.d. | n.d. | 38 | 5 | 68 | 12 |
| | cdTsdT | | | | | | | | |
| 405 | (OMedT)guAcAcAGuGAccGucG | 406 | uCGACGGUcACUGuGuACudTsdT | n.d. | n.d. | 38 | 5 | 84 | 11 |
| | cdTsdT | | | | | | | | |
| 407 | GcAuGAAuGccucucGAcudTsdT | 408 | AGuCGAGAGGcAUUcAuGCdTsdT | n.d. | n.d. | 38 | 31 | 38 | 7 |
| 409 | (OMedT)cAuGAAuGccucucGAc | 410 | AGuCGAGAGGcAUUcAUGCdTsdT | n.d. | n.d. | 38 | 5 | 40 | 6 |
| | cdTsdT | | | | | | | | |
| 411 | (OMedT)cAuGAAuGccucucGAc | 412 | pAGuCGAGAGGcAUUcAuGCdTsdT | n.d. | n.d. | 38 | 6 | 42 | 8 |
| | cdTsdT | | | | | | | | |
| 413 | (OMedT)cuuAAAGcuGGuucAuA | 414 | AuAUGAACcAGCUuuAAGudTsdT | n.d. | n.d. | 38 | 8 | 61 | 4 |
| | cdTsdT | | | | | | | | |
| 415 | ccuGAAGAuuGcuuGuAGedTsdT | 416 | GCuAcAAGcAAUCUUcAGGdTsdT | 52 | 8 | 39 | 6 | n.d. | n.d. |
| 417 | cGAcAGAGuuAGcAcGAcAdTsdT | 418 | UGUCGUGCuAACUCUGUCGdTsdT | 55 | 9 | 39 | 2 | n.d. | n.d. |
| 419 | AGUfACfACfAGUfGACfCfGUf | 420 | pUfCfGACfGGUfCfACfUfGUfGU | n.d. | n.d. | 39 | 5 | 62 | 5 |
| | CfGAdTsdT | | fACfUfdTsdT | | | | | | |
| 421 | GcAuGAAuGccucucGAcudTsdT | 422 | AGuCGAGAGGcAUUcAUGcdTsdT | n.d. | n.d. | 39 | 17 | 37 | 2 |
| 423 | (OMedT)guGGuGAGcuuAAuGA | 424 | AuUcAUuAAGCUcACcACcdTsdT | n.d. | n.d. | 39 | 18 | 66 | 10 |
| | AcdTsdT | | | | | | | | |
| 425 | cuAGAAAAuGccAuAcAGGdTsdT | 426 | CCUGuAUGGcAUUUUCuAGdTsdT | 44 | 5 | 40 | 11 | n.d. | n.d. |
| 427 | cuGcccGcGuuAAGAuuccdTsdT | 428 | GGAAUCUuAACGCGGGcAGdTsdT | 49 | 7 | 40 | 4 | n.d. | n.d. |
| 429 | (OMedT)cAuGAAuGccucucGAc | 430 | pAGuCGAGAGGcAUUcAUGCdTsdT | n.d. | n.d. | 40 | 14 | 35 | 5 |
| | udTsdT | | | | | | | | |
| 431 | (OMedT)guGGuGAGcuuAAuGA | 432 | pAuucAUuAAGCUcACcACcdTsdT | n.d. | n.d. | 40 | 6 | 74 | 5 |
| | AudTsdT | | | | | | | | |
| 433 | (OMedT)guGGuGAGcuuAAuGA | 434 | pAuUcAUuAAGCUcACcACcdTsdT | n.d. | n.d. | 40 | 17 | 73 | 17 |
| | AcdTsdT | | | | | | | | |
| 435 | (OMedT)guAcAcAGuGAccGucG | 436 | puCGACGGUcACUGuGuACudTsdT | n.d. | n.d. | 42 | 10 | 64 | 8 |
| | AdTsdT | | | | | | | | |
| 437 | AcuuAAAGcuGGuucAuAudTsdT | 438 | AuAUGAACcAGCUuuAAGudTsdT | n.d. | n.d. | 42 | 17 | 53 | 5 |
| 439 | cAGAAucAuccAucGGGAudTsdT | 440 | AUCCCGAUGGAUGAUUCUGdTsdT | 34 | 4 | 43 | 4 | n.d. | n.d. |
| 441 | GccAGAAAAcAucGuccuGdTsdT | 442 | cAGGACGAUGUUUUCUGGCdTsdT | 39 | 3 | 43 | 4 | n.d. | n.d. |
| 443 | GuuuGcAAGcAGAAGGcGcdTsdT | 444 | GCGCCUUCUGCUUGcAAACdTsdT | 40 | 4 | 43 | 5 | n.d. | n.d. |
| 445 | uGcuAGAAAAuGccAuAcAdTsdT | 446 | UGuAUGGcAUUUUCuAGcAdTsdT | 40 | 1 | 43 | 12 | n.d. | n.d. |
| 447 | AguAcAcAGuGAccGucGAdTsdT | 448 | puCGACGGUcACUGuGuACudTsdT | n.d. | n.d. | 43 | 6 | 62 | 3 |
| 449 | GGUfGGUfGAGCfUfUfAAUfG | 450 | AUfUfCfAUfUfAAGCfUfCfACfC | n.d. | n.d. | 43 | 16 | 46 | 12 |
| | AAUfdTsdT | | fACfCfdTsdT | | | | | | |
| 451 | AGGuGGuGAGcuuAAuGAAdTs | 452 | UUcAUuAAGCUcACcACCUdTsdT | 45 | 4 | 44 | 7 | n.d. | n.d. |
| | dT | | | | | | | | |
| 453 | GAcAGAGuuAGcAcGAcAudTsdT | 454 | AUGUCGUGCuAACUCUGUCdTsdT | 51 | 9 | 44 | 4 | n.d. | n.d. |
| 455 | AGuAcAcAGuGAccGucGAdTsdT | 456 | uCGACGGUcACUGUGuACUdTsdT | n.d. | n.d. | 44 | 37 | 35 | 4 |
| 457 | GcAuGAAuGccucucGAcudTsdT | 458 | pAGuCGAGAGGcAUUcAUGcdTsdT | n.d. | n.d. | 44 | 22 | 46 | 9 |
| 459 | GCfAUfGAAUfGCfCfUfCfUfCf | 460 | AGUfCfGAGAGGCfAUfUfCfAUfGCf | n.d. | n.d. | 44 | 17 | 34 | 2 |
| | GACfUfdTsdT | | dTsdT | | | | | | |
| 461 | GcGGGAGAAcGAAGuGAAAd | 462 | UUUcACUUCGUUCUCCCGCdTsdT | 45 | 5 | 45 | 8 | n.d. | n.d. |
| | TsdT | | | | | | | | |
| 463 | AguAcAcAGuGAccGucGAdTsdT | 464 | uCGACGGUcACUGuGuACudTsdT | n.d. | n.d. | 45 | 8 | 93 | 31 |
| 465 | (OMedT)guAcAcAGuGAccGucG | 466 | uCGACGGUcACUGuGuACudTsdT | n.d. | n.d. | 45 | 6 | 81 | 18 |
| | AdTsdT | | | | | | | | |
| 467 | GAGcuGuAcAGGAGAcuAAdTs | 468 | UuAGUCUCCUGuAcAGCUCdTsdT | 43 | 5 | 46 | 4 | n.d. | n.d. |
| | dT | | | | | | | | |
| 469 | ccucGAGAccAGcGAAcuGdTsdT | 470 | cAGUUCGCUGGUCUCGAGGdTsdT | 56 | 5 | 46 | 3 | n.d. | n.d. |
| 471 | AcuuAAAGcuGGuucAuAudTsdT | 472 | GuAUGAACcAGCUuuAAGudTsdT | n.d. | n.d. | 46 | 8 | 76 | 2 |
| 473 | AGccAGAAAAcAucGuccudTsdT | 474 | AGGACGAUGUUUUCUGGCUdTsdT | 46 | 6 | 47 | 2 | n.d. | n.d. |
| 475 | cuGGuuAcAGAcGGAAGAAdTs | 476 | UUCUUCCGUCUGuAACcAGdTsdT | 68 | 15 | 47 | 6 | n.d. | n.d. |
| | dT | | | | | | | | |
| 477 | AGAGuuucAcGGcccuAGAdTsdT | 478 | UCuAGGGCCGUGAAACUCUdTsdT | 80 | 9 | 47 | 5 | n.d. | n.d. |
| 479 | (OMedT)cuuAAAGcuGGuucAuA | 480 | pAuAUGAACcAGCUuuAAGudTsdT | n.d. | n.d. | 47 | 4 | 58 | 3 |
| | cdTsdT | | | | | | | | |
| 481 | uAcAcAGuGAccGucGAcudTsdT | 482 | AGUCGACGGUcACUGUGuAdTsdT | 51 | 5 | 48 | 10 | n.d. | n.d. |
| 483 | AAAGuGcGAGuGAucuAuAdTs | 484 | uAuAGAUcACUCGcACUUUdTsdT | 53 | 5 | 48 | 1 | n.d. | n.d. |
| | dT | | | | | | | | |
| 485 | cAGcGAAcuGAGGGuGAcAdTs | 486 | UGUcACCCUcAGUUCGCUGdTsdT | 59 | 10 | 48 | 10 | n.d. | n.d. |
| | dT | | | | | | | | |
| 487 | (OMedT)cAuGAAuGccucucGAc | 488 | AGUCGAGAGGcAUUcAUGCdTsdT | n.d. | n.d. | 48 | 23 | 35 | 4 |
| | udTsdT | | | | | | | | |
| 489 | (OMedT)cuuAAAGcuGGuucAuA | 490 | pAuAUGAACcAGCUuuAAGudTsdT | n.d. | n.d. | 48 | 5 | 81 | 2 |
| | udTsdT | | | | | | | | |
| 491 | ccGcGuuAAGAuucccGcAdTsdT | 492 | UGCGGGAAUCUuAACGCGGdTsdT | 51 | 5 | 49 | 8 | n.d. | n.d. |
| 493 | AcuccuGGuAGAAcGGAuGdTsdT | 494 | cAUCCGUUCuACcAGGAGUdTsdT | 39 | 5 | 50 | 2 | n.d. | n.d. |
| 495 | GcGuuAAGAuucccGcAuudTsdT | 496 | AAUGCGGGAAUCUuAACGCdTsdT | 49 | 4 | 50 | 6 | n.d. | n.d. |
| 497 | AAGcccGGAuAGcAuGAAudTsdT | 498 | AUUcAUGCuAUCCGGGCUUdTsdT | 50 | 8 | 50 | 7 | n.d. | n.d. |
| 499 | AGuAcAcAGuGAccGucGAdTsdT | 500 | puCGACGGUcACUGuGuACudTsdT | n.d. | n.d. | 50 | 12 | 68 | 17 |
| 501 | uucccGcAuuuuAAuGuuudTsdT | 502 | AAAcAUuAAAAUGCGGGAAdTsdT | 40 | 3 | 51 | 9 | n.d. | n.d. |
| 503 | AAcuccuGGuAGAAcGGAudTsdT | 504 | AUCCGUUCuACcAGGAGUUdTsdT | 40 | 5 | 51 | 2 | n.d. | n.d. |
| 505 | uuGuAGcAAGGuccGuGGudTsdT | 506 | ACcACGGACCUUGCuAcAAdTsdT | 71 | 17 | 51 | 4 | n.d. | n.d. |
| 507 | (OMedT)cuuAAAGcuGGuucAuA | 508 | AuAUGAACcAGCUuuAAGudTsdT | n.d. | n.d. | 52 | 26 | 88 | 3 |
| | udTsdT | | | | | | | | |
| 509 | AGuAcAcAGuGAccGucGAdTsdT | 510 | uCGACGGUcACUGuGuACudTsdT | n.d. | n.d. | 53 | 17 | 75 | 8 |
| 511 | cGuuAAGAuucccGcAuuudTsdT | 512 | AAAUGCGGGAAUCUuAACGdTsdT | 58 | 4 | 55 | 2 | n.d. | n.d. |
| 513 | AcAAAAuuAuuGAccuAGGdTsdT | 514 | CCuAGGUcAAuAAUUUUGUdTsdT | 48 | 5 | 56 | 6 | n.d. | n.d. |
| 515 | (OMedT)cuuAAAGcuGGuucAuA | 516 | GuAUGAACcAGCUuuAAGudTsdT | n.d. | n.d. | 56 | 16 | 96 | 6 |
| | udTsdT | | | | | | | | |
| 517 | GcuuGuAGcAAGGuccGuGdTsdT | 518 | cACGGACCUUGCuAcAAGCdTsdT | 50 | 9 | 58 | 16 | n.d. | n.d. |
| 519 | AAcuuAAAGcuGGuucAuAdTsdT | 520 | uAUGAACcAGCUUuAAGUUdTsdT | 56 | 7 | 58 | 14 | n.d. | n.d. |
| 521 | uGAccGucGAcuAcuGGAGdTsdT | 522 | CUCcAGuAGUCGACGGUcAdTsdT | 68 | 7 | 58 | 12 | n.d. | n.d. |
| 523 | AuucccGcAuuuuAAuGuudTsdT | 524 | AAcAUuAAAAUGCGGGAAUdTsdT | 49 | 3 | 59 | 7 | n.d. | n.d. |
| 525 | AAuGuGGuGGcuGcccGAGdTsdT | 526 | CUCGGGcAGCcACcAcAUUdTsdT | 80 | 3 | 59 | 4 | n.d. | n.d. |
| 527 | GccucucGAcuuAGccAGcdTsdT | 528 | GCUGGCuAAGUCGAGAGGCdTsdT | 54 | 2 | 60 | 6 | n.d. | n.d. |
| 529 | AcGAcAucAGuAuGAGcuGdTsdT | 530 | cAGCUcAuACUGAUGUCGUdTsdT | 64 | 9 | 60 | 8 | n.d. | n.d. |
| 531 | cuuGuAGcAAGGuccGuGGdTsdT | 532 | CcACGGACCUUGCuAcAAGdTsdT | 88 | 16 | 60 | 8 | n.d. | n.d. |
| 533 | GccAuGAuGAAucuccuccdTsdT | 534 | GGAGGAGAUUcAUcAUGGCdTsdT | 58 | 11 | 61 | 16 | n.d. | n.d. |
| 535 | ucAuccGAuGGcAcAAucAdTsdT | 536 | UGAUUGUGCcAUCGGAUGAdTsdT | 65 | 11 | 62 | 4 | n.d. | n.d. |
| 537 | GGAAAuGucAuccGAuGGcdTsdT | 538 | GCcAUCGGAUGAcAUUUCCdTsdT | 68 | 8 | 62 | 6 | n.d. | n.d. |
| 539 | cGuGGuccuGucAGuGGAAdTsdT | 540 | UUCcACUGAcAGGACcACGdTsdT | 70 | 11 | 62 | 4 | n.d. | n.d. |
| 541 | AAuuAuuGAccuAGGAuAudTsdT | 542 | AuAUCCuAGGUcAAuAAUUdTsdT | 89 | 11 | 62 | 7 | n.d. | n.d. |
| 543 | GccGAcAGAGuuAGcAcGAdTsdT | 544 | UCGUGCuAACUCUGUCGGCdTsdT | 60 | 5 | 63 | 8 | n.d. | n.d. |
| 545 | uGcuuGuAGcAAGGuccGudTsdT | 546 | ACGGACCUUGCuAcAAGcAdTsdT | 75 | 14 | 63 | 9 | n.d. | n.d. |
| 547 | AGuGGAAGcccGGAuAGcAdTs | 548 | UGCuAUCCGGGCUUCcACUdTsdT | 61 | 15 | 64 | 13 | n.d. | n.d. |
| | dT | | | | | | | | |
| 549 | AAGuGucAGcuGuAuccuudTsdT | 550 | AAGGAuAcAGCUGAcACUUdTsdT | 57 | 9 | 65 | 14 | n.d. | n.d. |
| 551 | cAGGAAAuGGuAcGGcuGcdTsdT | 552 | GcAGCCGuACcAUUUCCUGdTsdT | 83 | 13 | 65 | 3 | n.d. | n.d. |
| 553 | GucccuGccGAcAGAGuuAdTsdT | 554 | uAACUCUGUCGGcAGGGACdTsdT | 61 | 6 | 66 | 14 | n.d. | n.d. |
| 555 | cGcGuuAAGAuucccGcAudTsdT | 556 | AUGCGGGAAUCUuAACGCGdTsdT | 59 | 6 | 67 | 4 | n.d. | n.d. |
| 557 | AAGuGcGAGuGAucuAuAcdTsdT | 558 | GuAuAGAUcACUCGcACUUdTsdT | 63 | 5 | 67 | 13 | n.d. | n.d. |
| 559 | GAAuGccucucGAcuuAGcdTsdT | 560 | GCuAAGUCGAGAGGcAUUCdTsdT | 63 | 3 | 67 | 14 | n.d. | n.d. |
| 561 | cGAGAccAGcGAAcuGAGGdTs | 562 | CCUcAGUUCGCUGGUCUCGdTsdT | 72 | 10 | 68 | 8 | n.d. | n.d. |
| | dT | | | | | | | | |
| 563 | uGuAGcAAGGuccGuGGucdTsdT | 564 | GACcACGGACCUUGCuAcAdTsdT | 78 | 18 | 69 | 12 | n.d. | n.d. |
| 565 | uuAGcAcGAcAucAGuAuGdTsdT | 566 | cAuACUGAUGUCGUGCuAAdTsdT | 81 | 15 | 69 | 2 | n.d. | n.d. |
| 567 | cuGuAcAGGAGAcuAAGGGdTs | 568 | CCCUuAGUCUCCUGuAcAGdTsdT | 48 | 5 | 70 | 2 | n.d. | n.d. |
| | dT | | | | | | | | |
| 569 | GAGAAcGAAGuGAAAcuccdTs | 570 | GGAGUUUcACUUCGUUCUCdTsdT | 53 | 5 | 70 | 4 | n.d. | n.d. |
| | dT | | | | | | | | |
| 571 | GAAcuuGGcGcccAAuGAcdTsdT | 572 | GUcAUUGGGCGCcAAGUUCdTsdT | 68 | 5 | 70 | 12 | n.d. | n.d. |
| 573 | GcuGcccGcGuuAAGAuucdTsdT | 574 | GAAUCUuAACGCGGGcAGCdTsdT | 74 | 7 | 70 | 3 | n.d. | n.d. |
| 575 | AAGcuGGuucAuAucuuGAdTsdT | 576 | UcAAGAuAUGAACcAGCUUdTsdT | 80 | 6 | 70 | 15 | n.d. | n.d. |
| 577 | GcccGcGuuAAGAuucccGdTsdT | 578 | CGGGAAUCUuAACGCGGGCdTsdT | 77 | 11 | 71 | 8 | n.d. | n.d. |
| 579 | GAGGAAGucGcGccGcGcudTsdT | 580 | AGCGCGGCGCGACUUCCUCdTsdT | 87 | 7 | 72 | 5 | n.d. | n.d. |
| 581 | cucGAGAccAGcGAAcuGAdTsdT | 582 | UcAGUUCGCUGGUCUCGAGdTsdT | 87 | 15 | 72 | 4 | n.d. | n.d. |
| 583 | AGAGGuGGuGAGcuuAAuGdTs | 584 | cAUuAAGCUcACcACCUCUdTsdT | 65 | 6 | 73 | 21 | n.d. | n.d. |
| | dT | | | | | | | | |
| 585 | GAGGuGGuGAGcuuAAuGAdTs | 586 | UcAUuAAGCUcACcACCUCdTsdT | 62 | 5 | 75 | 16 | n.d. | n.d. |
| | dT | | | | | | | | |
| 587 | GAGuuucAcGGcccuAGAcdTsdT | 588 | GUCuAGGGCCGUGAAACUCdTsdT | 98 | 17 | 75 | 7 | n.d. | n.d. |
| 589 | cGGccuccAAcAGcuuAccdTsdT | 590 | GGuAAGCUGUUGGAGGCCGdTsdT | 63 | 4 | 76 | 26 | n.d. | n.d. |
| 591 | AGcccGGAuAGcAuGAAuGdTsdT | 592 | cAUUcAUGCuAUCCGGGCUdTsdT | 71 | 12 | 76 | 9 | n.d. | n.d. |
| 593 | (OMedT)cuuAAAGcuGGuucAuA | 594 | GuAUGAACcAGCUuuAAGudTsdT | n.d. | n.d. | 76 | 18 | 89 | 5 |
| | cdTsdT | | | | | | | | |
| 595 | GcGGGccuGGcGuuGAuccdTsdT | 596 | GGAUcAACGCcAGGCCCGCdTsdT | 72 | 14 | 77 | 9 | n.d. | n.d. |
| 597 | uGcccGcGuuAAGAuucccdTsdT | 598 | GGGAAUCUuAACGCGGGcAdTsdT | 74 | 10 | 77 | 4 | n.d. | n.d. |
| 599 | AGAuucccGcAuuuuAAuGdTsdT | 600 | cAUuAAAAUGCGGGAAUCUdTsdT | 75 | 6 | 77 | 6 | n.d. | n.d. |
| 601 | ucucGAcuuAGccAGccuGdTsdT | 602 | cAGGCUGGCuAAGUCGAGAdTsdT | 66 | 2 | 79 | 22 | n.d. | n.d. |
| 603 | cAAuGuGGuGGcuGcccGAdTsdT | 604 | UCGGGcAGCcACcAcAUUGdTsdT | 85 | 9 | 79 | 11 | n.d. | n.d. |
| 605 | AAAcuGuGGuuuGcAAGcAdTsdT | 606 | UGCUUGcAAACcAcAGUUUdTsdT | 66 | 6 | 81 | 16 | n.d. | n.d. |
| 607 | ccGcGucccuGccGAcAGAdTsdT | 608 | UCUGUCGGcAGGGACGCGGdTsdT | 69 | 5 | 81 | 12 | n.d. | n.d. |
| 609 | uGGGAucAcAucAGAuAAAdTs | 610 | UUuAUCUGAUGUGAUCCcAdTsdT | 59 | 6 | 83 | 14 | n.d. | n.d. |
| | dT | | | | | | | | |
| 611 | AAcAGAAucAuccAucGGGdTsdT | 612 | CCCGAUGGAUGAUUCUGUUdTsdT | 82 | 9 | 83 | 6 | n.d. | n.d. |
| 613 | AAuGccucucGAcuuAGccdTsdT | 614 | GGCuAAGUCGAGAGGcAUUdTsdT | 80 | 2 | 89 | 18 | n.d. | n.d. |
| 615 | uGuAcAGGAGAcuAAGGGAdT | 616 | UCCCUuAGUCUCCUGuAcAdTsdT | 104 | 18 | 90 | 5 | n.d. | n.d. |
| | sdT | | | | | | | | |
| 617 | uGuGGGcGGGAGAAcGAAGdT | 618 | CUUCGUUCUCCCGCCcAcAdTsdT | 91 | 4 | 92 | 9 | n.d. | n.d. |
| | sdT | | | | | | | | |
| 619 | ucuGuGGGcGGGAGAAcGAdTs | 620 | UCGUUCUCCCGCCcAcAGAdTsdT | 66 | 9 | 98 | 18 | n.d. | n.d. |
| | dT | | | | | | | | |
| 621 | AGAuuGcuuGuAGcAAGGudTsdT | 622 | ACCUUGCuAcAAGcAAUCUdTsdT | 96 | 18 | 99 | 14 | n.d. | n.d. |
| 623 | GcuAGAAAAuGccAuAcAGdTsdT | 624 | pCUGuAUGGcAUUUUCuAGCdTsdT | 40 | 6 | n.d. | n.d. | n.d. | n.d. |
| 625 | cAGAAucAuccAucGGGAudTsdT | 626 | pAUCCCGAUGGAUGAUUCuGdTsdT | 44 | 2 | n.d. | n.d. | n.d. | n.d. |
| 627 | cAGAAucAuccAucGGGAudTsdT | 628 | AUCCCGAUGGAUGAUUCuGdTsdT | 45 | 3 | n.d. | n.d. | n.d. | n.d. |
| 629 | uGGuucAuAucuuGAAcAudTsdT | 630 | AuGUUcAAGAuAUGAACcAdTsdT | 47 | 4 | n.d. | n.d. | n.d. | n.d. |
| 631 | cAGAAucAuccAucGGGAudTsdT | 632 | AUCCCGAuGGAuGAUUCuGdTsdT | 52 | 4 | n.d. | n.d. | n.d. | n.d. |
| 633 | cAGAAucAuccAucGGGAudTsdT | 634 | pAUCCCGAuGGAuGAUUCuGdTsdT | 52 | 2 | n.d. | n.d. | n.d. | n.d. |
| 635 | GcAAGGGAGcuGuAcAGGAdTs | 636 | uCCUGuAcAGCUCCCUUGcdTsdT | 52 | 2 | n.d. | n.d. | n.d. | n.d. |
| | dT | | | | | | | | |
| 637 | uGGuucAuAucuuGAAcAudTsdT | 638 | pAuGUUcAAGAuAUGAACcAdTsdT | 59 | 7 | n.d. | n.d. | n.d. | n.d. |
| 639 | GcuAGAAAAuGccAuAcAGdTsdT | 640 | pCuGuAUGGcAUUUUCuAGcdTsdT | 59 | 5 | n.d. | n.d. | n.d. | n.d. |
| 641 | cAGAAucAuccAucGGGAudTsdT | 642 | pAuCCCGAuGGAuGAUUCuGdTsdT | 79 | 4 | n.d. | n.d. | n.d. | n.d. |
| 643 | cAGAAucAuccAucGGGAudTsdT | 644 | AuCCCGAuGGAuGAUUCuGdTsdT | 80 | 3 | n.d. | n.d. | n.d. | n.d. |

TABLE 3

| | Activity testing for dose response in A549 cells, | | | |
|---|---|---|---|---|
| | means of two transfections | | | |
| | | | | Mean |
| SEQ ID NO | mean IC50 | mean IC80 | mean IC20 | remaining |
| pair | [nM] | [nM] | [nM] | mRNA [%] |
| 211/212 | 0.029977797 | n.d. | 0.000186213 | 26 |
| 213/214 | 0.036474049 | n.d. | 0.004817789 | 32 |
| 215/216 | 0.036883743 | n.d. | 0.002506471 | 21 |
| 217/218 | 0.038412935 | n.d. | 0.001997614 | 24 |
| 219/220 | 0.042070262 | n.d. | 0.003365483 | 27 |
| 221/222 | 0.047448964 | n.d. | 0.002966541 | 32 |
| 223/224 | 0.047601199 | 9.490428028 | 0.001377836 | 21 |
| 225/226 | 0.051666372 | n.d. | 0.001640154 | 28 |
| 227/228 | 0.05292601 | n.d. | 0.002206092 | 30 |
| 229/230 | 0.068932274 | n.d. | 0.013816572 | 27 |
| 231/232 | 0.088658026 | n.d. | 0.368197697 | 34 |
| 233/234 | 0.092451215 | n.d. | 0.017036454 | 29 |
| 235/236 | 0.129462023 | n.d. | 0.005158928 | 30 |
| 237/238 | 0.145498079 | n.d. | 0.017521556 | 39 |
| 239/240 | 0.175324535 | n.d. | 0.022587172 | 38 |
| 241/242 | 0.180643587 | n.d. | 0.019113081 | 37 |
| 243/244 | 0.227067567 | n.d. | 0.020383489 | 38 |
| 245/246 | 0.368197697 | n.d. | 0.070714381 | 41 |
| 247/248 | 0.488003751 | n.d. | 0.06993465 | 47 |

TABLE 4

| | Stability | | Stability | | Stability Human | | Stability | | | |
|---|---|---|---|---|---|---|---|---|---|---|
| | Cyno Serum | | Cyno BAL | | ARDS BAL | | Human Serum | | Human PBMC | |
| SEQ ID NO | sense | antisense | sense | antisense | sense | antisense | sense | antisense | assay | |
| pair | t½ [hr] | t½ [hr] | t½ [hr] | t½ [hr] | t½ [hr] | t½ [hr] | t½ [hr] | t½ [hr] | IFN-a | TNF-a |
| 233/234 | 8.7 | 9.6 | n.d. | n.d. | >48 | 6 | >24 | 9.7 | 0 | 0 |
| 211/212 | 24.5 | 4.2 | >48 | 40.5 | >48 | 1.2 | >48 | 3.9 | 1 | 0 |
| 213/214 | 42.7 | 7.5 | >48 | >48 | >48 | 6.4 | >48 | 20.2 | 0 | 0 |
| 229/230 | 12.9 | 16.4 | n.d. | n.d. | >48 | 18.4 | n.d. | n.d. | 0 | 0 |
| 235/236 | 12.4 | 9.8 | n.d. | n.d. | >48 | 8.6 | n.d. | n.d. | 0 | 0 |
| 225/226 | 22.9 | 1.8 | n.d. | n.d. | >48 | 2.7 | n.d. | n.d. | 0 | 0 |
| 219/220 | 15.7 | 11.6 | n.d. | n.d. | >48 | >48 | n.d. | n.d. | 0 | 2 |
| 223/224 | 10.7 | 16.1 | n.d. | n.d. | >48 | >48 | n.d. | n.d. | 0 | 0 |
| 231/232 | 9.3 | 1.7 | n.d. | n.d. | >48 | 0.4 | n.d. | n.d. | 0 | 0 |
| 241/242 | 10.3 | 2.5 | >48 | 1.6 | >48 | 0.4 | 48 | 1.7 | 0 | 0 |
| 215/216 | 14.6 | 4.9 | n.d. | n.d. | >48 | 0.5 | >24 | 5.9 | 0 | 0 |
| 217/218 | 12.2 | 5.6 | n.d. | n.d. | >48 | 0.5 | n.d. | n.d. | 0 | 0 |
| 299/300 | n.d. | n.d. | n.d. | n.d. | n.d. | n.d. | >24 | 21.3 | n.d. | n.d. |
| 301/302 | n.d. | n.d. | n.d. | n.d. | n.d. | n.d. | >24 | 20.4 | n.d. | n.d. |
| 439/440 | n.d. | n.d. | n.d. | n.d. | n.d. | n.d. | >24 | 6.7 | n.d. | n.d. |
| 461/462 | n.d. | n.d. | n.d. | n.d. | n.d. | n.d. | 21.8 | 5.1 | n.d. | n.d. |

TABLE 5

| | | | |
|---|---|---|---|
| SEQ ID NO | sense strand sequence (5′-3′) | SEQ ID NO | antisense strand sequence (5′-3′) |

| | | | |
|---|---|---|---|
| 645 | GGCGGGAGAAUGACGUGAA | 693 | UUCACGUCAUUCUCCCGCC |
| 645 | GGCGGGAGAAUGACGUGAA | 693 | UUCACGUCAUUCUCCCGCC |
| 646 | UGGGCGGGAGAAUGACGUG | 694 | CACGUCAUUCUCCCGCCCA |
| 647 | GUAGCAAAGUCCGAGGUCC | 695 | GGACCUCGGACUUUGCUAC |
| 648 | GACCGUGGUUUGUAAGCAG | 696 | CUGCUUACAAACCACGGUC |
| 649 | AGACCGUGGUUUGUAAGCA | 697 | UGCUUACAAACCACGGUCU |
| 650 | GUAAGACCGUGGUUUGUAA | 698 | UUACAAACCACGGUCUUAC |
| 651 | CAAAAUUAUUGAUCUAGGA | 699 | UCCUAGAUCAAUAAUUUUG |
| 652 | CUCAGUAAGACCGUGGUUU | 700 | AAACCACGGUCUUACUGAG |
| 653 | UCGGCUCUUAGAUACCUUC | 701 | GAAGGUAUCUAAGAGCCGA |
| 654 | AGCAUGAAUGUGUCUCGAC | 702 | GUCGAGACACAUUCAUGCU |
| 655 | AUUGAUCUAGGAUAUGCCA | 703 | UGGCAUAUCCUAGAUCAAU |
| 656 | GAAGAUCGCCUGUAGCAAA | 704 | UUUGCUACAGGCGAUCUUC |
| 657 | TGGCGGGAGAAUGACGUGAA | 693 | UUCACGUCAUUCUCCCGCC |
| 658 | TGUAGCAAAGUCCGAGGUCC | 695 | GGACCUCGGACUUUGCUAC |
| 659 | AUCGAUAUGAGCUGGUCAC | 705 | GUGACCAGCUCAUAUCGAU |
| 660 | GAUACUUGAACCAGUUCGA | 706 | UCGAACUGGUUCAAGUAUC |
| 661 | AUCGGCUCUUAGAUACCUU | 707 | AAGGUAUCUAAGAGCCGAU |
| 662 | GCCAUCAAGCAAUGCCGAC | 708 | GUCGGCAUUGCUUGAUGGC |
| 663 | UCAGUAAGACCGUGGUUUG | 709 | CAAACCACGGUCUUACUGA |
| 664 | UCGCCUGUAGCAAAGUCCG | 710 | CGGACUUUGCUACAGGCGA |
| 665 | GCCUGUAGCAAAGUCCGAG | 711 | CUCGGACUUUGCUACAGGC |
| 666 | GGAGCCCGAUGGGUCGGAA | 712 | UUCCGACCCAUCGGGCUCC |
| 667 | CCAUCAAGCAAUGCCGACA | 713 | UGUCGGCAUUGCUUGAUGG |
| 668 | CGGCUCUUAGAUACCUUCA | 714 | UGAAGGUAUCUAAGAGCCG |
| 669 | UAUUGAUCUAGGAUAUGCC | 715 | GGCAUAUCCUAGAUCAAUA |
| 670 | UAAGACCGUGGUUUGUAAG | 716 | CUUACAAACCACGGUCUUA |
| 671 | CAUCGAUAUGAGCUGGUCA | 717 | UGACCAGCUCAUAUCGAUG |
| 672 | UGUAGCAAAGUCCGAGGUC | 718 | GACCUCGGACUUUGCUACA |
| 673 | GGGCGGGAGAAUGACGUGA | 719 | UCACGUCAUUCUCCCGCCC |
| 674 | GAUCGCCUGUAGCAAAGUC | 720 | GACUUUGCUACAGGCGAUC |
| 675 | UCGAUAUGAGCUGGUCACC | 721 | GGUGACCAGCUCAUAUCGA |
| 676 | AGGAGCCCGAUGGGUCGGA | 722 | UCCGACCCAUCGGGCUCCU |
| 677 | UGGGAAAUGAAAGAACGCC | 723 | GGCGUUCUUUCAUUUCCCA |
| 678 | GGAAGACUGUAACCGGCUG | 724 | CAGCCGGUUACAGUCUUCC |
| 679 | AUCGCCUGUAGCAAAGUCC | 725 | GGACUUUGCUACAGGCGAU |
| 680 | CCCGAGUUUUCAUGUCCUC | 726 | GAGGACAUGAAAACUCGGG |
| 681 | CCGAUGGGUCGGAAGCAGG | 727 | CCUGCUUCCGACCCAUCGG |
| 682 | CGCCUGUAGCAAAGUCCGA | 728 | UCGGACUUUGCUACAGGCG |
| 683 | GAAGACUGUAACCGGCUGC | 729 | GCAGCCGGUUACAGUCUUC |
| 684 | AAGACUGUAACCGGCUGCA | 730 | UGCAGCCGGUUACAGUCUU |
| 685 | ACGUCAUCCGGUGGCACAA | 731 | UUGUGCCACCGGAUGACGU |
| 686 | GGAAACGUCAUCCGGUGGC | 732 | GCCACCGGAUGACGUUUCC |
| 687 | GAUUUGGAAACGUCAUCCG | 733 | CGGAUGACGUUUCCAAAUC |
| 688 | UGUCCUCAGCGGGUGUCGC | 734 | GCGACACCCGCUGAGGACA |
| 689 | AAACGUCAUCCGGUGGCAC | 735 | GUGCCACCGGAUGACGUUU |
| 690 | CAUCCGGUGGCACAAUCAG | 736 | CUGAUUGUGCCACCGGAUG |
| 691 | UGGAAACGUCAUCCGGUGG | 737 | CCACCGGAUGACGUUUCCA |
| 692 | CAUGUCCUCAGCGGGUGUC | 738 | GACACCCGCUGAGGACAUG |

TABLE 6

Activity testing
with 50 nM
siRNA in P388D1
cells

| | | | | | |
|---|---|---|---|---|---|
| | | | | mean | |
| | | | | remaining | standard |
| SEQ ID | | SEQ ID | | mRNA | deviation |
| NO | sequence (5′-3′) | NO | sequence (5′-3′) | [%] | [%] |

| | | | | | |
|---|---|---|---|---|---|
| 739 | GGcGGGAGAAuGAcGuGAAdTsdT | 740 | UUcACGUcAUUCUCCCGCCdTsdT | 33 | 4 |
| 741 | uGGGcGGGAGAAuGAcGuGdTsdT | 742 | cACGUcAUUCUCCCGCCcAdTsdT | 37 | 8 |
| 743 | GuAGcAAAGuccGAGGuccdTsdT | 744 | GGACCUCGGACUUUGCuACdTsdT | 29 | 4 |
| 745 | GAccGuGGuuuGuAAGcAGdTsdT | 746 | CUGCUuAcAAACcACGGUCdTsdT | 36 | 2 |
| 747 | AGAccGuGGuuuGuAAGcAdTsdT | 748 | UGCUuAcAAACcACGGUCUdTsdT | 31 | 1 |
| 749 | GuAAGAccGuGGuuuGuAAdTsdT | 750 | UuAcAAACcACGGUCUuACdTsdT | 38 | 1 |
| 751 | cAAAAuuAuuGAucuAGGAdTsdT | 752 | UCCuAGAUcAAuAAUUUUGdTsdT | 31 | 4 |
| 753 | cucAGuAAGAccGuGGuuudTsdT | 754 | AAACcACGGUCUuACUGAGdTsdT | 27 | 3 |
| 755 | ucGGcucuuAGAuAccuucdTsdT | 756 | GAAGGuAUCuAAGAGCCGAdTsdT | 33 | 3 |
| 757 | AGcAuGAAuGuGucucGAcdTsdT | 758 | GUCGAGAcAcAUUcAUGCUdTsdT | 37 | 4 |
| 759 | AuuGAucuAGGAuAuGccAdTsdT | 760 | UGGcAuAUCCuAGAUcAAUdTsdT | 34 | 1 |
| 761 | GAAGAucGccuGuAGcAAAdTsdT | 762 | UUUGCuAcAGGCGAUCUUCdTsdT | 37 | 1 |
| 763 | GuAGcAAAGuccGAGGuccdTsdT | 764 | pGGACCUCGGACUUUGCuACdTsdT | n.d. | n.d. |
| 765 | GGcGGGAGAAuGAcGuGAAdTsdT | 766 | puUcACGUcAUUCUCCCGCcdTsdT | n.d. | n.d. |
| 767 | GGcGGGAGAAuGAcGuGAAdTsdT | 768 | uucACGUcAUUCUCCCGCcdTsdT | n.d. | n.d. |
| 769 | GuAGcAAAGuccGAGGuccdTsdT | 770 | GGACCUCGGACUUUGCuAcdTsdT | n.d. | n.d. |
| 771 | GuAGcAAAGuccGAGGuccdTsdT | 772 | pGGACCUCGGACUuuGCuAcdTsdT | n.d. | n.d. |
| 773 | GGcGGGAGAAuGAcGuGAAdTsdT | 774 | uUcACGUcAUUCUCCCGCcdTsdT | n.d. | n.d. |
| 775 | GuAGcAAAGuccGAGGuccdTsdT | 776 | GGACCUCGGACUuuGCuAcdTsdT | n.d. | n.d. |
| 777 | (OMedT)GgcGGGAGAAuGAcGuGAAdTsdT | 778 | puucACGUcAUUCUCCCGCcdTsdT | n.d. | n.d. |
| 779 | GGcGGGAGAAuGAcGuGAAdTsdT | 780 | puUcACGUcAUUCUCCCGCcdTsdT | n.d. | n.d. |
| 781 | (OMedT)GgcGGGAGAAuGAcGuGAAdTsdT | 782 | puUcACGUcAUUCUCCCGCcdTsdT | n.d. | n.d. |
| 783 | GGcGGGAGAAuGAcGuGAAdTsdT | 784 | puucACGUcAUUCUCCCGCcdTsdT | n.d. | n.d. |
| 785 | (OMedT)GuAGcAAAGuccGAGGuccdTsdT | 786 | pGGACCUCGGACUUUGCuAcdTsdT | n.d. | n.d. |
| 787 | GuAGcAAAGuccGAGGuccdTsdT | 788 | pGGACCUCGGACUUUGCuAcdTsdT | n.d. | n.d. |
| 789 | AucGAuAuGAGcuGGucAcdTsdT | 790 | GUGACcAGCUcAuAUCGAUdTsdT | 39 | 5 |
| 791 | GAuAcuuGAAccAGuucGAdTsdT | 792 | UCGAACUGGUUcAAGuAUCdTsdT | 43 | 4 |
| 793 | AucGGcucuuAGAuAccuudTsdT | 794 | AAGGuAUCuAAGAGCCGAUdTsdT | 44 | 5 |
| 795 | GccAucAAGcAAuGccGAcdTsdT | 796 | GUCGGcAUUGCUUGAUGGCdTsdT | 45 | 3 |
| 797 | ucAGuAAGAccGuGGuuuGdTsdT | 798 | cAAACcACGGUCUuACUGAdTsdT | 47 | 2 |
| 799 | ucGccuGuAGcAAAGuccGdTsdT | 800 | CGGACUUUGCuAcAGGCGAdTsdT | 48 | 3 |
| 801 | GccuGuAGcAAAGuccGAGdTsdT | 802 | CUCGGACUUUGCuAcAGGCdTsdT | 50 | 2 |
| 803 | GGAGcccGAuGGGucGGAAdTsdT | 804 | UUCCGACCcAUCGGGCUCCdTsdT | 50 | 3 |
| 805 | ccAucAAGcAAuGccGAcAdTsdT | 806 | UGUCGGcAUUGCUUGAUGGdTsdT | 51 | 4 |
| 807 | cGGcucuuAGAuAccuucAdTsdT | 808 | UGAAGGuAUCuAAGAGCCGdTsdT | 52 | 3 |
| 809 | uAuuGAucuAGGAuAuGccdTsdT | 810 | GGcAuAUCCuAGAUcAAuAdTsdT | 53 | 3 |
| 811 | uAAGAccGuGGuuuGuAAGdTsdT | 812 | CUuAcAAACcACGGUCUuAdTsdT | 55 | 2 |
| 813 | cAucGAuAuGAGcuGGucAdTsdT | 814 | UGACcAGCUcAuAUCGAUGdTsdT | 56 | 8 |
| 815 | uGuAGcAAAGuccGAGGucdTsdT | 816 | GACCUCGGACUUUGCuAcAdTsdT | 56 | 7 |
| 817 | GGGcGGGAGAAuGAcGuGAdTsdT | 818 | UcACGUcAUUCUCCCGCCCdTsdT | 58 | 11 |
| 819 | GAucGccuGuAGcAAAGucdTsdT | 820 | GACUUUGCuAcAGGCGAUCdTsdT | 61 | 1 |
| 821 | ucGAuAuGAGcuGGucAccdTsdT | 822 | GGUGACcAGCUcAuAUCGAdTsdT | 66 | 7 |
| 823 | AGGAGcccGAuGGGucGGAdTsdT | 824 | UCCGACCcAUCGGGCUCCUdTsdT | 66 | 4 |
| 825 | uGGGAAAuGAAAGAAcGccdTsdT | 826 | GGCGUUCUUUcAUUUCCcAdTsdT | 67 | 10 |
| 827 | GGAAGAcuGuAAccGGcuGdTsdT | 828 | cAGCCGGUuAcAGUCUUCCdTsdT | 68 | 2 |
| 829 | AucGccuGuAGcAAAGuccdTsdT | 830 | GGACUUUGCuAcAGGCGAUdTsdT | 68 | 3 |
| 831 | cccGAGuuuucAuGuccucdTsdT | 832 | GAGGAcAUGAAAACUCGGGdTsdT | 70 | 8 |
| 833 | ccGAuGGGucGGAAGcAGGdTsdT | 834 | CCUGCUUCCGACCcAUCGGdTsdT | 72 | 7 |
| 835 | cGccuGuAGcAAAGuccGAdTsdT | 836 | UCGGACUUUGCuAcAGGCGdTsdT | 74 | 4 |
| 837 | GAAGAcuGuAAccGGcuGcdTsdT | 838 | GcAGCCGGUuAcAGUCUUCdTsdT | 74 | 4 |
| 839 | AAGAcuGuAAccGGcuGcAdTsdT | 840 | UGcAGCCGGUuAcAGUCUUdTsdT | 76 | 14 |
| 841 | AcGucAuccGGuGGcAcAAdTsdT | 842 | UUGUGCcACCGGAUGACGUdTsdT | 79 | 7 |
| 843 | GGAAAcGucAuccGGuGGcdTsdT | 844 | GCcACCGGAUGACGUUUCCdTsdT | 81 | 12 |
| 845 | GAuuuGGAAAcGucAuccGdTsdT | 846 | CGGAUGACGUUUCcAAAUCdTsdT | 82 | 12 |
| 847 | uGuccucAGcGGGuGucGcdTsdT | 848 | GCGAcACCCGCUGAGGAcAdTsdT | 85 | 11 |
| 849 | AAAcGucAuccGGuGGcAcdTsdT | 850 | GUGCcACCGGAUGACGUUUdTsdT | 86 | 7 |
| 851 | cAuccGGuGGcAcAAucAGdTsdT | 852 | CUGAUUGUGCcACCGGAUGdTsdT | 92 | 6 |
| 853 | uGGAAAcGucAuccGGuGGdTsdT | 854 | CcACCGGAUGACGUUUCcAdTsdT | 93 | 8 |
| 855 | cAuGuccucAGcGGGuGucdTsdT | 856 | GAcACCCGCUGAGGAcAUGdTsdT | 95 | 15 |

TABLE 7

| | | | | Activity testing for dose | | | | | |
|---|---|---|---|---|---|---|---|---|---|
| | Activity testing for dose response | | | response in P388D1 cells, | | | Activity testing for dose response in | | |
| | in P388D1 cells, screen 1 | | | screen 2 | | | P388D1 cells, screen 3 | | |
| | mean | mean | mean | mean | mean | mean | mean | mean | mean |
| | IC50 | IC80 | IC20 | IC50 | IC80 | IC20 | IC50 | IC80 | IC20 |
| | [nM] | [nM] | [nM] | [nM] | [nM] | [nM] | [nM] | [nM] | [nM] |
| 739/740 | 0.006686 | 436.770672 | 0.000070 | 0.390510 | n.d. | 0.020570 | n.d. | n.d. | n.d. |
| 741/742 | 0.007561 | 499.413244 | 0.000000 | n.d. | n.d. | n.d. | n.d. | n.d. | n.d. |
| 743/744 | 0.033253 | 113.944787 | 0.000085 | n.d. | n.d. | 0.159567 | n.d. | n.d. | n.d. |
| 745/746 | 0.036720 | n.d. | 0.000119 | n.d. | n.d. | n.d. | n.d. | n.d. | n.d. |
| 747/748 | 0.048008 | 447.430940 | 0.000032 | n.d. | n.d. | n.d. | n.d. | n.d. | n.d. |
| 749/750 | 0.063289 | n.d. | 0.000016 | n.d. | n.d. | n.d. | n.d. | n.d. | n.d. |
| 751/752 | 0.075323 | 1417.560125 | 0.000080 | n.d. | n.d. | n.d. | n.d. | n.d. | n.d. |
| 753/754 | 0.089565 | n.d. | n.d. | n.d. | n.d. | n.d. | n.d. | n.d. | n.d. |
| 755/756 | 0.121629 | n.d. | 0.000023 | n.d. | n.d. | n.d. | n.d. | n.d. | n.d. |
| 757/758 | 0.456645 | n.d. | 0.023866 | n.d. | n.d. | n.d. | n.d. | n.d. | n.d. |
| 759/760 | 0.743608 | n.d. | 0.001480 | n.d. | n.d. | n.d. | n.d. | n.d. | n.d. |
| 763/764 | n.d. | n.d. | n.d. | 0.247 | n.d. | 0.002716 | n.d. | n.d. | n.d. |
| 765/766 | n.d. | n.d. | n.d. | 0.342513 | n.d. | 0.005981 | n.d. | n.d. | n.d. |
| 767/768 | n.d. | n.d. | n.d. | 0.368050 | n.d. | 0.001958 | n.d. | n.d. | n.d. |
| 769/770 | n.d. | n.d. | n.d. | 0.540068 | n.d. | 0.001842 | n.d. | n.d. | n.d. |
| 771/772 | n.d. | n.d. | n.d. | 0.735010 | n.d. | 0.011205 | n.d. | n.d. | n.d. |
| 773/774 | n.d. | n.d. | n.d. | 2.647375 | n.d. | 0.020964 | n.d. | n.d. | n.d. |
| 775/776 | n.d. | n.d. | n.d. | n.d. | n.d. | 0.411416 | n.d. | n.d. | n.d. |
| 777/778 | n.d. | n.d. | n.d. | n.d. | n.d. | n.d. | 0.012867 | n.d. | 0.000014 |
| 779/780 | n.d. | n.d. | n.d. | n.d. | n.d. | n.d. | 0.023040 | 25.15 | 0.000390 |
| 781/782 | n.d. | n.d. | n.d. | n.d. | n.d. | n.d. | 0.034820 | 7.55 | 0.000459 |
| 783/784 | n.d. | n.d. | n.d. | 0.144145 | n.d. | 0.002183 | 0.046669 | n.d. | 0.000170 |
| 785/786 | n.d. | n.d. | n.d. | n.d. | n.d. | n.d. | 0.082807 | 1356895.35 | 0.000115 |
| 787/788 | n.d. | n.d. | n.d. | 0.235883 | n.d. | 0.001610 | 0.211901 | n.d. | 0.001776 |

TABLE 8

| | Stability | | Stability | | | |
|---|---|---|---|---|---|---|
| | Mouse Serum | | Rat Serum | | | |
| | | anti- | | anti- | Human PBMC | |
| | sense | sense | sense | sense | assay | |
| SEQ ID NO pair | t½ [hr] | t½ [hr] | t½ [hr] | t½ [hr] | IFN-a | TNF-a |
| 739/740 | 6.2 | 3.2 | n.d. | n.d. | 0 | 0 |
| 743/744 | 14.1 | 11.2 | n.d. | n.d. | 0 | 0 |
| 765/766 | 9 | 9.4 | n.d. | n.d. | 0 | 0 |
| 769/770 | 16.2 | 13.1 | n.d. | n.d. | 0 | 0 |
| 777/778 | 14.7 | 12.9 | 18.4 | 16.4 | 0 | 0 |
| 779/780 | 8.5 | 8.5 | 15.7 | 15.6 | 0 | 0 |
| 781/782 | 10.1 | 8.8 | 15.8 | 14.1 | 0 | 0 |
| 783/784 | 11.3 | 13.7 | 18 | 17.5 | 0 | 0 |
| 785/786 | 14.3 | 14.2 | 23.8 | 18.2 | 0 | 0 |
| 787/788 | 14.7 | 11.8 | 24.7 | 17.8 | 0 | 0 |

TABLE 9

| | | | |
|---|---|---|---|
| FPL Name | Function | Sequence | SEQ ID No. |

| | | | |
|---|---|---|---|
| QG2_hIKK2_1 | LE | gatgttttctggctttagatcccTTTTTgaagttaccgtttt | 857 |
| QG2_hIKK2_2 | LE | ctgttctccttgctgcaggacTTTTTctgagtcaaagcat | 858 |
| QG2_hIKK2_3 | CE | cctaggtcaataattttgtgtattaacctTTTTTctcttggaaagaaagt | 859 |
| QG2_hIKK2_4 | CE | tgatccagctccttggcatatTTTTTctcttggaaagaaagt | 860 |
| QG2_hIKK2_5 | LE | aatgatgtgcaaagactgcccTTTTTgaagttaccgtttt | 861 |
| QG2_hIKK2_6 | LE | tactgcagggtccccacgTTTTTctgagtcaaagcat | 862 |
| QG2_hIKK2_7 | CE | cagtagctctggggccaggTTTTTctcttggaaagaaagt | 863 |
| QG2_hIKK2_8 | LE | ggtcactgtgtacttctgctgctcTTTTTgaagttaccgtttt | 864 |
| QG2_hIKK2_9 | LE | gccgaagctccagtagtcgacTTTTTctgagtcaaagcat | 865 |
| QG2_hIKK2_10 | CE | tgcactcaaaggccagggtTTTTTctcttggaaagaaagt | 866 |
| QG2_hIKK2_11 | LE | ggccggaagcccgtgaTTTTTgaagttaccgtttt | 867 |
| QG2_hIKK2_12 | LE | ctgccagttggggaggaagTTTTTctgagtcaaagcat | 868 |
| QG2_hIKK2_13 | CE | tgaatgccactgcacgggTTTTTctcttggaaagaaagt | 869 |
| QG2_hIKK2_14 | LE | ctcactcttctgccgcactttTTTTTgaagttaccgtttt | 870 |
| QG2_hIKK2_15 | LE | tcttcgctaacaacaatgtccacTTTTTctgagtcaaagcat | 871 |
| QG2_hIKK2_16 | BL | aaaacttcaccgttccattcaag | 872 |
| QG2_hIKK2_17 | CE | tggggtagggtaaagagcttgTTTTTctcttggaaagaaagt | 873 |
| QG2_hIKK2_18 | LE | tcagccaggacactgttaagattatTTTTTgaagttaccgtttt | 874 |
| QG2_hIKK2_19 | LE | gcagccacttctccagtcgcTTTTTctgagtcaaagcat | 875 |

TABLE 10

| | | | |
|---|---|---|---|
| FPL Name | Function | Sequence | SEQ ID No. |

| | | | |
|---|---|---|---|
| hGAP001 | CE | gaatttgccatgggtggaatTTTTTctcttggaaagaaagt | 876 |
| hGAP002 | CE | ggagggatctcgctcctggaTTTTTctcttggaaagaaagt | 877 |
| hGAP003 | CE | ccccagccttctccatggtTTTTTctcttggaaagaaagt | 878 |
| hGAP004 | CE | gctcccccctgcaaatgagTTTTTctcttggaaagaaagt | 879 |
| hGAP005 | LE | agccttgacggtgccatgTTTTTaggcataggacccgtgtct | 880 |
| hGAP006 | LE | gatgacaagcttcccgttctcTTTTTaggcataggacccgtgtct | 881 |
| hGAP007 | LE | agatggtgatgggatttccattTTTTTaggcataggacccgtgtct | 882 |
| hGAP008 | LE | gcatcgccccacttgattttTTTTTaggcataggacccgtgtct | 883 |
| hGAP009 | LE | cacgacgtactcagcgccaTTTTTaggcataggacccgtgtct | 884 |
| hGAP010 | LE | ggcagagatgatgacccttttgTTTTTaggcataggacccgtgtct | 885 |
| hGAP011 | BL | ggtgaagacgccagtggactc | 886 |

TABLE 11

| | | | |
|---|---|---|---|
| FPL Name | Function | Sequence | SEQ ID No. |

| | | | |
|---|---|---|---|
| QG2/V2_mIKK-2_2 | LE | tctgctctttatacttctccaggtcTTTTTctgagtcaaagcat | 923 |
| QG2/V2_mIKK-2_3 | CE | tgaggtgatcccaaactcggTTTTTctcttggaaagaaagt | 924 |
| QG2/V2_mIKK-2_4 | CE | ccaagccagcagcaatttatcTTTTTctcttggaaagaaagt | 925 |
| QG2/V2_mIKK-2_5 | LE | agcctgctccatctcccgTTTTTgaagttaccgtttt | 887 |
| QG2/V2_mIKK-2_6 | LE | ccgcccacactgctccacTTTTTctgagtcaaagcat | 888 |
| QG2/V2_mIKK-2_7 | LE | tctactagatgcttcacgtcattctcTTTTTgaagttaccgtttt | 889 |
| QG2/V2_mIKK-2_8 | LE | tgcagtgccatcatccgcTTTTTctgagtcaaagcat | 890 |
| QG2/V2_mIKK-2_9 | LE | ctgcaggtccacaatgtcagtcTTTTTgaagttaccgtttt | 891 |
| QG2/V2_mIKK-2_10 | LE | cgacccatcgggctcctTTTTTctgagtcaaagcat | 892 |
| QG2/V2_mIKK-2_11 | BL | gggtgcccccctgcttc | 893 |
| QG2/V2_mIKK-2_12 | CE | gcttgttcctctaggtcatccaTTTTTctcttggaaagaaagt | 894 |
| QG2/V2_mIKK-2_13 | LE | gagtcttcggtagagctccctcTTTTTgaagttaccgtttt | 895 |
| QG2/V2_mIKK-2_14 | LE | ttggtctcttggcttctccctTTTTTctgagtcaaagcat | 896 |
| QG2/V2_mIKK-2_15 | CE | cctggctgtcaccttctgtcctTTTTTctcttggaaagaaagt | 897 |
| QG2/V2_mIKK-2_16 | LE | aagcagcagccgtaccatctTTTTTgaagttaccgtttt | 898 |
| QG2/V2_mIKK-2_17 | LE | ctcaaagctttggattgcctgTTTTTctgagtcaaagcat | 899 |
| QG2/V2_mIKK-2_18 | CE | gtgtataaatcacccgaactttcttTTTTTctcttggaaagaaagt | 900 |
| QG2/V2_mIKK-2_19 | BL | caaaccacggtcttactgagct | 901 |
| QG2/V2_mIKK-2_20 | CE | caactccagtgccttctgcttaTTTTTctcttggaaagaaagt | 902 |

TABLE 12

| | | | SEQ ID |
|---|---|---|---|
| FPL Name | Function | Sequence | No. |
| mGAPDH_QG2_2 | LE | cttcaccattttgtctacgggaTTTTTgaagttaccgtttt | 903 |
| mGAPDH_QG2_3 | LE | ccaaatccgttcacaccgacTTTTTctgagtcaaagcat | 904 |
| mGAPDH_QG2_4 | CE | ccaggcgcccaatacggTTTTTctcttggaaagaaagt | 905 |
| mGAPDH_QG2_5 | LE | caaatggcagccctggtgaTTTTTgaagttaccgtttt | 906 |
| mGAPDH_QG2_6 | LE | aacaatctccactttgccactgTTTTTctgagtcaaagcat | 907 |
| mGAPDH_QG2_7 | BL | tgaaggggtcgttgatggc | 908 |
| mGAPDH_QG2_8 | BL | catgtagaccatgtagttgaggtcaa | 909 |
| mGAPDH_QG2_9 | CE | ccgtgagtggagtcatactggaaTTTTTctcttggaaagaaagt | 910 |
| mGAPDH_QG2_10 | CE | ttgactgtgccgttgaatttgTTTTTctcttggaaagaaagt | 911 |
| mGAPDH_QG2_11 | LE | agcttcccattctcggccTTTTTgaagttaccgtttt | 912 |
| mGAPDH_QG2_12 | LE | gggcttcccgttgatgacaTTTTTctgagtcaaagcat | 913 |
| mGAPDH_QG2_13 | CE | cgctcctggaagatggtgatTTTTTctcttggaaagaaagt | 914 |
| mGAPDH_QG2_14 | BL | cccatttgatgttagtggggtct | 915 |
| mGAPDH_QG2_15 | CE | atactcagcaccggcctcacTTTTTctcttggaaagaaagt | 916 |

TABLE 13

| unmodified sequence | | | | modified sequence | | | |
|---|---|---|---|---|---|---|---|
| SEQ | Sense strand sequence | SEQ | Antisense strand sequence | SEQ | Sense strand sequence | SEQ | Antisense strand sequence |
| ID No | (5′-3′) | ID No | (5′-3′) | ID No | (5′-3′) | ID No | (5′-3′) |
| 1 | ACUUAAAGCUGGUUCAU | 110 | AUAUGAACCAGCUUUAAGU | 211 | AcuuAAAGcuGGuucAuAudTsdT | 212 | AuAUGAACcAGCUUuAAGUdTsdT |
| | AU | | | | | | |
| 2 | GCAUGAAUGCCUCUCGACU | 111 | AGUCGAGAGGCAUUCAUGC | 213 | GcAuGAAuGccucucGAcudTsdT | 214 | AGUCGAGAGGcAUUcAUGCdTsdT |
| 3 | GGUGGUGAGCUUAAUGA | 112 | AUUCAUUAAGCUCACCACC | 215 | GGuGGuGAGcuuAAuGAAudTsdT | 216 | AUUcAUuAAGCUcACcACCdTsdT |
| | AU | | | | | | |
| 3 | GGUGGUGAGCUUAAUGA | 112 | AUUCAUUAAGCUCACCACC | 217 | GguGGuGAGcuuAAuGAAudTsdT | 218 | AUUcAUuAAGCUcACcACCdTsdT |
| | AU | | | | | | |
| 1 | ACUUAAAGCUGGUUCAU | 110 | AUAUGAACCAGCUUUAAGU | 219 | ACfUfUfAAAGCfUfGGUfUfCfAUfAUfdTs | 220 | pAUfAUfGAACfCfAGCfUfUfUfAAGUfdT |
| | AU | | | | dT | | sdT |
| 4 | TGUGGUGAGCUUAAUGA | 112 | AUUCAUUAAGCUCACCACC | 221 | (OMedT)guGGuGAGcuuAAuGAAudTsdT | 222 | AUUcAUuAAGCUcACcACCdTsdT |
| | AU | | | | | | |
| 5 | GCAAGGGAGCUGUACAG | 113 | UCCUGUACAGCUCCCUUGC | 223 | GcAAGGGAGcuGuAcAGGAdTsdT | 224 | puCCUGuAcAGCUCCCUUGcdTsdT |
| | GA | | | | | | |
| 6 | TCAUGAAUGCCUCUCGACC | 111 | AGUCGAGAGGCAUUCAUGC | 225 | (OMedT)cAuGAAuGccucucGAccdTsdT | 226 | pAGuCGAGAGGcAUUcAUGcdTsdT |
| 7 | TGUGGUGAGCUUAAUGA | 112 | AUUCAUUAAGCUCACCACC | 227 | (OMedT)guGGuGAGcuuAAuGAAcdTsdT | 228 | AUUcAUuAAGCUcACcACCdTsdT |
| | AC | | | | | | |
| 8 | AGUACACAGUGACCGUCGA | 114 | UCGACGGUCACUGUGUACU | 229 | AguAcAcAGuGAccGucGAdTsdT | 230 | puCGACGGUcACUGUGuACudTsdT |
| 9 | TGUACACAGUGACCGUCGC | 114 | UCGACGGUCACUGUGUACU | 231 | (OMedT)guAcAcAGuGAccGucGcdTsdT | 232 | puCGACGGUcACUGUGuACUdTsdT |
| 5 | GCAAGGGAGCUGUACAG | 113 | UCCUGUACAGCUCCCUUGC | 233 | GcAAGGGAGcuGuAcAGGAdTsdT | 234 | UCCUGuAcAGCUCCCUUGCdTsdT |
| | GA | | | | | | |
| 10 | TCUUAAAGCUGGUUCAUAC | 110 | AUAUGAACCAGCUUUAAGU | 235 | (OMedT)cuuAAAGcuGGuucAuAcdTsdT | 236 | pAuAUGAACcAGCUUuAAGudTsdT |
| 5 | GCAAGGGAGCUGUACAG | 113 | UCCUGUACAGCUCCCUUGC | 237 | GcAAGGGAGcuGuAcAGGAdTsdT | 238 | puCCuGuAcAGCUCCCUuGcdTsdT |
| | GA | | | | | | |
| 9 | TGUACACAGUGACCGUCGC | 114 | UCGACGGUCACUGUGUACU | 239 | (OMedT)guAcAcAGuGAccGucGcdTsdT | 240 | puCGACGGUcACUGUGuACudTsdT |
| 8 | AGUACACAGUGACCGUCGA | 114 | UCGACGGUCACUGUGUACU | 241 | AGuAcAcAGuGAccGucGAdTsdT | 242 | UCGACGGUcACUGUGuACUdTsdT |
| 11 | TCAAGGGAGCUGUACAGGC | 113 | UCCUGUACAGCUCCCUUGC | 243 | (OMedT)cAAGGGAGcuGuAcAGGcdTsdT | 244 | puCCUGuAcAGCUCCCUUGcdTsdT |
| 12 | TGUACACAGUGACCGUCGA | 114 | UCGACGGUCACUGUGUACU | 245 | (OMedT)guAcAcAGuGAccGucGAdTsdT | 246 | puCGACGGUcACUGUGuACUdTsdT |
| 13 | TCAAGGGAGCUGUACAGGA | 113 | UCCUGUACAGCUCCCUUGC | 247 | (OMedT)cAAGGGAGcuGuAcAGGAdTsdT | 248 | puCCuGuAcAGCUCCCUuGcdTsdT |
| 8 | AGUACACAGUGACCGUCGA | 114 | UCGACGGUCACUGUGUACU | 249 | AGuAcAcAGuGAccGucGAdTsdT | 250 | uCGACGGUcACUGUGuACudTsdT |
| 9 | TGUACACAGUGACCGUCGC | 114 | UCGACGGUCACUGUGUACU | 251 | (OMedT)guAcAcAGuGAccGucGcdTsdT | 252 | uCGACGGUcACUGUGuACudTsdT |
| 2 | GCAUGAAUGCCUCUCGACU | 111 | AGUCGAGAGGCAUUCAUGC | 253 | GcAuGAAuGccucucGAcudTsdT | 254 | AGuCGAGAGGcAUUcAUGCdTsdT |
| 13 | TCAAGGGAGCUGUACAGGA | 113 | UCCUGUACAGCUCCCUUGC | 255 | (OMedT)cAAGGGAGcuGuAcAGGAdTsdT | 256 | UCCUGuAcAGCUCCCUUGCdTsdT |
| 13 | TCAAGGGAGCUGUACAGGA | 113 | UCCUGUACAGCUCCCUUGC | 257 | (OMedT)cAAGGGAGcuGuAcAGGAdTsdT | 258 | uCCuGuAcAGCUCCCUuGcdTsdT |
| 11 | TCAAGGGAGCUGUACAGGC | 113 | UCCUGUACAGCUCCCUUGC | 259 | (OMedT)cAAGGGAGcuGuAcAGGcdTsdT | 260 | puCCuGuAcAGCUCCCUuGcdTsdT |
| 12 | TGUACACAGUGACCGUCGA | 114 | UCGACGGUCACUGUGUACU | 261 | (OMedT)guAcAcAGuGAccGucGAdTsdT | 262 | UCGACGGUcACUGUGuACUdTsdT |
| 1 | ACUUAAAGCUGGUUCAU | 110 | AUAUGAACCAGCUUUAAGU | 263 | AcuuAAAGcuGGuucAuAudTsdT | 264 | AuAUGAACcAGCUUuAAGudTsdT |
| | AU | | | | | | |
| 9 | TGUACACAGUGACCGUCGC | 114 | UCGACGGUCACUGUGUACU | 265 | (OMedT)guAcAcAGuGAccGucGcdTsdT | 266 | UCGACGGUcACUGUGuACUdTsdT |
| 14 | TCAUGAAUGCCUCUCGACU | 118 | GGUCGAGAGGCAUUCAUGC | 267 | (OMedT)cAuGAAuGccucucGAcudTsdT | 268 | GGuCGAGAGGcAUUcAUGcdTsdT |
| 1 | ACUUAAAGCUGGUUCAU | 110 | AUAUGAACCAGCUUUAAGU | 269 | AcuuAAAGcuGGuucAuAudTsdT | 270 | pAuAUGAACcAGCUUuAAGudTsdT |
| | AU | | | | | | |
| 13 | TCAAGGGAGCUGUACAGGA | 113 | UCCUGUACAGCUCCCUUGC | 271 | (OMedT)cAAGGGAGcuGuAcAGGAdTsdT | 272 | puCCUGuAcAGCUCCCUUGcdTsdT |
| 11 | TCAAGGGAGCUGUACAGGC | 113 | UCCUGUACAGCUCCCUUGC | 273 | (OMedT)cAAGGGAGcuGuAcAGGcdTsdT | 274 | UCCUGuAcAGCUCCCUUGCdTsdT |
| 8 | AGUACACAGUGACCGUCGA | 114 | UCGACGGUCACUGUGUACU | 275 | AguAcAcAGuGAccGucGAdTsdT | 276 | puCGACGGUcACUGUGuACUdTsdT |
| 6 | TCAUGAAUGCCUCUCGACC | 111 | AGUCGAGAGGCAUUCAUGC | 277 | (OMedT)cAuGAAuGccucucGAccdTsdT | 278 | AGuCGAGAGGcAUUcAuGCdTsdT |
| 8 | AGUACACAGUGACCGUCGA | 114 | UCGACGGUCACUGUGUACU | 279 | AguAcAcAGuGAccGucGAdTsdT | 280 | uCGACGGUcACUGUGuACudTsdT |
| 12 | TGUACACAGUGACCGUCGA | 114 | UCGACGGUCACUGUGUACU | 281 | (OMedT)guAcAcAGuGAccGucGAdTsdT | 282 | uCGACGGUcACUGUGuACudTsdT |
| 2 | GCAUGAAUGCCUCUCGACU | 111 | AGUCGAGAGGCAUUCAUGC | 283 | GcAuGAAuGccucucGAcudTsdT | 284 | pAGuCGAGAGGcAUUcAUGCdTsdT |
| 1 | ACUUAAAGCUGGUUCAU | 110 | AUAUGAACCAGCUUUAAGU | 285 | AcuuAAAGcuGGuucAuAudTsdT | 286 | pAuAUGAACcAGCUuuAAGudTsdT |
| | AU | | | | | | |
| 1 | ACUUAAAGCUGGUUCAU | 110 | AUAUGAACCAGCUUUAAGU | 287 | ACfUfUfAAAGCfUfGGUfUfCfAUfAUfdTs | 288 | AUfAUfGAACfCfAGCfUfUfUfAAGUfdTs |
| | AU | | | | dT | | dT |
| 10 | TCUUAAAGCUGGUUCAUAC | 110 | AUAUGAACCAGCUUUAAGU | 289 | (OMedT)cuuAAAGcuGGuucAuAcdTsdT | 290 | AuAUGAACcAGCUUuAAGUdTsdT |
| 15 | GGAUGAGAAGACUGUUG | 115 | GACAACAGUCUUCUCAUCC | 291 | GGAuGAGAAGAcuGuuGucdTsdT | 292 | GAcAAcAGUCUUCUcAUCCdTsdT |
| 11 | TCAAGGGAGCUGUACAGGC | 113 | UCCUGUACAGCUCCCUUGC | 293 | (OMedT)cAAGGGAGcuGuAcAGGcdTsdT | 294 | uCCuGuAcAGCUCCCUuGcdTsdT |
| 2 | GCAUGAAUGCCUCUCGACU | 111 | AGUCGAGAGGCAUUCAUGC | 295 | GcAuGAAuGccucucGAcudTsdT | 296 | pAGuCGAGAGGcAUUcAuGCdTsdT |
| 3 | GGUGGUGAGCUUAAUGA | 112 | AUUCAUUAAGCUCACCACC | 297 | GGUfGGUfGAGCfUfUfAAUfGAAUfdTsdT | 298 | pAUfUfCfAUfUfAAGCfUfCfACfCfACfCfd |
| | AU | | | | | | TsdT |
| 16 | GCUAGAAAAUGCCAUACAG | 116 | CUGUAUGGCAUUUUCUAGC | 299 | GcuAGAAAAuGccAuAcAGdTsdT | 300 | CUGuAUGGcAUUUUCuAGCdTsdT |
| 17 | CUGAAGAUUGCUUGUAG | 117 | UGCUACAAGCAAUCUUCAG | 301 | cuGAAGAuuGcuuGuAGcAdTsdT | 302 | UGCuAcAAGcAAUCUUcAGdTsdT |
| | CA | | | | | | |
| 8 | AGUACACAGUGACCGUCGA | 114 | UCGACGGUCACUGUGUACU | 303 | AguAcAcAGuGAccGucGAdTsdT | 304 | uCGACGGUcACUGUGuACUdTsdT |
| 14 | TCAUGAAUGCCUCUCGACU | 111 | AGUCGAGAGGCAUUCAUGC | 305 | (OMedT)cAuGAAuGccucucGAcudTsdT | 306 | AGuCGAGAGGcAUUcAUGCdTsdT |
| 14 | TCAUGAAUGCCUCUCGACU | 111 | AGUCGAGAGGCAUUCAUGC | 307 | (OMedT)cAuGAAuGccucucGAcudTsdT | 308 | pAGuCGAGAGGcAUUcAUGcdTsdT |
| 6 | TCAUGAAUGCCUCUCGACC | 111 | AGUCGAGAGGCAUUCAUGC | 309 | (OMedT)cAuGAAuGccucucGAccdTsdT | 310 | AGuCGAGAGGcAUUcAUGcdTsdT |
| 9 | TGUACACAGUGACCGUCGC | 114 | UCGACGGUCACUGUGUACU | 311 | (OMedT)guAcAcAGuGAccGucGcdTsdT | 312 | uCGACGGUcACUGUGuACUdTsdT |
| 2 | GCAUGAAUGCCUCUCGACU | 118 | GGUCGAGAGGCAUUCAUGC | 313 | GcAuGAAuGccucucGAcudTsdT | 314 | GGuCGAGAGGcAUUcAUGcdTsdT |
| 14 | TCAUGAAUGCCUCUCGACU | 111 | AGUCGAGAGGCAUUCAUGC | 315 | (OMedT)cAuGAAuGccucucGAcudTsdT | 316 | AGuCGAGAGGcAUUcAUGcdTsdT |
| 18 | CCGACAGAGUUAGCACGAC | 119 | GUCGUGCUAACUCUGUCGG | 317 | ccGAcAGAGuuAGcAcGAcdTsdT | 318 | GUCGUGCuAACUCUGUCGGdTsdT |
| 8 | AGUACACAGUGACCGUCGA | 114 | UCGACGGUCACUGUGUACU | 319 | AguAcAcAGuGAccGucGAdTsdT | 320 | UCGACGGUcACUGUGuACUdTsdT |
| 4 | TGUGGUGAGCUUAAUGA | 112 | AUUCAUUAAGCUCACCACC | 321 | (OMedT)guGGuGAGcuuAAuGAAudTsdT | 322 | AuucAUuAAGCUcACcACcdTsdT |
| | AU | | | | | | |
| 3 | GGUGGUGAGCUUAAUGA | 112 | AUUCAUUAAGCUCACCACC | 323 | GGuGGuGAGcuuAAuGAAudTsdT | 324 | pAuucAUuAAGCUcACcACcdTsdT |
| | AU | | | | | | |
| 8 | AGUACACAGUGACCGUCGA | 114 | UCGACGGUCACUGUGUACU | 325 | AGuAcAcAGuGAccGucGAdTsdT | 326 | puCGACGGUcACUGUGuACUdTsdT |
| 2 | GCAUGAAUGCCUCUCGACU | 111 | AGUCGAGAGGCAUUCAUGC | 327 | GCfAUfGAAUfGCfCfUfCfUfCfGACfUfdTs | 328 | pAGUfCfGAGAGGCfAUfUfCfAUfGCfdTs |
| | | | | | dT | | dT |
| 14 | TCAUGAAUGCCUCUCGACU | 111 | AGUCGAGAGGCAUUCAUGC | 329 | (OMedT)cAuGAAuGccucucGAcudTsdT | 330 | pAGuCGAGAGGcAUUcAuGCdTsdT |
| 7 | TGUGGUGAGCUUAAUGA | 112 | AUUCAUUAAGCUCACCACC | 331 | (OMedT)guGGuGAGcuuAAuGAAcdTsdT | 332 | pAuucAUuAAGCUcACcACcdTsdT |
| | AC | | | | | | |
| 19 | AGUGUCAGCUGUAUCCUUC | 120 | GAAGGAUACAGCUGACACU | 333 | AGuGucAGcuGuAuccuucdTsdT | 334 | GAAGGAuAcAGCUGAcACUdTsdT |
| 20 | AGGCAAUUCAGAGCUUCGA | 121 | UCGAAGCUCUGAAUUGCCU | 335 | AGGcAAuucAGAGcuucGAdTsdT | 336 | UCGAAGCUCUGAAUUGCCUdTsdT |
| 3 | GGUGGUGAGCUUAAUGA | 112 | AUUCAUUAAGCUCACCACC | 337 | GGuGGuGAGcuuAAuGAAudTsdT | 338 | AuucAUuAAGCUcACcACcdTsdT |
| | AU | | | | | | |
| 12 | TGUACACAGUGACCGUCGA | 114 | UCGACGGUCACUGUGUACU | 339 | (OMedT)guAcAcAGuGAccGucGAdTsdT | 340 | puCGACGGUcACUGUGuACudTsdT |
| 14 | TCAUGAAUGCCUCUCGACU | 111 | AGUCGAGAGGCAUUCAUGC | 341 | (OMedT)cAuGAAuGccucucGAcudTsdT | 342 | AGuCGAGAGGcAUUcAuGCdTsdT |
| 3 | GGUGGUGAGCUUAAUGA | 112 | AUUCAUUAAGCUCACCACC | 343 | GguGGuGAGcuuAAuGAAudTsdT | 344 | AuucAUuAAGCUcACcACcdTsdT |
| | AU | | | | | | |
| 4 | TGUGGUGAGCUUAAUGA | 112 | AUUCAUUAAGCUCACCACC | 345 | (OMedT)guGGuGAGcuuAAuGAAudTsdT | 346 | AuUcAUuAAGCUcACcACcdTsdT |
| | AU | | | | | | |
| 4 | TGUGGUGAGCUUAAUGA | 112 | AUUCAUUAAGCUCACCACC | 347 | (OMedT)guGGuGAGcuuAAuGAAudTsdT | 348 | pAuUcAUuAAGCUcACcACcdTsdT |
| | AU | | | | | | |
| 7 | TGUGGUGAGCUUAAUGA | 112 | AUUCAUUAAGCUCACCACC | 349 | (OMedT)guGGuGAGcuuAAuGAAcdTsdT | 350 | AuucAUuAAGCUcACcACcdTsdT |
| | AC | | | | | | |
| 21 | TCUUAAAGCUGGUUCAUAU | 110 | AUAUGAACCAGCUUUAAGU | 351 | (OMedT)cuuAAAGcuGGuucAuAudTsdT | 352 | pAuAUGAACcAGCUUuAAGudTsdT |
| 21 | TCUUAAAGCUGGUUCAUAU | 110 | AUAUGAACCAGCUUUAAGU | 353 | (OMedT)cuuAAAGcuGGuucAuAudTsdT | 354 | AuAUGAACcAGCUUuAAGudTsdT |
| 22 | GAUCAGGGCAGUCUUUGCA | 122 | UGCAAAGACUGCCCUGAUC | 355 | GAucAGGGcAGucuuuGcAdTsdT | 356 | UGcAAAGACUGCCCUGAUCdTsdT |
| 23 | UCAGGAAAUGGUACGGC | 123 | CAGCCGUACCAUUUCCUGA | 357 | ucAGGAAAuGGuAcGGcuGdTsdT | 358 | cAGCCGuACcAUUUCCUGAdTsdT |
| | UG | | | | | | |
| 5 | GCAAGGGAGCUGUACAG | 113 | UCCUGUACAGCUCCCUUGC | 359 | GcAAGGGAGcuGuAcAGGAdTsdT | 360 | uCCuGuAcAGCUCCCUuGcdTsdT |
| | GA | | | | | | |
| 5 | GCAAGGGAGCUGUACAG | 113 | UCCUGUACAGCUCCCUUGC | 361 | GCfAAGGGAGCfUfGUfACfAGGAdTsdT | 362 | pUfCfCfUfGUfACfAGCfUfCfCfCfUfUfGC |
| | GA | | | | | | fdTsdT |
| 6 | TCAUGAAUGCCUCUCGACC | 118 | GGUCGAGAGGCAUUCAUGC | 363 | (OMedT)cAuGAAuGccucucGAccdTsdT | 364 | GGuCGAGAGGcAUUcAUGcdTsdT |
| 3 | GGUGGUGAGCUUAAUGA | 112 | AUUCAUUAAGCUCACCACC | 365 | GGuGGuGAGcuuAAuGAAudTsdT | 366 | AuUcAUuAAGCUcACcACcdTsdT |
| | AU | | | | | | |
| 12 | TGUACACAGUGACCGUCGA | 114 | UCGACGGUCACUGUGUACU | 367 | (OMedT)guAcAcAGuGAccGucGAdTsdT | 368 | uCGACGGUcACUGUGuACUdTsdT |
| 6 | TCAUGAAUGCCUCUCGACC | 111 | AGUCGAGAGGCAUUCAUGC | 369 | (OMedT)cAuGAAuGccucucGAccdTsdT | 370 | AGUCGAGAGGcAUUcAUGCdTsdT |
| 3 | GGUGGUGAGCUUAAUGA | 112 | AUUCAUUAAGCUCACCACC | 371 | GguGGuGAGcuuAAuGAAudTsdT | 372 | AuUcAUuAAGCUcACcACcdTsdT |
| | AU | | | | | | |
| 21 | TCUUAAAGCUGGUUCAUAU | 110 | AUAUGAACCAGCUUUAAGU | 373 | (OMedT)cuuAAAGcuGGuucAuAudTsdT | 374 | AuAUGAACcAGCUUuAAGUdTsdT |
| 10 | TCUUAAAGCUGGUUCAUAC | 110 | AUAUGAACCAGCUUUAAGU | 375 | (OMedT)cuuAAAGcuGGuucAuAcdTsdT | 376 | AuAUGAACcAGCUUuAAGudTsdT |
| 24 | UGGUUCAUAUCUUGAAC | 124 | AUGUUCAAGAUAUGAACCA | 377 | uGGuucAuAucuuGAAcAudTsdT | 378 | AUGUUcAAGAuAUGAACcAdTsdT |
| | AU | | | | | | |
| 25 | ACAGAAUCAUCCAUCGGGA | 125 | UCCCGAUGGAUGAUUCUGU | 379 | AcAGAAucAuccAucGGGAdTsdT | 380 | UCCCGAUGGAUGAUUCUGUdTsdT |
| 26 | GUACACAGUGACCGUCGAC | 126 | GUCGACGGUCACUGUGUAC | 381 | GuAcAcAGuGAccGucGAcdTsdT | 382 | GUCGACGGUcACUGUGuACdTsdT |
| 27 | UUGCUUGUAGCAAGGUCCG | 127 | CGGACCUUGCUACAAGCAA | 383 | uuGcuuGuAGcAAGGuccGdTsdT | 384 | CGGACCUUGCuAcAAGcAAdTsdT |
| 3 | GGUGGUGAGCUUAAUGA | 112 | AUUCAUUAAGCUCACCACC | 385 | GGuGGuGAGcuuAAuGAAudTsdT | 386 | pAuUcAUuAAGCUcACcACcdTsdT |
| | AU | | | | | | |
| 6 | TCAUGAAUGCCUCUCGACC | 111 | AGUCGAGAGGCAUUCAUGC | 387 | (OMedT)cAuGAAuGccucucGAccdTsdT | 388 | pAGuCGAGAGGcAUUcAUGCdTsdT |
| 3 | GGUGGUGAGCUUAAUGA | 112 | AUUCAUUAAGCUCACCACC | 389 | GguGGuGAGcuuAAuGAAudTsdT | 390 | pAuUcAUuAAGCUcACcACcdTsdT |
| | AU | | | | | | |
| 3 | GGUGGUGAGCUUAAUGA | 112 | AUUCAUUAAGCUCACCACC | 391 | GguGGuGAGcuuAAuGAAudTsdT | 392 | pAuucAUuAAGCUcACcACcdTsdT |
| | AU | | | | | | |
| 28 | CUGCCGACAGAGUUAGCAC | 128 | GUGCUAACUCUGUCGGCAG | 393 | cuGccGAcAGAGuuAGcAcdTsdT | 394 | GUGCuAACUCUGUCGGcAGdTsdT |
| 29 | GAAAGUGCGAGUGAUCU | 129 | AUAGAUCACUCGCACUUUC | 395 | GAAAGuGcGAGuGAucuAudTsdT | 396 | AuAGAUcACUCGcACUUUCdTsdT |
| 5 | GCAAGGGAGCUGUACAG | 113 | UCCUGUACAGCUCCCUUGC | 397 | GCfAAGGGAGCfUfGUfACfAGGAdTsdT | 398 | UfCfCfUfGUfACfAGCfUfCfCfCfUfUfGCf |
| | GA | | | | | | dTsdT |
| 8 | AGUACACAGUGACCGUCGA | 114 | UCGACGGUCACUGUGUACU | 399 | AGuAcAcAGuGAccGucGAdTsdT | 400 | puCGACGGUcACUGUGuACudTsdT |
| 8 | AGUACACAGUGACCGUCGA | 114 | UCGACGGUCACUGUGUACU | 401 | AGUfACfACfAGUfGACfCfGUfCfGAdTsdT | 402 | UfCfGACfGGUfCfACfUfGUfGUfACfUfdT |
| | | | | | | | sdT |
| 9 | TGUACACAGUGACCGUCGC | 114 | UCGACGGUCACUGUGUACU | 403 | (OMedT)guAcAcAGuGAccGucGcdTsdT | 404 | puCGACGGUcACUGuGuACudTsdT |
| 9 | TGUACACAGUGACCGUCGC | 114 | UCGACGGUCACUGUGUACU | 405 | (OMedT)guAcAcAGuGAccGucGcdTsdT | 406 | uCGACGGUcACUGuGuACudTsdT |
| 2 | GCAUGAAUGCCUCUCGACU | 111 | AGUCGAGAGGCAUUCAUGC | 407 | GcAuGAAuGccucucGAcudTsdT | 408 | AGuCGAGAGGcAUUcAuGCdTsdT |
| 6 | TCAUGAAUGCCUCUCGACC | 111 | AGUCGAGAGGCAUUCAUGC | 409 | (OMedT)cAuGAAuGccucucGAccdTsdT | 410 | AGuCGAGAGGcAUUcAUGCdTsdT |
| 6 | TCAUGAAUGCCUCUCGACC | 111 | AGUCGAGAGGCAUUCAUGC | 411 | (OMedT)cAuGAAuGccucucGAccdTsdT | 412 | pAGuCGAGAGGcAUUcAuGCdTsdT |
| 10 | TCUUAAAGCUGGUUCAUAC | 110 | AUAUGAACCAGCUUUAAGU | 413 | (OMedT)cuuAAAGcuGGuucAuAcdTsdT | 414 | AuAUGAACcAGCUuuAAGudTsdT |
| 30 | CCUGAAGAUUGCUUGUA | 130 | GCUACAAGCAAUCUUCAGG | 415 | ccuGAAGAuuGcuuGuAGcdTsdT | 416 | GCuAcAAGcAAUCUUcAGGdTsdT |
| | GC | | | | | | |
| 31 | CGACAGAGUUAGCACGACA | 131 | UGUCGUGCUAACUCUGUCG | 417 | cGAcAGAGuuAGcAcGAcAdTsdT | 418 | UGUCGUGCuAACUCUGUCGdTsdT |
| 8 | AGUACACAGUGACCGUCGA | 114 | UCGACGGUCACUGUGUACU | 419 | AGUfACfACfAGUfGACfCfGUfCfGAdTsdT | 420 | pUfCfGACfGGUfCfACfUfGUfGUfACfUfd |
| | | | | | | | TsdT |
| 2 | GCAUGAAUGCCUCUCGACU | 111 | AGUCGAGAGGCAUUCAUGC | 421 | GcAuGAAuGccucucGAcudTsdT | 422 | AGuCGAGAGGcAUUcAUGcdTsdT |
| 7 | TGUGGUGAGCUUAAUGA | 112 | AUUCAUUAAGCUCACCACC | 423 | (OMedT)guGGuGAGcuuAAuGAAcdTsdT | 424 | AuUcAUuAAGCUcACcACcdTsdT |
| | AC | | | | | | |
| 32 | CUAGAAAAUGCCAUACAGG | 132 | CCUGUAUGGCAUUUUCUAG | 425 | cuAGAAAAuGccAuAcAGGdTsdT | 426 | CCUGuAUGGcAUUUUCuAGdTsdT |
| 33 | CUGCCCGCGUUAAGAUUCC | 133 | GGAAUCUUAACGCGGGCAG | 427 | cuGcccGcGuuAAGAuuccdTsdT | 428 | GGAAUCUuAACGCGGGcAGdTsdT |
| 14 | TCAUGAAUGCCUCUCGACU | 111 | AGUCGAGAGGCAUUCAUGC | 429 | (OMedT)cAuGAAuGccucucGAcudTsdT | 430 | pAGuCGAGAGGcAUUcAUGCdTsdT |
| 4 | TGUGGUGAGCUUAAUGA | 112 | AUUCAUUAAGCUCACCACC | 431 | (OMedT)guGGuGAGcuuAAuGAAudTsdT | 432 | pAuucAUuAAGCUcACcACcdTsdT |
| | AU | | | | | | |
| 7 | TGUGGUGAGCUUAAUGA | 112 | AUUCAUUAAGCUCACCACC | 433 | (OMedT)guGGuGAGcuuAAuGAAcdTsdT | 434 | pAuUcAUuAAGCUcACcACcdTsdT |
| | AC | | | | | | |
| 12 | TGUACACAGUGACCGUCGA | 114 | UCGACGGUCACUGUGUACU | 435 | (OMedT)guAcAcAGuGAccGucGAdTsdT | 436 | puCGACGGUcACUGuGuACudTsdT |
| 1 | ACUUAAAGCUGGUUCAU | 110 | AUAUGAACCAGCUUUAAGU | 437 | AcuuAAAGcuGGuucAuAudTsdT | 438 | AuAUGAACcAGCUuuAAGudTsdT |
| | AU | | | | | | |
| 34 | CAGAAUCAUCCAUCGGGAU | 134 | AUCCCGAUGGAUGAUUCUG | 439 | cAGAAucAuccAucGGGAudTsdT | 440 | AUCCCGAUGGAUGAUUCUGdTsdT |
| 35 | GCCAGAAAACAUCGUCCUG | 135 | CAGGACGAUGUUUUCUGGC | 441 | GccAGAAAAcAucGuccuGdTsdT | 442 | cAGGACGAUGUUUUCUGGCdTsdT |
| 36 | GUUUGCAAGCAGAAGGC | 136 | GCGCCUUCUGCUUGCAAAC | 443 | GuuuGcAAGcAGAAGGcGcdTsdT | 444 | GCGCCUUCUGCUUGcAAACdTsdT |
| | GC | | | | | | |
| 37 | UGCUAGAAAAUGCCAUACA | 137 | UGUAUGGCAUUUUCUAGCA | 445 | uGcuAGAAAAuGccAuAcAdTsdT | 446 | UGuAUGGcAUUUUCuAGcAdTsdT |
| 8 | AGUACACAGUGACCGUCGA | 114 | UCGACGGUCACUGUGUACU | 447 | AguAcAcAGuGAccGucGAdTsdT | 448 | puCGACGGUcACUGuGuACudTsdT |
| 3 | GGUGGUGAGCUUAAUGA | 112 | AUUCAUUAAGCUCACCACC | 449 | GGUfGGUfGAGCfUfUfAAUfGAAUfdTsdT | 450 | AUfUfCfAUfUfAAGCfUfCfACfCfACfCfd |
| | AU | | | | | | TsdT |
| 38 | AGGUGGUGAGCUUAAUG | 138 | UUCAUUAAGCUCACCACCU | 451 | AGGuGGuGAGcuuAAuGAAdTsdT | 452 | UUcAUuAAGCUcACcACCUdTsdT |
| | AA | | | | | | |
| 39 | GACAGAGUUAGCACGACAU | 139 | AUGUCGUGCUAACUCUGUC | 453 | GAcAGAGuuAGcAcGAcAudTsdT | 454 | AUGUCGUGCuAACUCUGUCdTsdT |
| 8 | AGUACACAGUGACCGUCGA | 114 | UCGACGGUCACUGUGUACU | 455 | AGuAcAcAGuGAccGucGAdTsdT | 456 | uCGACGGUcACUGUGuACUdTsdT |
| 2 | GCAUGAAUGCCUCUCGACU | 111 | AGUCGAGAGGCAUUCAUGC | 457 | GcAuGAAuGccucucGAcudTsdT | 458 | pAGuCGAGAGGcAUUcAUGcdTsdT |
| 2 | GCAUGAAUGCCUCUCGACU | 111 | AGUCGAGAGGCAUUCAUGC | 459 | GCfAUfGAAUfGCfCfUfCfUfCfGACfUfdTs | 460 | AGUfCfGAGAGGCfAUfUfCfAUfGCfdTsdT |
| | | | | | dT | | |
| 40 | GCGGGAGAACGAAGUGA | 140 | UUUCACUUCGUUCUCCCGC | 461 | GcGGGAGAAcGAAGuGAAAdTsdT | 462 | UUUcACUUCGUUCUCCCGCdTsdT |
| | AA | | | | | | |
| 8 | AGUACACAGUGACCGUCGA | 114 | UCGACGGUCACUGUGUACU | 463 | AguAcAcAGuGAccGucGAdTsdT | 464 | uCGACGGUcACUGuGuACudTsdT |
| 12 | TGUACACAGUGACCGUCGA | 114 | UCGACGGUCACUGUGUACU | 465 | (OMedT)guAcAcAGuGAccGucGAdTsdT | 466 | uCGACGGUcACUGuGuACudTsdT |
| 41 | GAGCUGUACAGGAGACU | 141 | UUAGUCUCCUGUACAGCUC | 467 | GAGcuGuAcAGGAGAcuAAdTsdT | 468 | UuAGUCUCCUGuAcAGCUCdTsdT |
| | AA | | | | | | |
| 42 | CCUCGAGACCAGCGAACUG | 142 | CAGUUCGCUGGUCUCGAGG | 469 | ccucGAGAccAGcGAAcuGdTsdT | 470 | cAGUUCGCUGGUCUCGAGGdTsdT |
| 1 | ACUUAAAGCUGGUUCAU | 143 | GUAUGAACCAGCUUUAAGU | 471 | AcuuAAAGcuGGuucAuAudTsdT | 472 | GuAUGAACcAGCUuuAAGudTsdT |
| | AU | | | | | | |
| 43 | AGCCAGAAAACAUCGUCCU | 144 | AGGACGAUGUUUUCUGGCU | 473 | AGccAGAAAAcAucGuccudTsdT | 474 | AGGACGAUGUUUUCUGGCUdTsdT |
| 44 | CUGGUUACAGACGGAAG | 145 | UUCUUCCGUCUGUAACCAG | 475 | cuGGuuAcAGAcGGAAGAAdTsdT | 476 | UUCUUCCGUCUGuAACcAGdTsdT |
| | AA | | | | | | |
| 45 | AGAGUUUCACGGCCCUAGA | 146 | UCUAGGGCCGUGAAACUCU | 477 | AGAGuuucAcGGcccuAGAdTsdT | 478 | UCuAGGGCCGUGAAACUCUdTsdT |
| 10 | TCUUAAAGCUGGUUCAUAC | 110 | AUAUGAACCAGCUUUAAGU | 479 | (OMedT)cuuAAAGcuGGuucAuAcdTsdT | 480 | pAuAUGAACcAGCUuuAAGudTsdT |
| 46 | UACACAGUGACCGUCGACU | 147 | AGUCGACGGUCACUGUGUA | 481 | uAcAcAGuGAccGucGAcudTsdT | 482 | AGUCGACGGUcACUGUGuAdTsdT |
| 47 | AAAGUGCGAGUGAUCUA | 148 | UAUAGAUCACUCGCACUUU | 483 | AAAGuGcGAGuGAucuAuAdTsdT | 484 | uAuAGAUcACUCGcACUUUdTsdT |
| | UA | | | | | | |
| 48 | CAGCGAACUGAGGGUGACA | 149 | UGUCACCCUCAGUUCGCUG | 485 | cAGcGAAcuGAGGGuGAcAdTsdT | 486 | UGUcACCCUcAGUUCGCUGdTsdT |
| 14 | TCAUGAAUGCCUCUCGACU | 111 | AGUCGAGAGGCAUUCAUGC | 487 | (OMedT)cAuGAAuGccucucGAcudTsdT | 488 | AGUCGAGAGGcAUUcAUGCdTsdT |
| 21 | TCUUAAAGCUGGUUCAUAU | 110 | AUAUGAACCAGCUUUAAGU | 489 | (OMedT)cuuAAAGcuGGuucAuAudTsdT | 490 | pAuAUGAACcAGCUuuAAGudTsdT |
| 49 | CCGCGUUAAGAUUCCCGCA | 150 | UGCGGGAAUCUUAACGCGG | 491 | ccGcGuuAAGAuucccGcAdTsdT | 492 | UGCGGGAAUCUuAACGCGGdTsdT |
| 50 | ACUCCUGGUAGAACGGAUG | 151 | CAUCCGUUCUACCAGGAGU | 493 | AcuccuGGuAGAAcGGAuGdTsdT | 494 | cAUCCGUUCuACcAGGAGUdTsdT |
| 51 | GCGUUAAGAUUCCCGCAUU | 152 | AAUGCGGGAAUCUUAACGC | 495 | GcGuuAAGAuucccGcAuudTsdT | 496 | AAUGCGGGAAUCUuAACGCdTsdT |
| 52 | AAGCCCGGAUAGCAUGAAU | 153 | AUUCAUGCUAUCCGGGCUU | 497 | AAGcccGGAuAGcAuGAAudTsdT | 498 | AUUcAUGCuAUCCGGGCUUdTsdT |
| 8 | AGUACACAGUGACCGUCGA | 114 | UCGACGGUCACUGUGUACU | 499 | AGuAcAcAGuGAccGucGAdTsdT | 500 | puCGACGGUcACUGuGuACudTsdT |
| 53 | UUCCCGCAUUUUAAUGUUU | 154 | AAACAUUAAAAUGCGGGAA | 501 | uucccGcAuuuuAAuGuuudTsdT | 502 | AAAcAUuAAAAUGCGGGAAdTsdT |
| 54 | AACUCCUGGUAGAACGGAU | 155 | AUCCGUUCUACCAGGAGUU | 503 | AAcuccuGGuAGAAcGGAudTsdT | 504 | AUCCGUUCuACcAGGAGUUdTsdT |
| 55 | UUGUAGCAAGGUCCGUG | 156 | ACCACGGACCUUGCUACAA | 505 | uuGuAGcAAGGuccGuGGudTsdT | 506 | ACcACGGACCUUGCuAcAAdTsdT |
| | GU | | | | | | |
| 21 | TCUUAAAGCUGGUUCAUAU | 110 | AUAUGAACCAGCUUUAAGU | 507 | (OMedT)cuuAAAGcuGGuucAuAudTsdT | 508 | AuAUGAACcAGCUuuAAGudTsdT |
| 8 | AGUACACAGUGACCGUCGA | 114 | UCGACGGUCACUGUGUACU | 509 | AGuAcAcAGuGAccGucGAdTsdT | 510 | uCGACGGUcACUGuGuACudTsdT |
| 56 | CGUUAAGAUUCCCGCAUUU | 157 | AAAUGCGGGAAUCUUAACG | 511 | cGuuAAGAuucccGcAuuudTsdT | 512 | AAAUGCGGGAAUCUuAACGdTsdT |
| 57 | ACAAAAUUAUUGACCUA | 158 | CCUAGGUCAAUAAUUUUGU | 513 | AcAAAAuuAuuGAccuAGGdTsdT | 514 | CCuAGGUcAAuAAUUUUGUdTsdT |
| | GG | | | | | | |
| 21 | TCUUAAAGCUGGUUCAUAU | 143 | GUAUGAACCAGCUUUAAGU | 515 | (OMedT)cuuAAAGcuGGuucAuAudTsdT | 516 | GuAUGAACcAGCUuuAAGudTsdT |
| 58 | GCUUGUAGCAAGGUCCGUG | 159 | CACGGACCUUGCUACAAGC | 517 | GcuuGuAGcAAGGuccGuGdTsdT | 518 | cACGGACCUUGCuAcAAGCdTsdT |
| 59 | AACUUAAAGCUGGUUCA | 160 | UAUGAACCAGCUUUAAGUU | 519 | AAcuuAAAGcuGGuucAuAdTsdT | 520 | uAUGAACcAGCUUuAAGUUdTsdT |
| | UA | | | | | | |
| 60 | UGACCGUCGACUACUGGAG | 161 | CUCCAGUAGUCGACGGUCA | 521 | uGAccGucGAcuAcuGGAGdTsdT | 522 | CUCcAGuAGUCGACGGUcAdTsdT |
| 61 | AUUCCCGCAUUUUAAUGUU | 162 | AACAUUAAAAUGCGGGAAU | 523 | AuucccGcAuuuuAAuGuudTsdT | 524 | AAcAUuAAAAUGCGGGAAUdTsdT |
| 62 | AAUGUGGUGGCUGCCCGAG | 163 | CUCGGGCAGCCACCACAUU | 525 | AAuGuGGuGGcuGcccGAGdTsdT | 526 | CUCGGGcAGCcACcAcAUUdTsdT |
| 63 | GCCUCUCGACUUAGCCAGC | 164 | GCUGGCUAAGUCGAGAGGC | 527 | GccucucGAcuuAGccAGcdTsdT | 528 | GCUGGCuAAGUCGAGAGGCdTsdT |
| 64 | ACGACAUCAGUAUGAGCUG | 165 | CAGCUCAUACUGAUGUCGU | 529 | AcGAcAucAGuAuGAGcuGdTsdT | 530 | cAGCUcAuACUGAUGUCGUdTsdT |
| 65 | CUUGUAGCAAGGUCCGUGG | 166 | CCACGGACCUUGCUACAAG | 531 | cuuGuAGcAAGGuccGuGGdTsdT | 532 | CcACGGACCUUGCuAcAAGdTsdT |
| 66 | GCCAUGAUGAAUCUCCUCC | 167 | GGAGGAGAUUCAUCAUGGC | 533 | GccAuGAuGAAucuccuccdTsdT | 534 | GGAGGAGAUUcAUcAUGGCdTsdT |
| 67 | UCAUCCGAUGGCACAAUCA | 168 | UGAUUGUGCCAUCGGAUGA | 535 | ucAuccGAuGGcAcAAucAdTsdT | 536 | UGAUUGUGCcAUCGGAUGAdTsdT |
| 68 | GGAAAUGUCAUCCGAUG | 169 | GCCAUCGGAUGACAUUUCC | 537 | GGAAAuGucAuccGAuGGcdTsdT | 538 | GCcAUCGGAUGAcAUUUCCdTsdT |
| | GC | | | | | | |
| 69 | CGUGGUCCUGUCAGUGGAA | 170 | UUCCACUGACAGGACCACG | 539 | cGuGGuccuGucAGuGGAAdTsdT | 540 | UUCcACUGAcAGGACcACGdTsdT |
| 70 | AAUUAUUGACCUAGGAU | 171 | AUAUCCUAGGUCAAUAAUU | 541 | AAuuAuuGAccuAGGAuAudTsdT | 542 | AuAUCCuAGGUcAAuAAUUdTsdT |
| | AU | | | | | | |
| 71 | GCCGACAGAGUUAGCACGA | 172 | UCGUGCUAACUCUGUCGGC | 543 | GccGAcAGAGuuAGcAcGAdTsdT | 544 | UCGUGCuAACUCUGUCGGCdTsdT |
| 72 | UGCUUGUAGCAAGGUCCGU | 173 | ACGGACCUUGCUACAAGCA | 545 | uGcuuGuAGcAAGGuccGudTsdT | 546 | ACGGACCUUGCuAcAAGcAdTsdT |
| 73 | AGUGGAAGCCCGGAUAGCA | 174 | UGCUAUCCGGGCUUCCACU | 547 | AGuGGAAGcccGGAuAGcAdTsdT | 548 | UGCuAUCCGGGCUUCcACUdTsdT |
| 74 | AAGUGUCAGCUGUAUCCUU | 175 | AAGGAUACAGCUGACACUU | 549 | AAGuGucAGcuGuAuccuudTsdT | 550 | AAGGAuAcAGCUGAcACUUdTsdT |
| 75 | CAGGAAAUGGUACGGCU | 176 | GCAGCCGUACCAUUUCCUG | 551 | cAGGAAAuGGuAcGGcuGcdTsdT | 552 | GcAGCCGuACcAUUUCCUGdTsdT |
| | GC | | | | | | |
| 76 | GUCCCUGCCGACAGAGUUA | 177 | UAACUCUGUCGGCAGGGAC | 553 | GucccuGccGAcAGAGuuAdTsdT | 554 | uAACUCUGUCGGcAGGGACdTsdT |
| 77 | CGCGUUAAGAUUCCCGCAU | 178 | AUGCGGGAAUCUUAACGCG | 555 | cGcGuuAAGAuucccGcAudTsdT | 556 | AUGCGGGAAUCUuAACGCGdTsdT |
| 78 | AAGUGCGAGUGAUCUAU | 179 | GUAUAGAUCACUCGCACUU | 557 | AAGuGcGAGuGAucuAuAcdTsdT | 558 | GuAuAGAUcACUCGcACUUdTsdT |
| | AC | | | | | | |
| 79 | GAAUGCCUCUCGACUUAGC | 180 | GCUAAGUCGAGAGGCAUUC | 559 | GAAuGccucucGAcuuAGcdTsdT | 560 | GCuAAGUCGAGAGGcAUUCdTsdT |
| 80 | CGAGACCAGCGAACUGAGG | 181 | CCUCAGUUCGCUGGUCUCG | 561 | cGAGAccAGcGAAcuGAGGdTsdT | 562 | CCUcAGUUCGCUGGUCUCGdTsdT |
| 81 | UGUAGCAAGGUCCGUGG | 182 | GACCACGGACCUUGCUACA | 563 | uGuAGcAAGGuccGuGGucdTsdT | 564 | GACcACGGACCUUGCuAcAdTsdT |
| | UC | | | | | | |
| 82 | UUAGCACGACAUCAGUAUG | 183 | CAUACUGAUGUCGUGCUAA | 565 | uuAGcAcGAcAucAGuAuGdTsdT | 566 | cAuACUGAUGUCGUGCuAAdTsdT |
| 83 | CUGUACAGGAGACUAAG | 184 | CCCUUAGUCUCCUGUACAG | 567 | cuGuAcAGGAGAcuAAGGGdTsdT | 568 | CCCUuAGUCUCCUGuAcAGdTsdT |
| | GG | | | | | | |
| 84 | GAGAACGAAGUGAAACU | 185 | GGAGUUUCACUUCGUUCUC | 569 | GAGAAcGAAGuGAAAcuccdTsdT | 570 | GGAGUUUcACUUCGUUCUCdTsdT |
| | CC | | | | | | |
| 85 | GAACUUGGCGCCCAAUGAC | 186 | GUCAUUGGGCGCCAAGUUC | 571 | GAAcuuGGcGcccAAuGAcdTsdT | 572 | GUcAUUGGGCGCcAAGUUCdTsdT |
| 86 | GCUGCCCGCGUUAAGAUUC | 187 | GAAUCUUAACGCGGGCAGC | 573 | GcuGcccGcGuuAAGAuucdTsdT | 574 | GAAUCUuAACGCGGGcAGCdTsdT |
| 87 | AAGCUGGUUCAUAUCUU | 188 | UCAAGAUAUGAACCAGCUU | 575 | AAGcuGGuucAuAucuuGAdTsdT | 576 | UcAAGAuAUGAACcAGCUUdTsdT |
| | GA | | | | | | |
| 88 | GCCCGCGUUAAGAUUCCCG | 189 | CGGGAAUCUUAACGCGGGC | 577 | GcccGcGuuAAGAuucccGdTsdT | 578 | CGGGAAUCUuAACGCGGGCdTsdT |
| 89 | GAGGAAGUCGCGCCGCGCU | 190 | AGCGCGGCGCGACUUCCUC | 579 | GAGGAAGucGcGccGcGcudTsdT | 580 | AGCGCGGCGCGACUUCCUCdTsdT |
| 90 | CUCGAGACCAGCGAACUGA | 191 | UCAGUUCGCUGGUCUCGAG | 581 | cucGAGAccAGcGAAcuGAdTsdT | 582 | UcAGUUCGCUGGUCUCGAGdTsdT |
| 91 | AGAGGUGGUGAGCUUAA | 192 | CAUUAAGCUCACCACCUCU | 583 | AGAGGuGGuGAGcuuAAuGdTsdT | 584 | cAUuAAGCUcACcACCUCUdTsdT |
| | UG | | | | | | |
| 92 | GAGGUGGUGAGCUUAAU | 193 | UCAUUAAGCUCACCACCUC | 585 | GAGGuGGuGAGcuuAAuGAdTsdT | 586 | UcAUuAAGCUcACcACCUCdTsdT |
| | GA | | | | | | |
| 93 | GAGUUUCACGGCCCUAGAC | 194 | GUCUAGGGCCGUGAAACUC | 587 | GAGuuucAcGGcccuAGAcdTsdT | 588 | GUCuAGGGCCGUGAAACUCdTsdT |
| 94 | CGGCCUCCAACAGCUUACC | 195 | GGUAAGCUGUUGGAGGCCG | 589 | cGGccuccAAcAGcuuAccdTsdT | 590 | GGuAAGCUGUUGGAGGCCGdTsdT |
| 95 | AGCCCGGAUAGCAUGAAUG | 196 | CAUUCAUGCUAUCCGGGCU | 591 | AGcccGGAuAGcAuGAAuGdTsdT | 592 | cAUUcAUGCuAUCCGGGCUdTsdT |
| 10 | TCUUAAAGCUGGUUCAUAC | 143 | GUAUGAACCAGCUUUAAGU | 593 | (OMedT)cuuAAAGcuGGuucAuAcdTsdT | 594 | GuAUGAACcAGCUuuAAGudTsdT |
| 96 | GCGGGCCUGGCGUUGAUCC | 197 | GGAUCAACGCCAGGCCCGC | 595 | GcGGGccuGGcGuuGAuccdTsdT | 596 | GGAUcAACGCcAGGCCCGCdTsdT |
| 97 | UGCCCGCGUUAAGAUUCCC | 198 | GGGAAUCUUAACGCGGGCA | 597 | uGcccGcGuuAAGAuucccdTsdT | 598 | GGGAAUCUuAACGCGGGcAdTsdT |
| 98 | AGAUUCCCGCAUUUUAAUG | 199 | CAUUAAAAUGCGGGAAUCU | 599 | AGAuucccGcAuuuuAAuGdTsdT | 600 | cAUuAAAAUGCGGGAAUCUdTsdT |
| 99 | UCUCGACUUAGCCAGCCUG | 200 | CAGGCUGGCUAAGUCGAGA | 601 | ucucGAcuuAGccAGccuGdTsdT | 602 | cAGGCUGGCuAAGUCGAGAdTsdT |
| 100 | CAAUGUGGUGGCUGCCCGA | 201 | UCGGGCAGCCACCACAUUG | 603 | cAAuGuGGuGGcuGcccGAdTsdT | 604 | UCGGGcAGCcACcAcAUUGdTsdT |
| 101 | AAACUGUGGUUUGCAAG | 202 | UGCUUGCAAACCACAGUUU | 605 | AAAcuGuGGuuuGcAAGcAdTsdT | 606 | UGCUUGcAAACcAcAGUUUdTsdT |
| | CA | | | | | | |
| 102 | CCGCGUCCCUGCCGACAGA | 203 | UCUGUCGGCAGGGACGCGG | 607 | ccGcGucccuGccGAcAGAdTsdT | 608 | UCUGUCGGcAGGGACGCGGdTsdT |
| 103 | UGGGAUCACAUCAGAUA | 204 | UUUAUCUGAUGUGAUCCCA | 609 | uGGGAucAcAucAGAuAAAdTsdT | 610 | UUuAUCUGAUGUGAUCCcAdTsdT |
| | AA | | | | | | |
| 104 | AACAGAAUCAUCCAUCGGG | 205 | CCCGAUGGAUGAUUCUGUU | 611 | AAcAGAAucAuccAucGGGdTsdT | 612 | CCCGAUGGAUGAUUCUGUUdTsdT |
| 105 | AAUGCCUCUCGACUUAGCC | 206 | GGCUAAGUCGAGAGGCAUU | 613 | AAuGccucucGAcuuAGccdTsdT | 614 | GGCuAAGUCGAGAGGcAUUdTsdT |
| 106 | UGUACAGGAGACUAAGG | 207 | UCCCUUAGUCUCCUGUACA | 615 | uGuAcAGGAGAcuAAGGGAdTsdT | 616 | UCCCUuAGUCUCCUGuAcAdTsdT |
| | GA | | | | | | |
| 107 | UGUGGGCGGGAGAACGA | 208 | CUUCGUUCUCCCGCCCACA | 617 | uGuGGGcGGGAGAAcGAAGdTsdT | 618 | CUUCGUUCUCCCGCCcAcAdTsdT |
| | AG | | | | | | |
| 108 | UCUGUGGGCGGGAGAAC | 209 | UCGUUCUCCCGCCCACAGA | 619 | ucuGuGGGcGGGAGAAcGAdTsdT | 620 | UCGUUCUCCCGCCcAcAGAdTsdT |
| | GA | | | | | | |
| 109 | AGAUUGCUUGUAGCAAG | 210 | ACCUUGCUACAAGCAAUCU | 621 | AGAuuGcuuGuAGcAAGGudTsdT | 622 | ACCUUGCuAcAAGcAAUCUdTsdT |
| | GU | | | | | | |
| 16 | GCUAGAAAAUGCCAUACAG | 116 | CUGUAUGGCAUUUUCUAGC | 623 | GcuAGAAAAuGccAuAcAGdTsdT | 624 | pCUGuAUGGcAUUUUCuAGCdTsdT |
| 34 | CAGAAUCAUCCAUCGGGAU | 134 | AUCCCGAUGGAUGAUUCUG | 625 | cAGAAucAuccAucGGGAudTsdT | 626 | pAUCCCGAUGGAUGAUUCuGdTsdT |
| 34 | CAGAAUCAUCCAUCGGGAU | 134 | AUCCCGAUGGAUGAUUCUG | 627 | cAGAAucAuccAucGGGAudTsdT | 628 | AUCCCGAUGGAUGAUUCuGdTsdT |
| 24 | UGGUUCAUAUCUUGAAC | 124 | AUGUUCAAGAUAUGAACCA | 629 | uGGuucAuAucuuGAAcAudTsdT | 630 | AuGUUcAAGAuAUGAACcAdTsdT |
| | AU | | | | | | |
| 34 | CAGAAUCAUCCAUCGGGAU | 134 | AUCCCGAUGGAUGAUUCUG | 631 | cAGAAucAuccAucGGGAudTsdT | 632 | AUCCCGAuGGAuGAUUCuGdTsdT |
| 34 | CAGAAUCAUCCAUCGGGAU | 134 | AUCCCGAUGGAUGAUUCUG | 633 | cAGAAucAuccAucGGGAudTsdT | 634 | pAUCCCGAuGGAuGAUUCuGdTsdT |
| 5 | GCAAGGGAGCUGUACAG | 113 | UCCUGUACAGCUCCCUUGC | 635 | GcAAGGGAGcuGuAcAGGAdTsdT | 636 | uCCUGuAcAGCUCCCUUGcdTsdT |
| | GA | | | | | | |
| 24 | UGGUUCAUAUCUUGAAC | 124 | AUGUUCAAGAUAUGAACCA | 637 | uGGuucAuAucuuGAAcAudTsdT | 638 | pAuGUUcAAGAuAUGAACcAdTsdT |
| | AU | | | | | | |
| 16 | GCUAGAAAAUGCCAUACAG | 116 | CUGUAUGGCAUUUUCUAGC | 639 | GcuAGAAAAuGccAuAcAGdTsdT | 640 | pCuGuAUGGcAUUUUCuAGcdTsdT |
| 34 | CAGAAUCAUCCAUCGGGAU | 134 | AUCCCGAUGGAUGAUUCUG | 641 | cAGAAucAuccAucGGGAudTsdT | 642 | pAuCCCGAuGGAuGAUUCuGdTsdT |
| 34 | CAGAAUCAUCCAUCGGGAU | 134 | AUCCCGAUGGAUGAUUCUG | 643 | cAGAAucAuccAucGGGAudTsdT | 644 | AuCCCGAuGGAuGAUUCuGdTsdT |

TABLE 14

| | |
|---|---|
| unmodified sequence | modified sequence |

| | | SEQ | | SEQ | | SEQ | |
|---|---|---|---|---|---|---|---|
| SEQ ID | | ID | | ID | | ID | |
| NO | sequence (5′-3′) | NO | sequence (5′-3′) | NO | sequence (5′-3′) | NO | sequence (5′-3′) |
| 645 | GGCGGGAGAAUGACGUGAA | 693 | UUCACGUCAUUCUCCC | 739 | GGcGGGAGAAuGAcGuGAAdTsdT | 740 | UUcACGUcAUUCUCCCGCCdTsdT |
| | | | GCC | | | | |
| 646 | UGGGCGGGAGAAUGACGUG | 694 | CACGUCAUUCUCCCGC | 741 | uGGGcGGGAGAAuGAcGuGdTsdT | 742 | cACGUcAUUCUCCCGCCcAdTsdT |
| | | | CCA | | | | |
| 647 | GUAGCAAAGUCCGAGGUCC | 695 | GGACCUCGGACUUUGC | 743 | GuAGcAAAGuccGAGGuccdTsdT | 744 | GGACCUCGGACUUUGCuACdTsdT |
| | | | UAC | | | | |
| 648 | GACCGUGGUUUGUAAGCAG | 696 | CUGCUUACAAACCACG | 745 | GAccGuGGuuuGuAAGcAGdTsdT | 746 | CUGCUuAcAAACcACGGUCdTsdT |
| | | | GUC | | | | |
| 649 | AGACCGUGGUUUGUAAGCA | 697 | UGCUUACAAACCACGG | 747 | AGAccGuGGuuuGuAAGcAdTsdT | 748 | UGCUuAcAAACcACGGUCUdTsdT |
| | | | UCU | | | | |
| 650 | GUAAGACCGUGGUUUGUAA | 698 | UUACAAACCACGGUCU | 749 | GuAAGAccGuGGuuuGuAAdTsdT | 750 | UuAcAAACcACGGUCUuACdTsdT |
| | | | UAC | | | | |
| 651 | CAAAAUUAUUGAUCUAGGA | 699 | UCCUAGAUCAAUAAUU | 751 | cAAAAuuAuuGAucuAGGAdTsdT | 752 | UCCuAGAUcAAuAAUUUUGdTsdT |
| | | | UUG | | | | |
| 652 | CUCAGUAAGACCGUGGUUU | 700 | AAACCACGGUCUUACU | 753 | cucAGuAAGAccGuGGuuudTsdT | 754 | AAACcACGGUCUuACUGAGdTsdT |
| | | | GAG | | | | |
| 653 | UCGGCUCUUAGAUACCUUC | 701 | GAAGGUAUCUAAGAGC | 755 | ucGGcucuuAGAuAccuucdTsdT | 756 | GAAGGuAUCuAAGAGCCGAdTsdT |
| | | | CGA | | | | |
| 654 | AGCAUGAAUGUGUCUCGAC | 702 | GUCGAGACACAUUCAU | 757 | AGcAuGAAuGuGucucGAcdTsdT | 758 | GUCGAGAcAcAUUcAUGCUdTsdT |
| | | | GCU | | | | |
| 655 | AUUGAUCUAGGAUAUGCCA | 703 | UGGCAUAUCCUAGAUC | 759 | AuuGAucuAGGAuAuGccAdTsdT | 760 | UGGcAuAUCCuAGAUcAAUdTsdT |
| | | | AAU | | | | |
| 656 | GAAGAUCGCCUGUAGCAAA | 704 | UUUGCUACAGGCGAUC | 761 | GAAGAucGccuGuAGcAAAdTsdT | 762 | UUUGCuAcAGGCGAUCUUCdTsdT |
| | | | UUC | | | | |
| 647 | GUAGCAAAGUCCGAGGUCC | 695 | GGACCUCGGACUUUGC | 763 | GuAGcAAAGuccGAGGuccdTsdT | 764 | pGGACCUCGGACUUUGCuACdTs |
| | | | UAC | | | | dT |
| 645 | GGCGGGAGAAUGACGUGAA | 704 | UUCACGUCAUUCUCCC | 765 | GGcGGGAGAAuGAcGuGAAdTsdT | 766 | puUcACGUcAUUCUCCCGCcdTsdT |
| | | | GCC | | | | |
| 645 | GGCGGGAGAAUGACGUGAA | 704 | UUCACGUCAUUCUCCC | 767 | GGcGGGAGAAuGAcGuGAAdTsdT | 768 | uucACGUcAUUCUCCCGCcdTsdT |
| | | | GCC | | | | |
| 647 | GUAGCAAAGUCCGAGGUCC | 695 | GGACCUCGGACUUUGC | 769 | GuAGcAAAGuccGAGGuccdTsdT | 770 | GGACCUCGGACUUUGCuAcdTsdT |
| | | | UAC | | | | |
| 647 | GUAGCAAAGUCCGAGGUCC | 695 | GGACCUCGGACUUUGC | 771 | GuAGcAAAGuccGAGGuccdTsdT | 772 | pGGACCUCGGACUuuGCuAcdTsdT |
| | | | UAC | | | | |
| 645 | GGCGGGAGAAUGACGUGAA | 693 | UUCACGUCAUUCUCCC | 773 | GGcGGGAGAAuGAcGuGAAdTsdT | 774 | uUcACGUcAUUCUCCCGCcdTsdT |
| | | | GCC | | | | |
| 647 | GUAGCAAAGUCCGAGGUCC | 695 | GGACCUCGGACUUUGC | 775 | GuAGcAAAGuccGAGGuccdTsdT | 776 | GGACCUCGGACUuuGCuAcdTsdT |
| | | | UAC | | | | |
| 657 | TGGCGGGAGAAUGACGUG | 693 | UUCACGUCAUUCUCCC | 777 | (OMedT)GgcGGGAGAAuGAcGu | 778 | puucACGUcAUUCUCCCGCcdTsdT |
| | AA | | GCC | | GAAdTsdT | | |
| 645 | GGCGGGAGAAUGACGUGAA | 693 | UUCACGUCAUUCUCCC | 779 | GGcGGGAGAAuGAcGuGAAdTsdT | 780 | puUcACGUcAUUCUCCCGCcdTsdT |
| | | | GCC | | | | |
| 657 | TGGCGGGAGAAUGACGUG | 693 | UUCACGUCAUUCUCCC | 781 | (OMedT)GgcGGGAGAAuGAcGu | 782 | puUcACGUcAUUCUCCCGCcdTsdT |
| | AA | | GCC | | GAAdTsdT | | |
| 645 | GGCGGGAGAAUGACGUGAA | 693 | UUCACGUCAUUCUCCC | 783 | GGcGGGAGAAuGAcGuGAAdTsdT | 784 | puucACGUcAUUCUCCCGCcdTsdT |
| | | | GCC | | | | |
| 658 | TGUAGCAAAGUCCGAGGU | 695 | GGACCUCGGACUUUGC | 785 | (OMedT)GuAGcAAAGuccGAGGu | 786 | pGGACCUCGGACUUUGCuAcdTsdT |
| | CC | | UAC | | ccdTsdT | | |
| 647 | GUAGCAAAGUCCGAGGUCC | 695 | GGACCUCGGACUUUGC | 787 | GuAGcAAAGuccGAGGuccdTsdT | 788 | pGGACCUCGGACUUUGCuAcdTsdT |
| | | | UAC | | | | |
| 659 | AUCGAUAUGAGCUGGUCAC | 705 | GUGACCAGCUCAUAUC | 789 | AucGAuAuGAGcuGGucAcdTsdT | 790 | GUGACcAGCUcAuAUCGAUdTsdT |
| | | | GAU | | | | |
| 660 | GAUACUUGAACCAGUUCGA | 706 | UCGAACUGGUUCAAGU | 791 | GAuAcuuGAAccAGuucGAdTsdT | 792 | UCGAACUGGUUcAAGuAUCdTsdT |
| | | | AUC | | | | |
| 661 | AUCGGCUCUUAGAUACCUU | 707 | AAGGUAUCUAAGAGCC | 793 | AucGGcucuuAGAuAccuudTsdT | 794 | AAGGuAUCuAAGAGCCGAUdTsdT |
| | | | GAU | | | | |
| 662 | GCCAUCAAGCAAUGCCGAC | 708 | GUCGGCAUUGCUUGAU | 795 | GccAucAAGcAAuGccGAcdTsdT | 796 | GUCGGcAUUGCUUGAUGGCdTsdT |
| | | | GGC | | | | |
| 663 | UCAGUAAGACCGUGGUUUG | 709 | CAAACCACGGUCUUAC | 797 | ucAGuAAGAccGuGGuuuGdTsdT | 798 | cAAACcACGGUCUuACUGAdTsdT |
| | | | UGA | | | | |
| 664 | UCGCCUGUAGCAAAGUCCG | 710 | CGGACUUUGCUACAGG | 799 | ucGccuGuAGcAAAGuccGdTsdT | 800 | CGGACUUUGCuAcAGGCGAdTsdT |
| | | | CGA | | | | |
| 665 | GCCUGUAGCAAAGUCCGAG | 711 | CUCGGACUUUGCUACA | 801 | GccuGuAGcAAAGuccGAGdTsdT | 802 | CUCGGACUUUGCuAcAGGCdTsdT |
| | | | GGC | | | | |
| 666 | GGAGCCCGAUGGGUCGGAA | 712 | UUCCGACCCAUCGGGC | 803 | GGAGcccGAuGGGucGGAAdTsdT | 804 | UUCCGACCcAUCGGGCUCCdTsdT |
| | | | UCC | | | | |
| 667 | CCAUCAAGCAAUGCCGACA | 713 | UGUCGGCAUUGCUUGA | 805 | ccAucAAGcAAuGccGAcAdTsdT | 806 | UGUCGGcAUUGCUUGAUGGdTsdT |
| | | | UGG | | | | |
| 668 | CGGCUCUUAGAUACCUUCA | 714 | UGAAGGUAUCUAAGAG | 807 | cGGcucuuAGAuAccuucAdTsdT | 808 | UGAAGGuAUCuAAGAGCCGdTsdT |
| | | | CCG | | | | |
| 669 | UAUUGAUCUAGGAUAUGCC | 715 | GGCAUAUCCUAGAUCA | 809 | uAuuGAucuAGGAuAuGccdTsdT | 810 | GGcAuAUCCuAGAUcAAuAdTsdT |
| | | | AUA | | | | |
| 670 | UAAGACCGUGGUUUGUAAG | 716 | CUUACAAACCACGGUC | 811 | uAAGAccGuGGuuuGuAAGdTsdT | 812 | CUuAcAAACcACGGUCUuAdTsdT |
| | | | UUA | | | | |
| 671 | CAUCGAUAUGAGCUGGUCA | 717 | UGACCAGCUCAUAUCG | 813 | cAucGAuAuGAGcuGGucAdTsdT | 814 | UGACcAGCUcAuAUCGAUGdTsdT |
| | | | AUG | | | | |
| 672 | UGUAGCAAAGUCCGAGGUC | 718 | GACCUCGGACUUUGCU | 815 | uGuAGcAAAGuccGAGGucdTsdT | 816 | GACCUCGGACUUUGCuAcAdTsdT |
| | | | ACA | | | | |
| 673 | GGGCGGGAGAAUGACGUGA | 719 | UCACGUCAUUCUCCCG | 817 | GGGcGGGAGAAuGAcGuGAdTsdT | 818 | UcACGUcAUUCUCCCGCCCdTsdT |
| | | | CCC | | | | |
| 674 | GAUCGCCUGUAGCAAAGUC | 720 | GACUUUGCUACAGGCG | 819 | GAucGccuGuAGcAAAGucdTsdT | 820 | GACUUUGCuAcAGGCGAUCdTsdT |
| | | | AUC | | | | |
| 675 | UCGAUAUGAGCUGGUCACC | 721 | GGUGACCAGCUCAUAU | 821 | ucGAuAuGAGcuGGucAccdTsdT | 822 | GGUGACcAGCUcAuAUCGAdTsdT |
| | | | CGA | | | | |
| 676 | AGGAGCCCGAUGGGUCGGA | 722 | UCCGACCCAUCGGGCU | 823 | AGGAGcccGAuGGGucGGAdTsdT | 824 | UCCGACCcAUCGGGCUCCUdTsdT |
| | | | CCU | | | | |
| 677 | UGGGAAAUGAAAGAACGCC | 723 | GGCGUUCUUUCAUUUC | 825 | uGGGAAAuGAAAGAAcGccdTsdT | 826 | GGCGUUCUUUcAUUUCCcAdTsdT |
| | | | CCA | | | | |
| 678 | GGAAGACUGUAACCGGCUG | 724 | CAGCCGGUUACAGUCU | 827 | GGAAGAcuGuAAccGGcuGdTsdT | 828 | cAGCCGGUuAcAGUCUUCCdTsdT |
| | | | UCC | | | | |
| 679 | AUCGCCUGUAGCAAAGUCC | 725 | GGACUUUGCUACAGGC | 829 | AucGccuGuAGcAAAGuccdTsdT | 830 | GGACUUUGCuAcAGGCGAUdTsdT |
| | | | GAU | | | | |
| 680 | CCCGAGUUUUCAUGUCCUC | 726 | GAGGACAUGAAAACUC | 831 | cccGAGuuuucAuGuccucdTsdT | 832 | GAGGAcAUGAAAACUCGGGdTsdT |
| | | | GGG | | | | |
| 681 | CCGAUGGGUCGGAAGCAGG | 727 | CCUGCUUCCGACCCAU | 833 | ccGAuGGGucGGAAGcAGGdTsdT | 834 | CCUGCUUCCGACCcAUCGGdTsdT |
| | | | CGG | | | | |
| 682 | CGCCUGUAGCAAAGUCCGA | 728 | UCGGACUUUGCUACAG | 835 | cGccuGuAGcAAAGuccGAdTsdT | 836 | UCGGACUUUGCuAcAGGCGdTsdT |
| | | | GCG | | | | |
| 683 | GAAGACUGUAACCGGCUGC | 729 | GCAGCCGGUUACAGUC | 837 | GAAGAcuGuAAccGGcuGcdTsdT | 838 | GcAGCCGGUuAcAGUCUUCdTsdT |
| | | | UUC | | | | |
| 684 | AAGACUGUAACCGGCUGCA | 730 | UGCAGCCGGUUACAGU | 839 | AAGAcuGuAAccGGcuGcAdTsdT | 840 | UGcAGCCGGUuAcAGUCUUdTsdT |
| | | | CUU | | | | |
| 685 | ACGUCAUCCGGUGGCACAA | 731 | UUGUGCCACCGGAUGA | 841 | AcGucAuccGGuGGcAcAAdTsdT | 842 | UUGUGCcACCGGAUGACGUdTsdT |
| | | | CGU | | | | |
| 686 | GGAAACGUCAUCCGGUGGC | 732 | GCCACCGGAUGACGUU | 843 | GGAAAcGucAuccGGuGGcdTsdT | 844 | GCcACCGGAUGACGUUUCCdTsdT |
| | | | UCC | | | | |
| 687 | GAUUUGGAAACGUCAUCCG | 733 | CGGAUGACGUUUCCAA | 845 | GAuuuGGAAAcGucAuccGdTsdT | 846 | CGGAUGACGUUUCcAAAUCdTsdT |
| | | | AUC | | | | |
| 688 | UGUCCUCAGCGGGUGUCGC | 734 | GCGACACCCGCUGAGG | 847 | uGuccucAGcGGGuGucGcdTsdT | 848 | GCGAcACCCGCUGAGGAcAdTsdT |
| | | | ACA | | | | |
| 689 | AAACGUCAUCCGGUGGCAC | 735 | GUGCCACCGGAUGACG | 849 | AAAcGucAuccGGuGGcAcdTsdT | 850 | GUGCcACCGGAUGACGUUUdTsdT |
| | | | UUU | | | | |
| 690 | CAUCCGGUGGCACAAUCAG | 736 | CUGAUUGUGCCACCGG | 851 | cAuccGGuGGcAcAAucAGdTsdT | 852 | CUGAUUGUGCcACCGGAUGdTsdT |
| | | | AUG | | | | |
| 691 | UGGAAACGUCAUCCGGUGG | 737 | CCACCGGAUGACGUUU | 853 | uGGAAAcGucAuccGGuGGdTsdT | 854 | CcACCGGAUGACGUUUCcAdTsdT |
| | | | CCA | | | | |
| 692 | CAUGUCCUCAGCGGGUGUC | 738 | GACACCCGCUGAGGAC | 855 | cAuGuccucAGcGGGuGucdTsdT | 856 | GAcACCCGCUGAGGAcAUGdTsdT |
| | | | AUG | | | | |

TABLE 15

| | | | | | Mismatch | | |
|---|---|---|---|---|---|---|---|
| SEQ ID | | | | | pos. from | | Standard |
| NO pair | | | | Number of | 5′-end of | Remaining | deviation |
| 223/224 | Accession | Description | On/off target site (sense, 5′-3′) | mismatches | as | Rluc [%] | [%] |
| Anti | | | | | | | |
| sense | | | | | | | |
| ON | NM_001556.1 | Homo sapiens inhibitor of kappa light | GCAAGGGAGCTGTACAGGA | 0 | | 17 | 1 |
| | | polypeptide gene enhancer in B-cells, | | | | | |
| | | kinase beta (IKBKB), mRNA | | | | | |
| OFF 1 | NM_024667.2 | Homo sapiens vacuolar protein sorting | GAAATGAAGCTGTACAGGA | 3 | 13 15 18 | 92 | 7 |
| | | 37 homolog B (S. cerevisiae) | | | | | |
| | | (VPS37B), mRNA | | | | | |
| OFF 2 | NM_017643.2 | Homo sapiens mbt domain containing 1 | TCAATCCAGCTGTACAGGG | 5 | 1 13 14 15 | 105 | 12 |
| | | (MBTD1), mRNA | | | 19 | | |
| OFF 3 | NM_002438.2 | Homo sapiens mannose receptor, C type | CCAAGAGAGTTTTACAGGC | 5 | 1 8 10 14 | 117 | 17 |
| | | 1 (MRC1), mRNA | | | 19 | | |
| OFF 4 | NM_023079.3 | Homo sapiens ubiquitin-conjugating | CCAAGGTACCTTTACAGGA | 4 | 8 11 13 19 | 107 | 9 |
| | | enzyme E2Z (UBE2Z), mRNA | | | | | |
| OFF 5 | XM_001717374.1 | PREDICTED: Homo sapiens | ACACGGGAGCTGTAGAGGG | 4 | 1 5 16 19 | 109 | 8 |
| | | hypothetical protein LOC100134282 | | | | | |
| | | (LOC100134282), mRNA | | | | | |
| OFF 6 | NM_017432.3 | Homo sapiens prostate tumor | CCAAGGAAGCTGTACATGC | 4 | 1 3 13 19 | 113 | 12 |
| | | overexpressed 1 (PTOV1), mRNA | | | | | |
| OFF 7 | NM_198488.3 | Homo sapiens family with sequence | GCAAGGAAGCTGGACAGGG | 3 | 1 7 13 | 110 | 3 |
| | | similarity 83, member H (FAM83H), | | | | | |
| | | mRNA | | | | | |
| OFF 8 | NM_199320.2 | Homo sapiens PHD finger protein 17 | GCAAGGGGGCTGCACAGGA | 2 | 7 12 | 37 | 4 |
| | | (PHF17), transcript variant L, mRNA | | | | | |
| OFF 9 | NM_001005505.1 | Homo sapiens calcium channel, | TCAAGGAAGCTGTGCAGGG | 4 | 1 6 13 19 | 98 | 6 |
| | | voltage-dependent, alpha 2/delta | | | | | |
| | | subunit 2 (CACNA2D2), transcript | | | | | |
| | | variant 1, mRNA | | | | | |
| OFF 10 | NM_144492.2 | Homo sapiens claudin 14 (CLDN14), | GCAAGGGACCTGAACAGGA | 2 | 7 11 | 94 | 8 |
| | | transcript variant 1, mRNA | | | | | |
| OFF 11 | NM_058179.2 | Homo sapiens phosphoserine | TCAAGGGAGCAGTACTGGT | 4 | 1 4 9 19 | 79 | 9 |
| | | aminotransferase 1 (PSAT1), transcript | | | | | |
| | | variant 1, mRNA | | | | | |
| OFF 12 | NM_004187.3 | Homo sapiens lysine (K)-specific | GCTGGGGAGCTGAACAGGG | 4 | 1 7 16 17 | 110 | 7 |
| | | demethylase 5C (KDM5C), transcript | | | | | |
| | | variant 1, mRNA | | | | | |
| sense | | | | | | | |
| OFF 13 | NM_002340.5 | Homo sapiens lanosterol synthase (2,3- | TTCTGTCCAGCTCCCTTGC | 2 | 13 18 | 117 | 7 |
| | | oxidosqualene-lanosterol cyclase) | | | | | |
| | | (LSS), transcript variant 1, mRNA | | | | | |
| OFF 14 | NM_000460.2 | Homo sapiens thrombopoietin (THPO), | TCGTGTACAGCTCCCTTCC | 2 | 2 17 | 91 | 4 |
| | | mRNA | | | | | |

TABLE 16

| | | | | | Mismatch | | |
|---|---|---|---|---|---|---|---|
| SEQ ID | | | | | pos. from | | Standard |
| NO pair | | | | Number of | 5′-end of | Remaining | deviation |
| 235/236 | Accession | Description | On/off target site (sense, 5′-3′) | mismatches | as | Rluc [%] | [%] |
| antisense | | | | | | | |
| ON | NM_001556.1 | Homo sapiens inhibitor of kappa light | TCTTAAAGCTGGTTCATAC | 0 | | 23 | 3 |
| | | polypeptide gene enhancer in B-cells, | | | | | |
| | | kinase beta (IKBKB), mRNA | | | | | |
| OFF 1 | NM_019034.2 | Homo sapiens ras homolog gene family, | TCTCAGAACTGGTTCATAG | 5 | 1 12 14 16 | 91 | 3 |
| | | member F (in filopodia) (RHOF), | | | 19 | | |
| | | mRNA | | | | | |
| OFF 2 | NM_001141972.1 | Homo sapiens ATP binding domain 4 | ATATAAAGTTGGTTCATAG | 4 | 1 11 17 18 | 78 | 6 |
| | | (ATPBD4), transcript variant 2, mRNA | | | | | |
| OFF 3 | NM_024090.2 | Homo sapiens ELOVL family member | TCTTGATGTTGGTTCATAG | 5 | 1 11 13 15 | 92 | 9 |
| | | 6, elongation of long chain fatty acids | | | 19 | | |
| | | (FEN1/Elo2, SUR4/Elo3-like, yeast) | | | | | |
| | | (ELOVL6), transcript variant 1, mRNA | | | | | |
| OFF 4 | NM_024754.3 | Homo sapiens pentatricopeptide repeat | GTTCAAAGCTGCTTCATAC | 5 | 1 8 16 18 | 99 | 6 |
| | | domain 2 (PTCD2), mRNA | | | 19 | | |
| OFF 5 | NM_020141.3 | Homo sapiens transmembrane protein | GTTTAGAGCTGCTTCATAT | 4 | 8 14 18 19 | 87 | 14 |
| | | 167B (TMEM167B), mRNA | | | | | |
| OFF 6 | NM_014106.2 | Homo sapiens zinc finger protein 770 | TTTTTAAGCTGTTTCATAT | 4 | 8 15 18 19 | 93 | 6 |
| | | (ZNF770), mRNA | | | | | |
| OFF 7 | XM_001721730.1 | PREDICTED: Homo sapiens | GCTTAAAACTGGATCATAT | 3 | 7 12 19 | 90 | 8 |
| | | hypothetical protein LOC100129413 | | | | | |
| | | (LOC100129413), mRNA | | | | | |
| OFF 8 | NM_152243.2 | Homo sapiens CDC42 effector protein | CCTAACAGCTGGTTCCTAC | 5 | 1 4 14 16 | 92 | 6 |
| | | (Rho GTPase binding) 1 (CDC42EP1), | | | 19 | | |
| | | mRNA | | | | | |
| OFF 9 | NM_178812.3 | Homo sapiens metadherin (MTDH), | ACTTTAAGCAGGTTCAAAG | 4 | 1 3 10 15 | 90 | 4 |
| | | mRNA | | | | | |
| OFF 10 | NM_006621.4 | Homo sapiens S-adenosylhomocysteine | CCTCAAAGTTGGGTCATAC | 5 | 1 7 11 16 | 87 | 8 |
| | | hydrolase-like 1 (AHCYL1), mRNA | | | 19 | | |
| OFF 11 | NM_015056.2 | Homo sapiens ribosomal RNA | CTTTAAAGATGGGTCATAT | 4 | 7 11 18 19 | 91 | 13 |
| | | processing 1 homolog B (S. cerevisiae) | | | | | |
| | | (RRP1B), mRNA | | | | | |
| OFF 12 | NM_000945.3 | Homo sapiens protein phosphatase 3 | GCTTATAGCTGCTTCATTC | 5 | 1 2 8 14 19 | 96 | 5 |
| | | (formerly 2B), regulatory subunit B, | | | | | |
| | | alpha isoform (PPP3R1), mRNA | | | | | |
| sense | | | | | | | |
| OFF 13 | NM_018317.2 | Homo sapiens TBC1 domain family, | TGATGAACCAGATTTAAGC | 4 | 1 8 18 19 | 89 | 6 |
| | | member 19 (TBC1D19), mRNA | | | | | |
| OFF 14 | NM_004972.3 | Homo sapiens Janus kinase 2 (JAK2), | TTATGAACCAGATTTCAGG | 4 | 1 4 8 19 | 92 | 5 |
| | | mRNA | | | | | |

TABLE 17

| | | | | | Mismatch | | |
|---|---|---|---|---|---|---|---|
| SEQ ID | | | | | pos. from | | Standard |
| NO pair | | | | Number of | 5′-end of | Remaining | deviation |
| 219/220 | Accession | Description | On/off target site (sense, 5′-3′) | mismatches | as | Rluc [%] | [%] |
| antisense | | | | | | | |
| ON | NM_001556.1 | Homo sapiens inhibitor of kappa light | ACTTAAAGCTGGTTCATAT | 0 | | 18 | 4 |
| | | polypeptide gene enhancer in B-cells, | | | | | |
| | | kinase beta (IKBKB), mRNA | | | | | |
| OFF 1 | NM_019034.2 | Homo sapiens ras homolog gene family, | TCTCAGAACTGGTTCATAG | 5 | 1 12 14 16 | 80 | 5 |
| | | member F (in filopodia) (RHOF), | | | 19 | | |
| | | mRNA | | | | | |
| OFF 2 | NM_001141972.1 | Homo sapiens ATP binding domain 4 | ATATAAAGTTGGTTCATAG | 4 | 1 11 17 18 | 35 | 4 |
| | | (ATPBD4), transcript variant 2, mRNA | | | | | |
| OFF 3 | NM_024090.2 | Homo sapiens ELOVL family member | TCTTGATGTTGGTTCATAG | 5 | 1 11 13 15 | 70 | 7 |
| | | 6, elongation of long chain fatty acids | | | 19 | | |
| | | (FEN1/Elo2, SUR4/Elo3-like, yeast) | | | | | |
| | | (ELOVL6), transcript variant 1, mRNA | | | | | |
| OFF 4 | NM_024754.3 | Homo sapiens pentatricopeptide repeat | GTTCAAAGCTGCTTCATAC | 5 | 1 8 16 18 | 80 | 6 |
| | | domain 2 (PTCD2), mRNA | | | 19 | | |
| OFF 5 | NM_020141.3 | Homo sapiens transmembrane protein | GTTTAGAGCTGCTTCATAT | 4 | 8 14 18 19 | 35 | 5 |
| | | 167B (TMEM167B), mRNA | | | | | |
| OFF 6 | NM_014106.2 | Homo sapiens zinc finger protein 770 | TTTTTAAGCTGTTTCATAT | 4 | 8 15 18 19 | 81 | 5 |
| | | (ZNF770), mRNA | | | | | |
| OFF 7 | XM_001721730.1 | PREDICTED: Homo sapiens | GCTTAAAACTGGATCATAT | 3 | 7 12 19 | 85 | 4 |
| | | hypothetical protein LOC100129413 | | | | | |
| | | (LOC100129413), mRNA | | | | | |
| OFF 8 | NM_152243.2 | Homo sapiens CDC42 effector protein | CCTAACAGCTGGTTCCTAC | 5 | 1 4 14 16 | 83 | 8 |
| | | (Rho GTPase binding) 1 (CDC42EP1), | | | 19 | | |
| | | mRNA | | | | | |
| OFF 9 | NM_178812.3 | Homo sapiens metadherin (MTDH), | ACTTTAAGCAGGTTCAAAG | 4 | 1 3 10 15 | 83 | 7 |
| | | mRNA | | | | | |
| OFF 10 | NM_006621.4 | Homo sapiens S-adenosylhomocysteine | CCTCAAAGTTGGGTCATAC | 5 | 1 7 11 16 | 77 | 9 |
| | | hydrolase-like 1 (AHCYL1), mRNA | | | 19 | | |
| OFF 11 | NM_015056.2 | Homo sapiens ribosomal RNA | CTTTAAAGATGGGTCATAT | 4 | 7 11 18 19 | 85 | 12 |
| | | processing 1 homolog B (S. cerevisiae) | | | | | |
| | | (RRP1B), mRNA | | | | | |
| OFF 12 | NM_000945.3 | Homo sapiens protein phosphatase 3 | GCTTATAGCTGCTTCATTC | 5 | 1 2 8 14 19 | 79 | 14 |
| | | (formerly 2B), regulatory subunit B, | | | | | |
| | | alpha isoform (PPP3R1), mRNA | | | | | |
| sense | | | | | | | |
| OFF 13 | NM_018317.2 | Homo sapiens TBC1 domain family, | TGATGAACCAGATTTAAGC | 4 | 1 8 18 19 | 38 | 4 |
| | | member 19 (TBC1D19), mRNA | | | | | |
| OFF 14 | NM_004972.3 | Homo sapiens Janus kinase 2 (JAK2), | TTATGAACCAGATTTCAGG | 4 | 1 4 8 19 | 84 | 6 |
| | | mRNA | | | | | |

TABLE 18

| | | | | | Mismatch | | |
|---|---|---|---|---|---|---|---|
| SEQ ID | | | | | pos. from | | Standard |
| NO pair | | | | Number of | 5′-end of | Remaining | deviation |
| 229/230 | Accession | Description | On/off target site (sense, 5′-3′) | mismatches | as | Rluc [%] | [%] |
| antisense | | | | | | | |
| ON | NM_001556.1 | Homo sapiens inhibitor of kappa light | AGTACACAGTGACCGTCGA | 0 | | 27 | 4 |
| | | polypeptide gene enhancer in B-cells, | | | | | |
| | | kinase beta (IKBKB), mRNA | | | | | |
| OFF 1 | NM_174923.1 | Homo sapiens coiled-coil domain | TGTACACCGCGGCCGTCGC | 5 | 1 8 10 12 | 71 | 8 |
| | | containing 107 (CCDC107), mRNA | | | 19 | | |
| OFF 2 | NM_004635.3 | Homo sapiens mitogen-activated | AGTACGCAGTGACCGACGA | 2 | 4 14 | 41 | 9 |
| | | protein kinase-activated protein kinase | | | | | |
| | | 3 (MAPKAPK3), mRNA | | | | | |
| OFF 3 | NM_014714.3 | Homo sapiens intraflagellar transport | TGGACACAGTGACCGTCTT | 4 | 1 2 17 19 | 67 | 10 |
| | | 140 homolog (Chlamydomonas) | | | | | |
| | | (IFT140), mRNA | | | | | |
| OFF 4 | NM_013449.3 | Homo sapiens bromodomain adjacent to | GGTACACAGTGTCCGCCGA | 3 | 4 8 19 | 80 | 8 |
| | | zinc finger domain, 2A (BAZ2A), | | | | | |
| | | mRNA | | | | | |
| OFF 5 | NM_152449.2 | Homo sapiens LysM, putative | AGACCACAGTGACCGTGGA | 3 | 3 16 17 | 91 | 8 |
| | | peptidoglycan-binding, domain | | | | | |
| | | containing 4 (LYSMD4), mRNA | | | | | |
| OFF 6 | NM_001040429.2 | Homo sapiens protocadherin 17 | AGTACAACGTGACCATCGT | 4 | 1 5 12 13 | 87 | 6 |
| | | (PCDH17), mRNA | | | | | |
| OFF 7 | NM_006677.1 | Homo sapiens ubiquitin specific | CCTACACAGAGACCGTGGA | 4 | 3 10 18 19 | 92 | 10 |
| | | peptidase 19 (USP19), mRNA | | | | | |
| OFF 8 | NM_003202.3 | Homo sapiens transcription factor 7 (T- | TGTACAAAGAGACCGTCTA | 4 | 2 10 13 19 | 87 | 6 |
| | | cell specific, HMG-box) (TCF7), | | | | | |
| | | transcript variant 1, mRNA | | | | | |
| OFF 9 | NM_024101.5 | Homo sapiens melanophilin (MLPH), | TATACACAGTCACCGTCCC | 5 | 1 2 9 18 19 | 88 | 3 |
| | | transcript variant 1, mRNA | | | | | |
| OFF 10 | NM_024513.2 | Homo sapiens FYVE and coiled-coil | TGTACACAAAGACCATCGA | 4 | 5 10 11 19 | 99 | 5 |
| | | domain containing 1 (FYCO1), mRNA | | | | | |
| OFF 11 | NM_001001676.1 | Homo sapiens lipocalin 9 (LCN9), | AGAACACAGTGGCCGTCTC | 4 | 1 2 8 17 | 89 | 3 |
| | | mRNA | | | | | |
| OFF 12 | NM_004273.4 | Homo sapiens carbohydrate | AGTACAGAGTGCCCGTGGG | 4 | 1 3 8 13 | 95 | 7 |
| | | (chondroitin 6) sulfotransferase 3 | | | | | |
| | | (CHST3), mRNA | | | | | |
| sense | | | | | | | |
| OFF 13 | NM_032871.3 | Homo sapiens RELT tumor necrosis | CCGGCGGGCACTGTGTACA | 4 | 1 12 16 19 | 102 | 6 |
| | | factor receptor (RELT), transcript | | | | | |
| | | variant 1, mRNA | | | | | |
| OFF 14 | NM_138814.2 | Homo sapiens patatin-like | TCCCCGGTCACTGTGTACC | 3 | 1 16 17 | 95 | 7 |
| | | phospholipase domain containing 5 | | | | | |
| | | (PNPLA5), mRNA | | | | | |

TABLE 19

| | | | SEQ ID |
|---|---|---|---|
| FPL Name | Function | Sequence | No |
| GAPDH10 | BL | ggcaacaatatccactttaccag | 926 |
| GAPDH19 | BL | gacgtactcagcgccagcat | 927 |
| GAPDH1 | CE | gaggctgcgggctcaattTTTTTctcttggaaagaaagt | 928 |
| GAPDH4 | CE | tggcgacgcaaaagaagatgTTTTTctcttggaaagaaagt | 929 |
| GAPDH7 | CE | caaatccgttgactccgacctTTTTTctcttggaaagaaagt | 930 |
| GAPDH11 | CE | ggtcaatgaaggggtcattgatTTTTTctcttggaaagaaagt | 931 |
| GAPDH14 | CE | ccttgacggtgccatggaatTTTTTctcttggaaagaaagt | 932 |
| GAPDH20 | CE | aagacgccagtggactccacTTTTTctcttggaaagaaagt | 933 |
| GAPDH2 | LE | ggagcagagagcgaagcggTTTTTgaagttaccgtttt | 934 |
| GAPDH3 | LE | cggctgactgtcgaacaggaTTTTTctgagtcaaagcat | 935 |
| GAPDH5 | LE | gagcgatgtggctcggcTTTTTgaagttaccgtttt | 936 |
| GAPDH6 | LE | tcaccttccccatggtgtctTTTTTctgagtcaaagcat | 937 |
| GAPDH8 | LE | ccaggcgcccaatacgacTTTTTgaagttaccgtttt | 938 |
| GAPDH9 | LE | agttaaaagcagccctggtgaTTTTTctgagtcaaagcat | 939 |
| GAPDH12 | LE | tggaacatgtaaaccatgtagttgaTTTTTgaagttaccgtttt | 940 |
| GAPDH13 | LE | ttgccatgggtggaatcatatTTTTTctgagtcaaagcat | 941 |
| GAPDH15 | LE | tgacaagcttcccgttctcagTTTTTgaagttaccgtttt | 942 |
| GAPDH16 | LE | tggtgatgggatttccattgaTTTTTctgagtcaaagcat | 943 |
| GAPDH17 | LE | gggatctcgctcctggaagaTTTTTgaagttaccgtttt | 944 |
| GAPDH18 | LE | cgccccacttgattttggaTTTTTctgagtcaaagcat | 945 |

TABLE 20

| | | | SEQ ID |
|---|---|---|---|
| FPL Name | Function | Sequence | No |
| MAPKAPK33 | BL | ccgccgccccccc | 946 |
| MAPKAPK312 | BL | aagcctgccagtgatggtcta | 947 |
| MAPKAPK313 | BL | aatatgggggccgccag | 948 |
| MAPKAPK327 | BL | ttttgggtggtctccttagca | 949 |
| MAPKAPK31 | CE | cgggtccgccgggtTTTTTctcttggaaagaaagt | 950 |
| MAPKAPK32 | CE | ggagcaccgcccaagcTTTTTctcttggaaagaaagt | 951 |
| MAPKAPK36 | CE | caggcccagcacctgctTTTTTctcttggaaagaaagt | 952 |
| MAPKAPK39 | CE | agggcacacttctgtccagtgTTTTTctcttggaaagaaagt | 953 |
| MAPKAPK318 | CE | cgctcctgaatcctgctgaacTTTTTctcttggaaagaaagt | 954 |
| MAPKAPK321 | CE | tggcagtgccaatatcccgTTTTTctcttggaaagaaagt | 955 |
| MAPKAPK326 | CE | aagccaaaatcggtgagcttTTTTTctcttggaaagaaagt | 956 |
| MAPKAPK328 | CE | cagggtgtctgcagggcaTTTTTctcttggaaagaaagt | 957 |
| MAPKAPK34 | LE | cactgcgtacttcttgggctcTTTTTgaagttaccgtttt | 958 |
| MAPKAPK35 | LE | tggacaactggtagtcgtcggtTTTTTctgagtcaaagcat | 959 |
| MAPKAPK37 | LE | agcactttgccgttcacaccTTTTTgaagttaccgtttt | 960 |
| MAPKAPK38 | LE | cgccgatggaagcactccTTTTTctgagtcaaagcat | 961 |
| MAPKAPK310 | LE | ggggctgtcatacaggagcttcTTTTTgaagttaccgtttt | 962 |
| MAPKAPK311 | LE | cctcctgccgggccttTTTTTctgagtcaaagcat | 963 |
| MAPKAPK314 | LE | tcatacacatccaggatgcagacTTTTTgaagttaccgtttt | 964 |
| MAPKAPK315 | LE | gcttgccatggtgcatgttcTTTTTctgagtcaaagcat | 965 |
| MAPKAPK316 | LE | tccatgatgatgaggagacagcTTTTTgaagttaccgtttt | 966 |
| MAPKAPK317 | LE | aactcaccaccttccatgcatTTTTTctgagtcaaagcat | 967 |
| MAPKAPK319 | LE | gtgaaagcctggtcgccaTTTTTgaagttaccgtttt | 968 |
| MAPKAPK320 | LE | cattatctctgcagcttctctctcaTTTTTctgagtcaaagcat | 969 |
| MAPKAPK322 | LE | tatggctgtgcagaaactggaTTTTTgaagttaccgtttt | 970 |
| MAPKAPK323 | LE | gacatctcggtgggcaatgtTTTTTctgagtcaaagcat | 971 |
| MAPKAPK324 | LE | atgtgtagagtaggttttcaggcttTTTTTgaagttaccgtttt | 972 |
| MAPKAPK325 | LE | aagcactgcgtctttctccttagTTTTTctgagtcaaagcat | 973 |

TABLE 21

| | | | |
|---|---|---|---|
| | | | SEQ ID |
| FPL Name | Function | Sequence | No |

| | | | |
|---|---|---|---|
| PHF176 | BL | ctcagtctctatggcatgattcatat | 974 |
| PHF1715 | BL | ggctgaacccccagggc | 975 |
| PHF1718 | BL | acttggttccgctacgggt | 976 |
| PHF1725 | BL | ccccaaacttctcattgcagag | 977 |
| PHF1730 | BL | cttgacttcatcattctctgctaaga | 978 |
| PHF173 | CE | tggtgtattcatctagttcaggcaTTTTTctcttggaaagaaagt | 979 |
| PHF177 | CE | ttcgatccccaggccttcTTTTTctcttggaaagaaagt | 980 |
| PHF1712 | CE | gattccataacaggcctggtgTTTTTctcttggaaagaaagt | 981 |
| PHF1719 | CE | agcacagctaacgtggacccTTTTTctcttggaaagaaagt | 982 |
| PHF1722 | CE | gacaccttggtgatgggctcTTTTTctcttggaaagaaagt | 983 |
| PHF1726 | CE | gttcttcacagagcactgtatagaggTTTTTctcttggaaagaaagt | 984 |
| PHF1727 | CE | tggaaggctgtgcggcaTTTTTctcttggaaagaaagt | 985 |
| PHF1731 | CE | gctttgggcaataggacttgaaTTTTTctcttggaaagaaagt | 986 |
| PHF171 | LE | tggtcagttccagccatgcaTTTTTgaagttaccgtttt | 987 |
| PHF172 | LE | ttcccatctccttaaattcttcatTTTTTctgagtcaaagcat | 988 |
| PHF174 | LE | aattcctctaggaccctctccaTTTTTgaagttaccgtttt | 989 |
| PHF175 | LE | tgtcgtagcatcgctgctcaTTTTTctgagtcaaagcat | 990 |
| PHF178 | LE | gacatcacagacaacatcttcatcataTTTTTgaagttaccgtttt | 991 |
| PHF179 | LE | ctcaccatcaggagactggcaTTTTTctgagtcaaagcat | 992 |
| PHF1710 | LE | gaacaccatctcattgccgtcTTTTTgaagttaccgtttt | 993 |
| PHF1711 | LE | cacacagatgttgcatttgtcacaTTTTTctgagtcaaagcat | 994 |
| PHF1713 | LE | gctgccctctggtaccttgagTTTTTgaagttaccgtttt | 995 |
| PHF1714 | LE | acatgtccggcacagccaTTTTTctgagtcaaagcat | 996 |
| PHF1716 | LE | cttcggacacagcagacattttTTTTTgaagttaccgtttt | 997 |
| PHF1717 | LE | gggcttcatagctccacccttTTTTTctgagtcaaagcat | 998 |
| PHF1720 | LE | gctcacctcagggatccacagTTTTTgaagttaccgtttt | 999 |
| PHF1721 | LE | catcttctctgggctgccaatTTTTTctgagtcaaagcat | 1000 |
| PHF1723 | LE | cggctgctgggaatgtgtTTTTTgaagttaccgtttt | 1001 |
| PHF1724 | LE | gctgcacactagcgcccacTTTTTctgagtcaaagcat | 1002 |
| PHF1728 | LE | cggtcaaaagcacaggtcacaTTTTTgaagttaccgtttt | 1003 |
| PHF1729 | LE | tggtcttcatctccaggcccTTTTTctgagtcaaagcat | 1004 |

TABLE 22

| | | |
|---|---|---|
| FPL Name | Function | Sequence |

| | | | |
|---|---|---|---|
| IKBKB16 | BL | aaaacttcaccgttccattcaag | 1005 |
| IKBKB3 | CE | cctaggtcaataattttgtgtattaacctTTTTTctcttggaaagaaagt | 1006 |
| IKBKB4 | CE | tgatccagctccttggcatatTTTTTctcttggaaagaaagt | 1007 |
| IKBKB7 | CE | cagtagctctggggccaggTTTTTctcttggaaagaaagt | 1008 |
| IKBKB10 | CE | tgcactcaaaggccagggtTTTTTctcttggaaagaaagt | 1009 |
| IKBKB13 | CE | tgaatgccactgcacgggTTTTTctcttggaaagaaagt | 1010 |
| IKBKB17 | CE | tggggtagggtaaagagcttgTTTTTctcttggaaagaaagt | 1011 |
| IKBKB1 | LE | gatgttttctggctttagatcccTTTTTgaagttaccgtttt | 1012 |
| IKBKB2 | LE | ctgttctccttgctgcaggacTTTTTctgagtcaaagcat | 1013 |
| IKBKB5 | LE | aatgatgtgcaaagactgcccTTTTTgaagttaccgtttt | 1014 |
| IKBKB6 | LE | tactgcagggtccccacgTTTTTctgagtcaaagcat | 1015 |
| IKBKB8 | LE | ggtcactgtgtacttctgctgctcTTTTTgaagttaccgtttt | 1016 |
| IKBKB9 | LE | gccgaagctccagtagtcgacTTTTTctgagtcaaagcat | 1017 |
| IKBKB11 | LE | ggccggaagcccgtgaTTTTTgaagttaccgtttt | 1018 |
| IKBKB12 | LE | ctgccagttggggaggaagTTTTTctgagtcaaagcat | 1019 |
| IKBKB14 | LE | ctcactcttctgccgcactttTTTTTgaagttaccgtttt | 1020 |
| IKBKB15 | LE | tcttcgctaacaacaatgtccacTTTTTctgagtcaaagcat | 1021 |
| IKBKB18 | LE | tcagccaggacactgttaagattatTTTTTgaagttaccgtttt | 1022 |
| IKBKB19 | LE | gcagccacttctccagtcgcTTTTTctgagtcaaagcat | 1023 |

TABLE 23

| | | Gene | mean | mean | mean | mean max. |
|---|---|---|---|---|---|---|
| | Accession | Symbol | IC20 [nM] | IC50 [nM] | IC80 [nM] | inhibition [%] |
| antisense ON | NM_001556.1 | IKBKB | 0.0186 | 0.1160 | #N/A | 71 |
| antisense OFF 8 | NM_199320.2 | PHF17 | #N/A | #N/A | #N/A | #N/A |

TABLE 24

| | | | mean | mean | mean | mean max. |
|---|---|---|---|---|---|---|
| | Accession | | IC20 [nM] | IC50 [nM] | IC80 [nM] | inhibition [%] |
| antisense ON | NM_001556.1 | IKBKB | 0.0001 | 0.0286 | #N/A | 67 |
| antisense OFF 2 | NM_004635.3 | MAPKAPK3 | 0.0287 | 0.1575 | #N/A | 72 |

1. A double-stranded ribonucleic acid molecule capable of inhibiting the expression of IKK2 gene in vitro by at least 60%.

2. A double-stranded ribonucleic acid molecule capable of inhibiting the expression of the IKK2 gene in vitro by at least 70%.

3. A double-stranded ribonucleic acid molecule capable of inhibiting the expression of the IKK2 gene in vitro by at least 80%.

4. A double-stranded ribonucleic acid molecule of claim 1, wherein said double-stranded ribonucleic acid molecule comprises a sense strand and an antisense strand, the antisense strand being at least partially complementary to the sense strand, whereby the sense strand comprises a sequence, which has an identity of at least 90% to at least a portion of an mRNA encoding IKK2, wherein said sequence is (i) located in the region of complementarity of said sense strand to said antisense strand; and (ii) wherein said sequence is less than 30 nucleotides in length.

5. A double-stranded ribonucleic acid molecule of claim 1, comprising a sense strand and an antisense strand wherein said sense strand comprises a nucleic acid sequence selected from the group consisting of SEQ ID Nos: 1, 2, 3, 5, 6, 8, 9, and 10 and said antisense strand comprises a nucleic acid sequence selected from the group consisting of SEQ ID Nos: 110, 111, 112, 113 and 114.

6. A double-stranded ribonucleic acid molecule of claim 5, wherein said double-stranded ribonucleic acid molecule comprises a sequence pair selected from the group consisting of SEQ ID NOs: 1/110, 2/111, 3/112, 5/113, 6/111, 8/114, 9/114 and 10/110.

7. A double-stranded ribonucleic acid molecule of claim 5, wherein said double-stranded ribonucleic acid molecule comprises a sequence pair selected from the group consisting of SEQ ID NOs: 1/110, 2/111, 3/112, and 5/113.

8. A double-stranded ribonucleic acid molecule of claim 5, wherein said double-stranded ribonucleic acid molecule comprises a sequence pair selected from the group consisting of SEQ ID NOs: 16/111, 8/114, 9/114 and 10/110.

9. A double-stranded ribonucleic acid molecule of claim 6, wherein the antisense strand further comprises a 3′ overhang of 1-5 nucleotides in length.

10. A double-stranded ribonucleic acid molecule of claim 9, wherein the overhang of the antisense strand comprises uracil or nucleotides which are complementary to the mRNA encoding IKK2.

11. A double-stranded ribonucleic acid molecule of claim 10, wherein the sense strand further comprises a 3′ overhang of 1-5 nucleotides in length.

12. A double-stranded ribonucleic acid molecule of claim 11, wherein the overhang of the sense strand comprises uracil or nucleotides which are identical to the mRNA encoding IKK2.

13. A double-stranded ribonucleic acid molecule of claim 1, wherein said double-stranded ribonucleic acid molecule comprises at least one modified nucleotide.

14. A double-stranded ribonucleic acid molecule of claim 13, wherein said modified nucleotide is selected from the group consisting of a 2′-O-methyl modified nucleotide, a 5′-O-methyl modified nucleotide, a nucleotide comprising a 5′-phosphorothioate group, and a terminal nucleotide linked to a cholesteryl derivative or dodecanoic acid bisdecylamide group, a 2′-deoxy-2′-fluoro modified nucleotide, a 2′-deoxy-modified nucleotide, a locked nucleotide, an inverted deoxythymidine, an a basic nucleotide, 2′-amino-modified nucleotide, 2′-alkyl-modified nucleotide, morpholino nucleotide, a phosphoramidate, a 5′-phosphate group at the 5′-terminale end of the antisense strand and a non-natural base comprising nucleotide.

15. A double-stranded ribonucleic acid molecule of claim 14, wherein said modified nucleotide is a 2′-O-methyl modified nucleotide, a 5′-O-methyl modified nucleotide, a 2′ deoxy-2′ fluoro-modified nucleotide, a nucleotide comprising a 5′-phosphorothioate group, a deoxythymidine and 5′-phosphate group at the 5′-terminale end of the antisense strand.

16. A double-stranded ribonucleic acid molecule of claim 6, wherein said sense strand or said antisense strand comprise an overhang of 1-2 deoxythymidines.

17. A double-stranded ribonucleic acid molecule of claim 1, wherein said double-stranded ribonucleic acid molecule comprises the sequence pairs selected from the group consisting of SEQ ID NOs: 211/212, 213/214, 215/216, 217/218, 219/220, 223/224, 225/226, 229/230, 231/232, 233/234, 235/236 and 241/242.

18. A vector comprising a regulatory sequence operably linked to a nucleotide sequence that encodes at least the sense strand or an antisense strand of the double-stranded ribonucleic acid molecule as defined in any one of claim 1.

19. A cell, tissue or non-human organism comprising a double-stranded ribonucleic acid molecule as defined in claim 1.

20. A pharmaceutical composition comprising the double-stranded ribonucleic acid molecule as defined in claim 1 and a pharmaceutically acceptable carrier.
